(19) United States
(12) Patent Application Publication
(10) Pub. No.: US 2024/0132887 A1
(43) Pub. Date: Apr. 25, 2024

(54) PROTEIN ARGININE METHYLTRANSFERASE 9 INHIBITORS AND METHODS OF USE

(71) Applicants: City of Hope, Duarte, CA (US); Western University of Health Sciences, Pomona, CA (US)

(72) Inventors: Ling Li, Duarte, CA (US); Haojie Dong, Duarte, CA (US); Lei Zhang, Duarte, CA (US); Xin He, Duarte, CA (US); Yun Lyna Luo, Pomona, CA (US); Yi-Chun Lin, Pomona, CA (US)

(73) Assignees: City of Hope, Duarte, CA (US); Western University of Health Sciences, Pomona, CA (US)

(21) Appl. No.: 18/377,503

(22) Filed: Oct. 6, 2023

Related U.S. Application Data
(60) Provisional application No. 63/378,798, filed on Oct. 7, 2022.

(51) Int. Cl. C12N 15/113; C07K 16/40; C12N 9/22

(52) U.S. Cl. CPC C12N 15/113; C07K 16/40; C12N 9/22; C12N 2310/20

(57) ABSTRACT

Disclosed herein are, inter alia, inhibitors of protein arginine methyltransferase 9 and pharmaceutical compositions thereof, and methods comprising the use of protein arginine methyltransferase 9 inhibitors for the treatment of a protein arginine methyltransferase 9-modulated disease or disorder, such as a hematological cancer.

### CROSS-REFERENCE TO RELATED APPLICATIONS

This application claims the benefit and right of priority to U.S. Application No. 63/378,798 filed Oct. 7, 2022, the disclosure of which is incorporated by reference herein in its entirety and for all purposes.

### STATEMENT AS TO RIGHTS TO INVENTIONS MADE UNDER FEDERALLY SPONSORED RESEARCH AND DEVELOPMENT

This invention was made with government support under R01 CA248149 awarded by the National Institutes of Health. The government has certain rights in the invention.

### BACKGROUND

Cancer recurrence is a major clinical challenge in conventional chemotherapies, as residual tumor cells grow into new tumors. The overall outcomes for patients with acute myeloid leukemia (AML) following cytotoxic induction therapy including cytarabine (ara-C) and an anthracycline remain poor, with only about 40% of younger patients under the age of 60 and 10% of older patients over the age of 60 achieving long-term survival. Allogeneic hematopoietic stem cell transplantation (allo-HSCT), which supplies allogeneic T cells to attack residual disease, is the only cure for AML. However, allo-HSCT applicability is hampered by graft-versus-host disease (GvHD)-related toxicities, co-existing morbidities, and the shortage of donors. Treatment of AML with other immunotherapies, such as T cell-engaging antibodies, immune checkpoint inhibitors (ICIs) and CAR-T cells, remains challenging because of the lack of suitable target antigens and low expression of immune checkpoint proteins. New effective therapies are therefore urgently needed for the treatment of immune-cold cancers. The disclosure is directed to this, as well as other, important needs.

### SUMMARY

Provided herein, inter alia, are methods of treating a hematological cancer in a subject in need thereof. The disclosed methods comprise administering to the subject a therapeutically effective amount of a protein arginine methyltransferase 9 (PRMT9) inhibitor. In embodiments, the PRMT9 inhibitor is a short-hairpin RNA, a small interference RNA, a piwi-interacting RNA, a microRNA, a CRISPR Cas guide RNA, an antisense oligonucleotide, a small molecule compound, or an anti-PRMT9 antibody.

Provided herein are compounds of Formula (I), Formula (II), or Formula (III):

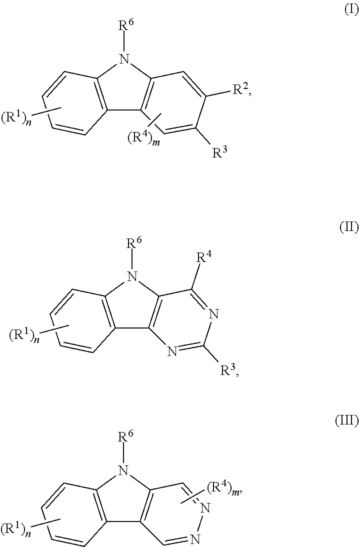

or a pharmaceutically acceptable salt thereof, wherein the substituents are as defined herein.

These and other embodiments are described in detail herein.

### BRIEF DESCRIPTION OF THE DRAWINGS

FIGS. 1A-1K illustrate protein arginine methyltransferase 9 (PRMT9) levels are elevated in a cancer stem cell-enriched acute myeloid leukemia (AML) subset. FIG. 1A: Heatmap showing all PRMT messenger ribonucleic acid (mRNA) levels in the most-deadly cancers in the United States (US), extracted from The Cancer Genome Atlas (TCGA) PanCancer Atlas Studies. AML (n=173), Lung adenocarcinoma (LUAD, n=510), Lung squamous cell carcinoma (LUSC, n=484), Colon Adenocarcinoma (COAD, n=438), Pancreatic adenocarcinoma (PAAD, n=177), Breast Cancer (BRCA, n=1082), Prostate adenocarcinoma (PRAD, n=493), Liver Hepatocellular Carcinoma (LIHC, n=366), Glioblastoma (GBM, n=160), Bladder Carcinoma (BLCA, n=407). Z-scores were calculated based on the average expression of each PRMT in different cancers. PRMT8 is not shown, as it is undetectable in most cancers. (https://www.cbioportal.org/). FIG. 1B: PRMT9 protein levels in AML lines relative to those seen in other cancer cell lines representing the most-deadly cancers in the US. Data was sourced from the DepMap portal (https://DepMap.org/portal/). AML (n=10), Lung Cancer (LC, n=75), Colon Adenocarcinoma (COAD, n=29), Pancreatic adenocarcinoma (PAAD, n=17), Breast Cancer (BRCA, n=29), Prostate adenocarcinoma (PRAD, n=5), Liver Hepatocellular Carcinoma (LIHC, n=12), Glioblastoma (GBM, n=11), Bladder Carcinoma (BLCA, n=9) and Acute Lymphoblastic Leukemia (ALL, n=8). P value was determined by two-sided Mann-Whitney test. FIG. 1C: Heatmap (left) showing all PRMT mRNA levels in normal hematopoietic from normal donors (n=7) or leukemia stem and progenitor subsets from AML patients (n=21) in GSE63270. Z-scores were calculated based on average expression of each PRMT in different subsets. Violin plots (right) show PRMT9 expression in AML leukemia stem cells (LSC) vs normal hematopoietic stem cells (HSCs) and in LSCs vs leukemia blasts. P value (LSC vs HSC) was determined by two-sided Mann-Whitney test. P value (LSC vs Blast) was determined by paired two-tailed t-test. FIG. 1D: Heatmap showing 50 representative genes from the MLL-AF9 (MA9) mouse leukemia LSC signature. The analysis is from Single-Cell RNA-seq of MA9 leukemic cells from bone marrow (BM) (the dataset is the same data shown in FIG. 1H “MA9 control group”). Heatmap was generated by Seurat package using the Z-score of each gene in either LSCs or blast cells. FIG. 1E: t-distributed stochastic neighbor embedding (t-SNE) plot showing LSC and blast clusters from (FIG. 1D). Shown are a representative LSC signature gene (Cbx5) and Prmt9 levels in both clusters. FIG. 1F: Bar graph showing average fold-change in all Prmt levels in an LSC versus a blast cluster based on scRNAseq (FIG. 1E). The most significantly upregulated gene is Prmt9. FIG. 1G: Flow cytometry-based intracellular staining of PRMT9 in CD34+CD38− and CD34+CD38+ populations in primary AML cells and in normal cells from donor peripheral blood stem cell (PBSCs). FIGS. 1H-1I: Western blot analysis of PRMT9 levels in AML CD34+ subsets versus normal PBSC counterparts from healthy donors (FIG. 1H). FIG. 11: PRMT9 levels of normal and AML samples (excluding the t[15;17] M3 subset, patient information in Table 1) were normalized to those seen in Molm13 cells in each western blot. P value (<0.0001) was determined by two-sided Mann-Whitney test. ****P<0.0001. FIG. 1J: Kaplan-Meier overall survival analysis of the in-house AML cohort (excluding the t[15;17]M3 subset, patient information in Table 2, n=94) after dichotomization for median PRMT9 mRNA levels normalized to β-actin. P value was determined by log-rank (Mantel-Cox) test. FIG. 1K: Kaplan-Meier overall survival analysis of an AML cohort (GSE12417, normal karyotype) after dichotomization for PRMT9 levels below (n=74) or above (89) 9.62 log 2-transformed intensity. The threshold is discovered by classifying 163 patients into 2 clusters (PRMT9-High: 74, PRMT9-Low: 89) via Partitioning Around Medoids (PAM). P value was determined by log-rank (Mantel-Cox) test.

FIGS. 2A-2R illustrate that PRMT9 levels are elevated in the cancer stem cell-enriched AML subset. FIGS. 2A-2H: Violin plot showing all the PRMTs mRNA levels in top deadly cancers in the U.S. extracted from TCGA PanCancer Atlas Studies. AML (n=173), Lung adenocarcinoma (LUAD, n=510), Lung squamous cell carcinoma (LUSC, n=484), Colon Adenocarcinoma (COAD, n=438), Pancreatic adenocarcinoma (PAAD, n=177), Breast Cancer (BRCA, n=1082), Prostate adenocarcinoma (PRAD, n=493), Liver Hepatocellular Carcinoma (LIHC, n=366), Glioblastoma (GBM, n=160), Bladder Carcinoma (BLCA, n=407). Each PRMT expression in AML was compared with each other cancers (only those with significant difference were indicated). The shaded violin plot indicates PRMTs (PRMT1-9, except PRMT8 which is omitted as it is undetectable in most of the cancers) level was significantly higher in AML than corresponded cancer type. (https://www.cbioportal.org/). P values (FIGS. 2A-2H) were determined by unpaired two-tailed t-tests. ****P<0.0001. FIGS. 2I-2N: Violin plot showing all the PRMTs protein levels in AML lines relative to those seen in other cancer lines representing the most-deadly cancers in the U.S. Cell lines number is indicated in the source data. AML (n=14), Lung Cancer (LC, n=77), Colon Adenocarcinoma (COAD, n=29), Pancreatic adenocarcinoma (PAAD, n=17), Breast Cancer (BRCA, n=29), Prostate adenocarcinoma (PRAD, n=5), Liver Hepatocellular Carcinoma (LIHC, n=12), Glioblastoma (GBM, n=11), Bladder Carcinoma (BLCA, n=9) and Acute Lymphoblastic Leukemia (ALL, n=8). Data was generated from the DepMap portal (https://DepMap.org/portal/). P values were determined by two-sided Mann-Whitney test. FIG. 2O: Relative PRMT9 expression in AML (n=10) and B-cell non-Hodgkin lymphoma (B-NHL) (n=8) lines relative to other available cancer lines (n=330). Data was sourced from the DepMap. P values were determined by two-sided Mann-Whitney test. FIG. 2P: Western blot analysis of PRMT9 expression in lines representing the most-deadly cancers. FIG. 2Q: Violin plot showing expression level of LSC signature genes in LSC (n=383) and Blast (n=1191) clusters based on MLL-AF9 cells with scRNAseq analyses. P value was determined by two-sided Mann-Whitney test. ****P<0.0001. FIG. 2R: Q-PCR analysis of PRMT9 levels in AML CD34+ cells from an in-house cohort of AML cases (n=94) and from PBSCs (n=19) from healthy donors. PRMT9 levels were normalized to β-actin mRNA levels. P value was determined by two-sided Mann-Whitney test. ****P<0.0001. Unless otherwise specified. ****p<0.0001, “ns” represents no significance.

FIGS. 3A-3R illustrate that PRMT9 levels are elevated in AML and dispensable for normal hematopoiesis. FIG. 3A: Relative PRMT9 mRNA levels reported in the BEAT AML dataset (http://vizome.org/aml/) of mononuclear cells (MNCs) from AML cases and healthy donors. P value was determined by two-sided Mann-Whitney test. FIG. 3B: PRMT9 expression levels in human primary AML cases with different cytogenetic karyotypes in BEAT AML cohort. NK: n=107, t (8;21): n=11, 11q23: n=15, t (15;17): n=15, inv (16): n=25, Complex: n=32. FIGS. 3C-3D: Relative PRMT9 expression in BEAT AML datasets of FLT3-ITD- (FIG. 3C) and NPM1- (FIG. 3D) mutant AML subsets relative to their WT counterparts. FLT3wt n=346, FLT3-ITD: n=105; NPM1wt: n=340, NPM1mut: n=108. P value was determined by two-sided Mann-Whitney test. “ns” represents no significance. FIG. 3E: Western blot analysis of PRMT9 expression levels in representative DLBCL lines relative to peripheral blood mononuclear cells (PBMC) control. FIG. 3F: Kaplan-Meier overall survival analysis of an AML cohort (TARGET-AML) after dichotomization for PRMT9 levels below (n=99) or above (n=57) 3.55 log 2-transformed intensity. The threshold is discovered by classifying 156 patients into 2 clusters via Partitioning Around Medoids (PAM). P value was determined by log-rank (Mantel-Cox) test. FIG. 3G: Pearson correlation of CREB1, STAT3, STAT5A, and GATA2 with PRMT9 levels across TCGA AML cohorts. Data were sourced from GEPIA (http://gepia.cancer-pku.cn/). FIG. 3H: Pearson correlation of CREB1 with PRMT9 levels across TCGA DLBCL cohorts. Data were sourced from GEPIA (http://gepia.cancer-pku.cn/). FIG. 3I: Violin plot showing expression level of Creb1 in LSC (n=383) and Blast (n=1444) based on MLL-AF9 cells with scRNAseq analyses. P value was determined by two-sided Mann-Whitney test. ****P<0.0001. FIG. 3J Western blot analysis of PRMT9 levels after CREB1 Knockdown (KD) via shCREB1. FIG. 3K: K562 anti-CREB1 and anti-H3K27Ac ChIP-SEQ analysis showing CREB1 and H3K27Ac enrichment in the same PRMT9 promoter region. FIG. 3L: Upper panel, diagram showing predicted CREB1 binding sites one TSS site in the PRMT9 promoter (Upstream of Exon1). Higher bars indicate Q-PCR-identified regions representing CBS (CREB1 Binding Site) and Ctrl sites after ChIP assay. Lower panel, ChIP-qPCR analysis of enrichment of CREB1 and H3K27Ac at the CBS site of the PRMT9 promoter and at a distal control site in Molm13 and normal PBSC CD34+ cells. IgG served as a negative control. Data are presented as mean±standard deviation (SD). P values were determined by two-tailed t-test. ****P<0.05, **p<0.01, ***p<0.001, ****p<0.0001. FIG. 3M: Hierarchical differentiation tree of human PRMT9 mRNA levels in human hematopoietic stem, progenitor and mature lineage cells by HemaExplorer. Human HSCs are from GSE17054; GMP and MEP cells are from GSE11864; monocytes are from GSE11864 and E-MEXP-1242. Data has been batch corrected/normalized. Data was sourced from Bloodspot. FIG. 3N: Hierarchical differentiation tree of mouse Prmt9 mRNA levels in BM subsets from normal mice including hematopoietic stem, progenitor and mature lineage cells. Prmt9 levels were normalized to Myeloid (Gr1+Mac1+) Prmt9 mRNA levels and log 2-transformed. FIG. 3O: Schema of the Prmt9 targeting strategy in conditional knockout (KO) mice. Upper panel shows location of genotyping primers of the floxed locus (primers 1 and 2) versus Prmt9 KO (primers 1 and 3). Lower left panel shows representative genotyping results of Prmt9wt, floxed and KO alleles. Lower right panel, assessment of Prmt9 protein levels based on western blotting of BM cells from Prmt9 WT and KO mice after pIpC induction. Total β-actin served as a reference control. FIGS. 3P-3Q: Frequency of hematopoietic progenitors (FIG. 3P) and mature lineage cells (FIG. 3Q) in mouse BM at 16 weeks after pIpC administration. Data are presented as mean±standard error of the mean (SEM). P values were determined by unpaired two-tailed Student's t test. (R) Competitive transplantation assay of CD45.2 Prmt9 conditional KO BM cells with normal CD45.1 BM cells in CD45.1 recipient mice. The percentage of CD45.2 peripheral blood leukocytes was checked every four weeks after pIpC administration. Results represent the mean±SEM. P values were determined by two-way Analysis of variance (ANOVA). Unless otherwise specified. *p<0.05, **p<0.01, ***p<0.001, ****p<0.0001, “ns” represents no significance.

FIGS. 4A-4R illustrate PRMT9 ablation that impairs cancer stem/progenitor cell growth and survival. FIG. 4A: PRMT9 protein expression in cKit⁺ BM cells from MLL-AF9 (MA9) knock-in mice and Cbfb-MYH11/Mpl (CMM) relative to normal cKit⁺ BM cells (n=2). FIGS. 4B-4C: CFC assay of MA9/Prmt9^{f/f}/MxCre and MA9-ITD Prmt9^{f/f}MxCre cells (500 cells/well) after Prmt9 KO. Shown in (FIG. 4B) is colony number determined 7 days after Prmt9 deletion induced by exposure of IFNα as previously described by Wang et al. FASEB J 32, 1537-1549, doi:10.1096/fj.201700740R (2018). Data represent mean±SD. P value (FIG. 4B) was determined by two-sided Mann-Whitney test. P=0.0079. **P<0.01. Prmt9 KO efficiency in analysis shown in (FIG. 4C), as indicated by western blot assay. FIGS. 4D-4E: MA9-ITD-lucifase cells (0.5×10⁶ cells per mouse) were injected into sub-lethally irradiated CD45.1 B6 mice. Following engraftment confirmation, mice were treated 7 days with pIpC and assessed for engraftment by in vivo bioluminescence imaging (FIG. 4E). (FIG. 4D) showing quantitative results from bioimaging; Data represent mean±SEM, P values were determined by two-way ANOVA. ****P 0.0001. FIGS. 4F-4G: Parallel analysis of MA9-ITD-luciferase cells engraftment assay. When one control mouse reached the endpoint, spleen size (FIG. 4F) of Prmt9 KO and control mice was checked, and engraftment was evaluated by flow cytometry based on CD45.2+% (MA9-ITD) cells in BM (FIG. 4G). Data represent mean±SEM. P value (FIG. 4G) was determined by two-sided Mann-Whitney test. FIG. 4H: Kaplan-Meier overall survival analysis of MA9-ITD-luciferase cells in mice engraftment (n=5/group) assay after Prmt9 KD. P value was determined by log-rank (Mantel-Cox) test. FIGS. 4I-4K: Molm13 cells were transduced with Mock or PRMT9 Wt or PRMT9 Mut (LDIG to AAAA) expression vectors resistant to PRMT9 shRNA-1 and then PRMT9 expression was detected by western blot analysis after transduction of cells with shPRMT9-1 lentivirus (FIG. 4I) (n=2). Cell viability (FIG. 4J) after PRMT9 KD, as evaluated by an 3-(4,5-dimethylthiazol-2-yl)-5-(3-carboxymethoxyphenyl)-2-(4-sulfophenyl)-2H-tetrazolium (MTS) assay, and apoptosis (FIG. 4K), based on flow cytometry after Annexin V staining. Data (FIG. 4J, FIG. 4K) represent mean±SD; P value from each comparison was determined by two-sided Mann-Whitney test. P=0.0022. **P<0.01. FIGS. 4L-40: Primary AML CD34+ (n=11) or normal PBSC CD34+ (n=3) control cells were transduced with shPRMT9-1 lentivirus and RFP+ cells sorted by flow cytometry. Cell viability (FIG. 4L), as evaluated by an MTS assay, and apoptosis (FIGS. 4M-4N), based on flow cytometry after Annexin V staining. PRMT9 mRNA levels were detected by Q-PCR analysis FIG. 4O). Data (FIG. 4L, FIG. 4N, FIG. 4O) represent mean±SD; P values (FIG. 4L, 4N) were determined by two-way ANOVA. ****P<0.0001. FIGS. 4P-4R: Molm13 cells were transduced with a DOX-inducible shPRMT9 construct or control vector and transplanted into NSG mice (control, n=8; shPRMT9, n=7). PRMT9 knock down efficiency was evaluated by western blot assay after DOX induction in cultured cells (FIG. 4P). Once engraftment was confirmed (>1% in PB), mice were treated with DOX to induce PRMT9 or control KD. After one mouse reached the endpoint, engraftment was evaluated based on percentage of hCD45 cells in BM (FIG. 4Q), Data represent mean±SEM, and P value was by two-sided Mann-Whitney test. In parallel analysis, mouse survival was analyzed over time after DOX treatment (FIG. 4R); P value was determined by log-rank (Mantel-Cox) test.

FIGS. 5A-5M illustrate PRMT9 ablation that impairs cancer stem/progenitor cell growth and survival. FIG. 5A: Representative image of colonies after Prmt9 KO in MA9-ITD cells. FIG. 5B: After transduced with Dox-inducible shCtrl and shPrmt9 lentivirus, cells were briefly induced with Dox overnight. Cells with equal intensity of RFP+ cells were sorted by flow cytometry. Representative flow plots show the sorting gate for MA9 cells. FIGS. 5C-5D: Prmt9 KD efficiency in Prmt9 inducible KD MA9-ITD, MA9 and CMM cells, were evaluated by western blot assay after DOX induction in cultured cells (FIG. 5C). CFC assay of indicated cells (500 cells/well) performed 7 days after DOX-induced Prmt9 KD (FIG. 5D). Results represent the mean±SD from 3 independent experiments. FIGS. 5E-5G: In vitro limiting dilution assay (LDA) assay to evaluate the LSC frequency in Prmt9 inducible KD engineered MA9 (FIG. 5E), MA9/FLT3-ITD+ (FIG. 5F) and CMM (FIG. 5G) AML cells. LSC frequency and p value were calculated using ELDA software. FIGS. 5H-5K: AML lines Molm13, MV4-11, HL-60, NB4, MA9.6-ITD, THP1, and B-NHL lines OCI-Ly3 and Rec were transduced with shPRMT9 lentivirus to KD endogenous PRMT9. Cell viability (FIG. 5J) was then evaluated by an MTS assay. Apoptosis (FIG. 5K) was assayed by flow cytometry after Annexin V staining. PRMT9 expression levels were detected by western blot analysis (FIG. 5H, FIG. 5I). Results (FIG. 5J, FIG. 5K) represent the mean±SD from at least 3 (J, n=4; K, n=3) independent experiments. FIG. 5L: Primary AML CD34+ cells were transduced with shPRMT9-1 lentivirus and analyzed for apoptosis as above. FIG. 5M: Schema of PRMT9 inducible KD Molm13 engraftment assay in NSG mice. Briefly, Molm13 cells were transduced with a DOX-inducible shPRMT9 construct or control vector and transplanted into NSG mice. Once engraftment was confirmed (>1% in PB), mice were treated with DOX to induce PRMT9 or control KD. After one mouse reached the endpoint, engraftment was evaluated based on percentage of hCD45 cells in BM. In parallel analysis, mouse survival was analyzed over time after DOX treatment.

FIGS. 6A-6W illustrate that PRMT9-mediated methylation of downstream substrates promotes cancer cell maintenance. FIG. 6A: Stable Isotope Labeling by/with Amino acids in Cell culture (SILAC)-based work-flow used for global detection of PRMT9 substrates. Control or inducible PRMT9 KD Molm13 cells were labeled with heavy or light L-arginine and L-Lysine and treated with Dox 3 days to induce PRMT9 KD. After protein quantification and digestion, 5% of mixed peptides were subjected to global proteomics and remaining peptides were enriched by antibodies to pan-Symmetric Di-Methyl Arginine (SDMA) and pan-Mono-Methyl Aginine (MMA) proteins. R-methylated peptides were analyzed by quantitative Liquid Chromatograph Triple Quadrupole Mass Spectrometry (LC-MS/MS) and normalized to total protein abundance. FIG. 6B: Histogram indicating the number of sites carrying Di-Methyl Arginine (DMA) or Mono-Methyl Aginine (MMA) within down- and up-regulated methyl sites differentially responding to PRMT9 KD. FIG. 6C: Interaction analysis of proteins carrying PRMT9-regulated R-methyl peptides, as visualized by Cytoscape 3.0. FIG. 6D: Bar graph showing percentage of target hits among all PRMT9-methylated proteins according to Gene Ontology (GO) enrichment categories. FIG. 6E: Polysome profiling of RNAs isolated from control and Dox-induced PRMT9 KD Molm13 cells treated 3 days with Dox. Positions of free ribosomal subunits (40S, 60S), monosomes (80S), and polysomes are indicated. Shown is representative trace of 1 of 3 biological replicates. FIG. 6F: Primary AML CD34+ (n=3) or normal PBSC CD34+ (n=3) cells were transduced with shCtrl or shPRMT9 lentivirus, sorted RFP+ cells, and then analyzed for protein synthesis using an O-propargyl-puromycin (OP-Puro)-based flow cytometry. Representative results were shown (left). Bar chart (right) summarizes the results in Ctrl vs. PRMT9-KD group. Data are presented as mean±SD. FIG. 6G: Western blot analysis verifies the downregulation of representative proteins from SILAC-based global proteomics analysis of Ctrl and PRMT9-KD Molm13 cells (n=2). FIG. 6H: Q-PCR analysis of c-Myc mRNA levels in RNAs extracted from indicated fractions in the ribosome profiling assay. FIG. 6I: Bar graph showing top down-regulated translation factors carrying indicated methylated R (highest confidence) site after PRMT9 KD based on the SILAC-based methyl peptide quantification assay. FIG. 6J: Schematic model showing methylated arginine at the Polyadenylate-binding protein 1 (PABPC1) C-terminal. R481, R493 and R506 methylation sites were identified here; R455/R460 methylation is reportedly catalyzed by PRMT4 (CARM1)¹¹⁹. FIG. 6K: In vitro methylation assay of Glutathione S-transferase (GST)-tagged PABPC1-CT (aa V436-V636) in the presence of PRMT9 protein and S-Adenosyl Methionine (SAM). Methylation was analyzed by western blotting using an anti-pan-MMA antibody (n=2). FIG. 6L: In vitro methylation assay of synthesized unmodified PABPC1 peptides (aaG491-T507) in the presence of PRMT9 protein and SAM. Methylation was analyzed by western blotting using anti-SDMA, MMA and Asymmetric Di-Methyl Arginine (ADMA) antibodies (n=3). FIGS. 6M-60: Molm13 cells were transduced with Mock or PABPC1 (WT, R493K, or 3RK) expression vectors resistant to PABPC1 shRNA, and PABPC1 expression was assessed by western blot after transduction of cells with shPABPC1 lentivirus (FIG. 6M), n=2. Protein synthesis, based on an OP-Puro-based flow cytometry assay (FIG. 6N). Cell viability (FIG. 6O), as assessed by an MTS assay. Data (FIG. 6O) are represented as mean±SD; P value was determined by two-sided Mann-Whitney test. P=0.0079, **P<0.01. “ns”: no significance. FIG. 6P: Schematic model of translational activities related to PABPC1 (stared positions): PABPC1 self-interaction; binding to the mRNA poly(A) tail; interaction with eRF3 by the PABPC1 C-terminus; interaction with eIF4G by the PABPC1 N-terminus. FIG. 6Q: PABPC1 self-interaction assay. HEK293T cells were co-transfected 2 days with human influenza hemagglutinin (HA)-tagged PABPC1 plus Flag-tagged PABPC1 (WT or R493K). Cell lysates were then subjected to pull-down assays using anti-Flag beads and interactors detected by immunoblot with anti-HA antibody. FIG. 6R: Poly(A) agarose pull-down assay. HEK293T cells were transfected with Flag-tagged PABPC1 (WT or R493K) and subjected to pull-down assays using poly(A) agarose beads. Interactors were detected by immunoblot with an anti-Flag antibody (n=2). FIG. 6S: PABPC1 and eRF3 interaction assay. HEK293T cells were co-transfected 2 days with Myc-tagged eRF3 and Flag-tagged PABPC1 (WT or R493K). Cell lysates were subjected to pull-down assays using anti-Flag beads, and interactors detected by immunoblotting with anti-Myc antibody. FIG. 6T: PABPC1 R493me antibody specificity assay. Indicated amounts of unmodified, SDMA-R493, or ADMA-R493 PABPC1 peptides (aaG491-T507) were synthesized and spotted onto nitrocellulose (NC) membranes for a dot blot assay. PABPC1 peptides were detected by anti-R493me-specific or control antibodies. FIG. 6U: Western blot analysis shows PABPC1 R493 methylation and PRMT9 levels after PRMT9 KD in Molm13 cells (n=2). FIGS. 6V-6W: PRMT9 and PABPC1 R493 methylation levels in AML CD34⁺ subsets versus their blast (CD34⁻CD33⁺) subset from cases (n=7) (FIG. 6V). P values (FIG. 6V) were determined by paired t-test. Pearson correlation of PABPC1 R493 methylation levels with PRMT9 levels in AML CD34+ subsets (FIG. 6W). P value was determined by linear regression analysis. Western blot analysis was shown in FIG. 9C.

FIGS. 7A-7S illustrate that PRMT9-mediated methylation of downstream substrates promotes cancer cell maintenance. FIG. 7A: iceLogo motif analysis showing a non-specific pattern of R-methyl peptides regulated by PRMT9 (foreground). The complete dataset of R-methyl peptides identified was used as background. FIGS. 7B-7D: Mouse normal cKit+BM and MA9-ITD cells were transduced with shCtrl or shPrmt9 lentivirus and sorted RFP+ cells. B-NHL (Rec and OCI-Ly3) and AML (Molm13, MA9.6ITD, MV4-11, THP1, NB4 and HL60) lines were transduced with shCtrl or shPRMT9 lentivirus and sorted RFP+ cells. Protein synthesis in mouse BM (FIG. 7B), and in B-NHL (FIG. 7C) and AML (FIG. 7D) cells after PRMT9 KD, as assessed by an OP-Puro-based flow cytometry assay. Bar chart (right panel of FIGS. 7B-7D) shows the statistics of results in Ctrl vs. PRMT9-KD group. Data are presented as mean±SD from 3 independent experiments. FIG. 7E: Q-PCR analysis of c-Myc, Runt-related transcription factor 1 (RUNX1), CCAAT/enhancer-binding protein alpha (CEBPA) and SAM domain- and HD domain-containing protein 1 (SAMHD1) mRNA levels after PRMT9 KD in Molm13 cells. Data are presented as mean±SD from 3 independent experiments. FIG. 7F: Q-PCR analysis of SAMHD1 mRNA levels in RNAs extracted from indicated fractions in a ribosome profiling assay. FIGS. 7G-7H: Representative mass spectra of R493, R506 and R481 methylation in SILAC-based methyl2402 peptide quantitative LC-MS/MS. FIG. 7I: HEK293T cells were transfected 2 days with Flag-tagged PABPC1-CT (Wt, R481K, R493K, R506K, or 3RK). Cell lysates were then subjected to immunoprecipitation using anti-Flag beads and immunoblotted using an anti-pan-MMA antibody (n=2). FIG. 7J: Purification of Myc-tagged PRMT9 by the Myc-Trap technique (ChromoTek). Purity of purified Myc-PRMT9 as assessed by Coomassie blue staining (left panel) and immunoblotting with antibodies against PRMT9, PRMT1, PRMT4 (CARM1) or PRMT5. FIG. 7K: In vitro methylation assay of GST-tagged PABPC1-CT in the presence of PRMT1 protein and SAM. Methylation was analyzed by western blotting with an anti-MMA antibody (n=2). FIG. 7L: In vitro methylation assay of GST-tagged PABPC1-CT in the presence of PRMT5/MEP50 mixed protein and SAM. Methylation was analyzed by western blotting with an anti-MMA antibody (left) (n=2). H4 catalyzed by PRMT5 and detected by H4R3me2S antibody was used as the positive control (right). FIG. 7M: Ex-vivo tritium methylation assay using PABPC1-WT and corresponding PABPC1-3RK (R481K/R493K/R506K) immunoprecipitated from 293T cells. PABPC1-WT or -3RK was incubated with PRMT9 protein and S-adenosyl-1-[methyl-3H]methionine at 30° C. for 1 h, separated by SDS-PAGE and proceeded to fluorography (n=2). FIG. 7N: PABPC1 and eIF4G interaction assay. HEK293T cells were co-transfected 2 days with HA2420 tagged eIF4G plus Flag-tagged PABPC1 (WT or R493K). Cell lysates were then subjected to pull2421 down analysis using anti-Flag beads and immunoblotted with an anti-HA antibody. FIG. 7O: HEK293T cells were transfected 2 days with Flag-tagged PABPC1-CT (WT, R481K, R493K, or R506K). Cell lysates were then subjected to pull-down analysis using anti-Flag beads and immunoblotted using anti-R493me antibody (n=2). FIG. 7P: PABPC1 R455/R460 methylation, as detected by an anti-R455/R460me antibody after PRMT9 KD in Molm13 cells (n=2). FIG. 7Q: PABPC1 R493 methylation, as detected by an anti-R493me antibody after PRMT9 overexpression in Molm13 cells (n=2). FIG. 7R: PABPC1 methylation, as detected by anti-R493me- and anti-R455/R460me-specific antibodies in Molm13 cells treated with the CARM1 inhibitor EPZ056544 at 5 micromolar (M) for 48 h (n=2). FIG. 7S: PABPC1 R493 methylation, as detected by anti-R493me-specific antibody in Molm13 cells treated with PRMT1i (MS023, 5 μM, 48 h), PRMT5i (EPZ015666, 5 μM, 96 h) or PRMT7i (SGC3027, 5 μM, 48 h). H4R3me2A, HSP70me (IP HSP70 and detected with anti-MMA antibody), pan-SDMA (detected by SYM10 antibody) were served as positive controls for MS023, SGC3027, and EPZ015666 treatment respectively (n=2).

FIGS. 8A-8D illustrate that CARM1 does not catalyze PABPC1 methylation at R493. FIG. 8A: Western blot analysis of PABPC1 R455/460, and R493 methylation in Molm13 cells after CARM1 KD (n=2). FIGS. 8B-8C: Molm13 cells were transduced with Mock or PABPC1 (WT or R455/460K) expression vectors resistant to PABPC1 shRNA, and PABPC1 expression was assessed by western blot after transduction of cells with shPABPC1 lentivirus (FIG. 8B). Cell viability was assessed by an MTS assay (FIG. 8C). Data are presented as mean±SD. P values (P=0.0079) each comparison was determined by two-sided Mann-Whitney test. **P<0.01. FIG. 8D: Protein synthesis of PABPC1-WT or R455/460K mutant Molm13 cells were assessed by an OP-Puro-based flow cytometry assay. Bar chart (right) shows the statistics of results in PABPC1-WT vs. R455/460K group. Data are presented as mean±SD. P value was determined by two sided Mann-Whitney test. “ns”: no significance.

FIGS. 9A-9E illustrate that PRMT9 expression is positively correlated with PABPC1 R493 methylation in LSCs and blasts. FIG. 9A: Gating strategy of representative primary human AML sample excluding T cells and B cells, and sorting leukemia stem/progenitor-enriched CD34+ subset (LSC) and CD34−CD33+ leukemia blasts (Blasts) based on CD34 and CD33 staining. After sorting, the purity of LSC and Blast cells were checked again by flow cytometry. FIG. 9B: Sorting strategy of other six primary AML samples for the LSC and Blasts. FIG. 9C: Western blot analysis of PABPC1 R493me, PRMT9 in LSC and Blasts from 7 primary AML samples. FIG. 9D: Representative sorting strategy of cKit+ and cKit− MA9 leukemia cells (GFP+) or CMM leukemia cells (GFP+) harvested from BM of AML developed mice. FIG. 9E: Western blot analysis of Pabpc1 R493me, Prmt9 in cKit+ and cKit− MA9 or CMM leukemia cells from AML developed mice (n=3).

FIGS. 10A-10P illustrate the identification of a PRMT9 inhibitor via an R493 methylation assay. FIG. 10A: Pyramid flowchart of pipeline to identify PRMT9 inhibitors from the National Cancer Institute (NCI) Developmental Therapeutics Program (DTP, 260,000 compounds) and ZINC libraries (700,000 compounds). FIG. 10B: Docking pose of the top 30 hits within the PRMT9 protein catalytic pocket. FIG. 10C: Effects of the top 20 compounds on Molm13 cell viability. Cells were treated 4 days with 1 and 5 μM of indicated compounds, and viability was assessed by MTS assay. FIG. 10D: Catalytic activity screen of 9 selected compounds using an in-house R493 methylation assay. PRMT9 protein was pretreated 1 h with indicated compounds using in vitro methylation buffer and then incubated with synthesized unmodified PABPC1 peptides and SAM. Catalytic activity was assayed in vitro based on a dot blot assay of binding of an anti-R493-specific antibody. #1 was named as “LD2” for further analysis. FIG. 10E: 3D docking model of compound LD2 in the PRMT9 catalytic pocket. Left panel shows LD2 inserted deeply into the pocket. Right shows key LD2 binding sites on PRMT9 protein. FIG. 10F: Carr-Purcell-Meiboom-Gill (CPMG) Nuclear Magnetic Resonance (NMR) spectrum for 40 μM LD2, LD2 in the presence of 0.67 μM PRMT9, 1 μM PRMT9 and 2 μM PRMT9. FIG. 10G: Saturation Transfer Differences (STD) NMR spectrum. FIG. 10GA: Reference and saturated spectra. FIG. 10 GB: STD spectrum showing difference between reference and saturated spectra. The peak with (*) in (FIG. 10GA) is from an impurity. FIGS. 10H-10I: Thermal shift assay (FIG. 10H) of Wt and Mut PRMT9 from cellular thermal shift assay (CETSA) analysis of Molm13 cells treated with 2.5 μM LD2. Catalytic activity inhibition curves are calculated based on intensity of blots normalized to intensity at 37° C. (n=2). A comparison was made between LD2 treated PRMT9-wt vs LD2 treated PRMT9-Mut. Data (FIG. 10I) are represented as mean±SD; P values (FIG. 10I) were determined by two-way ANOVA. FIG. 10J: Half maximal inhibitory concentration (IC-50) analysis of LD2 in normal PBSC 34+ cells and in various AML lines. Cells were treated 4 days with different LD2 concentrations, and cell viability was assessed by MTS assay. IC-50 values for PBSCs were >30 μM, and for AML lines, 2-7 μM. FIG. 10K: Protein synthesis in Molm13, MA9.6ITD and THP1 cells after treatment 2 days with Dimethyl sulfoxide (DMSO) vehicle (VEH) or 2.5 μM LD2, based on an OP-Puro-based flow cytometry assay. Bar chart (right) summarizes the results in VEH vs. LD2 group (n=3). Data are presented as mean±SD. FIG. 10L: PABPC1 R493 methylation, as detected by an anti-R493me antibody in lysates of Molm13 cells that had been treated 2 days with VEH (DMSO) or 2.5 μM LD2 (n=3). FIG. 10M: Mass-cytometry (CyTOF) analysis of Mono-Nuclear Cells (MNCs) from AML01 and AML03 samples after 4 days of treatment with vehicle or LD2 (2.5 μM) under physiological cytokine conditions (200 picograms/milliliters (pg/mL) Granulocyte-macrophage colony-stimulating factor (GM-CSF), 1 ng/mL Granulocyte colony-stimulating factor (G-CSF), 200 pg/mL stem cell factor (SCF), 1 ng/mL Interleukin 6 (IL-6), 200 pg/ml Macrophage inflammatory protein (MIP)-1α, and 50 pg/mL Leukemia inhibitory factor (LIF)). Spanning-tree Progression Analysis of Density-normalized Events (SPADE) clustering was performed to identify cell groups corresponding to immunophenotypic subsets across normal hematopoiesis lineages. The frequency of CD3⁺ T cells and CD34⁺CD45^{dim} AML blast cells were noted. FIG. 10N: Flow plots showing T cell (CD3⁺) and AML blast (CD34⁺CD45^{dim}) populations in the AML01 sample, before and after T cell depletion. FIG. 10O: T cell-depleted or bulk AML MNCs (n=3) were treated for 4 days with vehicle or LD2 (2.5 μM). The relative number of alive CD34⁺CD45^{dim} AML blast cells was assessed by flow cytometry. Data are presented as mean±SD from 3 independent experiments. FIG. OP: Bar graphs showing frequency of PRMT9 high (n=43) versus low (n=67) primary patient AML samples displaying the Cytotoxic T Lymphocyte (CTL) score high versus low signatures in large AML cohorts: GSE12417. P value was calculated using two-sided Fisher exact chi-squared test.

FIGS. 11A-11N illustrate identification of a PRMT9 inhibitor via an R493 methylation assay. FIG. 11A: Effects of top 212 hits (142 from the top 300 hits in DTP library and 70 from the top 100 hits in ZINC library) on Molm13 cell viability. Cells were treated for 4 days with VEH (DMSO) or 1 or 5 μM compounds, and relative cell viability was assessed by MTS assays. 101-200% in the heat bar indicates increased cell viability, while 0-99% indicates decreased variability. For each compound, left number represents its NSC or ZINC library number. Upper and lower dots represent relative cell viability after treatment with 1 μM and 5 μM, respectively, of the indicated compound. Circle diameter represents a p value of 1 or 5 μM compound compared with DMSO. Experiments were repeated in triplicate; P values were derived from t tests comparing DMSO with compound-treated groups. FIG. 11B: In-house R493 methylation assay. Synthesized unmodified PABPC1 peptides (aaG491-T507) were incubated 4 h with PRMT9 and SAM at 30° C. Catalytic activity was assessed by dot blot with anti-R493 specific antibody. PRMT9 or unmodified PABPC1 peptide alone served as negative controls, and synthesized R493me PABPC1 peptide as positive. FIG. 11C: Catalytic activity screen of top 20 compounds based on Molm13 viability assay with an in-house R493 methylation assay. PRMT9 protein was pretreated 1 h with indicated compounds (10 μM) and then incubated with synthesized unmodified PABPC1 peptides and SAM. Catalytic activity was assessed by dot blot with an anti-R493me specific antibody. FIGS. 11D-11E: Catalytic activity of NSC641396, NSC661221 and NSC645330 based on an in-house R493 methylation assay. PRMT9 protein was pretreated 1 h on ice with different concentrations of each compound and then incubated with synthesized unmodified PABPC1 peptides and SAM. Catalytic activity was assessed by dot blot with an anti-R493 antibody (FIG. 11D). Catalytic activity inhibition curves were calculated based on intensity of dots and normalized to DMSO controls (FIG. 11E). Data (FIG. 11E) are presented as mean±SD (n=3). FIGS. 11F-11G: NSC641396, NSC661221 and NSC645330 share a carbazole ring scaffold that aligns perfectly with their biphenylmethylamine moiety. A Tanimoto-based 2-dimensional similarity search was conducted after removing the quinone ring and introducing heteroatoms at different locations of the carbazole moiety (FIG. 11G). Note that LD2 is among the candidates. 3D docking model of NSC641396 in the PRMT9 catalytic pocket (FIG. 11F). FIG. 11H: IC-50 analysis of LD2 in B-NHL lines. Cells were treated for 4 days with different LD2 concentrations, and viability was assessed by an MTS assay. IC-50 values of B-NHL lines were 2.5-5 μM. Data (FIG. 11E) are presented as mean±SD (n=6). FIG. 11I: PRMT1 substrate (H4R3me2A, FLT3 R972/R973me), CARM1 substrate (PABPC1 R455/R460me), PRMT5 substrate (H3R8me2S), PRMT7 substrate (HDP70me) were assessed using corresponding methylation antibodies in Molm13 cells treated 2 days with DMSO or 2.5 μM LD2 (n=2). FIG. 11J: Ctrl and PRMT9-KD Molm13 cells were treated for 4 days with LD2 (2.5 μM), and cell viability was assessed using an MTS assay. Data (FIG. 11E) are presented as mean±SD from 4 independent experiments. FIG. 11K: CyTOF analysis of MNCs from the AML02 sample after treatment for 4 days with vehicle or LD2 (2.5 μM). SPADE clustering was performed to identify cell groups corresponding to immunophenotypic subsets across normal hematopoiesis lineages. The frequency of CD3+ T cells and CD34+CD45dim AML blast cells were noted. Color bar shows CD34 expression intensity. FIG. 11L: Relative leukemia (CD34+CD45dim) and T cell frequencies before and after LD2 treatment based on CyTOF analysis from 3 AML samples. FIG. 11M: Enriched T cells were treated 4 days with DMSO or LD2 (2.5 μM) for 4 days, and cell viability was assessed by an MTS assay. Data are presented as mean±SD from 3 independent experiments. FIG. 11N: Bar graphs showing frequency of PRMT9 high versus low primary patient AML samples displaying the Cytotoxic T Lymphocyte (CTL) score (defined as the average expression of CD8A, CD8B, GZMA, GZMB and PRF1) high versus low signatures in AML cohort SE14468. P value was calculated using two-sided Fisher exact chi-squared test.

FIGS. 12A-12B illustrate that LD2 at a low concentration specifically inhibits PRMT9 but not PRMT5. FIGS. 12A-12B illustrate dose-dependent inhibition of cellular PABPC1 R493 (FIG. 12A, PRMT9 substrate) and SmB/B′ (FIG. 12B, PRMT5 substrate) methylation level after 2 days of treatment with LD2 in Molm13 cells. Cells were treated with a dose titration of 0.5-40 μM LD2 for 48 h. The inhibition activity of LD2 to PRMT9 (right curve) was evaluated by calculating the PABPC1 R493 methylation signal normalizing to the PABPC1 total level (FIG. 12A). The inhibition activity of LD2 to PRMT5 (right curve) was evaluated by calculating the SmB′B′ methylation signal normalizing to the β-actin total level.

FIGS. 13A-13B illustrate Vina docking of LD2 with PRMT9, PRMT5, CARM1 and PRMT7. FIG. 13A: Vina docking of LD2 with PRMT9, PRMT5, CARM1 or PRMT7 was performed, and the average docking scores (kcal/mol, with standard deviation) were shown. FIG. 13B: Molecular dynamics simulation analyses of LD2 with PRMT9 or PRMT5. Smaller root-mean-square-fluctuation (RMSF) of ligand (LD2) in the binding pocket of PRMT9 compared with PRMT5 was shown.

FIG. 14 illustrates gating strategy of primary AML samples for ex-vivo treatment. After thawing the primary AML samples, the viable cells were enriched by removing dead cells (ensuring >95% live cells) and then treated MNCs with vehicle (0.1% DMSO), LD2 (2.5 μM). The gating strategy for the hematopoietic subsets in the three AML samples was shown including T cells (CD3+), B cells (CD19+/CD20+), and DCs (HLA-DR+CD34−CD33−CD3−CD19−CD20−CD14−CD56−), and leukemic cells (CD34+CD33+).

FIG. 15 illustrates apoptosis of primary AML cells after ex-vivo treatment. After 4 days treatment of vehicle (VEH) or LD2, survival of leukemic (CD34+CD33+) and immune subsets, including T cells (CD3+), B cells (CD19+/CD20+), and DCs (HLA-DR+CD34−CD33−CD3−CD19−CD20−CD14−CD56−), were accessed using Annexin-V staining. Histogram plots showed the percentage of apoptosis cells in DAPI negative cells in leukemic cells, T cells, B cells, and DCs gating as in FIG. 14.

FIG. 16 illustrates CyTOF analysis of PBMCs from a healthy donor. SPADE tree view analysis of a normal PBMC sample from one healthy donor. SPADE clustering was performed on all samples (normal and AML) simultaneously to generate the same tree view structure for all samples after vehicle or LD2 treatment. All cell events from each sample were mapped to the common tree structure. Each node of the SPADE tree is colored as a median expression of indicated phenotypic markers from low to high. Node size positively correlates with the number of cells mapping to the node. Immunophenotypic grouping of nodes was performed manually based on median marker expression level of each node. Shown is expression of all markers used for phenotyping (such as CD3, CD19, CD34).

FIG. 17 illustrates activation of T cells in primary AML samples after ex-vivo treatment. Intracellular staining of Interferon gamma (IFN-γ) in CD8+ T cells after LD2 treatment (or positive control) in primary AML samples described in FIG. 14. LD2 treatment in total of 4 days ex-vivo culture (under the BM stroma cytokine condition). For positive control, AML MNCs were first cultured without Phorbol 12-myristate 13-acetate (PMA)/ionomycin for 90 h, then exposed to PMA/ionomycin for last 6 h. The plots showed the percentage of IFN-γ CD8+ T cells in each group.

FIGS. 18A-18V illustrate that PRMT9 inhibition eradicates AML in vivo via a type-I IFN response. FIGS. 18A-18F: Control or inducible Prmt9-KD MA9-lucifase cells were injected into wild type B6 (1×10⁶ cells per mouse, A, n=7), Rag2^{-/-} (0.1×10⁶ cells per mouse, B, n=5) and NSGS (0.1×10⁶ cells per mouse, C, n=5) mice. Following engraftment confirmation, mice were continuously administered Dox in drinking water. MA9 cell engraftment was tracked by in vivo bioluminescence imaging over indicated days (FIGS. 18A-18C). Kaplan-Meier curves show overall survival of wild type B6 (FIG. 18D), Rag2^{-/-} (FIG. 18E), and NSGS (FIG. 18F) mice in each group. P values (FIGS. 18E-18F) were determined by log-rank (Mantel-Cox) test. FIG. 18G: Control or inducible Prmt9-KD CMM cells were injected into wild type B6 (1×10⁶ cells per mouse, A, n=7). Following engraftment confirmation, mice were treated with Dox as above. Kaplan-Meier curves show overall survival of mice in each group. P values were determined by log-rank (Mantel-Cox) test. FIG. 18H: Uniform Manifold Approximation and Projection (UMAP) plot showing different cell populations identified within Ctrl and Prmt9-KD groups merged in BM cells. FIG. 18I: UMAP plot showing representative expression of markers of each identified cell population in BM cells. FIG. 18J: UMAP plot showing Prmt9 expression in BM populations in Ctrl and Prmt9-KD groups. FIG. 18K: UMAP plot showing expression of Cd69, Ifng, and Gzmb in T cells of Ctrl (n=249) and Prmt9-KD (n=231) BM samples. Bar chart (right) shows the statistics of Ifng expression in T cells in Ctrl vs. Prmt9-KD group. Data are presented as mean±SEM. P value (right) was determined by unpaired, two-tailed t-tests. ****P<0.0001. FIG. 18L: Frequency of MA9 AML-specific CD8+ T cells using Major histocompatibility complex (MHC)-survivin peptide pentamer analysis (MHC-Cytomegalovirus (CMV) peptide as a control) in Prmt9-KD MA9 transplant mice (n=5) relative to Prmt9-WT controls (n=5) in BM. Data are presented as mean±SEM. P value was determined by two-sided Mann-Whitney test. FIG. 18M: Inducible Ctrl, Prmt9-KD, cGAS KO or Prmt9-KD+cGAS KO MA9/OVA cells were injected into wild type B6 mice (1×10⁶ cells per mouse, n=5). Following engraftment confirmation, mice were treated with Dox as above for 7 days to induce Prmt9 KD. MA9-OVA-specific T cells in BM were tracked using the H-2K^{b}-OVA₂₅₇₋₂₆₄ tetramer. Data are presented as mean±SEM. P value was by two-sided Mann-Whitney test. P=0.0079, **P<0.01. FIG. 18N: UMAP plot showing subpopulations identified among T cells from spleen within Ctrl and Prmt9-KD groups merged. FIG. 18O: Heatmap displaying relative expression of select genes in T cell clusters. Color code represents z scores of selected genes within clusters. FIG. 18P: UMAP plots showing distribution of annotated clusters in (M) within Ctrl and Prmt9-KD groups. FIGS. 18Q-18R: Percentage of each cell cluster in CD8+ (P) or CD4+ (Q) T cells annotated in (FIG. 18M). FIG. 18S: Survivors (>120 days) of MA9 Prmt9-KD cell challenged mice (n=4) were rechallenged with 1×10⁶ parental MA9 Prmt9-KD cells (without Dox induction). Control naïve C57BL/6 mice (n=5) inoculated with the same number of MA9 Prmt9-KD cells served as controls. Kaplan-Meier curves show overall survival of mice in naïve and survivor groups. P value was determined by log-rank (Mantel-Cox) test. FIG. 18T: Heatmap showing upregulated immune response genes interferon-stimulated genes (ISGs) in Prmt9-KD BM T cells. FIG. 18U: Gene set enrichment analysis (GSEA) analysis of differentially expressed genes in BM T cells after Prmt9 KD in MA9 cells in vivo. FIG. 18V: Inducible Prmt9 KD MA9-lucifase cells were injected into wild type (n=7) or Ifnar1 KO B6 mice (1×10⁶ cells per mouse, n=5). Following engraftment confirmation, mice were treated with Dox as above. Kaplan-Meier curves show the overall survival of mice in each group. P value was determined by log-rank (Mantel-Cox) test.

FIGS. 19A-19B illustrate that Prmt9-KD decreases AML burden and extends survival of advanced leukemic mice. FIG. 19A: Control or inducible Prmt9-KD MA9-lucifase cells were injected into wild type B6 (1×10⁶ cells per mouse, n=5/group). After 30 days when leukemia robustly developed, mice were continuously administered with Dox. Kaplan-Meier curves show the overall survival in each group after Dox induction (FIG. 19A). P value was determined by log-rank (Mantel-Cox) test. MA9 AML burden was tracked by in vivo bioluminescence imaging over indicated days (FIG. 19B) and the statistics for the quantitative results on day 30 and day 50 from bioluminescence imaging were shown (FIGS. 19B, right). Data are presented as mean±SEM. P values were determined by two-sided Mann-Whitney test. “ns” represents no significance.

FIGS. 20A-20R illustrate that PRMT9 inhibition eradicates AML in vivo via a type-I IFN response. FIG. 20A: After MA9 cells are engrafted in the BM, 1 day prior to in-vivo DOX administration to KD Prmt9, mice are administered with combined anti-CD4/CD8 treatment or anti-NK1.1 400 μg twice for the first week, and 200 μg twice weekly to deplete T cells or NK cells. Representative peripheral blood (PB) flow plot of T cells or NK cells depletion was shown after the first week antibodies treatment. FIG. 20B: Prmt9-KD or -WT MA9+ AML cells were implanted into WT B6 mice and evaluated AML progression in four groups: 1) Ctrl (Prmt9-WT), n=7; 2) Prmt9-KD, n=7; 3) Prmt9-KD with T cell depletion, n=5; 4) Prmt9-KD with NK cell depletion, n=5. Kaplan-Meier curves show the overall survival of mice in each group. P value was determined by log-rank (Mantel-Cox) test. FIG. 20C: UMAP plot showing cell distribution within identified cell populations in Ctrl and Prmt9-KD groups in BM cells. FIG. 20D: Relative frequency of MA9 AML cells in BM of Ctrl or Prmt9-KD mouse. FIGS. 20E-20G: UMAP plot showing different cell populations (FIG. 20E) and representative expression markers (FIG. 20F) identified within Ctrl and Prmt9-KD groups merged in spleen cells. FIG. 20G: UMAP plot showing cell distribution within identified spleen cell populations in Ctrl or Prmt9-KD mouse. FIG. 20H: Relative T cell frequency in spleen cells of Ctrl or Prmt9-KD mouse. FIG. 20I: UMAP plot showing expression of T cell marker genes in spleen Ctrl or Prmt9-KD mouse. FIG. 20J: Relative frequency of Cd44+ cells in BM T cells of Ctrl or Prmt9-KD mouse. FIG. 20K: Relative frequency of Tregs (Foxp3+) in BM CD4+ T cells of Ctrl or Prmt9-KD mouse. FIG. 20L: Representative flow plot of BM cells in MA9 parent cell rechallenge experiments which were still dead of leukemia. FIG. 20M: ‘Hallmarks’ signature Interferon Alpha Response plot of GSEA analysis of BM T cells. FIGS. 20N-200: UMAP plot (FIG. 20N) and histogram (FIG. 20O) showing Isg15 expression in each indicated BM cell subpopulation in Ctrl and Prmt9-KD groups. Ctrl: T cells, n=249; Monocytes/Macrophages, n=631; DCs, n=108; Granulocytes, n=3378; B cells, n=413. PRMT9-KD: T cells, n=231; Monocytes/Macrophages, n=469; DCs, n=57; Granulocytes, n=3906; B cells, n=134. Data are presented as mean±SEM. P values were determined by unpaired two-tailed t-tests. ****p<0.001. FIGS. 20P-20Q: UMAP plot (FIG. 20P) and histogram (FIG. 20Q) showing Ifit1 expression in each indicated BM cell population in Ctrl and Prmt9-KD groups. Ctrl: T cells, n=249; Monocytes/Macrophages, n=631; DCs, n=108; Granulocytes, n=3378; B cells, n=413. PRMT9-KD: T cells, n=231; Monocytes/Macrophages, n=469; DCs, n=57; Granulocytes, n=3906; B cells, n=134. Data are presented as mean±SEM. P values were determined by unpaired two-tailed t-tests. ****p<0.001. FIG. 20R: Histogram showing Ifit1 expression in each indicated BM cell populations in Ctrl and Prmt9-KD groups. Ctrl: T cells, n=249; Monocytes/Macrophages, n=631; DCs, n=108; Granulocytes, n=3378; B cells, n=413. PRMT9-KD: T cells, n=231; Monocytes/Macrophages, n=469; DCs, n=57; Granulocytes, n=3906; B cells, n=134. Data are presented as mean±SEM. P values were determined by unpaired two-tailed t-tests. ****p<0.001.

FIGS. 21A-21B illustrate the cell clustering of scRNAseq analysis. FIG. 21A: Inducible control or Prmt9 KD MA9-lucifase cells were injected into wild type B6 mice (1×10⁶ cells per mouse, n=5). Following engraftment confirmation, mice were treated with Dox in drinking water for 7 days. The plot shows the frequency of MA9, CD3+, B220+ and Gr1+/Mac1+ cells in BM of each mouse at the time of collection analyzed by flow-cytometry. BM and spleen cells from one representative mouse in each group were selected and subjected to single cell RNA-seq analysis. Data are presented as mean±SEM. P value of each comparison was determined by two-sided Mann-Whitney test. FIG. 21B: Heatmap showing representative expression of markers of identified clusters, including MA9 cells (MA9), monocytes/macrophages (Mono.), T cells (T), B cells (B), hematopoietic stem and progenitor cells (HSPCs), basophils/mast cells (Baso./Mast.), dendritic cells (DCs), natural killer cells (NK), granulocytes (Gran.), and erythroid cells (EryDs).

FIGS. 22A-22B illustrate that Survivin is more highly expressed in MA9 cells than in other immune subsets. FIG. 22A: UMAP plot showing Birc5 (encoding Survivin) expression in different MA9 leukemic BM subpopulations in Ctrl sample. FIG. 22B: Histogram showed elevated expression of Birc5 in MA9 leukemia cells relative to other immune subsets. MA9 cells, n=1826; T cells, n=249; Monocytes/Macrophages, n=631; DCs, n=108; Granulocytes, n=3378; B cells, n=413. Data are presented as mean±SEM, statistical comparisons were performed using unpaired two-tailed t-test, ****P<0.0001.

FIGS. 23A-23S illustrate that Anti-tumor immunity following PRMT9 inhibition requires cGAS activity in cancer cells. FIG. 23A: Heatmap showing upregulated immune response genes in Prmt9-KD MA9 cells. FIG. 23B: GSEA analysis of differentially expressed genes in MA9⁺ leukemia cells from mouse BM following Prmt9 KD in vivo. FIG. 23C: RNA-seq analysis of overlapped differentially expressed genes in PRMT9-KD versus Ctrl Molm13 and THP1 cells (FC>2). Color code represents z scores for differential gene expression. FIG. 23D: Q-PCR analysis of ISG15 expression in Molm13 cells after PRMT9 KD and following rescue with Wt PRMT9 or a catalytically dead mutant. Data are presented as mean±SD. P value was by two-sided Mann-Whitney test. P=0.0079, **P<0.01. FIG. 23E: Q-PCR analysis of indicated ISG genes in primary AML CD34⁺ cells treated 2 days with LD2 (2.5 μM). Data are presented as mean±SD from 3 independent experiments. FIG. 23F: Lucia reporter assay of WT, cGAS KO, or MAVS KO THP1 IRF reporter monocytes after 2 days of Dox treatment to induce PRMT9 KD. Data are presented as mean±SD. P value was by two-sided Mann-Whitney test. P=0.0079, **P<0.01. FIG. 23G: cGAMP levels in media of Ctrl, PRMT9-KD or PRMT9-KD+ENPP1 THP1 cells, as detected by Cyclic GAMP Enzyme Immunoassay Kit (Arbo Assay). Data are presented as mean±SD. FIG. 23H: Deoxyribonucleic acid (DNA) damage response detected by immunostaining for γH2AX in Ctrl and PRMT9 KD THP1 cells after Dox induction for 72 h (representative shown in left). Violin plots (right) show statistics of relative γH2AX intensity in each (Ctrl, PRMT9-KD, n=100 cells per group) group. Scale bar, 10 m. P value was determined by two-sided Mann-Whitney test. ****P<0.0001. FIG. 23I: dsDNA detected by immunostaining with antidsDNA antibody (AE-2) in Ctrl and PRMT9 KD THP1 cells after Dox induction for 72 h. Violin plots (right) show statistics of relative dsDNA intensity each (Ctrl, PRMT9-KD, n=50 cells per group) group. Scale bar, 10 m. P value was determined by two-sided Mann-Whitney test. ****P<0.0001. FIG. 23J: Inducible Ctrl, Prmt9-KD, cGAS KO or Prmt9-KD+cGAS KO MA9/OVA cells were injected into wild type B6 mice (1×10⁶ cells per mouse). Following engraftment confirmation, mice were treated with Dox in drinking water as described. Kaplan-Meier curves show the overall survival of mice in each group. P values were determined by log-rank (Mantel-Cox) test. FIG. 23K: cGAS-KO MA9 cells were transduced with inducible HA-tagged cGAS WT or ΔN. Expression of exogenous cGAS was assessed by western blot assay after Dox induction. FIGS. 23L-23M: cGAS-KO (n=5), cGAS-WT (n=5) or cGAS-ΔN MA9 (n=7) cells were transplanted into WT B6 mice (1×10⁶ cells per mouse). Following leukemia cell engraftment in ˜2 weeks (>1% in PB), mice were treated with Dox in drinking water as above to induce expression of cGAS variants until one mouse died in the cGAS-KO group. Engraftment of MA9 cells in BM was assessed by flow cytometry (FIG. 23L). Data are presented as mean±SEM. P values were determined by Kruskal-Wallis test. For another cohort, Kaplan-Meier curves show overall survival of mice in each group (FIG. 23M). P values were determined by log-rank (Mantel-Cox) test. ***P<0.001, ****P<0.0001. FIG. 23N: Relative cGAS mRNA levels in the BEAT AML dataset of MNCs from AML cases (n=451) and healthy donors (n=19). P value was determined by unpaired two-tailed t-test. ****P<0.0001. FIG. 23O: Inducible control or Prmt9 KD MA9-lucifase cells were injected into WT B6 mice (1×10⁶ cells per mouse). Following engraftment confirmation, mice were treated with Dox in drinking water as above. Cyclic guanosine monophosphate (GMP)-adenosine monophosphate (AMP) (cGAMP) levels in the BM microenvironment of Ctrl and PRMT9-KD mice were detected by Cyclic GAMP Enzyme Immunoassay Kit (Arbo Assay). Data are presented as mean±SD. FIG. 23P: Violin plots showing expression of Cd80, Cd86, and H2-ab1 in DCs based on scRNAseq analysis of Ctrl (n=108) and Prmt9-KD (n=57) BM. P values (<0.0001) were determined by unpaired, two-tailed t-tests. ****P<0.0001. FIGS. 23Q-23R: LD2-pretreated cGAS-KO or cGAS-WT MA9/OVA cells were co-cultured with BM-derived DCs. DCs were then purified and co-cultured with naive OT-I transgenic CD8+ T cells. IFN-γ production by CD8+ T cells was detected using a CBA Mouse IFN-γ Flex Set kit (FIG. 23Q), and IFN-β production was detected by an ELISA assay (FIG. 23R). Data are presented as mean±SD. FIG. 23S: Prmt9-KD or -WT MA9+ AML cells were implanted into Batf3-WT or KO mice and evaluated AML progression in three groups: 1) Prmt9-KD/Batf3-KO (n=5), 2) Prmt9-KD Batf3-WT (n=7), and 3) Prmt9-WT/Batf3-WT (n=7). Kaplan-Meier curves show the overall survival of mice in each group. P values were determined by log-rank (Mantel-Cox) test.

FIGS. 24A-24S illustrate that anti-tumor immunity following PRMT9 inhibition requires cGAS activity in cancer cells. FIG. 24A: UMAP plot showing Ifit1 expression in MA9 AML cells in Ctrl and Prmt9-KD BM based on single cell RNA-seq analysis. FIG. 24B: ‘Hallmarks’ signature Interferon Alpha Response plot of GSEA analysis of BM MA9 AML cells. FIGS. 24C-24D: Bulk RNA-seq analysis of co-differentially expressed genes in PRMT9-KD versus Ctrl Molm13 and THP1 cells. Heatmap (FIG. 24C) and ‘Hallmarks’ signature Interferon Alpha Response plot (FIG. 24D) showing co-upregulation of IFNα response genes in Molm13 and THP1 cells. The color code represents z scores for differential gene expression. FIG. 24E: Q-PCR analysis of IFI44 expression in Molm13 cells after PRMT9 KD and after rescue with Wt or catalytically dead (mutant) PRMT9. Data are presented as mean±SD. P value (0.0079) was determined by two-sided Mann-Whitney test. **P<0.01. FIGS. 24F-24G: Q-PCR analysis of selected ISG genes in AML (Molm13, THP1, and MA9) and B-NHL (Rec1, OCI-Ly3, and A20) lines after treated 2 days with LD2. Data are presented as mean±SD from 3 independent experiments. FIG. 24H: Lucia reporter assay of WT, cGAS KO, and MAVS KO THP1 monocytes harboring an IRF reporter after 2 days of LD2 treatment. Data are presented as mean±SD. P value (0.0079) was determined by two-sided Mann-Whitney test. **P<0.01. FIG. 24I: Western blot analysis of PRMT9 and Flag-tagged ENPP1 in engineered THP1 cells. FIG. 24J: Western blot analysis of γH2AX in Ctrl and PRMT9-KD THP1 cells. FIG. 24K: dsDNA detected by immunostaining with anti-dsDNA antibody (AE-2) in THP1 cells after VEH or LD2 treatment for 72 h. Violin plots (right) shows statistics of relative dsDNA intensity each (Ctrl, PRMT9-KD, n=50 cells per group) group. Scale bar, 10 m. P value was determined by two-sided Mann-Whitney test. ****P<0.0001. FIG. 24L: DNA damage response detected by immunostaining for γH2AX in Ctrl and PRMT9 KD THP1 cells after Dox induction for 48 h. Violin plots (right) show statistics of relative γH2AX intensity in each (Ctrl, PRMT9-KD, n=100 cells per group) group. Scale bar, 10 m. P value was determined by two-sided Mann-Whitney test. FIG. 24M: dsDNA detected by immunostaining with anti-dsDNA antibody (AE-2) in Ctrl and PRMT9 KD THP1 cells after Dox induction for 48 h. Violin plots (right) show statistics of relative dsDNA intensity each (Ctrl, PRMT9-KD, n=50 cells per group) group. Scale bar, 10 m. P value was determined by two-sided Mann-Whitney test. FIG. 24N: Western blot analysis of cGAS in Ctrl and cGAS-KO MA9 cells (n=2). FIG. 24O: Relative ENPP1 mRNA levels in the BEAT AML dataset of AML cases (n=451) and healthy donors (n=19). P value was determined by two-sided Mann-Whitney test. FIGS. 24P-24Q: cGAS (FIG. 24P) and ENPP1 (FIG. 24Q) expression in AML and B-NHL lines compared with lines representing the top 10 most deadly cancers. AML (n=14), B-NHL (n=9), Lung Cancer (LC, n=77), Colon Adenocarcinoma (COAD, n=30), Pancreatic adenocarcinoma (PAAD, n=20), Breast Cancer (BRCA, n=30), Prostate adenocarcinoma (PRAD, n=5), Liver Hepatocellular Carcinoma (LIHC, n=14), Glioblastoma (GBM, n=12), Bladder Carcinoma (BLCA, n=11) and Acute Lymphoblastic Leukemia (ALL, n=8). P values were determined by two-sided Mann-Whitney tests. ****P<0.0001. FIG. 24R: Histogram showing Cd80, Cd86, and H2-ab1 expression in Ctrl and Prmt9-KD monocytes/macrophages from BM, based on single cell RNA-seq analysis. Ctrl, n=631; Prmt9-KD, n=469. Data are presented as mean±SEM. P values were determined by unpaired two-tailed t-tests. ****P<0.0001. FIG. 24S: HEK293T cells were transfected with Flag-tagged 5′-3′ exoribonuclease 2 (XRN2) (WT or R946K). Cell lysates were prepared for immunoprecipitation using anti-Flag beads and interactors detected by immunoblot with an anti-p54 (nrb) antibody. 7H2AX was detected in the input lysate.

FIGS. 25A-25R illustrate that Loss of XRN2 methylation underlies cGAS activation in cancer cells. FIGS. 25A-25B: Western blot analysis of phospho-CHK1, phospho-CHK2, and γH2AX after PRMT9 KD (FIG. 25A) or LD2 treatment (FIG. 25B) in THP1 cells (n=2). FIG. 25C: Neutral comet assay of THP1 cells after PRMT9 KD for 48 h and 72 h. Scatter plots (right) shows the statistics of olive tail moments in each (Ctrl, PRMT9-KD, n=50 cell/group) group. Scale bar, 50 m. P value was determined by two-sided Mann-Whitney test. ****P<0.0001. FIG. 25D: Lucia reporter assay of IRF reporter THP1 after KO of indicated genes by electroporation of ribonucleoprotein complexes (RNPs) of Cas9 protein and guide RNAs (gRNAs) targeting candidate genes. Data are presented as mean±SD from 3 independent experiments. FIG. 25E: In vitro methylation assay of synthesized unmodified KHDRBS1 (Sam68) (aa326-339), XRN2 (aa937-950), or DDX3X (aa80-92) peptides in the presence of PRMT9 protein and SAM. Methylation was analyzed by western blotting using an anti-pan-MMA antibody (n=2). FIG. 25F: Western blot analysis of XRN2 and DDX3X after their respective KO in THP1 cells (n=2). FIG. 25G: Lucia reporter assay of XRN2-WT and XRN2-R946K mutant in WT and cGAS KO THP1 monocytes harboring an IRF reporter. gRNA-resistant XRN2-WT and -R946K constructs were ectopically expressed in WT THP1 IRF/Lucia reporter cells. A reporter assay was performed 2 days after electroporation of these cells with RNPs to target endogenous XRN2. Data are presented as mean±SD. P value (0.0079) was determined by two-sided Mann-Whitney test. **P<0.01. FIG. 25H: Lucia reporter assay of DDX3X-WT and -R88K mutant in WT THP1 monocytes harboring an TRF reporter. gRNA-resistant DDX3X WT or R88K constructs were ectopically expressed in WT THP1 IRF/Lucia reporter cells. A reporter assay was performed 2 days after electroporation of these cells with RNPs to target endogenous DDX3X. Data are presented as mean±SD. P value was determined by two-sided Mann-Whitney test. “ns”: no significance. FIGS. 25I-24J: Dot blots showed in vitro methylation of XRN2 peptides (aa937-950) with PRMT9 (FIG. 25I) or PRMT5 (FIG. 25J) with or without increased dose of LD2 (FIG. 25I) or EPZ015666 (FIG. 25J) respectively. FIG. 25K: XRN2-WT and XRN2-R946K mutant THP1 cells were prepared for immunoprecipitation using anti-Flag beads and interactors detected by immunoblot with an anti-p54 (nrb) antibody. Phosphor-CHK1 and γH2AX were also detected in the input lysate (n=2). FIGS. 25L-24M: R-loop signals detected by dot blots (FIG. 25L, n=2) or representative immunostaining (FIG. 25M) with S9.6 antibody in XRN2-WT and XRN2-R946K mutant THP1 cells. Scale bar, 10 m. FIGS. 25N-24O: R-loop signals detected by dot blots with S9.6 antibody in WT or RNASEH1 overexpressed THP1 cells treated with LD2 (2.5 μM) for 48 h and 72 h (FIG. 25N) or Dox induced PRMT9-KD for 48 h (FIG. 25O) (n=2). ssDNA was stained as the loading control. FIG. 25P: Cell cycle analysis of THP1 cells treated 48 h with LD2 (2.5 μM). Bar chart (right) shows the statistics of G2/M, S and G1 phase in VEH vs. LD2 group. Data are presented as mean±SD. P values (right) were determined by two-way ANOVA. FIG. 25Q: Western blot analysis of phosphor-CHK1 in WT or RNASEH1 overexpressed THP1 cells treated 72 h with LD2 (2.5 μM) (n=2). FIG. 25R: Lucia reporter assay of WT or RNASEH1 overexpressed THP1-Lucia cells treated 72 h with (2.5 μM) LD2. Data are presented as mean±SD. P value (0.0079) was determined by two-sided Mann-Whitney test. **P<0.01.

FIG. 26 illustrates the spectrum of XRN2 R946 methylation in SILAC analysis. It shows representative mass spectra of XRN2 R946 methylation in SILAC-based methyl-peptide quantitative LC-MS/MS.

FIGS. 27A-27M illustrate that combining LD2 with an immune checkpoint inhibitor ablates PRMT9-proficient cancers. FIGS. 27A-27B: UMAP plot (FIG. 27A) and histogram (FIG. 27B) showing Cd274 (PD-L1) and Pdcd1lg2 (PD-L2) expression in MA9+ AML cells, based on scRNAseq analysis of Ctrl (n=1827) and Prmt9-KD (n=1124) BM leukemic cells. Data are presented as mean±SEM. P values of (FIG. 27B) were determined by unpaired, two-tailed t-tests. ****P<0.0001. FIG. 27C: Representative tSNE display of CyTOF analyses of primary AML cells treated with LD2 (2.5 μM, 4 days), colored by expression of CD274 (PD-L1) based on CD34⁺CD45^{dim} subsets. FIG. 27D: CyTOF analysis of MNCs from the AML01 sample after treatment for 4 days with an anti-PD1 antibody or anti-PD-1+LD2. SPADE clustering shows cell groups corresponding to immunophenotypic subsets across normal hematopoiesis lineages. The frequency of CD3⁺ T cells and CD34⁺CD45^{dim} AML blast cells were noted. Color bar shows CD34 expression intensity. FIG. 27E: Relative leukemia (CD34⁺CD45^{dim}) cell frequencies in MNCs from the AML01 sample after treatment described in (FIG. 27D). FIGS. 27F-27G: Biaxial plots showing CD69 (FIG. 27F) and IFNγ (FIG. 27G) expression in CD8⁺ T cells in AML01 samples treated 4 days with vehicle, LD2, anti-PD1, and LD2+anti-PD-1. FIGS. 27H-27I: Balb/C mice were inoculated with 3×10⁶ A20 cells. When tumor volume reached 100 mm³, mice were treated with isotype control (vehicle), anti-PD1 mAb (BE0146 [BioXCell], 10 mg/kg, i.p. every other day for 2 weeks), LD2 (100 mg/kg, i.t., daily for 2 weeks) or combined LD2+anti-PD1 (n=5/group). Tumor volume was monitored through the end of the study (FIG. 27H) and pictures of 5 tumors in each group were acquired (FIG. 27I). Data (FIG. 27H) are presented as mean±SEM. P values were determined by two-way ANOVA. FIGS. 27J-27K: Representative image showing anti-mCD8 IHC staining in indicated groups. Scale bar, 100 m. FIG. 27D: Quantification of CD8 T cells, reported as the number of cells per gram of tumor tissue in indicated groups. Data (FIG. 27K) is presented as mean±SEM. P values were determined by two-sided Mann-Whitney test. FIGS. 27L-27M: Representative flow plots showing GZMB (FIG. 27L) and IFNγ (FIG. 27M) expression in CD8⁺ T cells in indicated treatment groups.

FIGS. 28A-28P illustrate that combining LD2 with an immune checkpoint inhibitor ablates PRMT9-proficient cancers. FIGS. 28A-28B: UMAP plot (FIG. 28A) and histogram (FIG. 28B) showing Ctla4, Tim3, Tigit, and Pd-1 expression in T cells based on single cell RNA-seq analysis of Ctrl (n=249) and Prmt9-KD (n=231) BM T cells. Data are presented as mean±SEM. P values were determined by unpaired two-tailed t-test. FIG. 28C: Frequency of PD-L1 positive cells among leukemic population (CD34+CD45dim) following treatment with/without LD2 (2.5 μM, 4 days). FIGS. 28D-28E: CyTOF analysis of MNCs from the AML03 sample after treatment for 4 days with an anti-PD-1 antibody or anti-PD-1+LD2. SPADE clustering shows cell groups corresponding to immunophenotypic subsets across normal hematopoiesis lineages. The frequency of CD3+ T cells and CD34+CD45dim AML blast cells were noted. Color bar shows CD34 expression intensity (FIG. 28D). FIG. 28E: Relative leukemia (CD34⁺CD45dim) cell frequencies in MNCs from the AML03 sample after 4 days of treatment with anti-PD-1 antibody or anti-PD-1+LD2. FIG. 28F: Western blot analysis of Prmt9 expression in normal mouse PBMCs or A20 cells (n=2). FIG. 28G: Weight of A20 tumors in each group (n=5/group). P value of each comparison was determined by two-sided Mann-Whitney test. FIGS. 28H-28I: NSGS mice (n=10) were inoculated with 1×10⁶ A20 cells. When tumor size reached a volume of 100 mm³, mice were treated with vehicle or LD2 (100 mg/kg, i.t., daily for 2 weeks). Tumor volume was monitored until the end of the study (FIG. 28H). Data (FIG. 28H, FIG. 28I) are presented as mean±SEM. P value (FIG. 28H) was determined by two-way ANOVA. Final tumor weights are shown (FIG. 28I). P value (FIG. 28I) was determined by two-sided Mann-Whitney test. FIG. 28J: Western blot analysis of PABPC1 R493 methylation in A20 tumors treated with vehicle or LD2 (n=2). FIGS. 28K-28L: Q-PCR analysis of Cd274 (FIG. 28J) and mIfit1 (FIG. 28K) cells in A20 tumors from mice treated with vehicle or LD2 (n=3). Data are presented as mean±SD. FIG. 28M: Representative image of anti-mCD3 IHC staining in indicated groups. Scale bar, 100 m. FIG. 28N: Quantification of CD3 T cells, based on the number of cells per gram of tumor tissue in indicated treatment groups. Data are presented as mean±SEM. P value of each comparison was determined by two-sided Mann-Whitney test. FIGS. 28O-28P: Quantification of GZMB+ (FIG. 28O) and IFNγ+ (FIG. 28P) cells among CD8+ T cells in indicated treatment groups. Data are presented as mean±SEM. P value of each comparison was determined by two-sided Mann-Whitney test. Unless otherwise specified. *p<0.05, **p<0.01, ***p<0.001.

FIGS. 29A-29K illustrate that Administration of LD2 combined with an immune checkpoint inhibitor ablates AML in vivo. FIGS. 29A-29C: MA9 cells were injected into wild type B6 (n=5/group). While leukemia cells engrafted, the AML-bearing mice was treated for 3 weeks with vehicle control (VEH), a single PD-1 inhibitor (PD-1i, BE0146 [BioXCell], 10 mg/kg, i.p. every other day for 3 weeks), LD2 (10 mg/kg/i.v./BID for 3 weeks) or the LD2 plus PD-1i (combination). LD2 was administered at 10 mg/kg/i.v./BID, based on preliminary pharmacokinetic (PK)/pharmacodynamic (PD) analysis. After 3 weeks treatment, leukemic progenitors (GFP+cKit+) engraftment in the BM were assessed by flow cytometry (FIG. 29A). The MA9 mice BM were stained with anti-CD8 together with murine survivin-specific pentamers to assess the MA9-specific CD8+ T cells in BM. Cytomegalovirus (CMV)-specific pentamers were used as the negative control. Histograms showed the statistics of the frequencies of survivin-specific and CMV-specific CD8+ T cells in BM (FIG. 29B). Representative flow plots of survivin and CMV staining of CD8+ T cells (FIG. 29C). Data are presented as mean±SEM. P values (FIG. 29A, FIG. 29B) were determined by two-sided Mann-Whitney test. For (FIG. 29B), P=0.0079, **P<0.01. FIG. 29D: In another batch of experiment as (FIG. 29A), after 3 weeks treatment, Kaplan-Meier curves showed the overall survival of mice (n=5/group) in each group. P values were determined by log-rank (Mantel-Cox) test. FIG. 29E: A secondary transplantation assay was performed to evaluate the LSC activity in each group in (FIG. 29A) by checking the MA9 cells engraftment in BM. Same amount of BM cells from first transplant mice (FIG. 29A) were injected into irradiated WT B6 mice. MA9(GFP+) cells in the BM were assessed when control mice fully developed leukemia. Data are presented as mean±SEM. P values were determined by two-sided Mann-Whitney test. FIGS. 29F-29I: Two million MNCs from an AML specimen were implanted intra-femorally into an irradiated MHC1/2 double-KO (DKO) NSG mouse. After 12 weeks, mice were divided into two groups and treat with VEH or LD2 (10 mg/kg/i.v./BID). After 3 weeks treatment, the numbers/frequency of leukemic CD34+ cells were checked (FIG. 29F, FIG. 29G) and frequencies of CD8+ T cells expressing CD69 (FIG. 29H) and IFNγ (FIG. 29I) in the LD2 group relative to VEH. Data are presented as mean±SEM. P values (FIGS. 29G-29I) were determined by two-sided Mann-Whitney test. FIGS. 29J-29K: PRMT9 KD gene signature levels in indicated immune checkpoint blockade (ICB) cohorts of complete response (CR) and progressive disease (PD) patients enrolled in clinical trials against melanoma and bladder cancer evaluating PD-1 or PD-L1 blocking monoclonal antibodies. The PRMT9 KD gene signature is composed of 102 differentially expressed genes common to 2 AML cell lines (fold change >2, P<0.05) (Table 5). Analysis was performed by applying the single sample GSEA (ssGSEA) method available in the GSEA software. Violin plots are used to compare the distribution of normalized enrichment score (NES) between groups. Statistical comparisons were performed using unpaired two-tailed Student t tests.

FIGS. 30A-30C illustrate the humanized AML mouse model establishment. FIGS. 30A-30B: Two million MNCs from an AML specimen were implanted intra-femorally into an irradiated MHC1/2 double-KO NSG mouse or NSG mouse (n=3). After transplant, engraftment of CD3+T cells as well as CD33+ cells in PB were checked by flow cytometry for 12 weeks. After 12 weeks, mice were culled and BM engraftment of human hematopoietic subsets including T cells, monocytes and DCs, as well as the immature CD33+CD34+CD45dim subset were checked by flow cytometry. Data are presented as mean±SEM. FIG. 30C: Representative gating strategy of human hematopoietic subsets of BM cells from humanized AML NSG-DKO mouse model.

FIG. 31 illustrates the data analysis of the PRMT9 DDR cGAS axis in the context of AML immune therapy. Single sample gene set enrichment analysis (ssGSEA) of AML clinical trial dataset GSE183415 which contains clinical responses to PD-1 inhibitors (Complete Response [CR] vs. No Response [NR]) using the following gene signature: PRMT9-KD signature, 390 ISG signature, Reactome DNA repair signature, Reactome Double strand break repair signature, and Reactome G2M DNA damage checkpoint signature. Data are presented as mean±SD, statistical comparisons were performed using unpaired two-tailed t-test, “ns” indicates no significance.

FIGS. 32A-32E illustrate that targeting arginine methylation suppresses cancer cell maintenance and elicits cGAS-mediated anti-cancer immunity. FIG. 32A: Proposed model depicting role of PRMT9 in cancer therapy. FIG. 32B: “REACTOME” signature “DNA Repair”, “DNA Double Strand Break Repair”, “G2M DNA Damage Checkpoint” plots of GSEA analysis of BM MA9 AML cells (Prmt9-KD vs. Ctrl). FIG. 32C: 390 ISGs signature plot of GSEA analysis of BM DCs (Prmt9-KD vs. Ctrl). FIG. 32D: ‘Hallmarks’ signature Interferon Gamma Response plot of GSEA analysis of BM T cells (Prmt9-KD vs. Ctrl). FIG. 32E: ‘Hallmarks’ signature Interferon Gamma Response plot of GSEA analysis of BM MA9 AML cells (Prmt9-KD vs. Ctrl).

FIG. 33 illustrates that PRMT9 does not catalyze cGAS methylation. In inducible PRMT9-KD Molm13 cells, cGAS protein was IP'd after three days of Dox induction and performed immunoblotting with mono-methylation (MMA) antibody or SDMA antibodies mixture (SYM10 μMillipore], panSDMA antibody mix [CST]). PABPC1 R493me was also blotted as a control for PRMT9 inhibition.

FIG. 34 shows the oligonucleotides used in the examples herein.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The abbreviations used herein have their conventional meaning within the chemical and biological arts. The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—NH₂, —CH₂—CH₂—NO₂, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” or “-” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

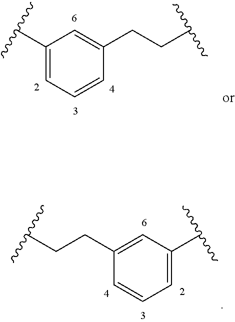 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

The terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A)oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:

(i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from

(a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth in the Examples, figures, or tables herein.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. The term “isomers” refers to compounds having the same number and kind of atoms, and hence the same molecular weight, but differing in respect to the structural arrangement or configuration of the atoms. The term “regioisomers” refers to compounds having the basic carbon skeleton unchanged but their functional groups or substituents change their position on a parent structure. The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure. The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, etc., including solid and lymphoid cancers.

The term “cancer” refers to all types of cancer, neoplasm or malignant tumors found in mammals (e.g. humans), including leukemias, lymphomas, carcinomas and sarcomas. Exemplary cancers that may be treated with a compound or method provided herein include kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “hematological cancer” or “blood cancer” refers to a cancer in, or that begins in, a blood-forming tissue, such as the bone marrow, or in the cells of the immune system. Examples of hematologic cancer are leukemia, lymphoma, multiple myeloma, myelodysplastic syndrome (MDS), and myeloproliferative disorder (MPD). In most blood cancers, the normal blood cell development process is interrupted by uncontrolled growth of cancerous cells.

The term “immune-cold cancer” or “non-T-cell-inflamed cancer” refers to a tumor or a cancer that is not infiltrated by T cells and is therefore not likely to trigger a strong immune response. Cold tumors tend to be surrounded by cells that are able to suppress the immune response and keep T cells from attacking the tumor cells and killing them. Cold tumors usually do not respond to immunotherapy.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, acute lymphoblastic leukemia, acute myeloid leukemia, chronic myelomonocytic leukemia, chronic myelogenous leukemia, chronic neutrophilic leukemia, immunoblastic large cell leukemia, megakaryoblastic leukemia, acute megakaryocytic leukemia, promyelocytic leukemia, erythroleukemia, chronic granulocytic leukemia, acute promyelocytic leukemia, adult T-cell leukemia, aleukemic leukemia, leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, precursor B-lymphoblastic lymphoma, lymphoplasmacytic lymphoma, hairy cell leukemia, primary central nervous system (CNS) lymphoma, and primary intraocular lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, precursor T-lymphoblastic lymphoma, adult T-cell leukemia/lymphoma, angioimmunoblastic T-cell lymphoma, extranodal natural killer/T-cell lymphoma, enteropathy-associated intestinal T-cell lymphoma, and anaplastic large cell lymphoma (ALCL).

The term “multiple myeloma” refers to is a cancer of plasma cells, a type of white blood cell that normally produces antibodies. Often, no symptoms are noticed initially. As it progresses, bone pain, anemia, kidney dysfunction, and infections may occur. Complications may include amyloidosis. The cause of multiple myeloma is unknown. Risk factors include obesity, radiation exposure, family history, and certain chemicals. The abnormal plasma cells produce abnormal antibodies, which can cause kidney problems and overly thick blood. The plasma cells can also form a mass in the bone marrow or soft tissue. Multiple myeloma is diagnosed based on blood or urine tests finding abnormal antibodies, bone marrow biopsy finding cancerous plasma cells, and medical imaging finding bone lesions. Another common finding is high blood calcium levels. Multiple myeloma is considered treatable, but generally incurable. Remissions may be brought about with steroids, chemotherapy, targeted therapy, and stem cell transplant. Bisphosphonates and radiation therapy are sometimes used to reduce pain from bone lesions.

The term “myeloproliferative disorder” or “MPD” refers to rare blood cancers that have many different symptoms, yet no clear cause. MPDs include, but are not limited to, chronic myelogenous leukemia, myelofibrosis, polycythemia vera, thrombocytosis, chronic neutrophilic leukemia, and eosinophilia. Chronic myelogenous leukemia (CML) occurs when the bone marrow produces a large number of immature white blood cells. Polycythemia vera is characterized by an increased number of red blood cells, often accompanied by an excess of platelets and white blood cells. The web of fibers inside the bone marrow becomes thick, like scar tissue. This causes fewer and fewer red blood cells to be made. Myelofibrosis occurs when the bone marrow produces many immature white and red blood cells. Thrombocythemia occurs when too many platelets are produced. In chronic neutrophilic leukemia the blood has an excess of neutrophils. In eosinophilia the blood has an excess of eosinophils. Any of these disorders can also lead to acute leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “glioblastoma” or “glioblastoma multiforte” refers to an aggressive malignant brain tumor that develops in the brain or spinal cord from astrocytes. The term “glioma” refers to a malignant brain tumor that develops in the brain or spinal cord from glial cells.

The term “myelodysplastic syndrome” refers to a group of disorders resulting from poorly formed or dysfunctional blood cells. Conditions related to myelodysplastic syndrome include but are not limited to acute myeloid leukemia, myeloproliferative neoplasm, multiple myeloma, myelofibrosis, sideroblastic anemia, chronic myeloid leukemia, and leukemia. The term “acute myeloid leukemia” refers to a type of blood and bone marrow cancer which effects white blood cells. The term “myeloproliferative neoplasm” refers to a group of rare blood cancers in which excess red blood cells, white blood cells or platelets are produced in the bone marrow. The term “multiple myeloma” refers to cancer of mature plasma cells in the bone marrow. The term “myelofibrosis” refers to a bone marrow disorder in which excessive scar tissue forms in the bone marrow and disrupts body's normal production of blood cells. The term “sideroblastic anemia” refers to a form of anemia in which the bone marrow produces ringed erythrocytes due to iron accumulation in their nucleus. The term “chronic myeloid leukemia” refers to a type of white blood cancer that is caused due to an acquired genetic defect. The term “leukemia” refers to a cancer that affects production and function of blood cells.

The terms “anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL₂), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. TLX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin Al (i.e. BTO-956 and DAIE), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” does not include preventing.

The terms “patient,” “patient in need thereof,” “subject,” or “subject in need thereof” refers to a living organism suffering from or prone to a disease or condition that can be treated by administration of a pharmaceutical composition as provided herein. Non-limiting examples include humans, other mammals, bovines, rats, mice, dogs, cats, monkeys, and other non-mammalian animals. In some embodiments, a patient is human. In embodiments, a patient in need thereof is human. In embodiments, a subject is human. In embodiments, a subject in need thereof is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art. Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

The term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In embodiments, the administering includes simultaneous or sequential administration of another active agent in addition to the recited active agents.

The term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The terms “specific”, “specifically”, “specificity”, or the like of a compound refers to the compound's ability to cause a particular action, such as inhibition, to a particular molecular target with minimal or no action to other proteins in the cell.

“Nucleic acid” refers to deoxyribonucleotides or ribonucleotides and polymers thereof in either single- or double-stranded forms, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

The term “nucleotide” typically refers to a compound containing a nucleoside or a nucleoside analogue and at least one phosphate group or a modified phosphate group linked to it by a covalent bond. Exemplary covalent bonds include, without limitation, an ester bond between the 3′, 2′ or 5′ hydroxyl group of a nucleoside and a phosphate group.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of nucleic acid molecules may be detected by standard PCR or Northern blot methods well known in the art. See, Sambrook et al., 1989 Molecular Cloning: A Laboratory Manual, 18.1-18.88. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

A “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The term “amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An “antisense nucleic acid” as referred to herein is a nucleic acid (e.g. DNA or RNA molecule) that is complementary to at least a portion of a specific target nucleic acid (e.g. an mRNA translatable into a protein) and is typically capable of reducing transcription of the target nucleic acid (e.g. mRNA from DNA) or reducing the translation or the amount of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone modified nucleotides. In the cell, the antisense nucleic acids may hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem. 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogues), short hairpin RNAs (shRNA), micro RNAs (miRNA), saRNAs (small activating RNAs) and small nucleolar RNAs (snoRNA) or certain of their derivatives or pre-cursors.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

A “saRNA,” or “small activating RNA” as provided herein refers to a nucleic acid that forms a double stranded RNA, which double stranded RNA has the ability to increase or activate expression of a gene or target gene when present in the same cell as the gene or target gene. The complementary portions of the nucleic acid that hybridize to form the double stranded molecule typically have substantial or complete identity. In one embodiment, a saRNA is a nucleic acid that has substantial or complete identity to a target gene and forms a double stranded saRNA. Typically, the nucleic acid is at least about 15-50 nucleotides in length (e.g., each complementary sequence of the double stranded saRNA is 15-50 nucleotides in length, and the double stranded saRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

A “shRNA,” “short hairpin RNA,” or “small hairpin RNA” as provided herein refers to an RNA molecule including a hairpin turn that has the ability to reduce or inhibit expression of a target gene or target nucleic acid when expressed in the same cell as the target gene or target nucleic acid. shRNA expression in a cell may be accomplished by delivery of the shRNA the cell using a plasmid or vector. Typically, the shRNA is cleaved by an enzyme (i.e. Dicer) to produce an siRNA product. The siRNA may then associate with RISC, thereby allowing target recognition.

A “PIWI-interacting RNA” or “piRNA” refers to a type of small non-coding RNA (sncRNA), which is 26-31 nucleotides in length and binds to PIWI proteins. Their features include the following characteristics: piRNAs are independent of the Dicer enzyme and are produced by a single-stranded precursor. The majority of piRNA clusters in somatic cells are unidirectional, whereas the majority of germline piRNA clusters are dual-stranded. Most mature primary piRNAs contain uridine at the 5′ end, and the 3′ ends of piRNAs are uniquely methylated 2-OH structures. piRNAs are unevenly distributed among various genomic sequences, including exons, introns, and repeat sequences. piRNAs are derived from transposons and from flanking genomic sequences. piRNAs are not degraded in circulation and are stably expressed in body fluids.

PIWI proteins are mainly expressed in the germline and human tumors. The human PIWI protein subfamily consists of PIWIL1, PIWIL2, PIWIL3 and PIWIL4. piRNAs are essential in many stages of spermatogenesis, and PIWIs are necessary to maintain the function of reproductive system stem cells. piRNAs interact with PIWI subfamily proteins, resulting in the development of the piRNA-induced silencing complex (piRISC), which detects and silences complementary sequences at the transcriptional (TGS) and post-transcriptional (PTGS) levels. The absence of piRNAs can lead to pathogenic effects in the reproductive system, such as birth defects and infertility piRNAs are thought to be essential regulators for germline preservation, and they can also influence gene expression in somatic cells. Dysregulation of piRNAs can both promote and repress the emergence and progression of human cancers through DNA methylation, transcriptional silencing, mRNA turnover, and translational control. piRNAs control the expression of essential genes and pathways associated with digestive cancer progression and have been reported as possible biomarkers for the diagnosis and treatment of digestive cancer.

A “gapmeR” as provided herein refers to a short DNA antisense oligonucleotide flanked by strands of RNA mimics. The mimics are typically composed of locked nucleic acids (LNAs), 2′-OMe, or 2′-F modified bases. LNA sequences are RNA analogues “locked” into an ideal Watson-Crick base pairing conformation. LNAs, 2′-OMe, or 2′-F modified bases are chemical analogs of natural RNA nucleic acids and allow for an increase in nuclease resistance, reduced immunogenicity, and a decrease in toxicity. Gapmers can also have a high binding affinity to the target mRNA. This high binding affinity reduces off-target effects, non-specific binding, and unwanted gene silencing. GapmeRs often utilize nucleotides modified with phosphorothioate (PS) groups. In humans, the gapmer DNA-mRNA duplex is degraded by RNase H. The degradation of the mRNA prevents protein synthesis. GapmeRs are designed to hybridize to a target RNA sequence and silence the gene through the induction of RNase H cleavage. Binding of the gapmer to the target has a higher affinity due to the modified RNA flanking regions, as well as resistance to degradation by nucleases. GapmeRs are currently being developed as therapeutics for a variety of cancers, viruses, and other chronic genetic disorders.

A “morpholinooligonucleotide,” “morpholino oligonucleotide,” “mporpholino,” “morpholino oligomer,” or “morpholino oligo” as used herein refers to synthetic antisense oligonucleotide of about 25 nucleotides in length designed to bind and block the translation initiation complex of messenger RNA (mRNA) sequences. Morpholinooligonucleotides contain DNA bases attached to a backbone of methylenemorpholine rings linked through phosphorodiamidate groups. Morpholinos act by “steric blocking”, binding to a target sequence within an RNA molecule, thereby inhibiting molecules that might otherwise interact with the RNA. By sterically blocking the translation initiation complex, morpholinos can knock down expression of many target sequences. Unlike many antisense types (e.g. siRNA, phosphorothioates), morpholinos generally do not cause degradation of their RNA targets; instead, they block the biological activity of the target RNA until that RNA is degraded naturally, which releases the morpholino. In addition, morpholinos can be used to modify and control normal splicing events by blocking sites involved in splicing pre-mRNA. Morpholinos must be actively delivered into most cells by a variety of methods, including scrape-loading of adherent cells, electroporation, and microinjection. A Morpholino oligo is radically different from natural nucleic acids, with methylenemorpholine rings replacing the ribose or deoxyribose sugar moieties and non-ionic phosphorodiamidate linkages replacing the anionic phosphates of DNA and RNA. Each morpholine ring suitably positions one of the standard DNA bases (A, C, G, T) for pairing, so that a 25-base morpholino oligo strongly and specifically binds to its complementary 25-base target site in a strand of RNA via Watson-Crick pairing. Because the uncharged backbone of the morpholino oligo is not recognized by enzymes, it is completely stable to nucleases.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In embodiments, the polynucleotide (e.g., gRNA) is a single-stranded ribonucleic acid. In aspects, the polynucleotide (e.g., gRNA) is from about 10 to about 200 nucleic acid residues in length. In aspects, the polynucleotide (e.g., gRNA) is from about 50 to about 150 nucleic acid residues in length. In aspects, the polynucleotide (e.g., gRNA) is from about 80 to about 140 nucleic acid residues in length. In aspects, the polynucleotide (e.g., gRNA) is from about 90 to about 130 nucleic acid residues in length. In aspects, the polynucleotide (e.g., gRNA) is from about 100 to about 120 nucleic acid residues in length. In aspects, the length of the polynucleotide (e.g., gRNA) is about 113 nucleic acid residues in length.

In general, a guide sequence (i.e., a DNA-targeting sequence) is any polynucleotide sequence having sufficient complementarity with a target polynucleotide sequence to hybridize with the target sequence (e.g., a genomic or mitochondrial DNA target sequence) and direct sequence-specific binding of a complex (e.g., CRISPR complex) to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In aspects, the degree of complementarity between a guide sequence and its corresponding target sequence, when optimally aligned using a suitable alignment algorithm, is at least about 80%, 85%, 90%, 95%, or 100%. In aspects, the degree of complementarity is at least 90%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In aspects, a guide sequence is about or more than about 10, 20, 30, 35, 40, 45, 50, 75, or more nucleotides in length. In aspects, a guide sequence is about 10 to about 150, about 15 to about 100 nucleotides in length. In aspects, a guide sequence is less than about 75, 50, 45, 40, 35, 30, 25, 20, 15, 12, or fewer nucleotides in length. In aspects, the guide sequence is about or more than about 20 nucleotides in length. The ability of a guide sequence to direct sequence-specific binding of a complex (e.g., CRISPR complex) to a target sequence may be assessed by any suitable assay. For example, the components of a CRISPR system sufficient to form a complex (e.g., CRISPR complex), including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a complex (e.g., CRISPR complex), including the guide sequence to be tested and a control guide sequence different from the test guide sequence, and comparing binding or rate of cleavage at the target sequence between the test and control guide sequence reactions. Other assays are possible, and will occur to those skilled in the art. The terms “sgRNA,” “single guide RNA,” and “single guide RNA sequence” are used interchangeably and refer to the polynucleotide sequence including the crRNA sequence and optionally the tracrRNA sequence. The crRNA sequence includes a guide sequence (i.e., “guide” or “spacer”) and a tracr mate sequence (i.e., direct repeat(s)”). The term “guide sequence” refers to the sequence that specifies the target site. In aspects, the two RNA can be encoded separately by a crRNA and tracrRNA as 2 RNA molecules which then form an RNA/RNA complex due to complementary base pairing between the crRNA and tracrRNA (i.e., before being competent to bind to nuclease-deficient RNA-guided DNA endonuclease enzyme). In aspects, a first nucleic acid includes a tracrRNA sequence, and a separate second nucleic acid includes a gRNA sequence lacking a tracrRNA sequence. In aspects, the first nucleic acid including the tracrRNA sequence and the second nucleic acid including the gRNA sequence interact with one another, and optionally are included in a complex (e.g., CRISPR complex).

In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence. In general, degree of complementarity is with reference to the optimal alignment of the tracr mate sequence and tracrRNA sequence, along the length of the shorter of the two sequences. Optimal alignment may be determined by any suitable alignment algorithm, and may further account for secondary structures, such as self-complementarity within either the tracrRNA sequence or tracr mate sequence. In aspects, the degree of complementarity between the tracrRNA sequence and tracr mate sequence along the length of the shorter of the two when optimally aligned is about or more than about 25%, 30%, 40%, 50%, 60%, 70%, 80%, 90%, 95%, 97.5%, 99%, or higher. In aspects, the tracrRNA sequence is about or more than about 5, 10, 15, 20, 30, 40, 50, or more nucleotides in length. In aspects, the tracrRNA sequence and tracr mate sequence are contained within a single transcript, such that hybridization between the two produces a transcript having a secondary structure, such as a hairpin.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

The term “nuclease-deficient RNA-guided DNA endonuclease enzyme” and the like refer, in the usual and customary sense, to an RNA-guided DNA endonuclease (e.g. a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., sgRNA), but lacks significant endonuclease activity (e.g. any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCas12a, dCpf1, ddCpf1, Cas-phi, a nuclease-deficient Cas9 variant, a nuclease-deficient Class II CRISPR endonuclease, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain, a winged helix domain, a helix-turn-helix motif, a helix-loop-helix domain, an H1 MB-box domain, a Wor3 domain, an OB-fold domain, an immunoglobulin domain, or a B3 domain. In aspects, the nuclease-deficient RNA-guided DNA endonuclease enzyme is a leucine zipper domain. In aspects, the nuclease-deficient RNA-guided DNA endonuclease enzyme is a winged helix domain. In aspects, the nuclease-deficient RNA-guided DNA endonuclease enzyme is a helix-turn-helix motif In aspects, the nuclease-deficient RNA-guided DNA endonuclease enzyme is a helix-loop-helix domain. In aspects, the nuclease-deficient RNA-guided DNA endonuclease enzyme is an H1 MB-box domain. In aspects, the nuclease-deficient RNA-guided DNA endonuclease enzyme is a Wor3 domain. In aspects, the nuclease-deficient RNA-guided DNA endonuclease enzyme is an GB-fold domain. In aspects, the nuclease-deficient RNA-guided DNA endonuclease enzyme is an immunoglobulin domain. In aspects, the nuclease-deficient RNA-guided DNA endonuclease enzyme is a B3 domain. In aspects, the nuclease-deficient RNA-guided DNA endonuclease enzyme is dCas9, dCas12a, ddCpf1, Cas-phi, a nuclease-deficient Cas9 variant, or a nuclease-deficient Class II CRISPR endonuclease. In aspects, the nuclease-deficient RNA-guided DNA endonuclease enzyme is dCas9. In aspects, the nuclease-deficient RNA-guided DNA endonuclease enzyme is dCas9 from S. pyogenes. In aspects, the nuclease-deficient RNA-guided DNA endonuclease enzyme is dCas9 from S. aureus. In aspects, the nuclease-deficient RNA-guided DNA endonuclease enzyme is dCas12a. In aspects, the nuclease-deficient RNA-guided DNA endonuclease enzyme is dCas12a from Lachnospiraceae bacterium. In aspects, the nuclease-deficient RNA-guided DNA endonuclease enzyme is dCas12. In aspects, the nuclease-deficient RNA-guided DNA endonuclease enzyme is ddCas12a. In aspects, the nuclease-deficient RNA-guided DNA endonuclease enzyme is Cas-phi.

The term “CRISPR-associated protein” or “CRISPR protein” refers to any CRISPR protein that functions as a nuclease-deficient RNA-guided DNA endonuclease enzyme, i.e., a CRISPR protein in which catalytic sites for endonuclease activity are defective or lack activity. Exemplary CRISPR proteins include dCas9, dCpf1, ddCpf1, dCas12, ddCas12, dCas12a Cas-phi, a nuclease-deficient Cas9 variant, a nuclease-deficient Class II CRISPR endonuclease, and the like.

The term “nuclease-deficient DNA endonuclease enzyme” refers to a DNA endonuclease (e.g. a mutated form of a naturally occurring DNA endonuclease) that targets a specific phosphodiester bond within a DNA polynucleotide, but that does not require an RNA guide. In embodiments, the “nuclease-deficient DNA endonuclease enzyme” is a zinc finger domain or a transcription activator-like effector (TALE).

In embodiments, the nuclease-deficient RNA-guided DNA endonuclease enzyme is dCas9. The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

In embodiments, the nuclease-deficient RNA-guided DNA endonuclease enzyme is a nuclease-deficient Cas9 variant. The term “nuclease-deficient Cas9 variant” refers to a Cas9 protein having one or more mutations that increase its binding specificity to PAM compared to wild type Cas9 and further include mutations that render the protein incapable of or having severely impaired endonuclease activity. Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). The binding specificity of nuclease-deficient Cas9 variants to PAM can be determined by any method known in the art. Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8, 2017, which are incorporated herein by reference in their entirety and for all purposes.

In embodiments, the nuclease-deficient RNA-guided DNA endonuclease enzyme is a nuclease-deficient Class II CRISPR endonuclease. The term “nuclease-deficient Class II CRISPR endonuclease” as used herein refers to any Class II CRISPR endonuclease having mutations resulting in reduced, impaired, or inactive endonuclease activity.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature. Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs. The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable light chain provided herein includes in N-terminal to C-terminal direction a CDR L1, a CDR L2 and a CDR L3.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

The term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

“Pharmaceutically acceptable excipient” and “pharmaceutically acceptable carrier” refer to a substance that aids the administration of an active agent to and absorption by a subject and can be included in the compositions described herein without causing a significant adverse toxicological effects on the subject. In embodiments, the pharmaceutically acceptable excipient include one or more pharmaceutically acceptable additives. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that additional pharmaceutical excipients may be useful. The term “pharmaceutically acceptable salt” refers to salts derived from a variety of organic and inorganic counter ions well known in the art and include, by way of example only, sodium, potassium, calcium, magnesium, ammonium, tetraalkylammonium, and the like.

A “detectable agent” or “detectable moiety” is a compound or composition detectable by appropriate means such as spectroscopic, photochemical, biochemical, immunochemical, chemical, magnetic resonance imaging, or other physical means. The RNA described herein and the expression level of the RNA described herein may be accomplished through the use of a detectable moiety in an assay or kit. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

The terms “protein arginine methyltransferase,” “PRMT,” “protein arginine methyltransferases,” and “PRMTs” are used in accordance with their plain and ordinary meanings and generally refers to a family of methyltransferase proteins characterized by the presence of a conserved core methyltransferase domain, including S-adenosyl-1-methionine (AdoMet)-binding sequences in motif I and post-motif I, and substrate-binding sequences in motif II, post-motif II (including the double E loop), and the THW loop. The mammalian family of PRMTs may include three groups based on their activities. Type I PRMTs (PRMT1-4, PRMT6, and PRMT8) transfer up to two methyl groups on the same terminal guanidino nitrogen of arginine to form ω-NG-monomethylarginine (MMA) and ω-NG,NG-asymmetric dimethylarginine (ADMA) residues. Type II PRMTs (PRMT5 and PRMT9) transfer methyl groups on different terminal guanidino nitrogen atoms to form MMA and ω-NG,N′G-symmetric dimethylarginine (SDMA) residues. Type III enzymes transfer only a single methyl group to form MMA residues (PRMT7). Both histone and non-histone proteins have been identified as PRMT substrates. Dysregulation of PRMT1, the major asymmetric dimethylating enzyme, may result in a variety of cancers, including breast, colon, bladder, lung, and various leukemias. In embodiments, overexpression of a PRMT increases cancer development (particularly breast cancer) through increased cellular proliferation, conferred resistance to DNA-damaging agents, and increased cell invasion. In embodiments, the PRMT modulates (e.g. increases) transcriptional regulation, signal transduction, nuclear/cytoplasmic shuttling, DNA repair, mRNA splicing, and/or male germ line gene imprinting.

The term “protein arginine methyltransferase 9” or “PRMT9” refers to an arginine methyltransferase that catalyzes the monomethylation (MMA) or symmetrical dimethylation (SDMA) of arginine residues in target proteins. In embodiments, the PRMT9 regulates alternative splicing of pre-mRNA. In embodiments, PRMT9 has two distinguishing features: an AdoMet-binding motif, and three N-terminal tetratricopeptide repeats (TPR), a hallmark amino acid sequence motif belonging to the PRMT family. In embodiments, PRMT9 is highly expressed in several types of cancer, including melanoma, testicular, pancreatic and lymphoma cancer. In embodiments, PRMT9 is found in a cellular complex with the splicing factors SF3B2 and SF3B4, also known as SAP145 and SAP49, respectively. Splicing factor SF3B2 is typically highly conserved in nature, may bind SF3B4 in a tight complex, and it may have functional roles in cell cycle progression.

The term “poly(A)-binding protein 1” or “PABP1” refers to a protein that simultaneously binds the mRNA 3′ poly(A) tail and interacts with the translation factor eIF (eukaryotic initiation factor) 4G, which is part of the cap-binding complex eIF4F (eIF4E, eIF4G and eIF4A). In embodiments, PABP1 brings the ends of the mRNA into functional proximity. In embodiments, the ‘closed-loop’ conformation of the mRNA enhances translation initiation by increasing ribosomal recruitment. In embodiments, PABP1 protects the mRNA from deadenylation, decapping and degradation. In embodiments, PABP1 is located in the nucleus (PABPN1). In embodiments, PABP1 is located in the cytoplasm (PABPC1). In embodiments, the poly(A)-binding protein complexed to the 3′ poly(A) tail of eukaryotic mRNA is required for poly(A) lengthening and the termination of translation. In embodiments, PABPC1 is concentrated at sites of high mRNA concentration. In embodiments, the sites of high mRNA concentration include stress granules, processing bodies, and locations of high translational activity. In embodiments, PABPC1 is associated with nonsense-mediated mRNA decay (NMD). In embodiments, PABPN1 binds to the poly(A) tails of pre-mRNAs to facilitate stability, export, transport, and degradation. In embodiments, PABPC1 contains four RNA-recognition motifs (RRMs). In embodiments, RRM1 and RRM2 bind both α-importin and the poly(A) tail of processed mRNA, thereby preventing mRNA from going back into the nucleus. In embodiments, PABP1 comprises four non-identical RRMs (RNA recognition motifs), a proline-rich region and a PABC (PABP C-terminal domain) (also known as MLLE). In embodiments, RRMs 1 and 2 bind eIF4G and PAIP1. In embodiments, RRMs 3 and 4 bind poly(A) with reduced affinity. In embodiments, RRMs 3 and 4 bind AU-rich RNA and mediate protein-protein interactions. In embodiments, the PABC domain interacts with PAM (PABP-interacting motif) 2 motif-containing proteins, e.g. eRF3, PAIP1, the negative regulator of translation PAIP2 and the PAN [poly(A) nuclease] 3 deadenylase. In embodiments, the PABC domain interacts with non-PAM2-containing proteins, e.g. the miRNA-pathway protein GW182.

The term “cyclic GMP-AMP synthase” or “cGAS” refers to a 520 amino acid protein consisting of a two-lobed catalytic domain and an extended amino-terminal (N-terminal) domain. In embodiments, cGAS recognizes and is activated by DNA ligands (canonically single-stranded or double-stranded molecules longer than 40 bp). In embodiments, cGAS assembles into a dimer, in which both cGAS protomers are bound to their own DNA ligand, with the DNA strands sandwiched between the two cGAS protomers. In embodiments, each protomer has two principal DNA binding sites, A and B. In embodiments, in the dimer, the DNA molecules are bound to site A of one cGAS protomer and to site B of the respective other protomer. In embodiments, active cGAS produces the cyclic dinucleotide 2′,3′-cyclic GMP-AMP (2′,3′-cGAMP). In embodiments, cGAMP binds to stimulator of interferon genes (STING), leading to TANK-binding kinase 1 (TBK1)-dependent phosphorylation (P) of interferon regulatory factor 3 (IRF3). In embodiments, the active TRF3 dimer translocates to the nucleus and activates transcription of type I interferon genes. In embodiments, cGAS-STING signalling leads to the expression of genes for pro-inflammatory cytokines through NF-κB. In embodiments, the cGAS-STING axis is activated by non-self DNA. In embodiments, the non-self DNA is DNA from DNA viruses or retroviruses, intracellular bacteria and protozoa. In embodiments, the cGAS-STING axis is activated by extracellular, mitochondrial and nuclear DNA that gain access to the cytosol. In embodiments, the cGAS-STING axis is activated by increased cytosolic DNA levels. In embodiments, cytosolic DNA Levels are increased by mitotic stress in cancers, radiation therapy, cellular senescence, and in autoimmune disorders. In embodiments, constitutive and systemic activation of cGAS-STING results in chronic inflammation and pathology. In embodiments, cGAS is localized at the cell membrane and in the nucleus.

The singular forms “a,” “an,” and “the” include plural referents unless the context clearly dictates otherwise. The term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Compounds

Provided herein are compounds that are effective in treating cancer. In embodiments, the compound is a compound having formulae (I), (II), or (III):

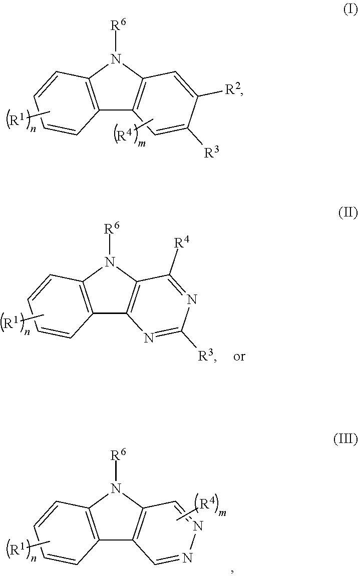

or a pharmaceutically acceptable salt thereof, wherein: R¹ is halogen, —CX¹₃, —CHX¹₂, —CH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, halogen, —CX²₃, —CHX²₂, —CH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}R^{2C}CNOR^{2D}, —NR^{2B}C(O)OR^{2D}, —NR^{2B}OR^{2D}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is hydrogen, halogen, —CX³₃, —CHX³₂, —CH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}R^{3C}CNOR^{3D}, NR^{3B}C(O)OR^{3D}, —NR^{3B}OR^{3D}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R² and R³ are optionally joined to form a substituted or unsubstituted cycloalkyl, substituted or unsubstituted heterocycloalkyl, substituted or unsubstituted aryl or substituted or unsubstituted heteroaryl; R⁴ is halogen, —CX⁴₃, —CHX⁴₂, —CH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}R^{4C}CNOR^{4D}, —NR^{4B}C(O)OR^{4D}, —NR^{4B}OR^{4D}, —OCX⁴₃, —OCHX⁴₂, —OCH₂X⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, and R^{4D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C}, R^{2B} and R^{2C}, R^{3B} and R^{3C}, R^{4B} and R^{4C}, and R^{5B} and R^{5C} substituents may optionally be joined to form a substituted or unsubstituted heterocycloalkyl or substituted or unsubstituted heteroaryl; n1, n2, n3, and n4 is an integer from 0 to 4; m1, m2, m3, m4, v1, v2, v3, and v4 are independently 1 or 2; X¹, X², X³, and X⁴ are independently halogen; n is an integer from 1 to 4; and m is an integer from 1 to 2.

In embodiments, the compound is a compound having formula (Ia):

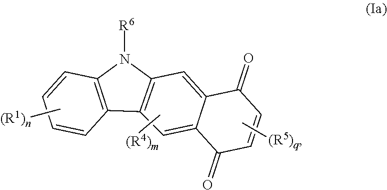

or a pharmaceutically acceptable salt thereof. In embodiments, variables n, m, R¹, R⁴, and R⁶ are described herein, including embodiments wherein:

R⁵ is halogen, —CX⁵₃, —CHX⁵₂, —CH₂X⁵, —OCX⁵₃, —OCHX⁵₂, —OCH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}R^{5C}CNOR^{5D}, —NR^{5B}C(O)OR^{5D}, —NR^{5B}OR^{5D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{5A}, R^{5B}, R^{5C}, and R^{5D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; n5 is an integer from 0 to 4; m5 and v5 are independently 1 or 2; X⁵ is halogen, and q is an integer from 1 to 2.

In embodiments, the compound is a compound having formula (Ib):

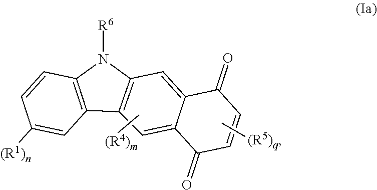

or a pharmaceutically acceptable salt thereof. In embodiments, variables n, m, R¹, R⁴, R⁵, and R⁶ are described herein, including embodiments.

In embodiments, R¹ is halogen, —CX¹₃, —CHX¹₂, —CH₂X¹, —OCX¹₃, —OCHX¹₂, —OCH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is halogen (e.g., —F, —Cl, Br, —I), —CX¹₃, —CHX¹₂, —CH₂X¹, —OCX¹₃, —OCHX¹₂, —OCH₂X¹, —CN, —S(O)₂R^{1A}, —SR^{1A}, —S(O)R^{1A}, —SO₂NR^{1A}R^{1B}, —NHC(O)NR^{1A}R^{1B}, —N(O)₂, —NR^{1A}R^{1B}, —NR^{1A}R^{1B}, —C(O)R^{1A}, —C(O)—OR^{1A}, —C(O)NR^{1A}R^{1B}, —C(O)NHNR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —N₃, (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X¹ is independently —F, —Cl, —Br, or —I.

In embodiments, R¹ is —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R¹ is —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R¹ is halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R¹¹-substituted or unsubstituted alkyl (e.g., C₁-C₈ alkyl, C₁-C₆ alkyl, or C₁-C₄ alkyl), R¹¹-substituted or unsubstituted heteroalkyl (e.g., 2 to 8 membered heteroalkyl, 2 to 6 membered heteroalkyl, or 2 to 4 membered heteroalkyl), R¹¹-substituted or unsubstituted cycloalkyl (e.g., C₃-C₈ cycloalkyl, C₃-C₆ cycloalkyl, or C₅-C₆ cycloalkyl), R¹¹-substituted or unsubstituted heterocycloalkyl (e.g., 3 to 8 membered heterocycloalkyl, 3 to 6 membered heterocycloalkyl, or 5 to 6 membered heterocycloalkyl), R¹¹-substituted or unsubstituted aryl (e.g., C₆-C₁₀ aryl, C₁₀ aryl, or phenyl), or R¹¹-substituted or unsubstituted heteroaryl (e.g., 5 to 10 membered heteroaryl, 5 to 9 membered heteroaryl, or 5 to 6 membered heteroaryl).

In embodiments, R¹ is halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R¹ is R¹¹-substituted or unsubstituted alkyl (e.g., C₁-C₈ alkyl, C₁-C₆ alkyl, or C₁-C₄ alkyl). In embodiments, R¹ is R¹¹-substituted alkyl (e.g., C₁-C₈ alkyl, C₁-C₆ alkyl, or C₁-C₄ alkyl). In embodiments, R¹ is an unsubstituted alkyl (e.g., C₁-C₈ alkyl, C₁-C₆ alkyl, or C₁-C₄ alkyl). In embodiments, R¹ is R¹¹-substituted or unsubstituted heteroalkyl (e.g., 2 to 8 membered heteroalkyl, 2 to 6 membered heteroalkyl, or 2 to 4 membered heteroalkyl). In embodiments, R¹ is R¹¹-substituted heteroalkyl (e.g., 2 to 8 membered heteroalkyl, 2 to 6 membered heteroalkyl, or 2 to 4 membered heteroalkyl). In embodiments, R¹ is an unsubstituted heteroalkyl (e.g., 2 to 8 membered heteroalkyl, 2 to 6 membered heteroalkyl, or 2 to 4 membered heteroalkyl). In embodiments, R¹ is R¹¹-substituted or unsubstituted cycloalkyl (e.g., C₃-C₈ cycloalkyl, C₃-C₆ cycloalkyl, or C₅-C₆ cycloalkyl 1). In embodiments, R¹ is R¹¹-substituted cycloalkyl (e.g., C₃-C₈ cycloalkyl, C₃-C₆ cycloalkyl, or C₅-C₆ cycloalkyl). In embodiments, R¹ is an unsubstituted cycloalkyl (e.g., C₃-C₈ cycloalkyl, C₃-C₆ cycloalkyl, or C₅-C₆ cycloalkyl). In embodiments, R¹ is R¹¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is R¹¹-substituted heterocycloalkyl (e.g., 3 to 8 membered heterocycloalkyl, 3 to 6 membered heterocycloalkyl, or 5 to 6 membered heterocycloalkyl). In embodiments, R¹ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹ is R¹¹-substituted or unsubstituted aryl (e.g., C₆-C₁₀ aryl, C₁₀ aryl, or phenyl). In embodiments, R¹ is R¹¹-substituted aryl (e.g., C₆-C₁₀ aryl, C₁₀ aryl, or phenyl). In embodiments, R¹ is an unsubstituted aryl (e.g., C₆-C₁₀ aryl, C₁₀ aryl, or phenyl). In embodiments, R¹ is R¹¹-substituted or unsubstituted heteroaryl (e.g., 5 to 10 membered heteroaryl, 5 to 9 membered heteroaryl, or 5 to 6 membered heteroaryl). In embodiments, R¹ is R¹¹-substituted heteroaryl (e.g., 5 to 10 membered heteroaryl, 5 to 9 membered heteroaryl, or 5 to 6 membered heteroaryl). In embodiments, R¹ is an unsubstituted heteroaryl (e.g., 5 to 10 membered heteroaryl, 5 to 9 membered heteroaryl, or 5 to 6 membered heteroaryl).

In embodiments, R¹¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R¹²-substituted or unsubstituted alkyl (e.g., C₁-C₈ alkyl, C₁-C₆ alkyl, or C₁-C₄ alkyl), R¹²-substituted or unsubstituted heteroalkyl (e.g., 2 to 8 membered heteroalkyl, 2 to 6 membered heteroalkyl, or 2 to 4 membered heteroalkyl), R²-substituted or unsubstituted cycloalkyl (e.g., C₃-C₈ cycloalkyl, C₃-C₆ cycloalkyl, or C₅-C₆ cycloalkyl), R²-substituted or unsubstituted heterocycloalkyl (e.g., 3 to 8 membered heterocycloalkyl, 3 to 6 membered heterocycloalkyl, or 5 to 6 membered heterocycloalkyl), R¹²-substituted or unsubstituted aryl (e.g., C₆-C₁₀ aryl, C₁₀ aryl, or phenyl), or R²-substituted or unsubstituted heteroaryl (e.g., 5 to 10 membered heteroaryl, 5 to 9 membered heteroaryl, or 5 to 6 membered heteroaryl).

In embodiments, R¹¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R¹¹ is R¹²-substituted or unsubstituted alkyl (e.g., C₁-C₈ alkyl, C₁-C₆ alkyl, or C₁-C₄ alkyl). In embodiments, R¹¹ is R¹²-substituted (e.g., C₁-C₈ alkyl, C₁-C₆ alkyl, or C₁-C₄ alkyl). In embodiments, R¹¹ is unsubstituted alkyl (e.g., C₁-C₈ alkyl, C₁-C₆ alkyl, or C₁-C₄ alkyl). In embodiments, R¹¹ is R¹²-substituted or unsubstituted heteroalkyl (e.g., 2 to 8 membered heteroalkyl, 2 to 6 membered heteroalkyl, or 2 to 4 membered heteroalkyl). In embodiments, R¹¹ is R¹²-substituted heteroalkyl (e.g., 2 to 8 membered heteroalkyl, 2 to 6 membered heteroalkyl, or 2 to 4 membered heteroalkyl). In embodiments, R¹¹ is unsubstituted heteroalkyl (e.g., 2 to 8 membered heteroalkyl, 2 to 6 membered heteroalkyl, or 2 to 4 membered heteroalkyl). In embodiments, R¹¹ is R¹²-substituted or unsubstituted cycloalkyl (e.g., C₃-C₈ cycloalkyl, C₃-C₆ cycloalkyl, or C₅-C₆ cycloalkyl). In embodiments, R¹¹ is R¹²-substituted cycloalkyl (e.g., C₃-C₈ cycloalkyl, C₃-C₆ cycloalkyl, or C₅-C₆ cycloalkyl). In embodiments, R¹¹ is unsubstituted cycloalkyl (e.g., C₃-C₈ cycloalkyl, C₃-C₆ cycloalkyl, or C₅-C₆ cycloalkyl). In embodiments, R¹¹ is R¹²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹¹ is R²-substituted heterocycloalkyl (e.g., 3 to 8 membered heterocycloalkyl, 3 to 6 membered heterocycloalkyl, or 5 to 6 membered heterocycloalkyl). In embodiments, R¹¹ is unsubstituted heterocycloalkyl (e.g., 3 to 8 membered heterocycloalkyl, 3 to 6 membered heterocycloalkyl, or 5 to 6 membered heterocycloalkyl). In embodiments, R¹¹ is R¹²-substituted or unsubstituted aryl (e.g., C₆-C₁₀ aryl, C₁₀ aryl, or phenyl). In embodiments, R¹¹ is R²-substituted aryl (e.g., C₆-C₁₀ aryl, C₁₀ aryl, or phenyl). In embodiments, R¹¹ is unsubstituted aryl (e.g., C₆-C₁₀ aryl, C₁₀ aryl, or phenyl). In embodiments, R¹¹ is R¹²-substituted or unsubstituted heteroaryl (e.g., 5 to 10 membered heteroaryl, 5 to 9 membered heteroaryl, or 5 to 6 membered heteroaryl). In embodiments, R¹¹ is R¹²-substituted heteroaryl (e.g., 5 to 10 membered heteroaryl, 5 to 9 membered heteroaryl, or 5 to 6 membered heteroaryl). In embodiments, R¹¹ is unsubstituted heteroaryl (e.g., 5 to 10 membered heteroaryl, 5 to 9 membered heteroaryl, or 5 to 6 membered heteroaryl).

In embodiments, R¹² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R¹³-substituted or unsubstituted alkyl (e.g., C₁-C₈ alkyl, C₁-C₆ alkyl, or C₁-C₄ alkyl), R¹³-substituted or unsubstituted heteroalkyl (e.g., 2 to 8 membered heteroalkyl, 2 to 6 membered heteroalkyl, or 2 to 4 membered heteroalkyl), R¹³-substituted or unsubstituted cycloalkyl (e.g., C₃-C₈ cycloalkyl, C₃-C₆ cycloalkyl, or C₅-C₆ cycloalkyl), R¹³-substituted or unsubstituted heterocycloalkyl (e.g., 3 to 8 membered heterocycloalkyl, 3 to 6 membered heterocycloalkyl, or 5 to 6 membered heterocycloalkyl), R¹³-substituted or unsubstituted aryl (e.g., C₆-C₁₀ aryl, C₁₀ aryl, or phenyl), or R¹³-substituted or unsubstituted heteroaryl (e.g., 5 to 10 membered heteroaryl, 5 to 9 membered heteroaryl, or 5 to 6 membered heteroaryl).

In embodiments, R¹² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R¹² is R¹³-substituted or unsubstituted alkyl (e.g., C₁-C₈ alkyl, C₁-C₆ alkyl, or C₁-C₄ alkyl). In embodiments, R¹² is R¹³-substituted (e.g., C₁-C₈ alkyl, C₁-C₆ alkyl, or C₁-C₄ alkyl). In embodiments, R¹² is unsubstituted alkyl (e.g., C₁-C₈ alkyl, C₁-C₆ alkyl, or C₁-C₄ alkyl). In embodiments, R¹² is R¹³-substituted or unsubstituted heteroalkyl (e.g., 2 to 8 membered heteroalkyl, 2 to 6 membered heteroalkyl, or 2 to 4 membered heteroalkyl). In embodiments, R¹² is R¹³-substituted heteroalkyl (e.g., 2 to 8 membered heteroalkyl, 2 to 6 membered heteroalkyl, or 2 to 4 membered heteroalkyl). In embodiments, R¹² is unsubstituted heteroalkyl (e.g., 2 to 8 membered heteroalkyl, 2 to 6 membered heteroalkyl, or 2 to 4 membered heteroalkyl). In embodiments, R¹² is R¹³-substituted or unsubstituted cycloalkyl (e.g., C₃-C₈ cycloalkyl, C₃-C₆ cycloalkyl, or C₅-C₆ cycloalkyl). In embodiments, R¹² is R¹³-substituted cycloalkyl (e.g., C₃-C₈ cycloalkyl, C₃-C₆ cycloalkyl, or C₅-C₆ cycloalkyl). In embodiments, R¹² is unsubstituted cycloalkyl (e.g., C₃-C₈ cycloalkyl, C₃-C₆ cycloalkyl, or C₅-C₆ cycloalkyl). In embodiments, R¹² is R¹³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹² is R¹³-substituted heterocycloalkyl (e.g., 3 to 8 membered heterocycloalkyl, 3 to 6 membered heterocycloalkyl, or 5 to 6 membered heterocycloalkyl). In embodiments, R¹² is unsubstituted heterocycloalkyl (e.g., 3 to 8 membered heterocycloalkyl, 3 to 6 membered heterocycloalkyl, or 5 to 6 membered heterocycloalkyl). In embodiments, R¹² is R¹³-substituted or unsubstituted aryl (e.g., C₆-C₁₀ aryl, C₁₀ aryl, or phenyl). In embodiments, R¹¹ is R¹³-substituted aryl (e.g., C₆-C₁₀ aryl, C₁₀ aryl, or phenyl). In embodiments, R¹² is unsubstituted aryl (e.g., C₆-C₁₀ aryl, C₁₀ aryl, or phenyl). In embodiments, R¹² is R¹³-substituted or unsubstituted heteroaryl (e.g., 5 to 10 membered heteroaryl, 5 to 9 membered heteroaryl, or 5 to 6 membered heteroaryl). In embodiments, R¹² is R¹³-substituted heteroaryl (e.g., 5 to 10 membered heteroaryl, 5 to 9 membered heteroaryl, or 5 to 6 membered heteroaryl). In embodiments, R¹² is unsubstituted heteroaryl (e.g., 5 to 10 membered heteroaryl, 5 to 9 membered heteroaryl, or 5 to 6 membered heteroaryl).

In embodiments, R¹³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R¹³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R¹³ is unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The definitions of R^{1A}, R^{1B}, R^{1C}, and R^{1D} are the same as the definition of R¹. The definitions of R^{11A}, R^{11B}, R^{11C}, and R^{11D} are the same as the definition of R¹¹. The definitions of R^{12A}, R^{12B}, R^{12C}, and R^{12D} are the same as the definition of R¹². The definitions of R^{13A}, R^{13B}, R^{13C}, and R^{13D} are the same as the definition of R¹³.

In embodiments, R^{1A} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{11A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{11A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{11A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{11A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{11A}-substituted (e.g., substituted with a substituent group, a size-limited substituent group, or lower substituent group) or unsubstituted aryl (e.g., C₆-C₁₀ aryl, C₁₀ aryl, or phenyl), or R^{11A}-substituted or unsubstituted heteroaryl (e.g., 5 to 10 membered heteroaryl, 5 to 9 membered heteroaryl, or 5 to 6 membered heteroaryl).

In embodiments, R^{11A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{12A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{12A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{12A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{12A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{12A}-substituted (e.g., substituted with a substituent group, a size-limited substituent group, or lower substituent group) or unsubstituted aryl (e.g., C₆-C₁₀ aryl, C₁₀ aryl, or phenyl), or R^{12A}-substituted or unsubstituted heteroaryl (e.g., 5 to 10 membered heteroaryl, 5 to 9 membered heteroaryl, or 5 to 6 membered heteroaryl).

In embodiments, R^{12A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{13A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{13A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{13A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{13A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{13A}-substituted (e.g., substituted with a substituent group, a size-limited substituent group, or lower substituent group) or unsubstituted aryl (e.g., C₆-C₁₀ aryl, C₁₀ aryl, or phenyl), or R^{13A}-substituted or unsubstituted heteroaryl (e.g., 5 to 10 membered heteroaryl, 5 to 9 membered heteroaryl, or 5 to 6 membered heteroaryl).

In embodiments, R^{13A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, RB is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{11B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{11B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{11B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{11B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{11B}-substituted (e.g., substituted with a substituent group, a size-limited substituent group, or lower substituent group) or unsubstituted aryl (e.g., C₆-C₁₀ aryl, C₁₀ aryl, or phenyl), or R^{11B}-substituted or unsubstituted heteroaryl (e.g., 5 to 10 membered heteroaryl, 5 to 9 membered heteroaryl, or 5 to 6 membered heteroaryl).

In embodiments, R^{11B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{12B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{12B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{12B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{12B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{12B}-substituted (e.g., substituted with a substituent group, a size-limited substituent group, or lower substituent group) or unsubstituted aryl (e.g., C₆-C₁₀ aryl, C₁₀ aryl, or phenyl), or R^{12B}-substituted or unsubstituted heteroaryl (e.g., 5 to 10 membered heteroaryl, 5 to 9 membered heteroaryl, or 5 to 6 membered heteroaryl).

In embodiments, R^{12B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{13B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{13B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{13B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{13B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{13B}-substituted (e.g., substituted with a substituent group, a size-limited substituent group, or lower substituent group) or unsubstituted aryl (e.g., C₆-C₁₀ aryl, C₁₀ aryl, or phenyl), or R^{13B}-substituted or unsubstituted heteroaryl (e.g., 5 to 10 membered heteroaryl, 5 to 9 membered heteroaryl, or 5 to 6 membered heteroaryl).

In embodiments, R^{13B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{1C} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{11C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{11C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{11C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{11C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{11C}-substituted (e.g., substituted with a substituent group, a size-limited substituent group, or lower substituent group) or unsubstituted aryl (e.g., C₆-C₁₀ aryl, C₁₀ aryl, or phenyl), or R^{11C}-substituted or unsubstituted heteroaryl (e.g., 5 to 10 membered heteroaryl, 5 to 9 membered heteroaryl, or 5 to 6 membered heteroaryl).

In embodiments, R^{11C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{12C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{12C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{12C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{12C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{2C}-substituted (e.g., substituted with a substituent group, a size-limited substituent group, or lower substituent group) or unsubstituted aryl (e.g., C₆-C₁₀ aryl, C₁₀ aryl, or phenyl), or R^{12C}-substituted or unsubstituted heteroaryl (e.g., 5 to 10 membered heteroaryl, 5 to 9 membered heteroaryl, or 5 to 6 membered heteroaryl).

In embodiments, R^{12C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{13C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{13C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{13C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{13C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{13C}-substituted (e.g., substituted with a substituent group, a size-limited substituent group, or lower substituent group) or unsubstituted aryl (e.g., C₆-C₁₀ aryl, C₁₀ aryl, or phenyl), or R^{13C}-substituted or unsubstituted heteroaryl (e.g., 5 to 10 membered heteroaryl, 5 to 9 membered heteroaryl, or 5 to 6 membered heteroaryl).

In embodiments, R^{13C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{1D} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{11D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{11D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{11D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{11D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{11D}-substituted (e.g., substituted with a substituent group, a size-limited substituent group, or lower substituent group) or unsubstituted aryl (e.g., C₆-C₁₀ aryl, C₁₀ aryl, or phenyl), or R^{11D}-substituted or unsubstituted heteroaryl (e.g., 5 to 10 membered heteroaryl, 5 to 9 membered heteroaryl, or 5 to 6 membered heteroaryl).

In embodiments, R^{11D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{12D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{12D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{12D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{12D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{12D}-substituted (e.g., substituted with a substituent group, a size-limited substituent group, or lower substituent group) or unsubstituted aryl (e.g., C₆-C₁₀ aryl, C₁₀ aryl, or phenyl), or R^{12D}-substituted or unsubstituted heteroaryl (e.g., 5 to 10 membered heteroaryl, 5 to 9 membered heteroaryl, or 5 to 6 membered heteroaryl).

In embodiments, R^{12D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{13D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{13D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{13D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{13D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{13D}-substituted (e.g., substituted with a substituent group, a size-limited substituent group, or lower substituent group) or unsubstituted aryl (e.g., C₆-C₁₀ aryl, C₁₀ aryl, or phenyl), or R^{13D}-substituted or unsubstituted heteroaryl (e.g., 5 to 10 membered heteroaryl, 5 to 9 membered heteroaryl, or 5 to 6 membered heteroaryl).

In embodiments, R^{13D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{1B} and R^{1C} substituents may optionally be joined to form a substituted or unsubstituted heterocycloalkyl or substituted or unsubstituted heteroaryl. In embodiments, R^{1B} and R^{1C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl). In embodiments, R^{1B} and R^{1C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R^{1B} and R^{1C} substituents may optionally be joined to form a substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl).

In embodiments, R¹ is halogen, —NH₂, —COOH, —C(O)CH₃, substituted or unsubstituted C₁-C₄ alkyl or substituted or unsubstituted 2 to 4 membered heteroalkyl. In embodiments, R¹ is halogen. In embodiments, R¹ is —F, —Cl, —Br, —I. In embodiments, R¹ is —NH₂. In embodiments, R¹ is —COOH. In embodiments, R¹ is —C(O)CH₃. In embodiments, R¹ is substituted or unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is —OCH₃.

In embodiments, R² is independently hydrogen, halogen, —CX²₃, —CHX²₂, —CH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, —OCX²₃, —OCH₂X², —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently hydrogen, halogen (e.g., —F, —Cl, Br, —I), —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X² is independently —F, —Cl, —Br, or —I.

In embodiments, R² is independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R² is independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R² is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R²¹-substituted or unsubstituted alkyl (e.g., C₁-C₈ alkyl, C₁-C₆ alkyl, or C₁-C₄ alkyl), R²¹-substituted or unsubstituted heteroalkyl (e.g., 2 to 8 membered heteroalkyl, 2 to 6 membered heteroalkyl, or 2 to 4 membered heteroalkyl), R²¹-substituted or unsubstituted cycloalkyl (e.g., C₃-C₈ cycloalkyl, C₃-C₆ cycloalkyl, or C₅-C₆ cycloalkyl), R²¹-substituted or unsubstituted heterocycloalkyl (e.g., 3 to 8 membered heterocycloalkyl, 3 to 6 membered heterocycloalkyl, or 5 to 6 membered heterocycloalkyl), R²¹-substituted or unsubstituted aryl (e.g., C₆-C₁₀ aryl, C₁₀ aryl, or phenyl), or R²¹-substituted or unsubstituted heteroaryl (e.g., 5 to 10 membered heteroaryl, 5 to 9 membered heteroaryl, or 5 to 6 membered heteroaryl).

In embodiments, R² is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R² is R²¹-substituted or unsubstituted alkyl (e.g., C₁-C₈ alkyl, C₁-C₆ alkyl, or C₁-C₄ alkyl). In embodiments, R² is R²¹-substituted alkyl (e.g., C₁-C₈ alkyl, C₁-C₆ alkyl, or C₁-C₄ alkyl). In embodiments, R² is an unsubstituted alkyl (e.g., C₁-C₈ alkyl, C₁-C₆ alkyl, or C₁-C₄ alkyl). In embodiments, R² is R²¹-substituted or unsubstituted heteroalkyl (e.g., 2 to 8 membered heteroalkyl, 2 to 6 membered heteroalkyl, or 2 to 4 membered heteroalkyl). In embodiments, R² is R²¹-substituted heteroalkyl (e.g., 2 to 8 membered heteroalkyl, 2 to 6 membered heteroalkyl, or 2 to 4 membered heteroalkyl). In embodiments, R² is an unsubstituted heteroalkyl (e.g., 2 to 8 membered heteroalkyl, 2 to 6 membered heteroalkyl, or 2 to 4 membered heteroalkyl). In embodiments, R² is R²¹-substituted or unsubstituted cycloalkyl (e.g., C₃-C₈ cycloalkyl, C₃-C₆ cycloalkyl, or C₅-C₆ cycloalkyl 1). In embodiments, R² is R²¹-substituted cycloalkyl (e.g., C₃-C₈ cycloalkyl, C₃-C₆ cycloalkyl, or C₅-C₆ cycloalkyl). In embodiments, R² is an unsubstituted cycloalkyl (e.g., C₃-C₈ cycloalkyl, C₃-C₆ cycloalkyl, or C₅-C₆ cycloalkyl). In embodiments, R² is R²¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R² is R²¹-substituted heterocycloalkyl (e.g., 3 to 8 membered heterocycloalkyl, 3 to 6 membered heterocycloalkyl, or 5 to 6 membered heterocycloalkyl). In embodiments, R² is an unsubstituted heterocycloalkyl (e.g., 3 to 8 membered heterocycloalkyl, 3 to 6 membered heterocycloalkyl, or 5 to 6 membered heterocycloalkyl). In embodiments, R² is R²¹-substituted or unsubstituted aryl (e.g., C₆-C₁₀ aryl, C₁₀ aryl, or phenyl). In embodiments, R² is R²¹-substituted aryl (e.g., C₆-C₁₀ aryl, C₁₀ aryl, or phenyl). In embodiments, R² is an unsubstituted aryl (e.g., C₆-C₁₀ aryl, C₁₀ aryl, or phenyl). In embodiments, R² is R²¹-substituted or unsubstituted heteroaryl (e.g., 5 to 10 membered heteroaryl, 5 to 9 membered heteroaryl, or 5 to 6 membered heteroaryl). In embodiments, R² is R²¹-substituted heteroaryl (e.g., 5 to 10 membered heteroaryl, 5 to 9 membered heteroaryl, or 5 to 6 membered heteroaryl). In embodiments, R² is an unsubstituted heteroaryl (e.g., 5 to 10 membered heteroaryl, 5 to 9 membered heteroaryl, or 5 to 6 membered heteroaryl).

R²¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R²²-substituted or unsubstituted alkyl (e.g., C₁-C₈ alkyl, C₁-C₆ alkyl, or C₁-C₄ alkyl), R²²-substituted or unsubstituted heteroalkyl (e.g., 2 to 8 membered heteroalkyl, 2 to 6 membered heteroalkyl, or 2 to 4 membered heteroalkyl), R²²-substituted or unsubstituted cycloalkyl (e.g., C₃-C₈ cycloalkyl, C₃-C₆ cycloalkyl, or C₅-C₆ cycloalkyl), R²²-substituted or unsubstituted heterocycloalkyl (e.g., 3 to 8 membered heterocycloalkyl, 3 to 6 membered heterocycloalkyl, or 5 to 6 membered heterocycloalkyl), R²²-substituted or unsubstituted aryl (e.g., C₆-C₁₀ aryl, C₁₀ aryl, or phenyl), or R²²-substituted or unsubstituted heteroaryl (e.g., 5 to 10 membered heteroaryl, 5 to 9 membered heteroaryl, or 5 to 6 membered heteroaryl).

In embodiments, R²¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I, R²²-substituted or unsubstituted alkyl (e.g., C₁-C₈ alkyl, C₁-C₆ alkyl, or C₁-C₄ alkyl). In embodiments, R²¹ is R²²-substituted (e.g., C₁-C₈ alkyl, C₁-C₆ alkyl, or C₁-C₄ alkyl). In embodiments, R²¹ is unsubstituted alkyl (e.g., C₁-C₈ alkyl, C₁-C₆ alkyl, or C₁-C₄ alkyl). In embodiments, R²¹ is R²²-substituted or unsubstituted heteroalkyl (e.g., 2 to 8 membered heteroalkyl, 2 to 6 membered heteroalkyl, or 2 to 4 membered heteroalkyl). In embodiments, R²¹ is R²²-substituted heteroalkyl (e.g., 2 to 8 membered heteroalkyl, 2 to 6 membered heteroalkyl, or 2 to 4 membered heteroalkyl). In embodiments, R²¹ is unsubstituted heteroalkyl (e.g., 2 to 8 membered heteroalkyl, 2 to 6 membered heteroalkyl, or 2 to 4 membered heteroalkyl). In embodiments, R²¹ is R²²-substituted or unsubstituted cycloalkyl (e.g., C₃-C₈ cycloalkyl, C₃-C₆ cycloalkyl, or C₅-C₆ cycloalkyl). In embodiments, R²¹ is R²²-substituted cycloalkyl (e.g., C₃-C₈ cycloalkyl, C₃-C₆ cycloalkyl, or C₅-C₆ cycloalkyl). In embodiments, R²¹ is unsubstituted cycloalkyl (e.g., C₃-C₈ cycloalkyl, C₃-C₆ cycloalkyl, or C₅-C₆ cycloalkyl). In embodiments, R²¹ is R²²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²¹ is R²²-substituted heterocycloalkyl (e.g., 3 to 8 membered heterocycloalkyl, 3 to 6 membered heterocycloalkyl, or 5 to 6 membered heterocycloalkyl). In embodiments, R²¹ is unsubstituted heterocycloalkyl (e.g., 3 to 8 membered heterocycloalkyl, 3 to 6 membered heterocycloalkyl, or 5 to 6 membered heterocycloalkyl). In embodiments, R²¹ is R²²-substituted or unsubstituted aryl (e.g., C₆-C₁₀ aryl, C₁₀ aryl, or phenyl). In embodiments, R²¹ is R²²-substituted aryl (e.g., C₆-C₁₀ aryl, C₁₀ aryl, or phenyl). In embodiments, R²¹ is unsubstituted aryl (e.g., C₆-C₁₀ aryl, C₁₀ aryl, or phenyl). In embodiments, R²¹ is R²²-substituted or unsubstituted heteroaryl (e.g., 5 to 10 membered heteroaryl, 5 to 9 membered heteroaryl, or 5 to 6 membered heteroaryl). In embodiments, R²¹ is R²²-substituted heteroaryl (e.g., 5 to 10 membered heteroaryl, 5 to 9 membered heteroaryl, or 5 to 6 membered heteroaryl). In embodiments, R²¹ is unsubstituted heteroaryl (e.g., 5 to 10 membered heteroaryl, 5 to 9 membered heteroaryl, or 5 to 6 membered heteroaryl).

In embodiments, R²² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R²³-substituted or unsubstituted alkyl (e.g., C₁-C₈ alkyl, C₁-C₆ alkyl, or C₁-C₄ alkyl), R²³-substituted or unsubstituted heteroalkyl (e.g., 2 to 8 membered heteroalkyl, 2 to 6 membered heteroalkyl, or 2 to 4 membered heteroalkyl), R²³-substituted or unsubstituted cycloalkyl (e.g., C₃-C₈ cycloalkyl, C₃-C₆ cycloalkyl, or C₅-C₆ cycloalkyl), R²³-substituted or unsubstituted heterocycloalkyl (e.g., 3 to 8 membered heterocycloalkyl, 3 to 6 membered heterocycloalkyl, or 5 to 6 membered heterocycloalkyl), R²³-substituted or unsubstituted aryl (e.g., C₆-C₁₀ aryl, C₁₀ aryl, or phenyl), or R²³-substituted or unsubstituted heteroaryl (e.g., 5 to 10 membered heteroaryl, 5 to 9 membered heteroaryl, or 5 to 6 membered heteroaryl).

In embodiments, R²² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R²² is R²³-substituted or unsubstituted alkyl (e.g., C₁-C₈ alkyl, C₁-C₆ alkyl, or C₁-C₄ alkyl). In embodiments, R²² is R²³-substituted (e.g., C₁-C₈ alkyl, C₁-C₆ alkyl, or C₁-C₄ alkyl). In embodiments, R²² is unsubstituted alkyl (e.g., C₁-C₈ alkyl, C₁-C₆ alkyl, or C₁-C₄ alkyl). In embodiments, R²² is R²³-substituted or unsubstituted heteroalkyl (e.g., 2 to 8 membered heteroalkyl, 2 to 6 membered heteroalkyl, or 2 to 4 membered heteroalkyl). In embodiments, R²² is R²³-substituted heteroalkyl (e.g., 2 to 8 membered heteroalkyl, 2 to 6 membered heteroalkyl, or 2 to 4 membered heteroalkyl). In embodiments, R²² is unsubstituted heteroalkyl (e.g., 2 to 8 membered heteroalkyl, 2 to 6 membered heteroalkyl, or 2 to 4 membered heteroalkyl). In embodiments, R²² is R²³-substituted or unsubstituted cycloalkyl (e.g., C₃-C₈ cycloalkyl, C₃-C₆ cycloalkyl, or C₅-C₆ cycloalkyl). In embodiments, R²² is R²³-substituted cycloalkyl (e.g., C₃-C₈ cycloalkyl, C₃-C₆ cycloalkyl, or C₅-C₆ cycloalkyl). In embodiments, R²² is unsubstituted cycloalkyl (e.g., C₃-C₈ cycloalkyl, C₃-C₆ cycloalkyl, or C₅-C₆ cycloalkyl). In embodiments, R²² is R²³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²² is R²³-substituted heterocycloalkyl (e.g., 3 to 8 membered heterocycloalkyl, 3 to 6 membered heterocycloalkyl, or 5 to 6 membered heterocycloalkyl). In embodiments, R²² is unsubstituted heterocycloalkyl (e.g., 3 to 8 membered heterocycloalkyl, 3 to 6 membered heterocycloalkyl, or 5 to 6 membered heterocycloalkyl). In embodiments, R²² is R²³-substituted or unsubstituted aryl (e.g., C₆-C₁₀ aryl, C₁₀ aryl, or phenyl). In embodiments, R²² is R²³-substituted aryl (e.g., C₆-C₁₀ aryl, C₁₀ aryl, or phenyl). In embodiments, R²² is unsubstituted aryl (e.g., C₆-C₁₀ aryl, C₁₀ aryl, or phenyl). In embodiments, R²² is R²³-substituted or unsubstituted heteroaryl (e.g., 5 to 10 membered heteroaryl, 5 to 9 membered heteroaryl, or 5 to 6 membered heteroaryl). In embodiments, R²² is R²³-substituted heteroaryl (e.g., 5 to 10 membered heteroaryl, 5 to 9 membered heteroaryl, or 5 to 6 membered heteroaryl). In embodiments, R²² is unsubstituted heteroaryl (e.g., 5 to 10 membered heteroaryl, 5 to 9 membered heteroaryl, or 5 to 6 membered heteroaryl).

In embodiments, R²³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R²³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R²³ is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The definitions of R^{2A}, R^{2B}, R^{2C}, and R^{2D} are the same as the definition of R². The definitions of R^{21A}, R^{21B}, R^{21C}, and R^{21D} are the same as the definition of R²¹. The definitions of R^{22A}R^{22B}, R^{22C}, and R^{22D} are the same as the definition of R²². The definitions of R^{23A}, R^{23B}, R^{23C}, and R^{23D} are the same as the definition of R²³.

In embodiments, R^{2A} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{21A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{21A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{21A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{21A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{21A}-substituted (e.g., substituted with a substituent group, a size-limited substituent group, or lower substituent group) or unsubstituted aryl (e.g., C₆-C₁₀ aryl, C₁₀ aryl, or phenyl), or R^{21A}-substituted or unsubstituted heteroaryl (e.g., 5 to 10 membered heteroaryl, 5 to 9 membered heteroaryl, or 5 to 6 membered heteroaryl).

In embodiments, R^{21A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{22A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{22A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{22A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{22A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{22A}-substituted (e.g., substituted with a substituent group, a size-limited substituent group, or lower substituent group) or unsubstituted aryl (e.g., C₆-C₁₀ aryl, C₁₀ aryl, or phenyl), or R^{22A}-substituted or unsubstituted heteroaryl (e.g., 5 to 10 membered heteroaryl, 5 to 9 membered heteroaryl, or 5 to 6 membered heteroaryl).

In embodiments, R^{22A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{23A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{23A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{23A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{23A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{23A}-substituted (e.g., substituted with a substituent group, a size-limited substituent group, or lower substituent group) or unsubstituted aryl (e.g., C₆-C₁₀ aryl, C₁₀ aryl, or phenyl), or R^{23A}-substituted or unsubstituted heteroaryl (e.g., 5 to 10 membered heteroaryl, 5 to 9 membered heteroaryl, or 5 to 6 membered heteroaryl).

In embodiments, R^{23A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2B} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{21B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{21B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{21B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{21B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{21B}-substituted (e.g., substituted with a substituent group, a size-limited substituent group, or lower substituent group) or unsubstituted aryl (e.g., C₆-C₁₀ aryl, C₁₀ aryl, or phenyl), or R^{21B}-substituted or unsubstituted heteroaryl (e.g., 5 to 10 membered heteroaryl, 5 to 9 membered heteroaryl, or 5 to 6 membered heteroaryl).

In embodiments, R^{21B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{22B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{22B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{22B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{22B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{22B}-substituted (e.g., substituted with a substituent group, a size-limited substituent group, or lower substituent group) or unsubstituted aryl (e.g., C₆-C₁₀ aryl, C₁₀ aryl, or phenyl), or R^{22B}-substituted or unsubstituted heteroaryl (e.g., 5 to 10 membered heteroaryl, 5 to 9 membered heteroaryl, or 5 to 6 membered heteroaryl).

In embodiments, R^{22B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{23B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{23B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{23B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{23B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{23B}-substituted (e.g., substituted with a substituent group, a size-limited substituent group, or lower substituent group) or unsubstituted aryl (e.g., C₆-C₁₀ aryl, C₁₀ aryl, or phenyl), or R^{23B}-substituted or unsubstituted heteroaryl (e.g., 5 to 10 membered heteroaryl, 5 to 9 membered heteroaryl, or 5 to 6 membered heteroaryl).

In embodiments, R^{23B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2C} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{21C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{21C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{21C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{21C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{21C}-substituted (e.g., substituted with a substituent group, a size-limited substituent group, or lower substituent group) or unsubstituted aryl (e.g., C₆-C₁₀ aryl, C₁₀ aryl, or phenyl), or R^{21C}-substituted or unsubstituted heteroaryl (e.g., 5 to 10 membered heteroaryl, 5 to 9 membered heteroaryl, or 5 to 6 membered heteroaryl).

In embodiments, R^{21C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{22C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{22C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{22C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{22C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{22C}-substituted (e.g., substituted with a substituent group, a size-limited substituent group, or lower substituent group) or unsubstituted aryl (e.g., C₆-C₁₀ aryl, C₁₀ aryl, or phenyl), or R^{22C}-substituted or unsubstituted heteroaryl (e.g., 5 to 10 membered heteroaryl, 5 to 9 membered heteroaryl, or 5 to 6 membered heteroaryl).

In embodiments, R^{22C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{23C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{23C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{23C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{23C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{23C}-substituted (e.g., substituted with a substituent group, a size-limited substituent group, or lower substituent group) or unsubstituted aryl (e.g., C₆-C₁₀ aryl, C₁₀ aryl, or phenyl), or R^{23C}-substituted or unsubstituted heteroaryl (e.g., 5 to 10 membered heteroaryl, 5 to 9 membered heteroaryl, or 5 to 6 membered heteroaryl).

In embodiments, R^{23C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2D} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{21D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{21D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{21D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{21D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{21D}-substituted (e.g., substituted with a substituent group, a size-limited substituent group, or lower substituent group) or unsubstituted aryl (e.g., C₆-C₁₀ aryl, C₁₀ aryl, or phenyl), or R^{21D}-substituted or unsubstituted heteroaryl (e.g., 5 to 10 membered heteroaryl, 5 to 9 membered heteroaryl, or 5 to 6 membered heteroaryl).

In embodiments, R^{21D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{22D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{22D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{22D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{22D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{22D}-substituted (e.g., substituted with a substituent group, a size-limited substituent group, or lower substituent group) or unsubstituted aryl (e.g., C₆-C₁₀ aryl, C₁₀ aryl, or phenyl), or R^{22D}-substituted or unsubstituted heteroaryl (e.g., 5 to 10 membered heteroaryl, 5 to 9 membered heteroaryl, or 5 to 6 membered heteroaryl).

In embodiments, R^{22D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{23D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{23D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{23D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{23D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{23D}-substituted (e.g., substituted with a substituent group, a size-limited substituent group, or lower substituent group) or unsubstituted aryl (e.g., C₆-C₁₀ aryl, C₁₀ aryl, or phenyl), or R^{23D}-substituted or unsubstituted heteroaryl (e.g., 5 to 10 membered heteroaryl, 5 to 9 membered heteroaryl, or 5 to 6 membered heteroaryl).

In embodiments, R^{23D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{2B} and R^{2C} substituents may optionally be joined to form a substituted or unsubstituted heterocycloalkyl or substituted or unsubstituted heteroaryl. In embodiments, R^{2B} and R^{2C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl). In embodiments, R^{2B} and R^{2C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R^{2B} and R^{2C} substituents may optionally be joined to form a substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl).

In embodiments, R² is hydrogen, substituted or unsubstituted C₁-C₄-alkyl, or substituted or unsubstituted 2 to 4 membered heteroalkyl. In embodiments, R² is hydrogen. In embodiments, R² is substituted or unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl.

In embodiments, R³ is independently hydrogen, halogen, —CX³₃, —CHX³₂, —CH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3A}R^{3B}, —C(O)R^{3D}, —NR^{3A}R^{3B}, —C(O)OR^{3D}, —C(O)NR^{3A}R^{3B}, —C(O)NHNR^{3A}R^{3B}, —OR^{3A}, —NR^{3B}SO₂R^{3A}, —NR^{3A}C(O)R^{3B}, —NR^{3B}C(O)OR^{3D}, —NR^{3B}OR^{3D}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is independently hydrogen, halogen (e.g., —F, —Cl, Br, —I), —CX³₃, —CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —C(O)NHNR^{3A}R^{3B}, —OR^{3A}, —NR^{3A}SO₂R^{3B}, —NR^{3A}C(O)R^{3B}, —NR^{3A}C(O)OR^{3B}, —NR^{3A}OR^{3B}, —N₃, (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X³ is independently —F, —Cl, —Br, or —I.

In embodiments, R³ is independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R³ is independently hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R³ is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R³¹-substituted or unsubstituted alkyl (e.g., C₁-C₈ alkyl, C₁-C₆ alkyl, or C₁-C₄ alkyl), R³¹-substituted or unsubstituted heteroalkyl (e.g., 2 to 8 membered heteroalkyl, 2 to 6 membered heteroalkyl, or 2 to 4 membered heteroalkyl), R³¹-substituted or unsubstituted cycloalkyl (e.g., C₃-C₈ cycloalkyl, C₃-C₆ cycloalkyl, or C₅-C₆ cycloalkyl), R³¹-substituted or unsubstituted heterocycloalkyl (e.g., 3 to 8 membered heterocycloalkyl, 3 to 6 membered heterocycloalkyl, or 5 to 6 membered heterocycloalkyl), R³¹-substituted or unsubstituted aryl (e.g., C₆-C₁₀ aryl, C₁₀ aryl, or phenyl), or R³¹-substituted or unsubstituted heteroaryl (e.g., 5 to 10 membered heteroaryl, 5 to 9 membered heteroaryl, or 5 to 6 membered heteroaryl).

In embodiments, R³ is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R³ is R³¹-substituted or unsubstituted alkyl (e.g., C₁-C₈ alkyl, C₁-C₆ alkyl, or C₁-C₄ alkyl). In embodiments, R³ is R³¹-substituted alkyl (e.g., C₁-C₈ alkyl, C₁-C₆ alkyl, or C₁-C₄ alkyl). In embodiments, R³ is an unsubstituted alkyl (e.g., C₁-C₈ alkyl, C₁-C₆ alkyl, or C₁-C₄ alkyl). In embodiments, R³ is R³¹-substituted or unsubstituted heteroalkyl (e.g., 2 to 8 membered heteroalkyl, 2 to 6 membered heteroalkyl, or 2 to 4 membered heteroalkyl). In embodiments, R³ is R³¹-substituted heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl). In embodiments, R³ is R³¹-substituted or unsubstituted cycloalkyl (e.g., C₃-C₈ cycloalkyl, C₃-C₆ cycloalkyl, or C₅-C₆ cycloalkyl 1). In embodiments, R³ is R³¹-substituted cycloalkyl (e.g., C₃-C₈ cycloalkyl, C₃-C₆ cycloalkyl, or C₅-C₆ cycloalkyl). In embodiments, R³ is an unsubstituted cycloalkyl (e.g., C₃-C₈ cycloalkyl, C₃-C₆ cycloalkyl, or C₅-C₆ cycloalkyl). In embodiments, R³ is R³¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³ is R³¹-substituted heterocycloalkyl (e.g., 3 to 8 membered heterocycloalkyl, 3 to 6 membered heterocycloalkyl, or 5 to 6 membered heterocycloalkyl). In embodiments, R³ is an unsubstituted heterocycloalkyl (e.g., 3 to 8 membered heterocycloalkyl, 3 to 6 membered heterocycloalkyl, or 5 to 6 membered heterocycloalkyl). In embodiments, R³ is R³¹-substituted or unsubstituted aryl (e.g., C₆-C₁₀ aryl, C₁₀ aryl, or phenyl). In embodiments, R³ is R³¹-substituted aryl (e.g., C₆-C₁₀ aryl, C₁₀ aryl, or phenyl). In embodiments, R³ is an unsubstituted aryl (e.g., C₆-C₁₀ aryl, C₁₀ aryl, or phenyl). In embodiments, R³ is R³¹-substituted or unsubstituted heteroaryl (e.g., 5 to 10 membered heteroaryl, 5 to 9 membered heteroaryl, or 5 to 6 membered heteroaryl). In embodiments, R³ is R³¹-substituted heteroaryl (e.g., 5 to 10 membered heteroaryl, 5 to 9 membered heteroaryl, or 5 to 6 membered heteroaryl). In embodiments, R³ is an unsubstituted heteroaryl (e.g., 5 to 10 membered heteroaryl, 5 to 9 membered heteroaryl, or 5 to 6 membered heteroaryl).

R³¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R³²-substituted or unsubstituted alkyl (e.g., C₁-C₈ alkyl, C₁-C₆ alkyl, or C₁-C₄ alkyl), R³²-substituted or unsubstituted heteroalkyl (e.g., 2 to 8 membered heteroalkyl, 2 to 6 membered heteroalkyl, or 2 to 4 membered heteroalkyl), R³²-substituted or unsubstituted cycloalkyl (e.g., C₃-C₈ cycloalkyl, C₃-C₆ cycloalkyl, or C₅-C₆ cycloalkyl), R³²-substituted or unsubstituted heterocycloalkyl (e.g., 3 to 8 membered heterocycloalkyl, 3 to 6 membered heterocycloalkyl, or 5 to 6 membered heterocycloalkyl), R³²-substituted or unsubstituted aryl (e.g., C₆-C₁₀ aryl, C₁₀ aryl, or phenyl), or R³²-substituted or unsubstituted heteroaryl (e.g., 5 to 10 membered heteroaryl, 5 to 9 membered heteroaryl, or 5 to 6 membered heteroaryl).

In embodiments, R³¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R³¹ is R³²-substituted or unsubstituted alkyl (e.g., C₁-C₈ alkyl, C₁-C₆ alkyl, or C₁-C₄ alkyl). In embodiments, R³¹ is R³²-substituted (e.g., C₁-C₈ alkyl, C₁-C₆ alkyl, or C₁-C₄ alkyl). In embodiments, R³¹ is unsubstituted alkyl (e.g., C₁-C₈ alkyl, C₁-C₆ alkyl, or C₁-C₄ alkyl). In embodiments, R³¹ is R³²-substituted or unsubstituted heteroalkyl (e.g., 2 to 8 membered heteroalkyl, 2 to 6 membered heteroalkyl, or 2 to 4 membered heteroalkyl). In embodiments, R³¹ is R³²-substituted heteroalkyl (e.g., 2 to 8 membered heteroalkyl, 2 to 6 membered heteroalkyl, or 2 to 4 membered heteroalkyl). In embodiments, R³¹ is unsubstituted heteroalkyl (e.g., 2 to 8 membered heteroalkyl, 2 to 6 membered heteroalkyl, or 2 to 4 membered heteroalkyl). In embodiments, R³¹ is R³²-substituted or unsubstituted cycloalkyl (e.g., C₃-C₈ cycloalkyl, C₃-C₆ cycloalkyl, or C₅-C₆ cycloalkyl). In embodiments, R³¹ is R³²-substituted cycloalkyl (e.g., C₃-C₈ cycloalkyl, C₃-C₆ cycloalkyl, or C₅-C₆ cycloalkyl). In embodiments, R³¹ is unsubstituted cycloalkyl (e.g., C₃-C₈ cycloalkyl, C₃-C₆ cycloalkyl, or C₅-C₆ cycloalkyl). In embodiments, R³¹ is R³²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³¹ is R³²-substituted heterocycloalkyl (e.g., 3 to 8 membered heterocycloalkyl, 3 to 6 membered heterocycloalkyl, or 5 to 6 membered heterocycloalkyl). In embodiments, R³¹ is unsubstituted heterocycloalkyl (e.g., 3 to 8 membered heterocycloalkyl, 3 to 6 membered heterocycloalkyl, or 5 to 6 membered heterocycloalkyl). In embodiments, R³¹ is R³²-substituted or unsubstituted aryl (e.g., C₆-C₁₀ aryl, C₁₀ aryl, or phenyl). In embodiments, R³¹ is R³²-substituted aryl (e.g., C₆-C₁₀ aryl, C₁₀ aryl, or phenyl). In embodiments, R³¹ is unsubstituted aryl (e.g., C₆-C₁₀ aryl, C₁₀ aryl, or phenyl). In embodiments, R³¹ is R³²-substituted or unsubstituted heteroaryl (e.g., 5 to 10 membered heteroaryl, 5 to 9 membered heteroaryl, or 5 to 6 membered heteroaryl). In embodiments, R³¹ is R³²-substituted heteroaryl (e.g., 5 to 10 membered heteroaryl, 5 to 9 membered heteroaryl, or 5 to 6 membered heteroaryl). In embodiments, R³¹ is unsubstituted heteroaryl (e.g., 5 to 10 membered heteroaryl, 5 to 9 membered heteroaryl, or 5 to 6 membered heteroaryl).

R³² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R³³-substituted or unsubstituted alkyl (e.g., C₁-C₈ alkyl, C₁-C₆ alkyl, or C₁-C₄ alkyl), R³³-substituted or unsubstituted heteroalkyl (e.g., 2 to 8 membered heteroalkyl, 2 to 6 membered heteroalkyl, or 2 to 4 membered heteroalkyl), R³³-substituted or unsubstituted cycloalkyl (e.g., C₃-C₈ cycloalkyl, C₃-C₆ cycloalkyl, or C₅-C₆ cycloalkyl), R³³-substituted or unsubstituted heterocycloalkyl (e.g., 3 to 8 membered heterocycloalkyl, 3 to 6 membered heterocycloalkyl, or 5 to 6 membered heterocycloalkyl), R³³-substituted or unsubstituted aryl (e.g., C₆-C₁₀ aryl, C₁₀ aryl, or phenyl), or R³³-substituted or unsubstituted heteroaryl (e.g., 5 to 10 membered heteroaryl, 5 to 9 membered heteroaryl, or 5 to 6 membered heteroaryl).

In embodiments, R³² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R³² is R³³-substituted or unsubstituted alkyl (e.g., C₁-C₈ alkyl, C₁-C₆ alkyl, or C₁-C₄ alkyl). In embodiments, R³² is R³³-substituted (e.g., C₁-C₈ alkyl, C₁-C₆ alkyl, or C₁-C₄ alkyl). In embodiments, R³² is unsubstituted alkyl (e.g., C₁-C₈ alkyl, C₁-C₆ alkyl, or C₁-C₄ alkyl). In embodiments, R³² is R³³-substituted or unsubstituted heteroalkyl (e.g., 2 to 8 membered heteroalkyl, 2 to 6 membered heteroalkyl, or 2 to 4 membered heteroalkyl). In embodiments, R³² is R³³-substituted heteroalkyl (e.g., 2 to 8 membered heteroalkyl, 2 to 6 membered heteroalkyl, or 2 to 4 membered heteroalkyl). In embodiments, R³² is unsubstituted heteroalkyl (e.g., 2 to 8 membered heteroalkyl, 2 to 6 membered heteroalkyl, or 2 to 4 membered heteroalkyl). In embodiments, R³² is R³³-substituted or unsubstituted cycloalkyl (e.g., C₃-C₈ cycloalkyl, C₃-C₆ cycloalkyl, or C₅-C₆ cycloalkyl). In embodiments, R³² is R³³-substituted cycloalkyl (e.g., C₃-C₈ cycloalkyl, C₃-C₆ cycloalkyl, or C₅-C₆ cycloalkyl). In embodiments, R³² is unsubstituted cycloalkyl (e.g., C₃-C₈ cycloalkyl, C₃-C₆ cycloalkyl, or C₅-C₆ cycloalkyl). In embodiments, R³² is R³³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³² is R³³-substituted heterocycloalkyl (e.g., 3 to 8 membered heterocycloalkyl, 3 to 6 membered heterocycloalkyl, or 5 to 6 membered heterocycloalkyl). In embodiments, R³² is unsubstituted heterocycloalkyl (e.g., 3 to 8 membered heterocycloalkyl, 3 to 6 membered heterocycloalkyl, or 5 to 6 membered heterocycloalkyl). In embodiments, R³² is R³³-substituted or unsubstituted aryl (e.g., C₆-C₁₀ aryl, C₁₀ aryl, or phenyl). In embodiments, R³² is R³³-substituted aryl (e.g., C₆-C₁₀ aryl, C₁₀ aryl, or phenyl). In embodiments, R³² is unsubstituted aryl (e.g., C₆-C₁₀ aryl, C₁₀ aryl, or phenyl). In embodiments, R³² is R³³-substituted or unsubstituted heteroaryl (e.g., 5 to 10 membered heteroaryl, 5 to 9 membered heteroaryl, or 5 to 6 membered heteroaryl). In embodiments, R³² is R³³-substituted heteroaryl (e.g., 5 to 10 membered heteroaryl, 5 to 9 membered heteroaryl, or 5 to 6 membered heteroaryl). In embodiments, R³² is unsubstituted heteroaryl (e.g., 5 to 10 membered heteroaryl, 5 to 9 membered heteroaryl, or 5 to 6 membered heteroaryl).

In embodiments, R³³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R³³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R³³ is independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The definitions of R^{3A}, R^{3B}, R^{3C}, and R^{3D} are the same as the definition of R³. The definitions of R^{31A}, R^{31B}, R^{31C}, and R^{31D} are the same as the definition of R³¹. The definitions of R^{32A}, R^{32B}, R^{32C}, and R^{32D} are the same as the definition of R³². The definitions of R^{33A}, R^{33B}, R^{33C}, and R^{33D} are the same as the definition of R³³.

In embodiments, R^{3A} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{31A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{31A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{31A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{31A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{31A}-substituted (e.g., substituted with a substituent group, a size-limited substituent group, or lower substituent group) or unsubstituted aryl (e.g., C₆-C₁₀ aryl, C₁₀ aryl, or phenyl), or R^{31A}-substituted or unsubstituted heteroaryl (e.g., 5 to 10 membered heteroaryl, 5 to 9 membered heteroaryl, or 5 to 6 membered heteroaryl).

In embodiments, R^{31A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{32A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{32A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{32A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{32A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{32A}-substituted (e.g., substituted with a substituent group, a size-limited substituent group, or lower substituent group) or unsubstituted aryl (e.g., C₆-C₁₀ aryl, C₁₀ aryl, or phenyl), or R^{32A}-substituted or unsubstituted heteroaryl (e.g., 5 to 10 membered heteroaryl, 5 to 9 membered heteroaryl, or 5 to 6 membered heteroaryl).

In embodiments, R^{32A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{33A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{33A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{33A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{33A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{33A}-substituted (e.g., substituted with a substituent group, a size-limited substituent group, or lower substituent group) or unsubstituted aryl (e.g., C₆-C₁₀ aryl, C₁₀ aryl, or phenyl), or R^{33A}-substituted or unsubstituted heteroaryl (e.g., 5 to 10 membered heteroaryl, 5 to 9 membered heteroaryl, or 5 to 6 membered heteroaryl).

In embodiments, R^{33A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{3B} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{31B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{31B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{31B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{31B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{31B}-substituted (e.g., substituted with a substituent group, a size-limited substituent group, or lower substituent group) or unsubstituted aryl (e.g., C₆-C₁₀ aryl, C₁₀ aryl, or phenyl), or R^{31B}-substituted or unsubstituted heteroaryl (e.g., 5 to 10 membered heteroaryl, 5 to 9 membered heteroaryl, or 5 to 6 membered heteroaryl).

In embodiments, R^{31B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{32B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{32B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{32B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{32B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{32B}-substituted (e.g., substituted with a substituent group, a size-limited substituent group, or lower substituent group) or unsubstituted aryl (e.g., C₆-C₁₀ aryl, C₁₀ aryl, or phenyl), or R^{32B}-substituted or unsubstituted heteroaryl (e.g., 5 to 10 membered heteroaryl, 5 to 9 membered heteroaryl, or 5 to 6 membered heteroaryl).

In embodiments, R^{32B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{33B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{33B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{33B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{33B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{33B}-substituted (e.g., substituted with a substituent group, a size-limited substituent group, or lower substituent group) or unsubstituted aryl (e.g., C₆-C₁₀ aryl, C₁₀ aryl, or phenyl), or R^{33B}-substituted or unsubstituted heteroaryl (e.g., 5 to 10 membered heteroaryl, 5 to 9 membered heteroaryl, or 5 to 6 membered heteroaryl).

In embodiments, R^{33B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{3C} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{31C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{31C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{31C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R³¹-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{31C}-substituted (e.g., substituted with a substituent group, a size-limited substituent group, or lower substituent group) or unsubstituted aryl (e.g., C₆-C₁₀ aryl, C₁₀ aryl, or phenyl), or R^{31C}-substituted or unsubstituted heteroaryl (e.g., 5 to 10 membered heteroaryl, 5 to 9 membered heteroaryl, or 5 to 6 membered heteroaryl).

In embodiments, R^{31C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{32C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{32C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{32C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{32C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{32C}-substituted (e.g., substituted with a substituent group, a size-limited substituent group, or lower substituent group) or unsubstituted aryl (e.g., C₆-C₁₀ aryl, C₁₀ aryl, or phenyl), or R^{32C}-substituted or unsubstituted heteroaryl (e.g., 5 to 10 membered heteroaryl, 5 to 9 membered heteroaryl, or 5 to 6 membered heteroaryl).

In embodiments, R^{32C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{33C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{33C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{33C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{33C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{33C}-substituted (e.g., substituted with a substituent group, a size-limited substituent group, or lower substituent group) or unsubstituted aryl (e.g., C₆-C₁₀ aryl, C₁₀ aryl, or phenyl), or R^{33C}-substituted or unsubstituted heteroaryl (e.g., 5 to 10 membered heteroaryl, 5 to 9 membered heteroaryl, or 5 to 6 membered heteroaryl).

In embodiments, R^{33C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{3D} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{31D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{31D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{31D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{31D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{31D}-substituted (e.g., substituted with a substituent group, a size-limited substituent group, or lower substituent group) or unsubstituted aryl (e.g., C₆-C₁₀ aryl, C₁₀ aryl, or phenyl), or R^{31D}-substituted or unsubstituted heteroaryl (e.g., 5 to 10 membered heteroaryl, 5 to 9 membered heteroaryl, or 5 to 6 membered heteroaryl).

In embodiments, R^{31D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{32D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{32D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{32D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{32D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{32D}-substituted (e.g., substituted with a substituent group, a size-limited substituent group, or lower substituent group) or unsubstituted aryl (e.g., C₆-C₁₀ aryl, C₁₀ aryl, or phenyl), or R^{32D}-substituted or unsubstituted heteroaryl (e.g., 5 to 10 membered heteroaryl, 5 to 9 membered heteroaryl, or 5 to 6 membered heteroaryl).

In embodiments, R^{12D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{33D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{33D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{33D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{33D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{33D}-substituted (e.g., substituted with a substituent group, a size-limited substituent group, or lower substituent group) or unsubstituted aryl (e.g., C₆-C₁₀ aryl, C₁₀ aryl, or phenyl), or R^{33D}-substituted or unsubstituted heteroaryl (e.g., 5 to 10 membered heteroaryl, 5 to 9 membered heteroaryl, or 5 to 6 membered heteroaryl).

In embodiments, R^{33D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{3B} and R^{3C} substituents may optionally be joined to form a substituted or unsubstituted heterocycloalkyl or substituted or unsubstituted heteroaryl. In embodiments, R^{3B} and R^{3C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl). In embodiments, R^{3B} and R^{3C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R^{3B} and R^{3C} substituents may optionally be joined to form a substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl).

In embodiments, R³ is hydrogen, substituted or unsubstituted C₁-C₃ alkyl or substituted or unsubstituted 2 to 4 membered heteroalkyl. In embodiments, R³ is hydrogen. In embodiments, R³ is substituted C₁-C₄ alkyl. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is substituted 2 to 4 membered heteroalkyl. In embodiments, R³ is unsubstituted 2 to 4 membered heteroalkyl.

In embodiments, R⁴ is independently halogen, —CX⁴₃, —CHX⁴₂, —CH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, —OCX⁴₃, —OCHX⁴₂, —OCH₂X⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is independently halogen (e.g., —F, —Cl, Br, —I), —CX⁴₃, —CHX⁴₂, —CH₂X⁴, —OCX⁴₃, —OCHX⁴₂, —OCH₂X⁴, —CN, —S(O)₂R^{4A}, —SR^{4A}, —S(O)R^{4A}, —SO₂NR^{4A}R^{4B}, —NHC(O)NR^{4A}R^{4B}, —N(O)₂, —NR^{4A}R^{4B}, —NHNR^{4A}R^{4B}, —C(O)R^{4A}, —C(O)—OR^{4A}, —C(O)NR^{4A}R^{4B}, —C(O)NHNR^{4A}R^{4B}, —OR^{4A}, —NR^{4A}SO₂R^{4B}, —NR^{4A}C(O)R^{4B}, —NR^{4A}C(O)OR^{4B}, —NR^{4A}OR^{4B}, —N₃, (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X⁴ is independently —F, —Cl, —Br, or —I.

In embodiments, R⁴ is independently —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R⁴ is independently —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R⁴ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl).

In embodiments, R⁴ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁴ is R⁴¹-substituted or unsubstituted alkyl (e.g., C₁-C₈ alkyl, C₁-C₆ alkyl, or C₁-C₄ alkyl). In embodiments, R⁴ is R⁴¹-substituted alkyl (e.g., C₁-C₈ alkyl, C₁-C₆ alkyl, or C₁-C₄ alkyl). In embodiments, R⁴ is an unsubstituted alkyl (e.g., C₁-C₈ alkyl, C₁-C₆ alkyl, or C₁-C₄ alkyl). In embodiments, R⁴ is R⁴¹-substituted or unsubstituted heteroalkyl (e.g., 2 to 8 membered heteroalkyl, 2 to 6 membered heteroalkyl, or 2 to 4 membered heteroalkyl). In embodiments, R⁴ is R⁴¹-substituted heteroalkyl (e.g., 2 to 8 membered heteroalkyl, 2 to 6 membered heteroalkyl, or 2 to 4 membered heteroalkyl). In embodiments, R⁴ is an unsubstituted heteroalkyl (e.g., 2 to 8 membered heteroalkyl, 2 to 6 membered heteroalkyl, or 2 to 4 membered heteroalkyl). In embodiments, R⁴ is R⁴¹-substituted or unsubstituted cycloalkyl (e.g., C₃-C₈ cycloalkyl, C₃-C₆ cycloalkyl, or C₅-C₆ cycloalkyl 1). In embodiments, R⁴ is R⁴¹-substituted cycloalkyl (e.g., C₃-C₈ cycloalkyl, C₃-C₆ cycloalkyl, or C₅-C₆ cycloalkyl). In embodiments, R⁴ is an unsubstituted cycloalkyl (e.g., C₃-C₈ cycloalkyl, C₃-C₆ cycloalkyl, or C₅-C₆ cycloalkyl). In embodiments, R⁴ is R⁴¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is R⁴¹-substituted heterocycloalkyl (e.g., 3 to 8 membered heterocycloalkyl, 3 to 6 membered heterocycloalkyl, or 5 to 6 membered heterocycloalkyl). In embodiments, R⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is R⁴¹-substituted or unsubstituted aryl (e.g., C₆-C₁₀ aryl, C₁₀ aryl, or phenyl). In embodiments, R⁴ is R⁴¹-substituted aryl (e.g., C₆-C₁₀ aryl, C₁₀ aryl, or phenyl). In embodiments, R⁴ is an unsubstituted aryl (e.g., C₆-C₁₀ aryl, C₁₀ aryl, or phenyl). In embodiments, R⁴ is R⁴¹-substituted or unsubstituted heteroaryl (e.g., 5 to 10 membered heteroaryl, 5 to 9 membered heteroaryl, or 5 to 6 membered heteroaryl). In embodiments, R⁴ is R⁴¹-substituted heteroaryl (e.g., 5 to 10 membered heteroaryl, 5 to 9 membered heteroaryl, or 5 to 6 membered heteroaryl). In embodiments, R⁴ is an unsubstituted heteroaryl (e.g., 5 to 10 membered heteroaryl, 5 to 9 membered heteroaryl, or 5 to 6 membered heteroaryl).

In embodiments, R⁴¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁴²-substituted or unsubstituted alkyl (e.g., C₁-C₈ alkyl, C₁-C₆ alkyl, or C₁-C₄ alkyl), R⁴²-substituted or unsubstituted heteroalkyl (e.g., 2 to 8 membered heteroalkyl, 2 to 6 membered heteroalkyl, or 2 to 4 membered heteroalkyl), R⁴²-substituted or unsubstituted cycloalkyl (e.g., C₃-C₈ cycloalkyl, C₃-C₆ cycloalkyl, or C₅-C₆ cycloalkyl), R⁴²-substituted or unsubstituted heterocycloalkyl (e.g., 3 to 8 membered heterocycloalkyl, 3 to 6 membered heterocycloalkyl, or 5 to 6 membered heterocycloalkyl), R⁴²-substituted or unsubstituted aryl (e.g., C₆-C₁₀ aryl, C₁₀ aryl, or phenyl), or R⁴²-substituted or unsubstituted heteroaryl (e.g., 5 to 10 membered heteroaryl, 5 to 9 membered heteroaryl, or 5 to 6 membered heteroaryl).

In embodiments, R⁴¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁴¹ is R⁴²-substituted or unsubstituted alkyl (e.g., C₁-C₈ alkyl, C₁-C₆ alkyl, or C₁-C₄ alkyl). In embodiments, R⁴¹ is R⁴²-substituted (e.g., C₁-C₈ alkyl, C₁-C₆ alkyl, or C₁-C₄ alkyl). In embodiments, R⁴¹ is unsubstituted alkyl (e.g., C₁-C₈ alkyl, C₁-C₆ alkyl, or C₁-C₄ alkyl). In embodiments, R⁴¹ is R⁴²-substituted or unsubstituted heteroalkyl (e.g., 2 to 8 membered heteroalkyl, 2 to 6 membered heteroalkyl, or 2 to 4 membered heteroalkyl). In embodiments, R⁴¹ is R⁴²-substituted heteroalkyl (e.g., 2 to 8 membered heteroalkyl, 2 to 6 membered heteroalkyl, or 2 to 4 membered heteroalkyl). In embodiments, R⁴¹ is unsubstituted heteroalkyl (e.g., 2 to 8 membered heteroalkyl, 2 to 6 membered heteroalkyl, or 2 to 4 membered heteroalkyl). In embodiments, R⁴¹ is R⁴²-substituted or unsubstituted cycloalkyl (e.g., C₃-C₈ cycloalkyl, C₃-C₆ cycloalkyl, or C₅-C₆ cycloalkyl). In embodiments, R⁴¹ is R⁴²-substituted cycloalkyl (e.g., C₃-C₈ cycloalkyl, C₃-C₆ cycloalkyl, or C₅-C₆ cycloalkyl). In embodiments, R⁴¹ is unsubstituted cycloalkyl (e.g., C₃-C₈ cycloalkyl, C₃-C₆ cycloalkyl, or C₅-C₆ cycloalkyl). In embodiments, R⁴¹ is R⁴²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴¹ is R⁴²-substituted heterocycloalkyl (e.g., 3 to 8 membered heterocycloalkyl, 3 to 6 membered heterocycloalkyl, or 5 to 6 membered heterocycloalkyl). In embodiments, R⁴¹ is unsubstituted heterocycloalkyl (e.g., 3 to 8 membered heterocycloalkyl, 3 to 6 membered heterocycloalkyl, or 5 to 6 membered heterocycloalkyl). In embodiments, R⁴¹ is R⁴²-substituted or unsubstituted aryl (e.g., C₆-C₁₀ aryl, C₁₀ aryl, or phenyl). In embodiments, R⁴¹ is R⁴²-substituted aryl (e.g., C₆-C₁₀ aryl, C₁₀ aryl, or phenyl). In embodiments, R⁴¹ is unsubstituted aryl (e.g., C₆-C₁₀ aryl, C₁₀ aryl, or phenyl). In embodiments, R⁴¹ is R⁴²-substituted or unsubstituted heteroaryl (e.g., 5 to 10 membered heteroaryl, 5 to 9 membered heteroaryl, or 5 to 6 membered heteroaryl). In embodiments, R⁴¹ is R⁴²-substituted heteroaryl (e.g., 5 to 10 membered heteroaryl, 5 to 9 membered heteroaryl, or 5 to 6 membered heteroaryl). In embodiments, R⁴¹ is unsubstituted heteroaryl (e.g., 5 to 10 membered heteroaryl, 5 to 9 membered heteroaryl, or 5 to 6 membered heteroaryl).

In embodiments, R⁴² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁴³-substituted or unsubstituted alkyl (e.g., C₁-C₈ alkyl, C₁-C₆ alkyl, or C₁-C₄ alkyl), R⁴³-substituted or unsubstituted heteroalkyl (e.g., 2 to 8 membered heteroalkyl, 2 to 6 membered heteroalkyl, or 2 to 4 membered heteroalkyl), R⁴³-substituted or unsubstituted cycloalkyl (e.g., C₃-C₈ cycloalkyl, C₃-C₆ cycloalkyl, or C₅-C₆ cycloalkyl), R⁴³-substituted or unsubstituted heterocycloalkyl (e.g., 3 to 8 membered heterocycloalkyl, 3 to 6 membered heterocycloalkyl, or 5 to 6 membered heterocycloalkyl), R⁴³-substituted or unsubstituted aryl (e.g., C₆-C₁₀ aryl, C₁₀ aryl, or phenyl), or R⁴³-substituted or unsubstituted heteroaryl (e.g., 5 to 10 membered heteroaryl, 5 to 9 membered heteroaryl, or 5 to 6 membered heteroaryl).

In embodiments, R⁴² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁴² is R⁴³-substituted or unsubstituted alkyl (e.g., C₁-C₈ alkyl, C₁-C₆ alkyl, or C₁-C₄ alkyl). In embodiments, R⁴² is R⁴³-substituted (e.g., C₁-C₈ alkyl, C₁-C₆ alkyl, or C₁-C₄ alkyl). In embodiments, R⁴² is unsubstituted alkyl (e.g., C₁-C₈ alkyl, C₁-C₆ alkyl, or C₁-C₄ alkyl). In embodiments, R⁴² is R⁴³-substituted or unsubstituted heteroalkyl (e.g., 2 to 8 membered heteroalkyl, 2 to 6 membered heteroalkyl, or 2 to 4 membered heteroalkyl). In embodiments, R⁴² is R⁴³-substituted heteroalkyl (e.g., 2 to 8 membered heteroalkyl, 2 to 6 membered heteroalkyl, or 2 to 4 membered heteroalkyl). In embodiments, R⁴² is unsubstituted heteroalkyl (e.g., 2 to 8 membered heteroalkyl, 2 to 6 membered heteroalkyl, or 2 to 4 membered heteroalkyl). In embodiments, R⁴² is R⁴³-substituted or unsubstituted cycloalkyl (e.g., C₃-C₈ cycloalkyl, C₃-C₆ cycloalkyl, or C₅-C₆ cycloalkyl). In embodiments, R⁴² is R⁴³-substituted cycloalkyl (e.g., C₃-C₈ cycloalkyl, C₃-C₆ cycloalkyl, or C₅-C₆ cycloalkyl). In embodiments, R⁴² is unsubstituted cycloalkyl (e.g., C₃-C₈ cycloalkyl, C₃-C₆ cycloalkyl, or C₅-C₆ cycloalkyl). In embodiments, R⁴² is R⁴³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴² is R⁴³-substituted heterocycloalkyl (e.g., 3 to 8 membered heterocycloalkyl, 3 to 6 membered heterocycloalkyl, or 5 to 6 membered heterocycloalkyl). In embodiments, R⁴² is unsubstituted heterocycloalkyl (e.g., 3 to 8 membered heterocycloalkyl, 3 to 6 membered heterocycloalkyl, or 5 to 6 membered heterocycloalkyl). In embodiments, R⁴² is R⁴³-substituted or unsubstituted aryl (e.g., C₆-C₁₀ aryl, C₁₀ aryl, or phenyl). In embodiments, R⁴² is R⁴³-substituted aryl (e.g., C₆-C₁₀ aryl, C₁₀ aryl, or phenyl). In embodiments, R⁴² is unsubstituted aryl (e.g., C₆-C₁₀ aryl, C₁₀ aryl, or phenyl). In embodiments, R⁴² is R⁴³-substituted or unsubstituted heteroaryl (e.g., 5 to 10 membered heteroaryl, 5 to 9 membered heteroaryl, or 5 to 6 membered heteroaryl). In embodiments, R⁴² is R⁴³-substituted heteroaryl (e.g., 5 to 10 membered heteroaryl, 5 to 9 membered heteroaryl, or 5 to 6 membered heteroaryl). In embodiments, R⁴² is unsubstituted heteroaryl (e.g., 5 to 10 membered heteroaryl, 5 to 9 membered heteroaryl, or 5 to 6 membered heteroaryl).

R⁴³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R⁴³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁴³ is unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The definitions of R^{4A}, R^{4B}, R^{4C}, and R^{4D} are the same as the definition of R⁴. The definitions of R^{41A}, R^{41B}, R^{41C}, and R^{41D} are the same as the definition of R⁴¹. The definitions of R^{42A}, R^{42B}, R^{42C}, and R^{42D} are the same as the definition of R⁴². The definitions of R^{43A}, R^{43B}, R^{43C}, and R^{43D} are the same as the definition of R⁴³.

In embodiments, R^{4A} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{41A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{41A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{41A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{41A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{41A}-substituted (e.g., substituted with a substituent group, a size-limited substituent group, or lower substituent group) or unsubstituted aryl (e.g., C₆-C₁₀ aryl, C₁₀ aryl, or phenyl), or R^{41A}-substituted or unsubstituted heteroaryl (e.g., 5 to 10 membered heteroaryl, 5 to 9 membered heteroaryl, or 5 to 6 membered heteroaryl).

In embodiments, R^{41A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{42A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{42A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{42A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{42A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{42A}-substituted (e.g., substituted with a substituent group, a size-limited substituent group, or lower substituent group) or unsubstituted aryl (e.g., C₆-C₁₀ aryl, C₁₀ aryl, or phenyl), or R^{42A}-substituted or unsubstituted heteroaryl (e.g., 5 to 10 membered heteroaryl, 5 to 9 membered heteroaryl, or 5 to 6 membered heteroaryl).

In embodiments, R^{42A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{43A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{43A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{43A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{43A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{43A}-substituted (e.g., substituted with a substituent group, a size-limited substituent group, or lower substituent group) or unsubstituted aryl (e.g., C₆-C₁₀ aryl, C₁₀ aryl, or phenyl), or R^{43A}-substituted or unsubstituted heteroaryl (e.g., 5 to 10 membered heteroaryl, 5 to 9 membered heteroaryl, or 5 to 6 membered heteroaryl).

In embodiments, R^{43A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{4B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{41B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{41B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{41B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{41B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{41B}-substituted (e.g., substituted with a substituent group, a size-limited substituent group, or lower substituent group) or unsubstituted aryl (e.g., C₆-C₁₀ aryl, C₁₀ aryl, or phenyl), or R^{41B}-substituted or unsubstituted heteroaryl (e.g., 5 to 10 membered heteroaryl, 5 to 9 membered heteroaryl, or 5 to 6 membered heteroaryl).

In embodiments, R^{41B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{42B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{42B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{42B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{42B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{42B}-substituted (e.g., substituted with a substituent group, a size-limited substituent group, or lower substituent group) or unsubstituted aryl (e.g., C₆-C₁₀ aryl, C₁₀ aryl, or phenyl), or R^{42B}-substituted or unsubstituted heteroaryl (e.g., 5 to 10 membered heteroaryl, 5 to 9 membered heteroaryl, or 5 to 6 membered heteroaryl).

In embodiments, R^{42B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{43B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{43B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{43B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{43B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{43B}-substituted (e.g., substituted with a substituent group, a size-limited substituent group, or lower substituent group) or unsubstituted aryl (e.g., C₆-C₁₀ aryl, C₁₀ aryl, or phenyl), or R^{43B}-substituted or unsubstituted heteroaryl (e.g., 5 to 10 membered heteroaryl, 5 to 9 membered heteroaryl, or 5 to 6 membered heteroaryl).

In embodiments, R^{43B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{4C} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{41C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{41C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{41C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{41C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{41C}-substituted (e.g., substituted with a substituent group, a size-limited substituent group, or lower substituent group) or unsubstituted aryl (e.g., C₆-C₁₀ aryl, C₁₀ aryl, or phenyl), or R^{41C}-substituted or unsubstituted heteroaryl (e.g., 5 to 10 membered heteroaryl, 5 to 9 membered heteroaryl, or 5 to 6 membered heteroaryl).

In embodiments, R^{41C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{42C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{42C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{42C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{42C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{42C}-substituted (e.g., substituted with a substituent group, a size-limited substituent group, or lower substituent group) or unsubstituted aryl (e.g., C₆-C₁₀ aryl, C₁₀ aryl, or phenyl), or R^{42C}-substituted or unsubstituted heteroaryl (e.g., 5 to 10 membered heteroaryl, 5 to 9 membered heteroaryl, or 5 to 6 membered heteroaryl).

In embodiments, R^{42C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{43C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{43C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{43C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{43C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{43C}-substituted (e.g., substituted with a substituent group, a size-limited substituent group, or lower substituent group) or unsubstituted aryl (e.g., C₆-C₁₀ aryl, C₁₀ aryl, or phenyl), or R^{43C}-substituted or unsubstituted heteroaryl (e.g., 5 to 10 membered heteroaryl, 5 to 9 membered heteroaryl, or 5 to 6 membered heteroaryl).

In embodiments, R^{43C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{4D} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{41D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{41D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{41D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{41D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{41D}-substituted (e.g., substituted with a substituent group, a size-limited substituent group, or lower substituent group) or unsubstituted aryl (e.g., C₆-C₁₀ aryl, C₁₀ aryl, or phenyl), or R^{41D}-substituted or unsubstituted heteroaryl (e.g., 5 to 10 membered heteroaryl, 5 to 9 membered heteroaryl, or 5 to 6 membered heteroaryl).

In embodiments, R^{41D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{42D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{42D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{42D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{42D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{42D}-substituted (e.g., substituted with a substituent group, a size-limited substituent group, or lower substituent group) or unsubstituted aryl (e.g., C₆-C₁₀ aryl, C₁₀ aryl, or phenyl), or R^{42D}-substituted or unsubstituted heteroaryl (e.g., 5 to 10 membered heteroaryl, 5 to 9 membered heteroaryl, or 5 to 6 membered heteroaryl).

In embodiments, R^{42D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{43D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{43D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{43D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{43D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{43D}-substituted (e.g., substituted with a substituent group, a size-limited substituent group, or lower substituent group) or unsubstituted aryl (e.g., C₆-C₁₀ aryl, C₁₀ aryl, or phenyl), or R^{43D}-substituted or unsubstituted heteroaryl (e.g., 5 to 10 membered heteroaryl, 5 to 9 membered heteroaryl, or 5 to 6 membered heteroaryl).

In embodiments, R^{43D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{4B} and R^{4C} substituents may optionally be joined to form a substituted or unsubstituted heterocycloalkyl or substituted or unsubstituted heteroaryl. In embodiments, R^{4B} and R^{4C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl). In embodiments, R^{4B} and R^{4C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R^{4B} and R^{4C} substituents may optionally be joined to form a substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl).

In embodiments, R⁴ is —COOH, —OH, substituted or unsubstituted C₁-C₃ alkyl or substituted or unsubstituted 2 to 4 membered heteroalkyl. In embodiments, R⁴ is —COOH. In embodiments, R⁴ is —OH. In embodiments, R⁴ is substituted or unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is substituted 2 to 4 membered heteroalkyl. In embodiments, R⁴ is unsubstituted 2 to 4 membered heteroalkyl.

In embodiments, R⁵ is independently halogen, —CX⁵₃, —CHX⁵₂, —CH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}R^{5C}CNOR^{5D}, —NR^{5B}C(O)OR^{5D}, —NR^{5B}OR^{5D}, —OCX⁵₃, —OCHX⁵₂, —OCH₂X⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is independently halogen (e.g., —F, —Cl, Br, —I), —CX⁵₃, —CHX⁵₂, —CH₂X⁵, —OCX⁵₃, —OCHX⁵₂, —OCH₂X⁵, —CN, —N₃, —S(O)₂R^{5A}, —SR^{5A}, —S(O)R^{5A}, —SO₂NR^{5A}R^{5B}, —NHC(O)NR^{5A}R^{5B}, —N(O)₂, —NR^{5A}R^{5B}, —NR^{5A}R^{5B}, —C(O)R^{5A}, —C(O)—OR^{5A}, —C(O)NR^{5A}R^{5B}, —C(O)NHNR^{5A}R^{5B}, —OR^{5A}, —NR^{5A}SO₂R^{5B}, —NR^{5A}C(O)R^{5B}, —NR^{5A}C(O)OR^{5B}, —NR^{5A}OR^{5B} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered). X⁵ is independently —F, —Cl, —Br, or —I.

In embodiments, R⁵ is independently —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted (e.g., substituted with a substituent group, a size-limited substituent group, or lower substituent group) or unsubstituted alkyl (e.g., C₁-C₈, C₁-C₆, or C₁-C₄), substituted (e.g., substituted with a substituent group, a size-limited substituent group, or lower substituent group) or unsubstituted heteroalkyl (e.g., 2 to 8 membered, 2 to 6 membered, or 2 to 4 membered), substituted (e.g., substituted with a substituent group, a size-limited substituent group, or lower substituent group) or unsubstituted cycloalkyl (e.g., C₃-C₈, C₃-C₆, or C₅-C₆), substituted (e.g., substituted with a substituent group, a size-limited substituent group, or lower substituent group) or unsubstituted heterocycloalkyl (e.g., 3 to 8 membered, 3 to 6 membered, or 5 to 6 membered), substituted (e.g., substituted with a substituent group, a size-limited substituent group, or lower substituent group) or unsubstituted aryl (e.g., C₆-C₁₀, C₆, or phenyl), or substituted (e.g., substituted with a substituent group, a size-limited substituent group, or lower substituent group) or unsubstituted heteroaryl (e.g., 5 to 10 membered, 5 to 9 membered, or 5 to 6 membered).

In embodiments, R⁵ is independently —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₆, or phenyl), or unsubstituted heteroaryl (e.g., 5 to 10 membered, 5 to 9 membered, or 5 to 6 membered).

In embodiments, R⁵ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁵¹-substituted or unsubstituted alkyl (e.g., C₁-C₈ alkyl, C₁-C₆ alkyl, or C₁-C₄ alkyl), R⁵¹-substituted or unsubstituted heteroalkyl (e.g., 2 to 8 membered heteroalkyl, 2 to 6 membered heteroalkyl, or 2 to 4 membered heteroalkyl), R⁵¹-substituted or unsubstituted cycloalkyl (e.g., C₃-C₈ cycloalkyl, C₃-C₆ cycloalkyl, or C₅-C₆ cycloalkyl), R⁵¹-substituted or unsubstituted heterocycloalkyl (e.g., 3 to 8 membered heterocycloalkyl, 3 to 6 membered heterocycloalkyl, or 5 to 6 membered heterocycloalkyl), R⁵¹-substituted or unsubstituted aryl (e.g., C₆-C₁₀ aryl, C₁₀ aryl, or phenyl), or R⁵¹-substituted or unsubstituted heteroaryl (e.g., 5 to 10 membered heteroaryl, 5 to 9 membered heteroaryl, or 5 to 6 membered heteroaryl).

In embodiments, R⁵ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁵ is R⁵¹-substituted or unsubstituted alkyl (e.g., C₁-C₈ alkyl, C₁-C₆ alkyl, or C₁-C₄ alkyl). In embodiments, R⁵ is R⁵¹-substituted alkyl (e.g., C₁-C₈ alkyl, C₁-C₆ alkyl, or C₁-C₄ alkyl). In embodiments, R⁵ is an unsubstituted alkyl (e.g., C₁-C₈ alkyl, C₁-C₆ alkyl, or C₁-C₄ alkyl). In embodiments, R⁵ is R⁵¹-substituted or unsubstituted heteroalkyl (e.g., 2 to 8 membered heteroalkyl, 2 to 6 membered heteroalkyl, or 2 to 4 membered heteroalkyl). In embodiments, R⁵ is R⁵¹-substituted heteroalkyl (e.g., 2 to 8 membered heteroalkyl, 2 to 6 membered heteroalkyl, or 2 to 4 membered heteroalkyl). In embodiments, R⁵ is an unsubstituted heteroalkyl (e.g., 2 to 8 membered heteroalkyl, 2 to 6 membered heteroalkyl, or 2 to 4 membered heteroalkyl). In embodiments, R⁵ is R⁵¹-substituted or unsubstituted cycloalkyl (e.g., C₃-C₈ cycloalkyl, C₃-C₆ cycloalkyl, or C₅-C₆ cycloalkyl 1). In embodiments, R⁵ is R⁵¹-substituted cycloalkyl (e.g., C₃-C₈ cycloalkyl, C₃-C₆ cycloalkyl, or C₅-C₆ cycloalkyl). In embodiments, R⁵ is an unsubstituted cycloalkyl (e.g., C₃-C₈ cycloalkyl, C₃-C₆ cycloalkyl, or C₅-C₆ cycloalkyl). In embodiments, R⁵ is R⁵¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R⁵¹-substituted heterocycloalkyl (e.g., 3 to 8 membered heterocycloalkyl, 3 to 6 membered heterocycloalkyl, or 5 to 6 membered heterocycloalkyl). In embodiments, R⁵ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R⁵¹-substituted or unsubstituted aryl (e.g., C₆-C₁₀ aryl, C₁₀ aryl, or phenyl). In embodiments, R⁵ is R⁵¹-substituted aryl (e.g., C₆-C₁₀ aryl, C₁₀ aryl, or phenyl). In embodiments, R⁵ is an unsubstituted aryl (e.g., C₆-C₁₀ aryl, C₁₀ aryl, or phenyl). In embodiments, R⁵ is R⁵¹-substituted or unsubstituted heteroaryl (e.g., 5 to 10 membered heteroaryl, 5 to 9 membered heteroaryl, or 5 to 6 membered heteroaryl). In embodiments, R⁵ is R⁵¹-substituted heteroaryl (e.g., 5 to 10 membered heteroaryl, 5 to 9 membered heteroaryl, or 5 to 6 membered heteroaryl). In embodiments, R⁵ is an unsubstituted heteroaryl (e.g., 5 to 10 membered heteroaryl, 5 to 9 membered heteroaryl, or 5 to 6 membered heteroaryl).

R⁵¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁵²-substituted or unsubstituted alkyl (e.g., C₁-C₈ alkyl, C₁-C₆ alkyl, or C₁-C₄ alkyl), R⁵²-substituted or unsubstituted heteroalkyl (e.g., 2 to 8 membered heteroalkyl, 2 to 6 membered heteroalkyl, or 2 to 4 membered heteroalkyl), R⁵²-substituted or unsubstituted cycloalkyl (e.g., C₃-C₈ cycloalkyl, C₃-C₆ cycloalkyl, or C₅-C₆ cycloalkyl), R⁵²-substituted or unsubstituted heterocycloalkyl (e.g., 3 to 8 membered heterocycloalkyl, 3 to 6 membered heterocycloalkyl, or 5 to 6 membered heterocycloalkyl), R⁵²-substituted or unsubstituted aryl (e.g., C₆-C₁₀ aryl, C₁₀ aryl, or phenyl), or R⁵²-substituted or unsubstituted heteroaryl (e.g., 5 to 10 membered heteroaryl, 5 to 9 membered heteroaryl, or 5 to 6 membered heteroaryl).

In embodiments, R⁵¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁵¹ is R⁵²-substituted or unsubstituted alkyl (e.g., C₁-C₈ alkyl, C₁-C₆ alkyl, or C₁-C₄ alkyl). In embodiments, R⁵¹ is R⁵²-substituted (e.g., C₁-C₈ alkyl, C₁-C₆ alkyl, or C₁-C₄ alkyl). In embodiments, R⁵¹ is unsubstituted alkyl (e.g., C₁-C₈ alkyl, C₁-C₆ alkyl, or C₁-C₄ alkyl). In embodiments, R⁵¹ is R⁵²-substituted or unsubstituted heteroalkyl (e.g., 2 to 8 membered heteroalkyl, 2 to 6 membered heteroalkyl, or 2 to 4 membered heteroalkyl). In embodiments, R⁵¹ is R⁵²-substituted heteroalkyl (e.g., 2 to 8 membered heteroalkyl, 2 to 6 membered heteroalkyl, or 2 to 4 membered heteroalkyl). In embodiments, R⁵¹ is unsubstituted heteroalkyl (e.g., 2 to 8 membered heteroalkyl, 2 to 6 membered heteroalkyl, or 2 to 4 membered heteroalkyl). In embodiments, R⁵¹ is R⁵²-substituted or unsubstituted cycloalkyl (e.g., C₃-C₈ cycloalkyl, C₃-C₆ cycloalkyl, or C₅-C₆ cycloalkyl). In embodiments, R⁵¹ is R⁵²-substituted cycloalkyl (e.g., C₃-C₈ cycloalkyl, C₃-C₆ cycloalkyl, or C₅-C₆ cycloalkyl). In embodiments, R⁵¹ is unsubstituted cycloalkyl (e.g., C₃-C₈ cycloalkyl, C₃-C₆ cycloalkyl, or C₅-C₆ cycloalkyl). In embodiments, R⁵¹ is R⁵²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵¹ is R⁵²-substituted heterocycloalkyl (e.g., 3 to 8 membered heterocycloalkyl, 3 to 6 membered heterocycloalkyl, or 5 to 6 membered heterocycloalkyl). In embodiments, R⁵¹ is unsubstituted heterocycloalkyl (e.g., 3 to 8 membered heterocycloalkyl, 3 to 6 membered heterocycloalkyl, or 5 to 6 membered heterocycloalkyl). In embodiments, R⁵¹ is R⁵²-substituted or unsubstituted aryl (e.g., C₆-C₁₀ aryl, C₁₀ aryl, or phenyl). In embodiments, R⁵¹ is R⁵²-substituted aryl (e.g., C₆-C₁₀ aryl, C₁₀ aryl, or phenyl). In embodiments, R⁵¹ is unsubstituted aryl (e.g., C₆-C₁₀ aryl, C₁₀ aryl, or phenyl). In embodiments, R⁵¹ is R⁵²-substituted or unsubstituted heteroaryl (e.g., 5 to 10 membered heteroaryl, 5 to 9 membered heteroaryl, or 5 to 6 membered heteroaryl). In embodiments, R⁵¹ is R⁵²-substituted heteroaryl (e.g., 5 to 10 membered heteroaryl, 5 to 9 membered heteroaryl, or 5 to 6 membered heteroaryl). In embodiments, R⁵¹ is unsubstituted heteroaryl (e.g., 5 to 10 membered heteroaryl, 5 to 9 membered heteroaryl, or 5 to 6 membered heteroaryl).

In embodiments, R⁵² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl).

In embodiments, R⁵² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁵² is R⁵³-substituted or unsubstituted alkyl (e.g., C₁-C₈ alkyl, C₁-C₆ alkyl, or C₁-C₄ alkyl). In embodiments, R⁵² is R⁵³-substituted (e.g., C₁-C₈ alkyl, C₁-C₆ alkyl, or C₁-C₄ alkyl). In embodiments, R⁵² is unsubstituted alkyl (e.g., C₁-C₈ alkyl, C₁-C₆ alkyl, or C₁-C₄ alkyl). In embodiments, R⁵² is R⁵³-substituted or unsubstituted heteroalkyl (e.g., 2 to 8 membered heteroalkyl, 2 to 6 membered heteroalkyl, or 2 to 4 membered heteroalkyl). In embodiments, R⁵² is R⁵³-substituted heteroalkyl (e.g., 2 to 8 membered heteroalkyl, 2 to 6 membered heteroalkyl, or 2 to 4 membered heteroalkyl). In embodiments, R⁵² is unsubstituted heteroalkyl (e.g., 2 to 8 membered heteroalkyl, 2 to 6 membered heteroalkyl, or 2 to 4 membered heteroalkyl). In embodiments, R⁵² is R⁵³-substituted or unsubstituted cycloalkyl (e.g., C₃-C₈ cycloalkyl, C₃-C₆ cycloalkyl, or C₅-C₆ cycloalkyl). In embodiments, R⁵² is R⁵³-substituted cycloalkyl (e.g., C₃-C₈ cycloalkyl, C₃-C₆ cycloalkyl, or C₅-C₆ cycloalkyl). In embodiments, R⁵² is unsubstituted cycloalkyl (e.g., C₃-C₈ cycloalkyl, C₃-C₆ cycloalkyl, or C₅-C₆ cycloalkyl). In embodiments, R⁵² is R⁵³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵² is R⁵³-substituted heterocycloalkyl (e.g., 3 to 8 membered heterocycloalkyl, 3 to 6 membered heterocycloalkyl, or 5 to 6 membered heterocycloalkyl). In embodiments, R⁵² is unsubstituted heterocycloalkyl (e.g., 3 to 8 membered heterocycloalkyl, 3 to 6 membered heterocycloalkyl, or 5 to 6 membered heterocycloalkyl). In embodiments, R⁵² is R⁵³-substituted or unsubstituted aryl (e.g., C₆-C₁₀ aryl, C₁₀ aryl, or phenyl). In embodiments, R⁵² is R⁵³-substituted aryl (e.g., C₆-C₁₀ aryl, C₁₀ aryl, or phenyl). In embodiments, R⁵² is unsubstituted aryl (e.g., C₆-C₁₀ aryl, C₁₀ aryl, or phenyl). In embodiments, R⁵² is R⁵³-substituted or unsubstituted heteroaryl (e.g., 5 to 10 membered heteroaryl, 5 to 9 membered heteroaryl, or 5 to 6 membered heteroaryl). In embodiments, R⁵² is R⁵³-substituted heteroaryl (e.g., 5 to 10 membered heteroaryl, 5 to 9 membered heteroaryl, or 5 to 6 membered heteroaryl). In embodiments, R⁵² is unsubstituted heteroaryl (e.g., 5 to 10 membered heteroaryl, 5 to 9 membered heteroaryl, or 5 to 6 membered heteroaryl).

In embodiments, R⁵³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R⁵³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁵³ is unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The definitions of R^{5A}, R^{5B}, R^{5C}, and R^{5D} are the same as the definition of R⁵. The definitions of R^{51A}, R^{51B}, R^{51C}, and R^{51D} are the same as the definition of R⁵¹. The definitions of R^{52A}, R^{52B}, R^{52C}, and R^{52D} are the same as the definition of R⁵². The definitions of R^{53A}, R^{53B}, R^{53C}, and R^{53D} are the same as the definition of R⁵³.

In embodiments, R^{5A} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{51A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{51A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{51A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{51A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{51A}-substituted (e.g., substituted with a substituent group, a size-limited substituent group, or lower substituent group) or unsubstituted aryl (e.g., C₆-C₁₀ aryl, C₁₀ aryl, or phenyl), or R^{51A}-substituted or unsubstituted heteroaryl (e.g., 5 to 10 membered heteroaryl, 5 to 9 membered heteroaryl, or 5 to 6 membered heteroaryl).

In embodiments, R^{51A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{52A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{52A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{52A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{52A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{52A}-substituted (e.g., substituted with a substituent group, a size-limited substituent group, or lower substituent group) or unsubstituted aryl (e.g., C₆-C₁₀ aryl, C₁₀ aryl, or phenyl), or R^{52A}-substituted or unsubstituted heteroaryl (e.g., 5 to 10 membered heteroaryl, 5 to 9 membered heteroaryl, or 5 to 6 membered heteroaryl).

In embodiments, R^{52A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{53A}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{53A}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{53A}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{53A}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{53A}-substituted (e.g., substituted with a substituent group, a size-limited substituent group, or lower substituent group) or unsubstituted aryl (e.g., C₆-C₁₀ aryl, C₁₀ aryl, or phenyl), or R^{53A}-substituted or unsubstituted heteroaryl (e.g., 5 to 10 membered heteroaryl, 5 to 9 membered heteroaryl, or 5 to 6 membered heteroaryl).

In embodiments, R^{53A} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{5B} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{51B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{51B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{51B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{51B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{51B}-substituted (e.g., substituted with a substituent group, a size-limited substituent group, or lower substituent group) or unsubstituted aryl (e.g., C₆-C₁₀ aryl, C₁₀ aryl, or phenyl), or R^{51B}-substituted or unsubstituted heteroaryl (e.g., 5 to 10 membered heteroaryl, 5 to 9 membered heteroaryl, or 5 to 6 membered heteroaryl).

In embodiments, R^{51B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{52B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{52B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{52B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{52B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{52B}-substituted (e.g., substituted with a substituent group, a size-limited substituent group, or lower substituent group) or unsubstituted aryl (e.g., C₆-C₁₀ aryl, C₁₀ aryl, or phenyl), or R^{52B}-substituted or unsubstituted heteroaryl (e.g., 5 to 10 membered heteroaryl, 5 to 9 membered heteroaryl, or 5 to 6 membered heteroaryl).

In embodiments, R^{52B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{53B}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{53B}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{53B}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{53B}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{53B}-substituted (e.g., substituted with a substituent group, a size-limited substituent group, or lower substituent group) or unsubstituted aryl (e.g., C₆-C₁₀ aryl, C₁₀ aryl, or phenyl), or R^{53B}-substituted or unsubstituted heteroaryl (e.g., 5 to 10 membered heteroaryl, 5 to 9 membered heteroaryl, or 5 to 6 membered heteroaryl).

In embodiments, R^{53B} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{5C} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{51C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{51C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{51C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{51C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{51C}-substituted (e.g., substituted with a substituent group, a size-limited substituent group, or lower substituent group) or unsubstituted aryl (e.g., C₆-C₁₀ aryl, C₁₀ aryl, or phenyl), or R^{51C}-substituted or unsubstituted heteroaryl (e.g., 5 to 10 membered heteroaryl, 5 to 9 membered heteroaryl, or 5 to 6 membered heteroaryl).

In embodiments, R^{51C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{52C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{52C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{52C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{52C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{52C}-substituted (e.g., substituted with a substituent group, a size-limited substituent group, or lower substituent group) or unsubstituted aryl (e.g., C₆-C₁₀ aryl, C₁₀ aryl, or phenyl), or R^{52C}-substituted or unsubstituted heteroaryl (e.g., 5 to 10 membered heteroaryl, 5 to 9 membered heteroaryl, or 5 to 6 membered heteroaryl).

In embodiments, R^{52C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{53C}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{53C}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{53C}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{53C}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{53C}-substituted (e.g., substituted with a substituent group, a size-limited substituent group, or lower substituent group) or unsubstituted aryl (e.g., C₆-C₁₀ aryl, C₁₀ aryl, or phenyl), or R^{53C}-substituted or unsubstituted heteroaryl (e.g., 5 to 10 membered heteroaryl, 5 to 9 membered heteroaryl, or 5 to 6 membered heteroaryl).

In embodiments, R^{53C} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{5D} is independently hydrogen, halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{51D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{51D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{51D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{51D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{51D}-substituted (e.g., substituted with a substituent group, a size-limited substituent group, or lower substituent group) or unsubstituted aryl (e.g., C₆-C₁₀ aryl, C₁₀ aryl, or phenyl), or R^{51D}-substituted or unsubstituted heteroaryl (e.g., 5 to 10 membered heteroaryl, 5 to 9 membered heteroaryl, or 5 to 6 membered heteroaryl).

In embodiments, R^{51D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{52D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{52D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{52D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{52D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{52D}-substituted (e.g., substituted with a substituent group, a size-limited substituent group, or lower substituent group) or unsubstituted aryl (e.g., C₆-C₁₀ aryl, C₁₀ aryl, or phenyl), or R^{52D}-substituted or unsubstituted heteroaryl (e.g., 5 to 10 membered heteroaryl, 5 to 9 membered heteroaryl, or 5 to 6 membered heteroaryl).

In embodiments, R^{52D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R^{53D}-substituted (e.g., substituted with a substituent group, a size-limited substituent group, or lower substituent group) or unsubstituted alkyl (e.g., C₁-C₈ alkyl, C₁-C₆ alkyl, or C₁-C₄ alkyl), R^{53D}-substituted (e.g., substituted with a substituent group, a size-limited substituent group, or lower substituent group) or unsubstituted heteroalkyl (e.g., 2 to 8 membered heteroalkyl, 2 to 6 membered heteroalkyl, or 2 to 4 membered heteroalkyl), R^{53D}-substituted (e.g., substituted with a substituent group, a size-limited substituent group, or lower substituent group) or unsubstituted cycloalkyl (e.g., C₃-C₈ cycloalkyl, C₃-C₆ cycloalkyl, or C₅-C₆ cycloalkyl 1), R^{53D}-substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), R^{53D}-substituted (e.g., substituted with a substituent group, a size-limited substituent group, or lower substituent group) or unsubstituted aryl (e.g., C₆-C₁₀ aryl, C₁₀ aryl, or phenyl), or R^{53D}-substituted or unsubstituted heteroaryl (e.g., 5 to 10 membered heteroaryl, 5 to 9 membered heteroaryl, or 5 to 6 membered heteroaryl).

In embodiments, R^{53D} is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{5B} and R^{5C} substituents may optionally be joined to form a substituted or unsubstituted heterocycloalkyl or substituted or unsubstituted heteroaryl. In embodiments, R^{5B} and R^{5C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl). In embodiments, R^{5B} and R^{5C} substituents may optionally be joined to form a substituted (e.g., substituted with a substituent group, a size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R^{5B} and R^{5C} substituents may optionally be joined to form a substituted (e.g., substituted with a substituent group, a size-limited substituent group, or lower substituent group) or unsubstituted heteroaryl. (e.g., 5 to 10 membered heteroaryl, 5 to 9 membered heteroaryl, or 5 to 6 membered heteroaryl).

In embodiments, R⁶ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is R⁶¹-substituted or unsubstituted alkyl (e.g., C₁-C₈ alkyl, C₁-C₆ alkyl, or C₁-C₄ alkyl). In embodiments, R⁶ is R⁶¹-substituted alkyl (e.g., C₁-C₈ alkyl, C₁-C₆ alkyl, or C₁-C₄ alkyl). In embodiments, R⁶ is an unsubstituted alkyl (e.g., C₁-C₈ alkyl, C₁-C₆ alkyl, or C₁-C₄ alkyl). In embodiments, R⁶ is R⁶¹-substituted or unsubstituted heteroalkyl (e.g., 2 to 8 membered heteroalkyl, 2 to 6 membered heteroalkyl, or 2 to 4 membered heteroalkyl). In embodiments, R⁶ is R⁶¹-substituted heteroalkyl (e.g., 2 to 8 membered heteroalkyl, 2 to 6 membered heteroalkyl, or 2 to 4 membered heteroalkyl). In embodiments, R⁶ is an unsubstituted heteroalkyl (e.g., 2 to 8 membered heteroalkyl, 2 to 6 membered heteroalkyl, or 2 to 4 membered heteroalkyl). In embodiments, R⁶ is R⁶¹-substituted or unsubstituted cycloalkyl (e.g., C₃-C₈ cycloalkyl, C₃-C₆ cycloalkyl, or C₅-C₆ cycloalkyl). In embodiments, R⁶ is R⁶¹-substituted cycloalkyl (e.g., C₃-C₈ cycloalkyl, C₃-C₆ cycloalkyl, or C₅-C₆ cycloalkyl). In embodiments, R⁶ is an unsubstituted cycloalkyl (e.g., C₃-C₈ cycloalkyl, C₃-C₆ cycloalkyl, or C₅-C₆ cycloalkyl). In embodiments, R⁶ is R⁶¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is R⁶¹-substituted heterocycloalkyl (e.g., 3 to 8 membered heterocycloalkyl, 3 to 6 membered heterocycloalkyl, or 5 to 6 membered heterocycloalkyl). In embodiments, R⁶ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is R⁶¹-substituted or unsubstituted aryl (e.g., C₆-C₁₀ aryl, C₁₀ aryl, or phenyl). In embodiments, R⁶ is R⁶¹-substituted aryl (e.g., C₆-C₁₀ aryl, C₁₀ aryl, or phenyl). In embodiments, R⁶ is an unsubstituted aryl (e.g., C₆-C₁₀ aryl, C₁₀ aryl, or phenyl). In embodiments, R⁶ is R⁶¹-substituted or unsubstituted heteroaryl (e.g., 5 to 10 membered heteroaryl, 5 to 9 membered heteroaryl, or 5 to 6 membered heteroaryl). In embodiments, R⁶ is R⁶¹-substituted heteroaryl (e.g., 5 to 10 membered heteroaryl, 5 to 9 membered heteroaryl, or 5 to 6 membered heteroaryl). In embodiments, R⁶ is an unsubstituted heteroaryl (e.g., 5 to 10 membered heteroaryl, 5 to 9 membered heteroaryl, or 5 to 6 membered heteroaryl).

In embodiments, R⁶¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁶²-substituted or unsubstituted alkyl (e.g., C₁-C₈ alkyl, C₁-C₆ alkyl, or C₁-C₄ alkyl), R⁶²-substituted or unsubstituted heteroalkyl (e.g., 2 to 8 membered heteroalkyl, 2 to 6 membered heteroalkyl, or 2 to 4 membered heteroalkyl), R⁶²-substituted or unsubstituted cycloalkyl (e.g., C₃-C₈ cycloalkyl, C₃-C₆ cycloalkyl, or C₅-C₆ cycloalkyl), R⁶²-substituted or unsubstituted heterocycloalkyl (e.g., 3 to 8 membered heterocycloalkyl, 3 to 6 membered heterocycloalkyl, or 5 to 6 membered heterocycloalkyl), R⁶²-substituted or unsubstituted aryl (e.g., C₆-C₁₀ aryl, C₁₀ aryl, or phenyl), or R⁶²-substituted or unsubstituted heteroaryl (e.g., 5 to 10 membered heteroaryl, 5 to 9 membered heteroaryl, or 5 to 6 membered heteroaryl).

In embodiments, R⁶¹ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁶¹ is R⁶²-substituted or unsubstituted alkyl (e.g., C₁-C₈ alkyl, C₁-C₆ alkyl, or C₁-C₄ alkyl). In embodiments, R⁶¹ is R⁶²-substituted (e.g., C₁-C₈ alkyl, C₁-C₆ alkyl, or C₁-C₄ alkyl). In embodiments, R⁶¹ is unsubstituted alkyl (e.g., C₁-C₈ alkyl, C₁-C₆ alkyl, or C₁-C₄ alkyl). In embodiments, R⁶¹ is R⁶²-substituted or unsubstituted heteroalkyl (e.g., 2 to 8 membered heteroalkyl, 2 to 6 membered heteroalkyl, or 2 to 4 membered heteroalkyl). In embodiments, R⁶¹ is R⁶²-substituted heteroalkyl (e.g., 2 to 8 membered heteroalkyl, 2 to 6 membered heteroalkyl, or 2 to 4 membered heteroalkyl). In embodiments, R⁶¹ is unsubstituted heteroalkyl (e.g., 2 to 8 membered heteroalkyl, 2 to 6 membered heteroalkyl, or 2 to 4 membered heteroalkyl). In embodiments, R⁶¹ is R⁶²-substituted or unsubstituted cycloalkyl (e.g., C₃-C₈ cycloalkyl, C₃-C₆ cycloalkyl, or C₅-C₆ cycloalkyl). In embodiments, R⁶¹ is R⁶²-substituted cycloalkyl (e.g., C₃-C₈ cycloalkyl, C₃-C₆ cycloalkyl, or C₅-C₆ cycloalkyl). In embodiments, R⁶¹ is unsubstituted cycloalkyl (e.g., C₃-C₈ cycloalkyl, C₃-C₆ cycloalkyl, or C₅-C₆ cycloalkyl). In embodiments, R⁶¹ is R⁶²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶¹ is R⁶²-substituted heterocycloalkyl (e.g., 3 to 8 membered heterocycloalkyl, 3 to 6 membered heterocycloalkyl, or 5 to 6 membered heterocycloalkyl). In embodiments, R⁶¹ is unsubstituted heterocycloalkyl (e.g., 3 to 8 membered heterocycloalkyl, 3 to 6 membered heterocycloalkyl, or 5 to 6 membered heterocycloalkyl). In embodiments, R⁶¹ is R⁶²-substituted or unsubstituted aryl (e.g., C₆-C₁₀ aryl, C₁₀ aryl, or phenyl). In embodiments, R⁶¹ is R⁶²-substituted aryl (e.g., C₆-C₁₀ aryl, C₁₀ aryl, or phenyl). In embodiments, R⁶¹ is unsubstituted aryl (e.g., C₆-C₁₀ aryl, C₁₀ aryl, or phenyl). In embodiments, R⁶¹ is R⁶²-substituted or unsubstituted heteroaryl (e.g., 5 to 10 membered heteroaryl, 5 to 9 membered heteroaryl, or 5 to 6 membered heteroaryl). In embodiments, R⁶¹ is R⁶²-substituted heteroaryl (e.g., 5 to 10 membered heteroaryl, 5 to 9 membered heteroaryl, or 5 to 6 membered heteroaryl). In embodiments, R⁶¹ is unsubstituted heteroaryl (e.g., 5 to 10 membered heteroaryl, 5 to 9 membered heteroaryl, or 5 to 6 membered heteroaryl).

In embodiments, R⁶² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, R⁶³-substituted or unsubstituted alkyl (e.g., C₁-C₈ alkyl, C₁-C₆ alkyl, or C₁-C₄ alkyl), R⁶³-substituted or unsubstituted heteroalkyl (e.g., 2 to 8 membered heteroalkyl, 2 to 6 membered heteroalkyl, or 2 to 4 membered heteroalkyl), R⁶³-substituted or unsubstituted cycloalkyl (e.g., C₃-C₈ cycloalkyl, C₃-C₆ cycloalkyl, or C₅-C₆ cycloalkyl), R⁶³-substituted or unsubstituted heterocycloalkyl (e.g., 3 to 8 membered heterocycloalkyl, 3 to 6 membered heterocycloalkyl, or 5 to 6 membered heterocycloalkyl), R⁶³-substituted or unsubstituted aryl (e.g., C₆-C₁₀ aryl, C₁₀ aryl, or phenyl), or R⁶³-substituted or unsubstituted heteroaryl (e.g., 5 to 10 membered heteroaryl, 5 to 9 membered heteroaryl, or 5 to 6 membered heteroaryl).

In embodiments, R⁶² is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁶² is R⁶³-substituted or unsubstituted alkyl (e.g., C₁-C₈ alkyl, C₁-C₆ alkyl, or C₁-C₄ alkyl). In embodiments, R⁶² is R⁶³-substituted (e.g., C₁-C₈ alkyl, C₁-C₆ alkyl, or C₁-C₄ alkyl). In embodiments, R⁶² is unsubstituted alkyl (e.g., C₁-C₈ alkyl, C₁-C₆ alkyl, or C₁-C₄ alkyl). In embodiments, R⁶² is R⁶³-substituted or unsubstituted heteroalkyl (e.g., 2 to 8 membered heteroalkyl, 2 to 6 membered heteroalkyl, or 2 to 4 membered heteroalkyl). In embodiments, R⁶² is R⁶³-substituted heteroalkyl (e.g., 2 to 8 membered heteroalkyl, 2 to 6 membered heteroalkyl, or 2 to 4 membered heteroalkyl). In embodiments, R⁶² is unsubstituted heteroalkyl (e.g., 2 to 8 membered heteroalkyl, 2 to 6 membered heteroalkyl, or 2 to 4 membered heteroalkyl). In embodiments, R⁶² is R⁶³-substituted or unsubstituted cycloalkyl (e.g., C₃-C₈ cycloalkyl, C₃-C₆ cycloalkyl, or C₅-C₆ cycloalkyl). In embodiments, R⁶² is R⁶³-substituted cycloalkyl (e.g., C₃-C₈ cycloalkyl, C₃-C₆ cycloalkyl, or C₅-C₆ cycloalkyl). In embodiments, R⁶² is unsubstituted cycloalkyl (e.g., C₃-C₈ cycloalkyl, C₃-C₆ cycloalkyl, or C₅-C₆ cycloalkyl). In embodiments, R⁶² is R⁶³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶² is R⁶³-substituted heterocycloalkyl (e.g., 3 to 8 membered heterocycloalkyl, 3 to 6 membered heterocycloalkyl, or 5 to 6 membered heterocycloalkyl). In embodiments, R⁶² is unsubstituted heterocycloalkyl (e.g., 3 to 8 membered heterocycloalkyl, 3 to 6 membered heterocycloalkyl, or 5 to 6 membered heterocycloalkyl). In embodiments, R⁶² is R⁶³-substituted or unsubstituted aryl (e.g., C₆-C₁₀ aryl, C₁₀ aryl, or phenyl). In embodiments, R⁶² is R⁶³-substituted aryl (e.g., C₆-C₁₀ aryl, C₁₀ aryl, or phenyl). In embodiments, R⁶² is unsubstituted aryl (e.g., C₆-C₁₀ aryl, C₁₀ aryl, or phenyl). In embodiments, R⁶² is R⁶³-substituted or unsubstituted heteroaryl (e.g., 5 to 10 membered heteroaryl, 5 to 9 membered heteroaryl, or 5 to 6 membered heteroaryl). In embodiments, R⁶² is R⁶³-substituted heteroaryl (e.g., 5 to 10 membered heteroaryl, 5 to 9 membered heteroaryl, or 5 to 6 membered heteroaryl). In embodiments, R⁶² is unsubstituted heteroaryl (e.g., 5 to 10 membered heteroaryl, 5 to 9 membered heteroaryl, or 5 to 6 membered heteroaryl).

In embodiments, R⁶³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R⁶³ is independently halogen, —CF₃, —CCl₃, —CBr₃, —CI₃, —OH, —NH₂, —COOH, —CONH₂, —NO₂, —N₃, —SH, —SO₃H, —SO₄H, —SO₂NH₂, —NHNH₂, —ONH₂, —NHC(O)NHNH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —OCH₂F, —OCH₂Cl, —OCH₂Br, or —OCH₂I.

In embodiments, R⁶³ is unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R⁶ is hydrogen, substituted or unsubstituted C₁-C₄ alkyl or substituted or unsubstituted 2 to 4 membered heteroalkyl. In embodiments, R⁶ is hydrogen. In embodiments, R⁶ is substituted or unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is substituted 2 to 4 membered heteroalkyl. In embodiments, R⁶ is unsubstituted 2 to 4 membered heteroalkyl.

In embodiments, n is an integer from 1 to 4. In embodiments, n is 1. In embodiments, n is 2. In embodiments, n is 3. In embodiments, n is 4. In embodiments, n1 is an integer from 0 to 4. In embodiments, n1 is 0. In embodiments, n1 is 1. In embodiments, n1 is 2. In embodiments, n1 is 3. In embodiments, n1 is 4. In embodiments, n2 is an integer from 0 to 4. In embodiments, n2 is 0. In embodiments, n2 is 1. In embodiments, n2 is 2. In embodiments, n2 is 3. In embodiments, n2 is 4. In embodiments, n3 is an integer from 0 to 4. In embodiments, n3 is 0. In embodiments, n3 is 1. In embodiments, n3 is 2. In embodiments, n3 is 3. In embodiments, n3 is 4. In embodiments, n4 is an integer from 0 to 4. In embodiments, n4 is 0. In embodiments, n4 is 1. In embodiments, n4 is 2. In embodiments, n4 is 3. In embodiments, n4 is 4. In embodiments, n5 is an integer from 0 to 4. In embodiments, n5 is 0. In embodiments, n5 is 1. In embodiments, n5 is 2. In embodiments, n5 is 3. In embodiments, n5 is 4. In embodiments, m is an integer from 1 to 2. In embodiments, m is 1. In embodiments, m is 2. In embodiments, m1 is 1 or 2. In embodiments, m1 is 1. In embodiments, m1 is 2. In embodiments, m2 is 1 or 2. In embodiments, m2 is 1. In embodiments, m2 is 2. In embodiments, m3 is 1 or 2. In embodiments, m3 is 1. In embodiments, m3 is 2. In embodiments, m4 is 1 or 2. In embodiments, m4 is 1. In embodiments, m4 is 2. In embodiments, m5 is 1 or 2. In embodiments, m5 is 1. In embodiments, m5 is 2. In embodiments, v1 is 1 or 2. In embodiments, v1 is 1. In embodiments, v1 is 2. In embodiments, v2 is 1 or 2. In embodiments, v2 is 1. In embodiments, v2 is 2. In embodiments, v3 is 1 or 2. In embodiments, v3 is 1. In embodiments, v3 is 2. In embodiments, v4 is 1 or 2. In embodiments, v4 is 1. In embodiments, v4 is 2. In embodiments, v5 is 1 or 2. In embodiments, v5 is 1. In embodiments, v5 is 2.

In embodiments, X¹ is halogen. In embodiments, halogen is —F, —Cl, —Br, —I. In embodiments, X¹ is —F. In embodiments, X¹ is —Cl. In embodiments, X¹ is —Br. In embodiments, X¹ is —I. In embodiments, X² is halogen. In embodiments, halogen is —F, —Cl, —Br, —I. In embodiments, X² is —F. In embodiments, X² is —Cl. In embodiments, X² is —Br. In embodiments, X² is —I. In embodiments, X³ is halogen. In embodiments, halogen is —F, —Cl, —Br, —I. In embodiments, X³ is —F. In embodiments, X³ is —Cl. In embodiments, X³ is —Br. In embodiments, X³ is —I. In embodiments, X⁴ is halogen. In embodiments, halogen is —F, —Cl, —Br, —I. In embodiments, X⁴ is —F. In embodiments, X⁴ is —Cl. In embodiments, X⁴ is —Br. In embodiments, X⁴ is —I. In embodiments, X⁵ is halogen. In embodiments, halogen is —F, —Cl, —Br, —I. In embodiments, X⁵ is —F. In embodiments, X⁵ is —Cl. In embodiments, X⁵ is —Br. In embodiments, X⁵ is —I.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently unsubstituted alkyl, unsubstituted heteroalkyl, unsubstituted cycloalkyl, unsubstituted heterocycloalkyl, unsubstituted aryl, or unsubstituted heteroaryl.

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) or unsubstituted alkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) alkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently unsubstituted alkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted or unsubstituted alkyl (e.g., C₁-C₈ alkyl, C₁-C₆ alkyl, or C₁-C₄ alkyl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted alkyl (e.g., C₁-C₈ alkyl, C₁-C₆ alkyl, or C₁-C₄ alkyl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently unsubstituted alkyl (e.g., C₁-C₈ alkyl, C₁-C₆ alkyl, or C₁-C₄ alkyl).

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} and are independently substituted (e.g., substituted with a substituent group, a size-limited substituent group, or lower substituent group) or unsubstituted heteroalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) heteroalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently unsubstituted heteroalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted or unsubstituted heteroalkyl (e.g., 2 to 8 membered, 2 to 6 membered, 4 to 6 membered, 2 to 3 membered, or 4 to 5 membered). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D}, are independently substituted heteroalkyl (e.g., 2 to 8 membered, 2 to 6 membered, or 2 to 4 membered). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted heteroalkyl (e.g., 2 to 8 membered, 2 to 6 membered, or 2 to 4 membered).

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) or unsubstituted cycloalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) cycloalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted cycloalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted or unsubstituted cycloalkyl (e.g., C₃-C₈ cycloalkyl, C₃-C₆ cycloalkyl, or C₅-C₆ cycloalkyl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted cycloalkyl (e.g., C₃-C₈ cycloalkyl, C₃-C₆ cycloalkyl, or C₅-C₆ cycloalkyl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently unsubstituted cycloalkyl (e.g., C₃-C₈ cycloalkyl, C₃-C₆ cycloalkyl, or C₅-C₆ cycloalkyl).

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) or unsubstituted heterocycloalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) heterocycloalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted heterocycloalkyl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted or unsubstituted heterocycloalkyl (e.g., 3 to 8 membered, 3 to 6 membered, or 5 to 6 membered). In embodiments, R¹, R^{1A}, R^{1B}, R^{1C}, R^{1D}, R², R^{2A}, R^{2B}, R^{2C}, R^{2D}, R³, R^{3A}, R^{3B}, R³, R^{3D}, R⁴, R^{4A}, R^{4A}, R^{4B}, R^{4C}, R^{4D}, R⁵, R^{5A}, R^{5B}, R^{5C}, and R^{5D} are independently substituted heterocycloalkyl (e.g., 3 to 8 membered, 3 to 6 membered, or 5 to 6 membered). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted heterocycloalkyl (e.g., 3 to 8 membered, 3 to 6 membered, or 5 to 6 membered).

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) or unsubstituted aryl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) aryl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted aryl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted or unsubstituted aryl (e.g., C₆-C₁₀ aryl, C₁₀ aryl, or phenyl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted aryl (e.g., C₆-C₁₀ aryl, C₁₀ aryl, or phenyl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted aryl (e.g., C₆-C₁₀ aryl, C₁₀ aryl, or phenyl).

In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) or unsubstituted heteroaryl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted (e.g., substituted with a substituent group, a size-limited substituent group, or lower substituent group) heteroaryl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted heteroaryl. In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted or unsubstituted heteroaryl (e.g., 5 to 10 membered heteroaryl, 5 to 9 membered heteroaryl, or 5 to 6 membered heteroaryl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently substituted heteroaryl (e.g., 5 to 10 membered heteroaryl, 5 to 9 membered heteroaryl, or 5 to 6 membered heteroaryl). In embodiments, R¹, R^{1A}, R^{1B}, R^{1C}, R^{1D}, R², R^{2A}, R^{2B}, R^{2C}, R^{2D}, R³, R^{3A}, R^{3B}, R^{3C}, R^{3D}, R⁴, R^{4A}, R^{4A}, R^{4B}, R^{4C}, R^{4D}, R⁵, R^{5A}, R^{5B}, R^{5C}, and R^{5D} are independently an unsubstituted heteroaryl (e.g., 5 to 10 membered heteroaryl, 5 to 9 membered heteroaryl, or 5 to 6 membered heteroaryl).

In embodiments, the compound is:

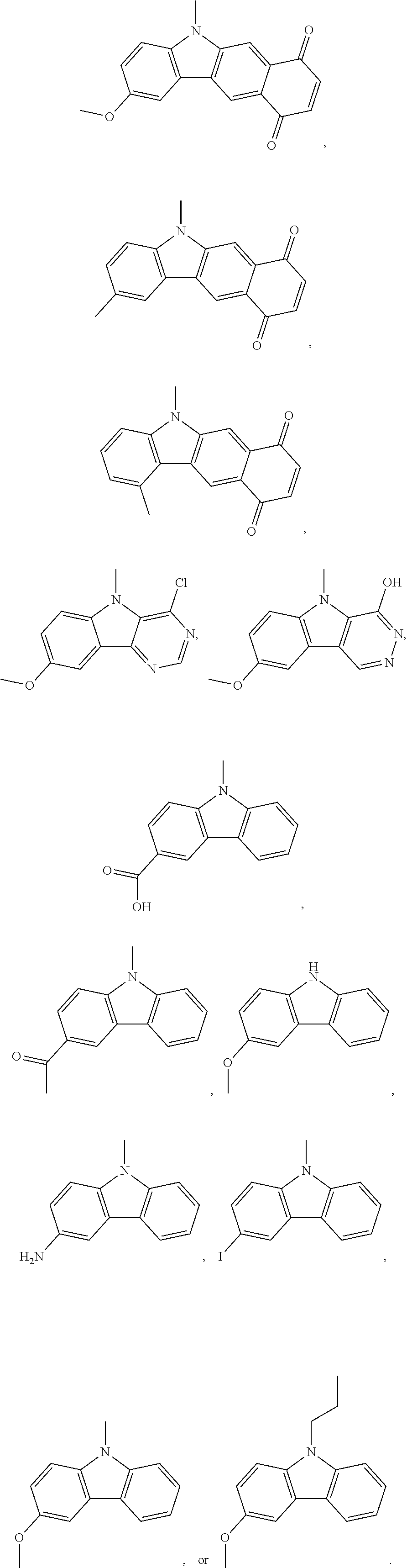

In embodiments, the compound is useful as a comparator compound. In embodiments, the comparator compound can be used to assess the activity of a test compound as set forth in an assay described herein (e.g., in the examples section, figures, or tables).

Methods of Treatment

Provided herein are methods of treating a hematological cancer in a subject in need thereof. These methods are effective in treating cold hematological cancers, such as leukemia or lymphoma. The disclosed methods comprise administering to the subject an effective amount of a PRMT9 inhibitor. In embodiments, the PRMT9 inhibitor is a short-hairpin RNA (shRNA), a small interference RNA (siRNA), a piwi-interacting RNA (piRNA), a microRNA (miRNA), an antisense oligonucleotide, such as a GapmeR or a morpholinooligonucleotide, a CRISPR Cas guide RNA (gRNA), a small molecule compound, or an anti-PRMT9 antibody. In embodiments, the PRMT9 inhibitor is a short-hairpin RNA (shRNA). In embodiments, the PRMT9 inhibitor is a small interference RNA (siRNA). In embodiments, the PRMT9 inhibitor is a piwi-interacting RNA (piRNA). In embodiments, the PRMT9 inhibitor is a microRNA (miRNA). In embodiments, the PRMT9 inhibitor is an antisense oligonucleotide. In embodiments, the PRMT9 inhibitor is a GapmeR. In embodiments, the PRMT9 inhibitor is a morpholinooligonucleotide. In embodiments, the PRMT9 inhibitor is a CRISPR Cas guide RNA (gRNA). In embodiments, the CRISPR Cas guide RNA is CRISPR Cas 12 guide RNA. In embodiments, the CRISPR Cas guide RNA is CRISPR Cas 9 guide RNA. In embodiments, the PRMT9 inhibitor is an anti-PRMT9 antibody. In embodiments, the PRMT9 inhibitor is an anti-di-methylated poly (A) binding protein cytoplasmic 1 (PABPC1) antibody.

In embodiments, the PRMT9 inhibitor is a small molecule compound of the structural Formulae (I), (II), (III), (Ia), or (Ib) or a pharmaceutically acceptable salt thereof, as described herein. In embodiments, provided herein are methods of inhibiting protein arginine methyltransferase 9 (PRMT9), the method comprising contacting PRMT9 with a compound as described herein, including embodiments, of the structural Formulae (I), (II), (III), (Ia), or (Ib) or a pharmaceutically acceptable salt thereof. In embodiments, provided herein are methods of treating or preventing a disease or disorder mediated by PRMT9 with a PRMT9 inhibitor as described herein, including a compound of the structural Formulae (I), (II), (III), (Ia), or (Ib) or a pharmaceutically acceptable salt thereof. In embodiments, the method includes administering a therapeutically effective amount of a PRMT9 inhibitor as described herein, including a compound of the structural Formulae (I), (II), (III), (Ia), or (Ib) or a pharmaceutically acceptable salt thereof.

In embodiments, the disease or disorder is cancer. In embodiments, there is provided a method of treating a cancer in a subject in need thereof, wherein the method comprises administering to the subject a therapeutically effective amount of a protein arginine methyltransferase 9 (PRMT9) inhibitor described herein, including embodiments (e.g., structural Formulae (I), (II), (III), (Ia), or (Ib), or a pharmaceutically acceptable salt thereof).

In accordance with the present invention, a compound or pharmaceutical salt thereof can be used to treat or prevent a proliferative condition or disorder, including a cancer, such as a hematological cancer. The present invention also provides methods of treating or preventing cancer-related diseases, disorders or conditions, including, for example, immunogenic tumors, non-immunogenic tumors, dormant tumors, virus-induced cancers, adenocarcinomas, lymphomas, carcinomas, melanomas, leukemias, myelomas, sarcomas, teratocarcinomas, chemically-induced cancers, metastasis, and angiogenesis. The invention contemplates reducing tolerance to a tumor cell or cancer cell antigen, e.g., by modulating activity of a regulatory T-cell and/or a CD8+ T-cell (see, e.g., Ramirez-Montagut, et al. (2003) Oncogene 22:3180-87; and Sawaya, et al. (2003) New Engl. J. Med. 349:1501-09).

In embodiments, the cancer is an immune-cold cancer. In embodiments, the tumor or cancer is a cancer of the blood or bone marrow. In embodiments, the tumor or cancer is leukemia. In embodiments, the cancer is a lymphoma.

In embodiments, the leukemia is acute nonlymphocytic leukemia, chronic lymphocytic leukemia, acute granulocytic leukemia, acute lymphoblastic leukemia, acute myeloid leukemia, chronic myelomonocytic leukemia, chronic myelogenous leukemia, chronic neutrophilic leukemia, immunoblastic large cell leukemia, megakaryoblastic leukemia, acute megakaryocytic leukemia, promyelocytic leukemia, erythroleukemia, chronic granulocytic leukemia, acute promyelocytic leukemia, adult T-cell leukemia, aleukemic leukemia, leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

In embodiments, the lymphoma is non-Hodgkin lymphoma, Hodgkin's disease, small lymphocytic lymphoma, mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, precursor B-lymphoblastic lymphoma, lymphoplasmacytic lymphoma, hairy cell leukemia, primary central nervous system (CNS) lymphoma, and primary intraocular lymphoma, cunateous T-cell lymphoma, peripheral T-cell lymphoma, anaplastic large cell lymphoma, mycosis fungoides, precursor T-lymphoblastic lymphoma, adult T-cell leukemia/lymphoma, angioimmunoblastic T-cell lymphoma, extranodal natural killer/T-cell lymphoma, enteropathy-associated intestinal T-cell lymphoma, or anaplastic large cell lymphoma (ALCL).

In embodiments, the cancer is multiple myeloma.

In embodiments, the cancer is a myeloproliferative disorder. In embodiments, the myeloproliferative disorder is chronic myelogenous leukemia. In embodiments, the myeloproliferative disorder is myelofibrosis. In embodiments, the myeloproliferative disorder is polycythemia vera. In embodiments, the myeloproliferative disorder is thrombocytosis. In embodiments, the myeloproliferative disorder is chronic neutrophilic leukemia. In embodiments, the myeloproliferative disorder is eosinophilia.

In embodiments, the methods of treating cancer disclosed herein may further include co-administering a chemotherapeutic agent or anticancer agent in combination with a PRMT9 inhibitor as described herein, including a compound of structural Formulae (I), (II), (III), (Ia), or (Ib), or a pharmaceutically acceptable salt thereof. In embodiments, the chemotherapeutic agent or anticancer agent is an antiproliferative/antineoplastic drug, an antimetabolite, an antitumour antibiotic, an antimitotic agent, a topoisomerase inhibitor, a cytostatic agent, an oestrogen receptor down regulator, an antiandrogen, a LHRH antagonist or LHRH agonist, a progestogen, an aromatase inhibitor, an inhibitor of 5.alpha.-reductase, an agent which inhibits cancer cell invasion, an inhibitor of growth factor function, a farnesyl transferase inhibitor, a tyrosine kinase inhibitor, a serine/threonine kinase inhibitor, an inhibitor of the epidermal growth factor family, an inhibitor of the platelet-derived growth factor family, an inhibitor of the hepatocyte growth factor family; an antiangiogenic agent, a vascular damaging agent, an agent used in antisense therapy, an anti-ras antisense, an agent used in a gene therapy, an immunotherapeutic agent, or an antibody.

In embodiments drawn to methods of treating a hematological cancer, the administration of a therapeutically effective amount of a PRMT9 inhibitor as described herein results in a cancer survival rate greater than the cancer survival rate observed by administering an anti-cancer agent. In further embodiments drawn to methods of treating cancer, the administration of a therapeutically effective amount of a compound described herein (e.g., PRMT9 inhibitor) results in a reduction of tumor size or a slowing of tumor growth greater than reduction of tumor size or tumor growth observed following administration of an anti-cancer agent.

The present invention contemplates the administration of the compounds described herein, and compositions (e.g., pharmaceutical salts, pharmaceutical composition) thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the compounds disclosed herein over a defined period of time. In embodiments, the administration is oral, lingual, sublingual, parenteral, rectal, topical, transdermal or pulmonary administration.

The compounds of the present invention may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED50.

In addition, an effective dose of the compounds of the present invention may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the compounds contemplated by the present invention may be administered at dosage levels of about 0.01 mg/kg to about 50 mg/kg, or about 1 mg/kg to about 25 mg/kg, of subject body weight per day, one, two, three, four or more times a day, to obtain the desired therapeutic effect. For administration of an oral agent, the compositions can be provided in the form of tablets, capsules and the like containing from 0.05 to 1000 milligrams of the active ingredient, particularly 0.05, 0.1, 0.5, 1.0, 1.5, 2.0, 2.5, 5.0, 7.5, 10.0, 15.0, 20.0, 25.0, 50.0, 75.0, 100.0, 125.0, 150.0, 175.0, 200.0, 250.0, 300.0, 400.0, 500.0, 600.0, 750.0, 800.0, 900.0, and 1000.0 milligrams of the active ingredient. A pharmaceutically acceptable carrier(s), diluent(s) and/or excipient(s) may be present in an amount of from about 0.1 g to about 2.0 g.

Pharmaceutical Compositions

Additionally provided herein are pharmaceutical compositions for preventing or treating a hematological cancer in a subject in need thereof. The disclosed pharmaceutical compositions comprise an effective amount of a PRMT9 inhibitor and one or more excipients or additives.

In embodiments, the PRMT9 inhibitor is a short-hairpin RNA (shRNA), a small interference RNA (siRNA), a piwi-interacting RNA (piRNA), a microRNA (miRNA), an antisense oligonucleotide, such as a GapmeR or a morpholinooligonucleotide, a CRISPR Cas guide RNA (gRNA), a small molecule compound, or an anti-PRMT9 antibody. In embodiments, the PRMT9 inhibitor is a short-hairpin RNA (shRNA). In embodiments, the PRMT9 inhibitor is a small interference RNA (siRNA). In embodiments, the PRMT9 inhibitor is a piwi-interacting RNA (piRNA). In embodiments, the PRMT9 inhibitor is a microRNA (miRNA). In embodiments, the PRMT9 inhibitor is an antisense oligonucleotide. In embodiments, the PRMT9 inhibitor is a GapmeR. In embodiments, the PRMT9 inhibitor is a morpholinooligonucleotide. In embodiments, the PRMT9 inhibitor is a CRISPR Cas guide RNA (gRNA). In embodiments, the CRISPR Cas guide RNA is CRISPR Cas 12 guide RNA. In embodiments, the CRISPR Cas guide RNA is CRISPR Cas 9 guide RNA. In embodiments, the PRMT9 inhibitor is an anti-PRMT9 antibody. In embodiments, the PRMT9 inhibitor is an anti-di-methylated poly (A) binding protein cytoplasmic 1 (PABPC1) antibody.

In embodiments, the PRMT9 inhibitor is a small molecule compound of the structural Formulae (I), (II), (III), (Ia), or (Ib) or a pharmaceutically acceptable salt thereof, as described herein. In an aspect, there is provided a pharmaceutical composition, including a compound as described herein, including embodiments, e.g., structural Formula (I), (II), (III), (Ia), or (Ib) and a pharmaceutically acceptable excipient.

The compounds as described herein of the present disclosure may be in the form of compositions suitable for administration to a subject. In general, such compositions are “pharmaceutical compositions” comprising a PRMT9 inhibitor described herein and one or more pharmaceutically acceptable or physiologically acceptable excipients (e.g., acceptable diluents or carriers). In embodiments, the PRMT (inhibitor is present in a therapeutically effective amount. The pharmaceutical compositions may be used in the methods of the present disclosure; thus, for example, the pharmaceutical compositions can be administered ex vivo or in vivo to a subject in order to practice the therapeutic and prophylactic methods and uses described herein. The pharmaceutical compositions of the present disclosure can be formulated to be compatible with the intended method or route of administration; exemplary routes of administration are set forth herein.

The pharmaceutical compositions containing the PRMT9 inhibitor may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients that are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound described herein contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-hydroxyethyl)piperazine-N′-(2-ethanesulfonic acid) (HEPES); 2-(N-morpholino)ethanesulfonic acid (MES); 2-(N-morpholino)ethanesulfonic acid sodium salt (MES); 3-(N-morpholino)propanesulfonic acid (MOPS); and N-tris[hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a Wnt/catenin signaling pathway inhibitor, including implants (e.g., implantable pumps) and catheter systems, slow injection pumps and devices, all of which are well known to the skilled artisan.

Depot injections, which are generally administered subcutaneously or intramuscularly, may also be utilized to release a compound disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, NJ) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

Kits

In embodiments, provided herein are kits that comprise a PRMT9 inhibitor as described herein, excipients, and instructions for use. The kits are generally in the form of a physical structure housing various components, as described below, and may be utilized, for example, in practicing the methods described above.

A kit may include one or more of the PRMT9 inhibitors disclosed herein (e.g., provided in a sterile container), which may be in the form of a pharmaceutical composition suitable for administration to a subject. In embodiments, the PRMT9 inhibitors described herein can be provided in a form that is ready for use (e.g., a tablet or capsule) or in a form requiring, for example, reconstitution or dilution (e.g., a powder) prior to administration. When the compound is in a form that needs to be reconstituted or diluted by a user, the kit may also include diluents (e.g., sterile water), buffers, pharmaceutically acceptable excipients, and the like, packaged with, or separately from, the compound. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial). Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

Application of currently available immunotherapies to treat immune-cold cancers remains challenging. Protein arginine methyltransferase 9 (PRMT9) activities are elevated in patients with immune-cold cancers, correlating with poor prognosis and decreased response to immune checkpoint inhibitor (ICI). Herein, we show that targeting PRMT9 eliminates PRMT9-proficient/immune-cold cancers, including acute myeloid leukemia (AML) and diffuse large B-cell lymphoma (DLBCL), via multiple mechanisms including type-I interferon (IFN-I)-associated immunity. Specifically, ablating cancer intrinsic PRMT9 decreases arginine methylation of regulators implicated in RNA translation and DNA damage response, thereby suppressing cell proliferation. cGAS activation, which is seen following DNA damage, underlies cancer-cell-nonautonomous IFN-I response induced by PRMT9 inhibition. Genetically activating cGAS in AML cells blocked leukemia development. We also report synergy between PRMT9 inhibitor and anti-PD1 in eradicating the PRMT9-proficient cancers. We demonstrate that cancer cell-derived PRMT9 governs cell growth and immune evasion, and that combining a PRMT9 inhibitor, optionally with an ICI, is a novel anti-cancer strategy.

PRMT9 levels are elevated in AML leukemia stem cells

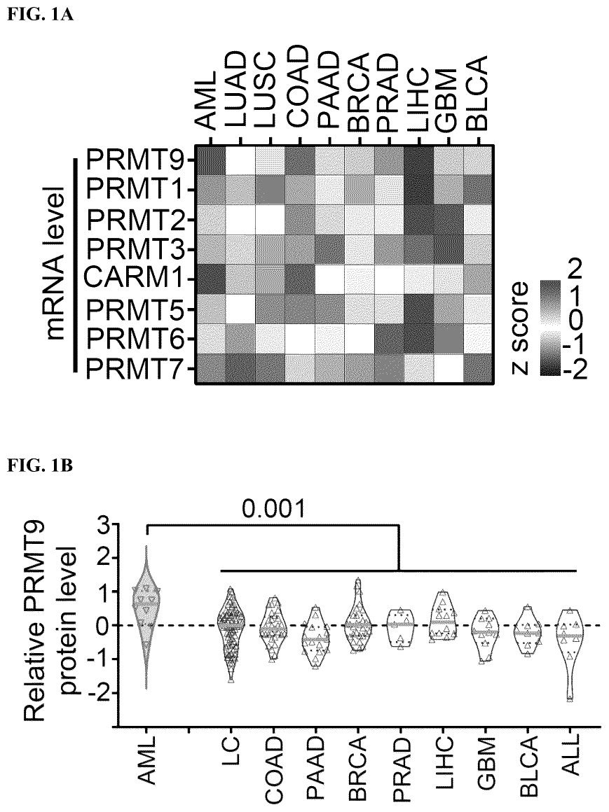

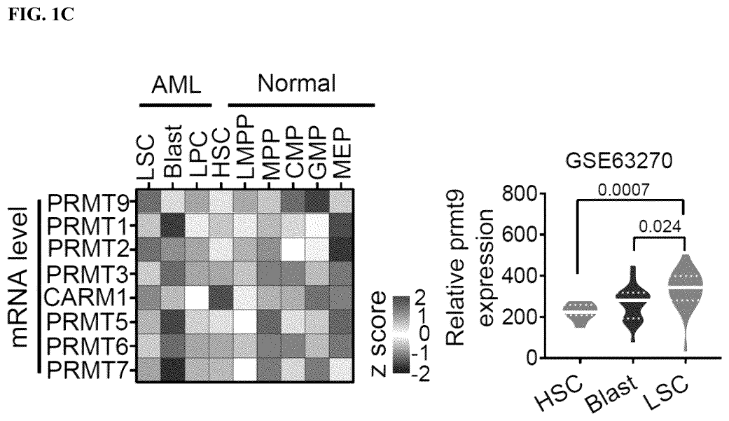

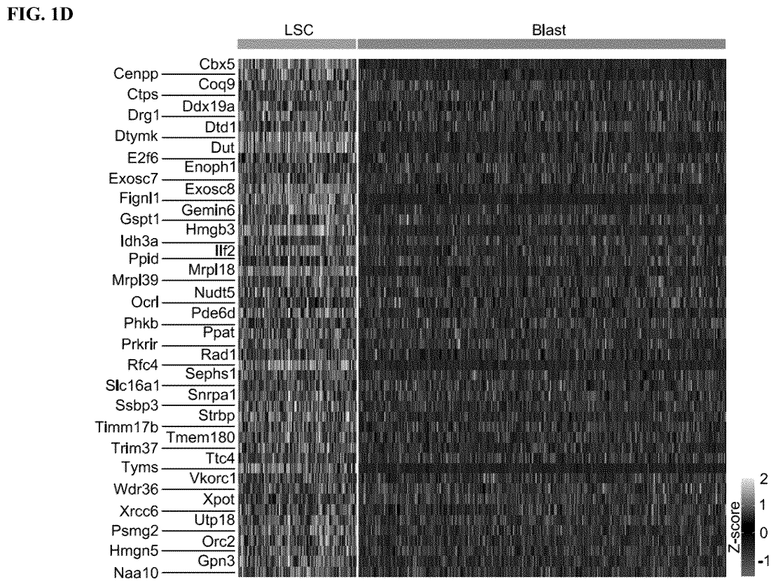

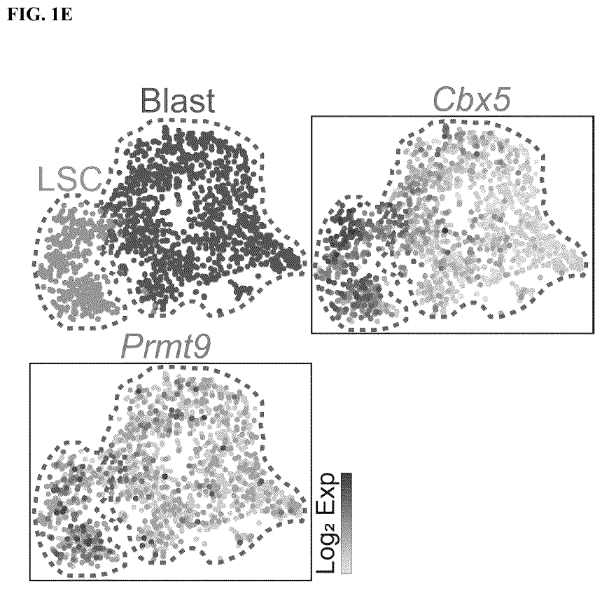

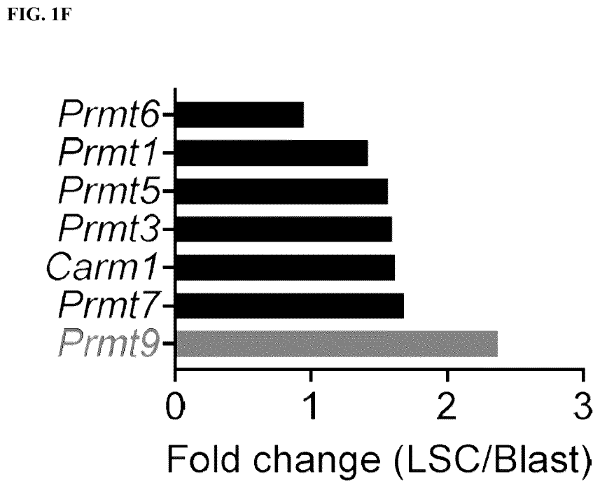

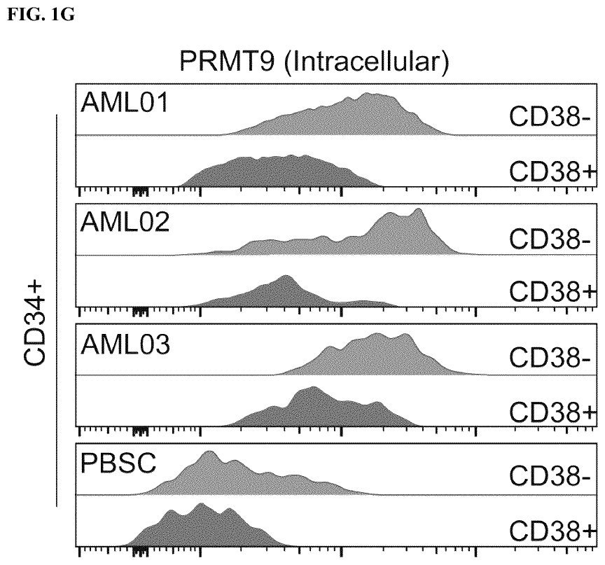

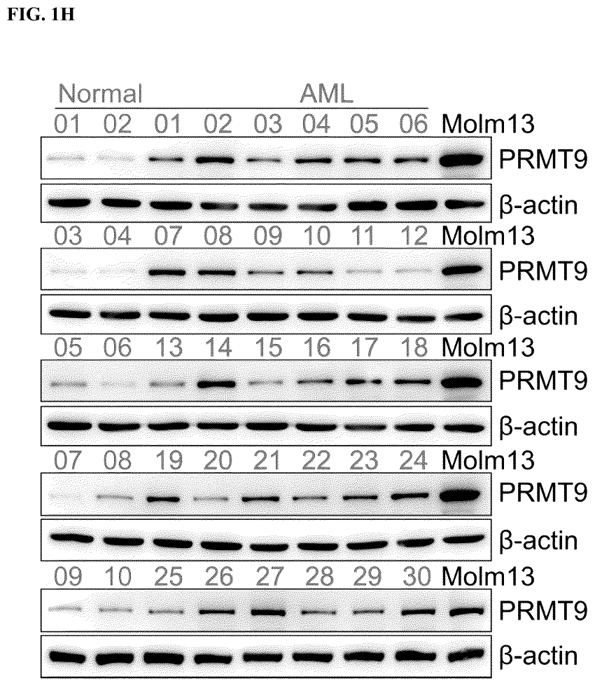

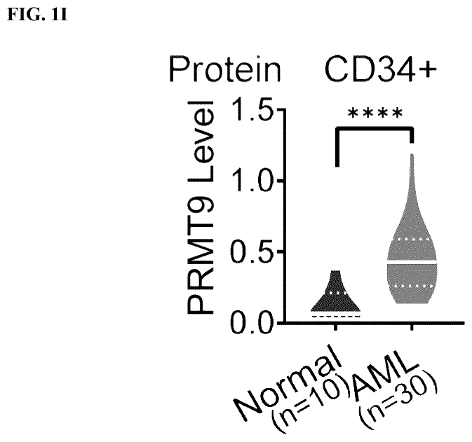

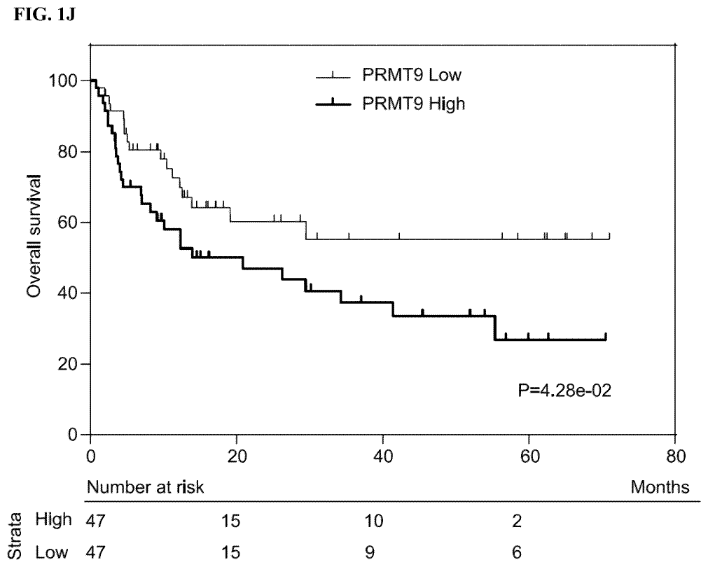

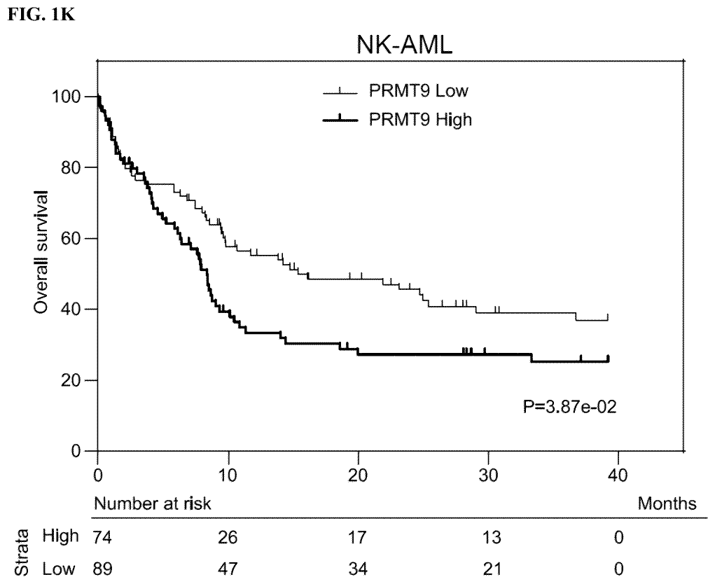

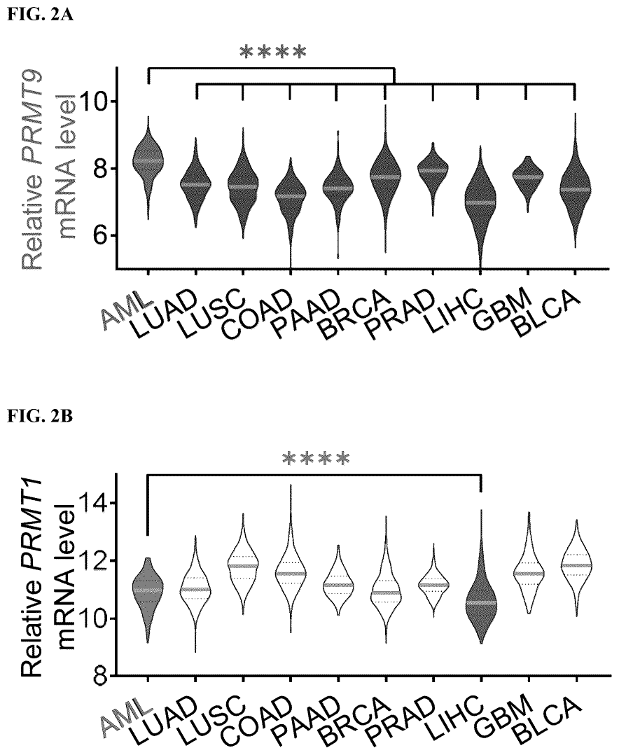

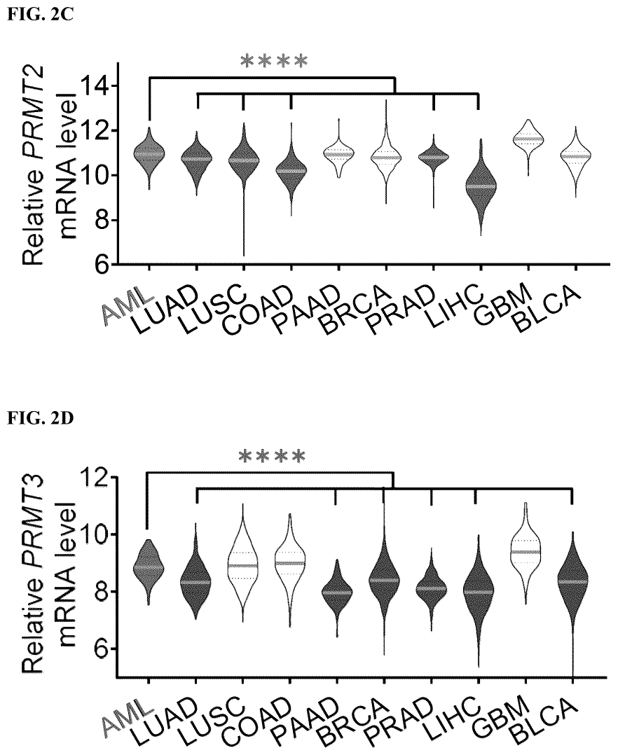

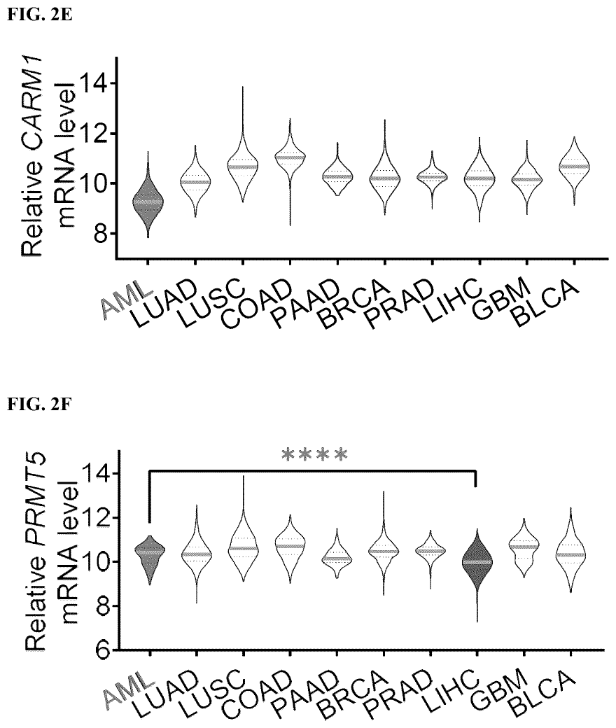

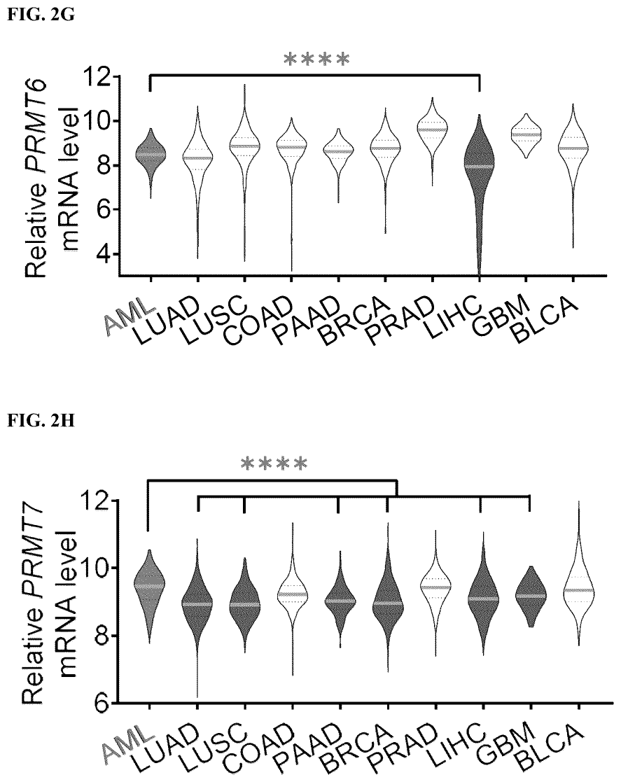

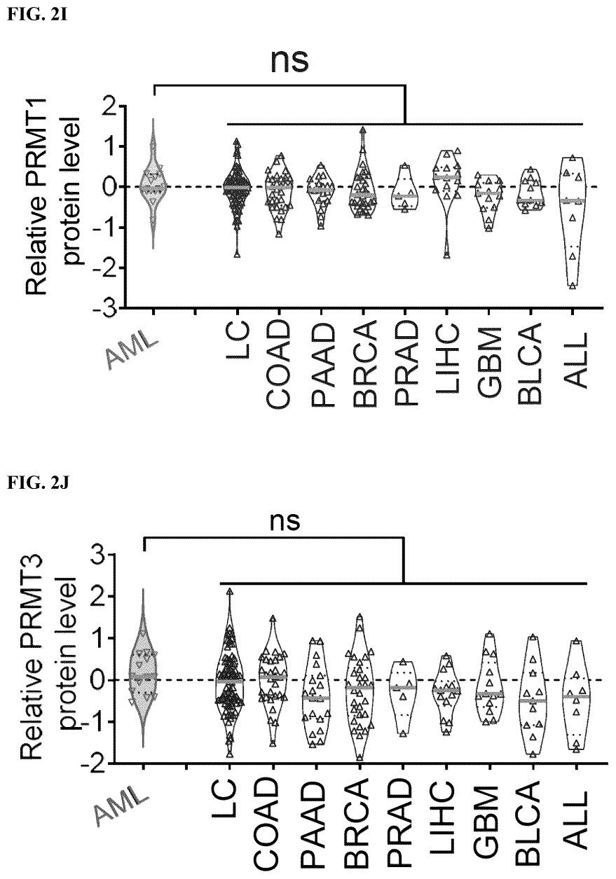

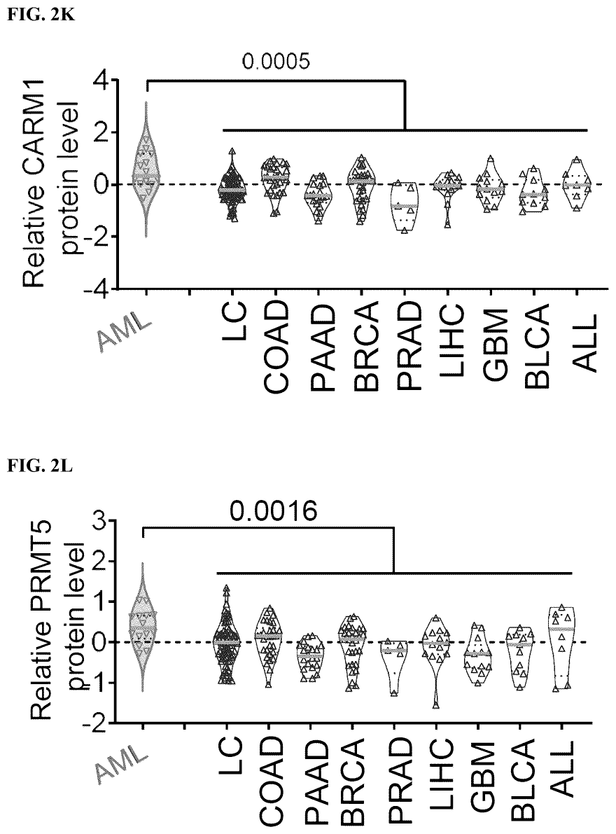

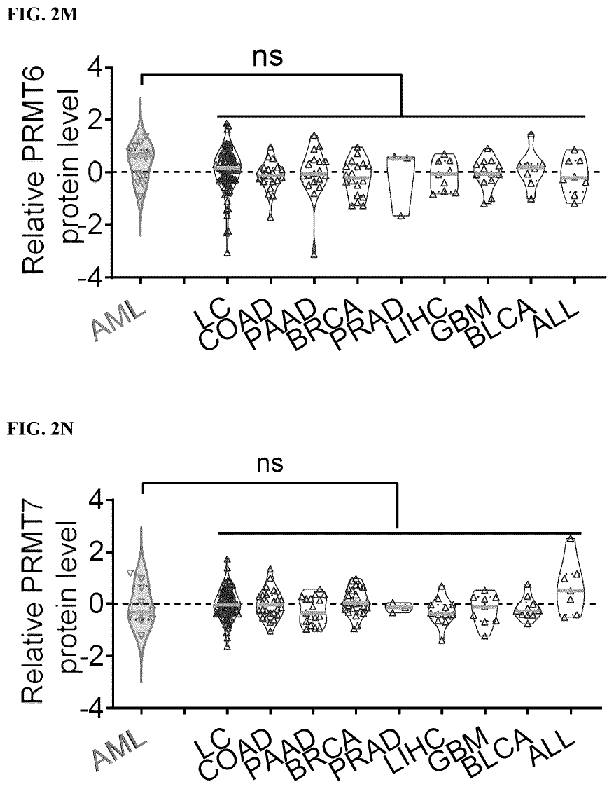

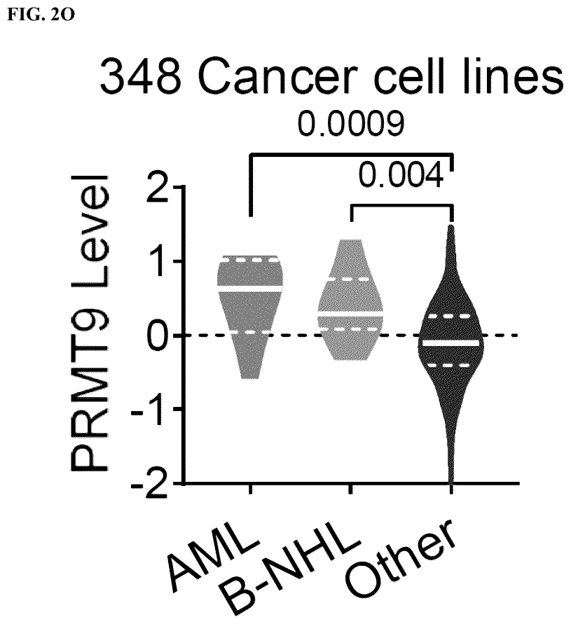

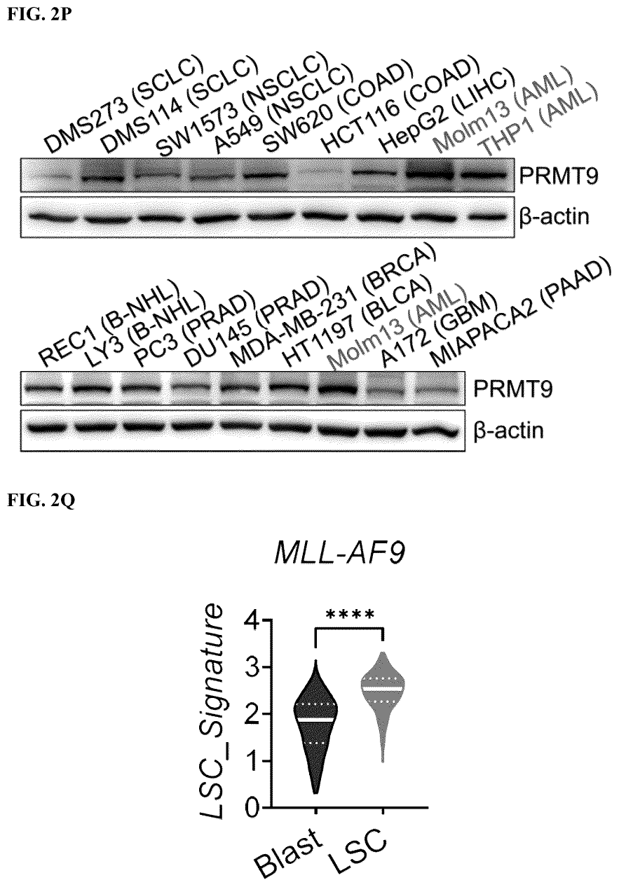

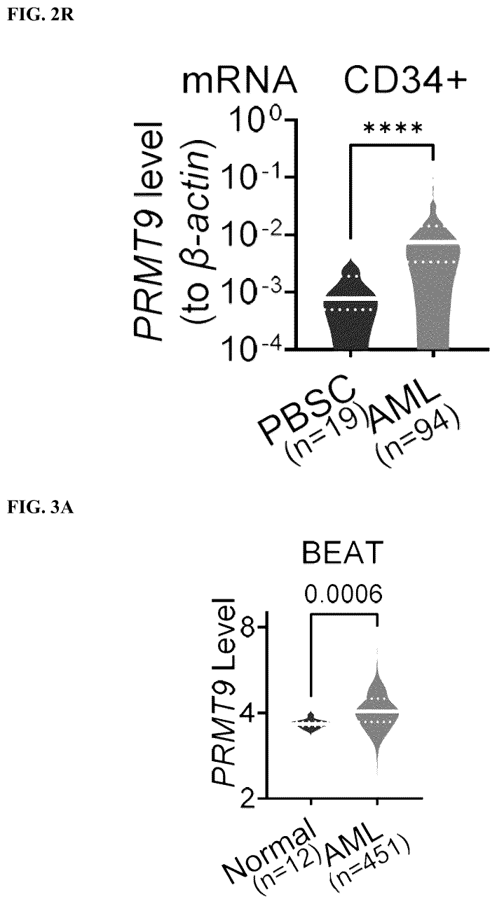

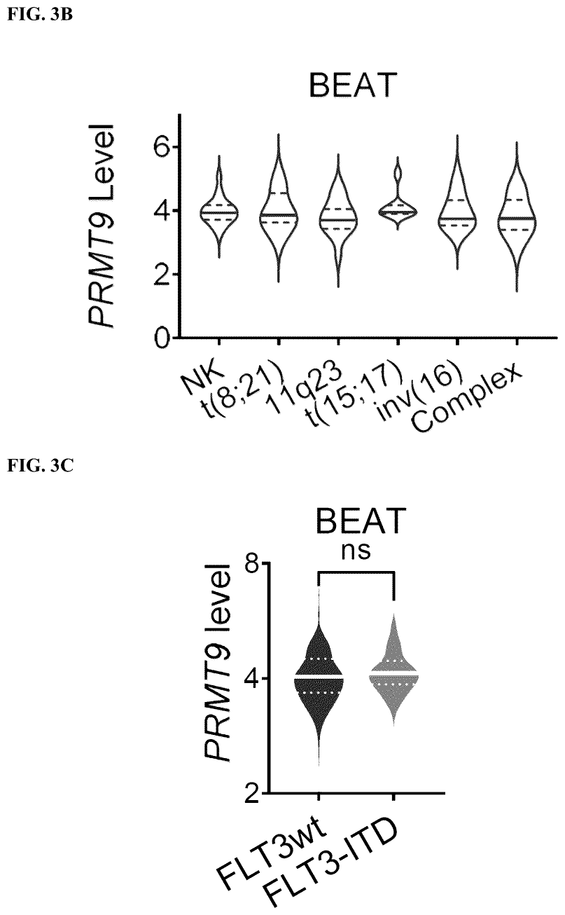

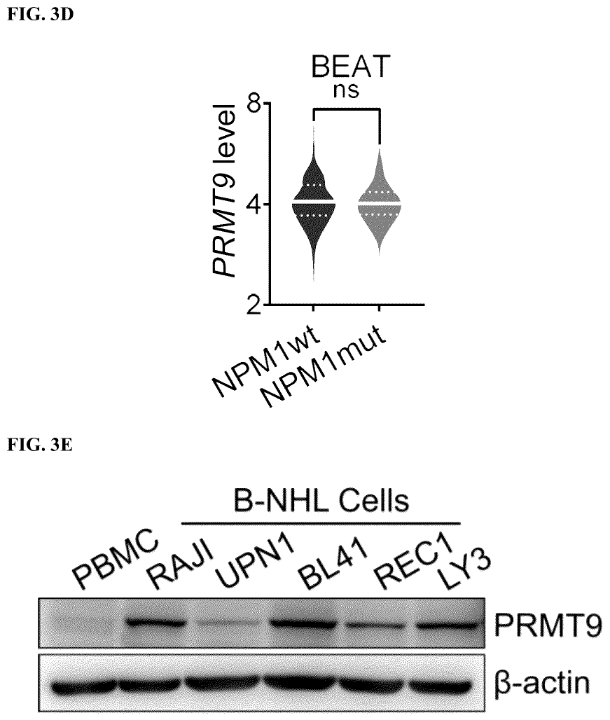

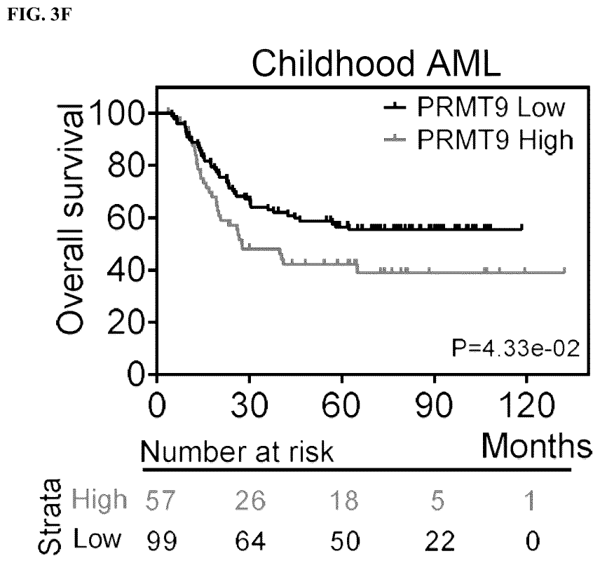

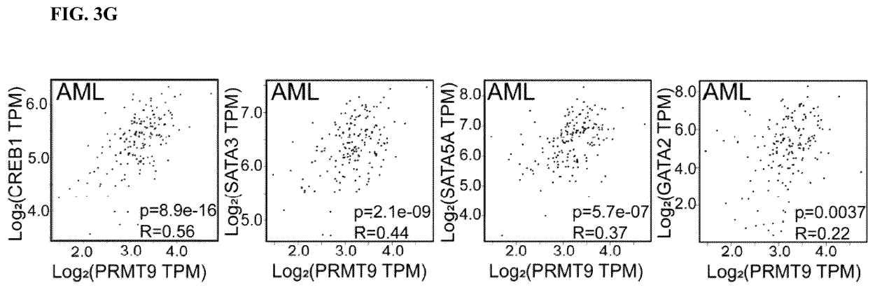

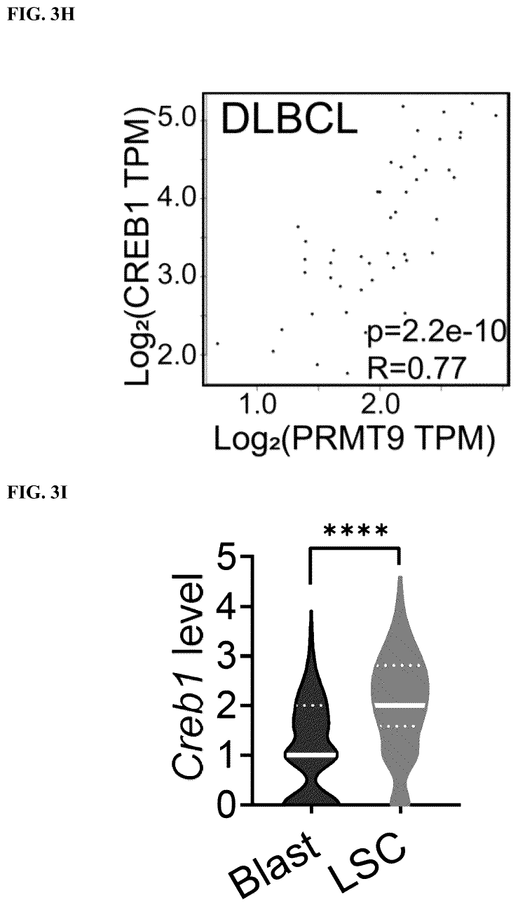

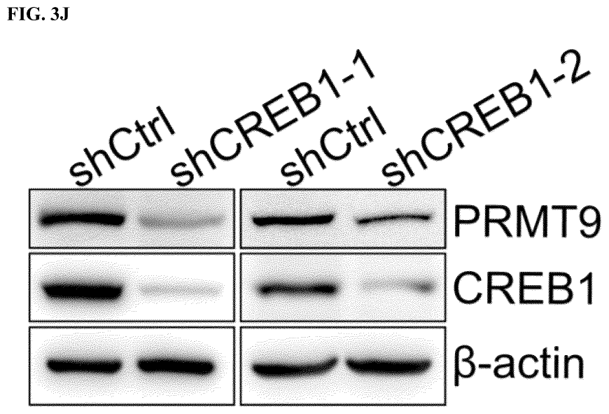

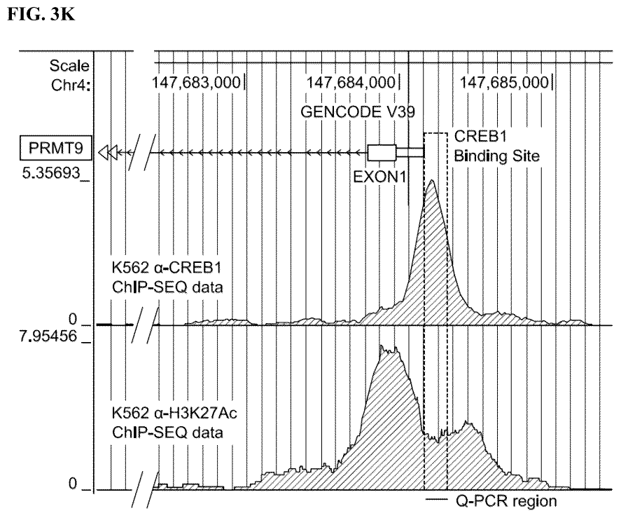

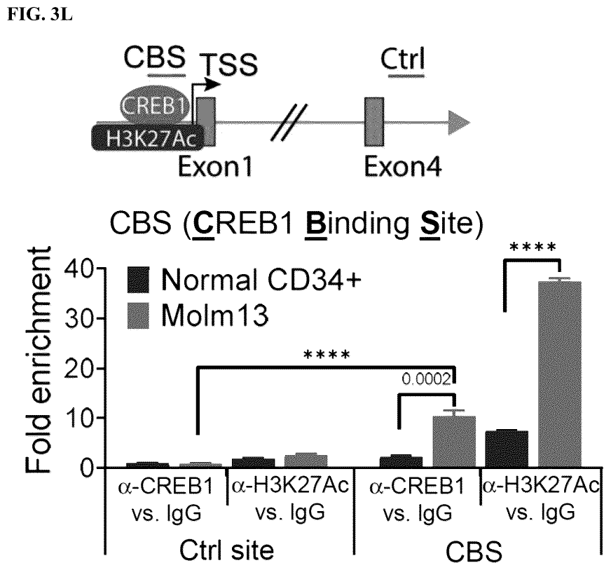

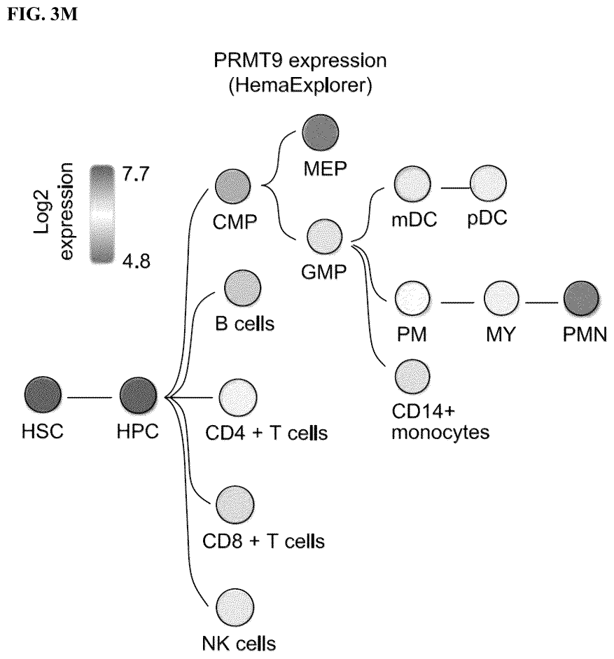

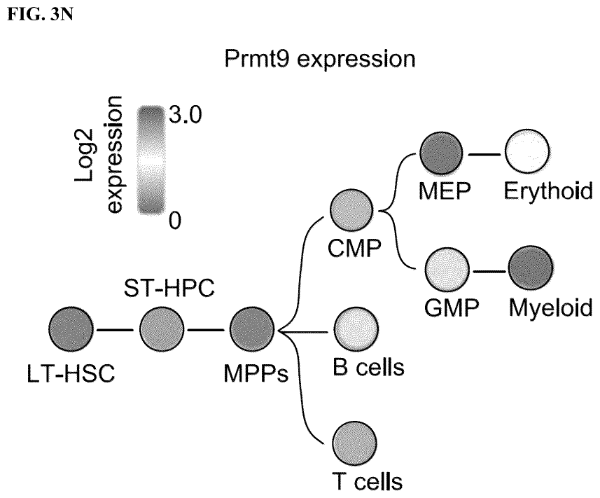

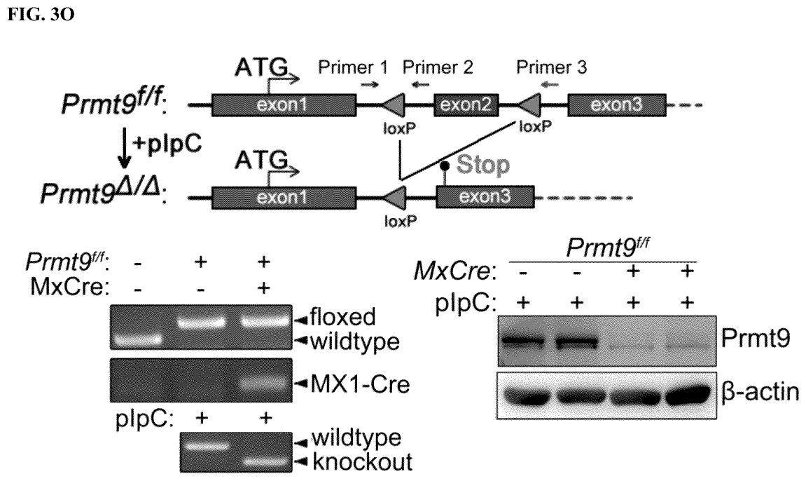

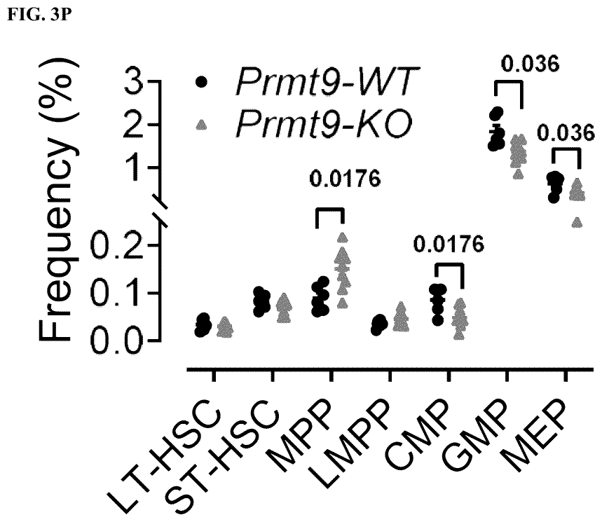

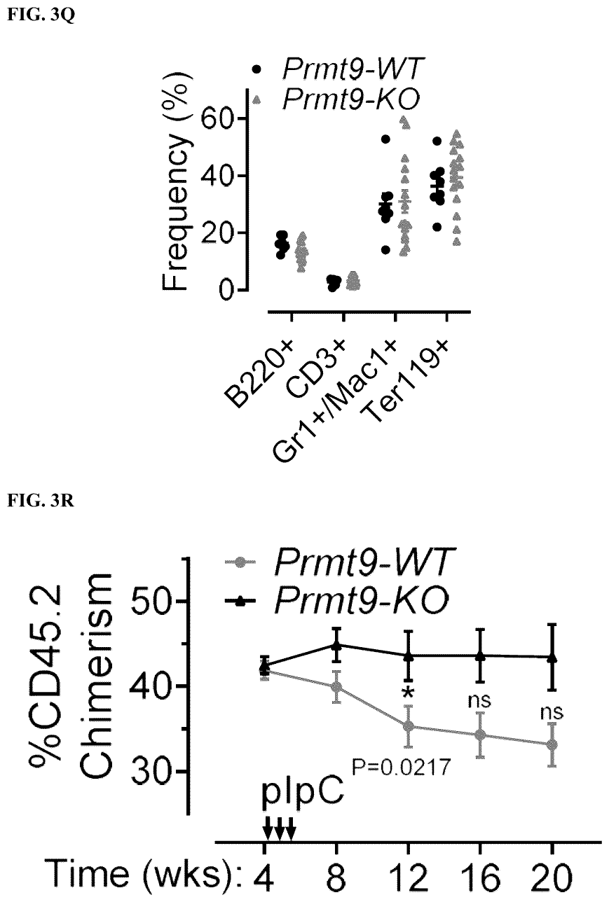

To profile PRMT (1-9) expression in cancers, their levels were assessed using RNAseq datasets (TCGA PanCancer Atlas) and a proteomic dataset including 375 cell lines across various cancer types. (Nusinow et al. Cell 180, 387-402 e316, doi:10.1016/j.cell.2019.12.023 (2020); Cerami, E. et al. TCancer discovery 2, 401-404, doi:10.1158/2159-8290.CD-12-0095 (2012)) Among the most deadly cancers, AML showed the highest PRMT9 mRNA levels (FIGS. 1A-1B; FIG. 2A). Siegel et al, Cancer statistics, 2022. CA: a cancer journal for clinicians 72, 7-33, doi:10.3322/caac.21708 (2022). Other PRMTs, such as PRMT1 and PRMT5, showed comparable levels in AML and other cancer types (FIG. 1A; FIGS. 2B-2N). The elevated PRMT9 protein levels in AML relative to other cancer types were also observed, based on evaluation of existing proteomics results (FIG. 1B, FIG. 2O) and by western blot analysis of other relevant cancer lines (FIG. 2P). Given the importance of cancer stem cells for AML pathogenesis, all PRMT levels were next assessed using a dataset including transcriptome results from normal hematopoietic stem and progenitor (HSPC) subsets as well as leukemia stem/progenitor subsets (GSE63270). Notably, PRMT9 levels were higher in LSCs relative to either normal HSCs or leukemia blasts (FIG. 1C). To validate PRMT9 elevation in LSCs, single-cell RNA sequencing (scRNAseq) analysis was performed on murine leukemic BM cells from an MLL-AF9 (MA9/GFP)-transduced c-Kit⁺ cell transplanted mouse, which develops AML. Graph-based clustering and dimensionality reduction were used with t-SNE to identify and visualize transcriptionally homogeneous cell clusters. Accordingly, among the leukemia cells, two main clusters were identified as LSCs and blasts, with the LSC cluster enriched based on a previously reported LSC signature (e.g., CBX5) of MLL leukemia mice (FIGS. 1D-1E; FIG. 2Q). Notably, when individual PRMT levels were compared in LSCs versus blasts, Prmt9 was the only Prmt whose levels were more than one-fold increase in LSCs relative to blasts (FIG. 1F). Next, PRMT9 protein levels were assessed in stem/progenitor subsets from AML specimens and samples from normal healthy donors (peripheral blood stem cell (PBSC)) using a PRMT9 antibody intracellular staining method. Notably, elevated PRMT9 protein levels were seen in AML in an LSC-enriched (CD34⁺CD38⁻) relative to a leukemia committed progenitor (CD34⁺CD38⁺) subset or to either of the normal HSPC subsets (CD34⁺CD38⁺ or CD34⁺CD38⁻) (FIG. 1G). Western blotting was also used to analyze an extended cohort of AML samples to measure PRMT9 protein levels in AML CD34⁺ subsets versus normal counterparts from healthy donors and found PRMT9 levels were consistently higher in AML (n=30) relative to normal counterparts (n=10) (FIGS. 1H-1I). Q-PCR analysis of CD34⁺ cells from another cohort of AML cases (n=94) and normal donors (n=19) confirmed PRMT9 upregulation in AML (FIG. 2R). Analysis of a publicly accessible AML dataset (n=463) showed similar results (FIG. 3A). PRMT9 levels were not associated with any particular cytogenetic abnormality or mutation, such as FLT3-ITD or NPM1, in this dataset (FIG. 3B-3D). PRMT9 protein levels were also higher in diffuse large B-cell lymphoma (DLBCL) lines, which represent a major non-Hodgkin's lymphoma (NHL) subtype are less responsive to ICI treatment (FIG. 2O, FIG. 3E).

To determine whether high PRMT9 expression levels are associated with prognosis of AML patients receiving standard chemotherapy, a cohort of patients were segregated into two groups based on PRMT9 expression levels at time of diagnosis (FIG. 1J). It was found that higher PRMT9 levels predicted shorter overall survival (FIG. 1J). Consistently, analysis of adult and pediatric AML datasets (GSE12417, TARGET-AML) yielded similar results (FIGS. 1K and 3F).

To define the mechanisms underlying PRMT9 mRNA upregulation, ChIP-seq data were analyzed in ChIPBase 2.0 and observed binding sites of hematopoiesis related transcription factors (CREB1, STAT5A, STAT3, GATA2) within 5 kb upstream of the PRMT9 transcription start site (TSS). Among them, CREB1, a known AML prognosticator, showed strongest correlation with PRMT9 expression in AML and DLBCL cohorts (FIG. 3G,3H). Interestingly, an increase of Creb1 was observed in LSCs relative to Blasts from the murine MA9 leukemia scRNAseq analysis (FIG. 3I). Notably, CREB1 knockdown (KD) decreased PRMT9 levels in Molm13 cells (FIG. 3J). Significant enrichment of CREB1 was next verified in Molm13 cells at the predicted PRMT9 promoter region relative to a control site (FIGS. 3K-3L). Moreover, in Molm13 cells, the PRMT9 promoter region showed significant enrichment of H3K27Ac (a mark of active transcription) relative to normal PBSC CD34⁺ cells (FIG. 3L).

PRMT9 is Dispensable for Normal Hematopoiesis

To investigate PRMT9 function in normal hematopoiesis, Prmt9 levels were assessed in subsets of BM cells from human and normal mouse. Analyses of human hematopoietic cell datasets revealed that PRMT9 levels were relatively higher in stem/progenitor cells than in mature lineages (FIG. 3M). Consistently, analysis of Prmt9 levels in mouse BM cells revealed a similar trend (FIG. 3N). Next, a conditional Prmt9 knockout (KO) model was developed by crossing Prmt9^{f/f} with Mx1-Cre mice (Mx1-Cre Prmt9^{f/f}, or Prmt9-cKO). In this mouse model, Prmt9 exon2 is flanked by loxP sites, and exon2 deletion by Cre creates a frameshift resulting in a functionally null allele (FIG. 3O). Following polyinosinic:polycytidylic acid (pIpC) administration and confirmation of Prmt9 deletion (FIG. 3O), Prmt9-cKO was monitored and littermate (Prmt9^{f/f}) control mice (8 weeks old) for changes in hematopoiesis. At 16 weeks after pIpC administration, no significant differences were observed in complete blood count (CBC) between two genotypes. BM analysis revealed that Prmt9 KO only modestly increased the MPP population and slightly decreased frequency of CMP, GMP and MEP populations (FIG. 3P), while no significant change was observed in populations of mature cells, such as T cells (FIG. 3Q). To evaluate repopulation capacity of HSPCs with Prmt9 KO, a competitive transplantation assay was performed using CD45.2 Prmt9-cKO mice or littermate controls as donors plus equal numbers of BM MNCs from CD45.1 WT mice as competitors. To do so, lethally-irradiated CD45.1 mice was transplanted with CD45.2/CD45.1 cell mixtures and then administered with pIpC. Notably, Prmt9-KO modestly decreased CD45.2 chimerism in recipients relative to controls (FIG. 3R). The data indicate that Prmt9 function is largely dispensable for normal steady-state hematopoiesis but required for hematopoietic stem/progenitor cells (HSPCs) self-renewal under transplantation stress.

PRMT9 Ablation Impairs Cancer Stem/Progenitor Cell Growth and Survival

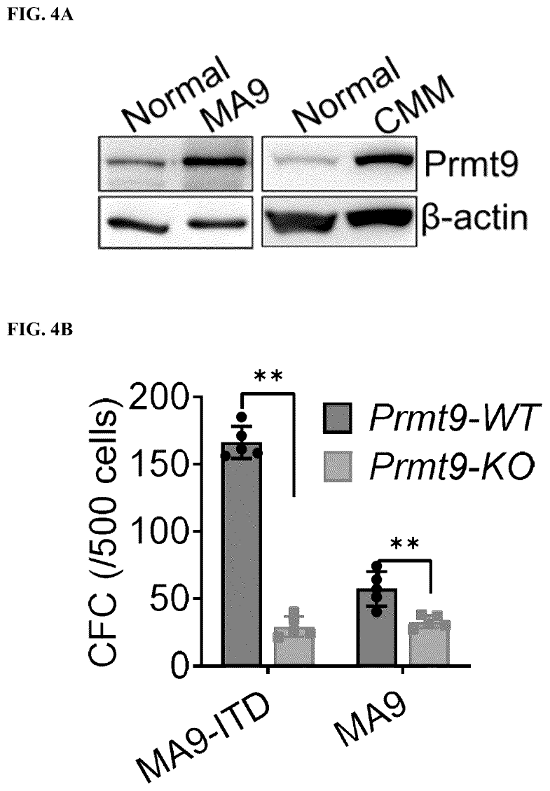

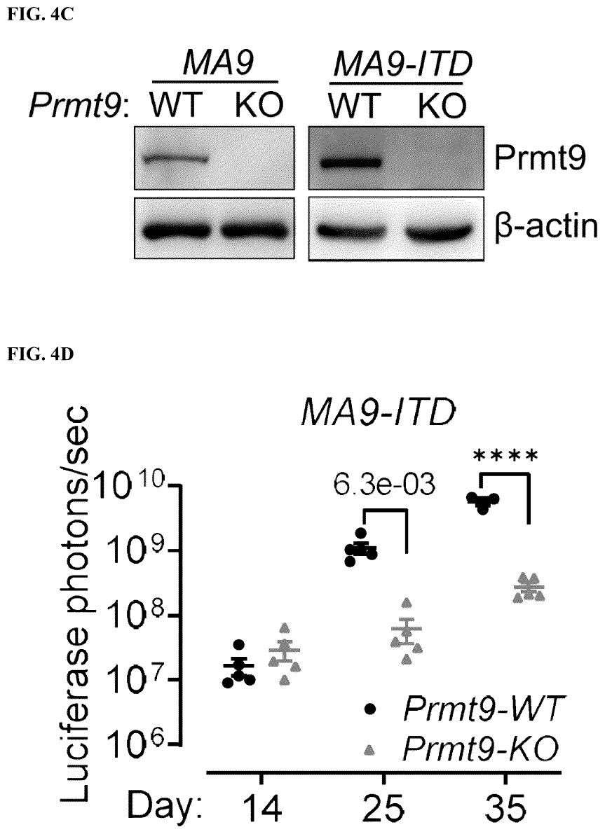

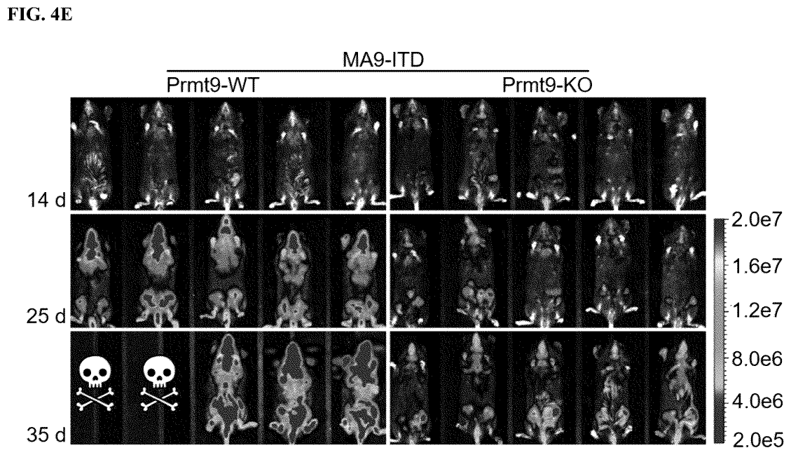

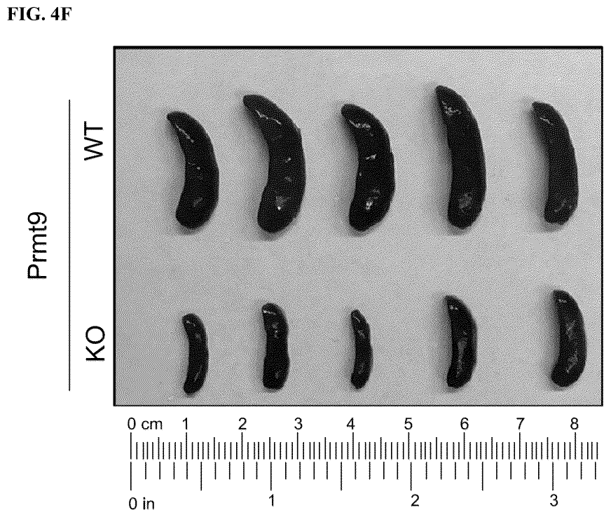

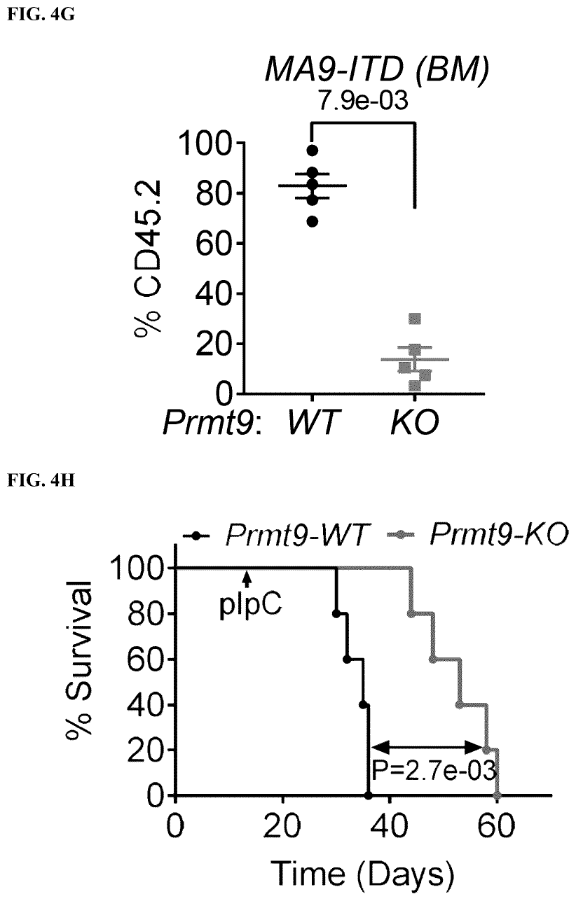

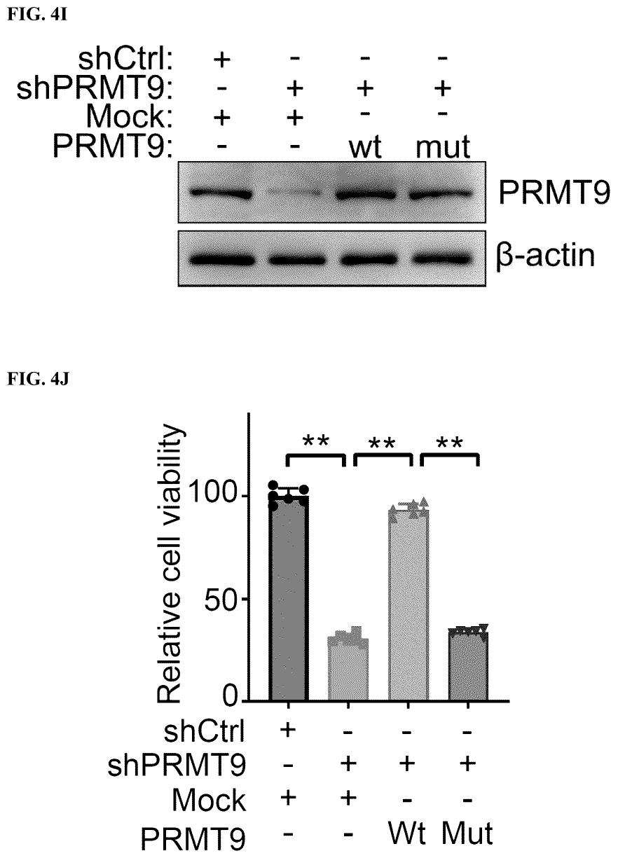

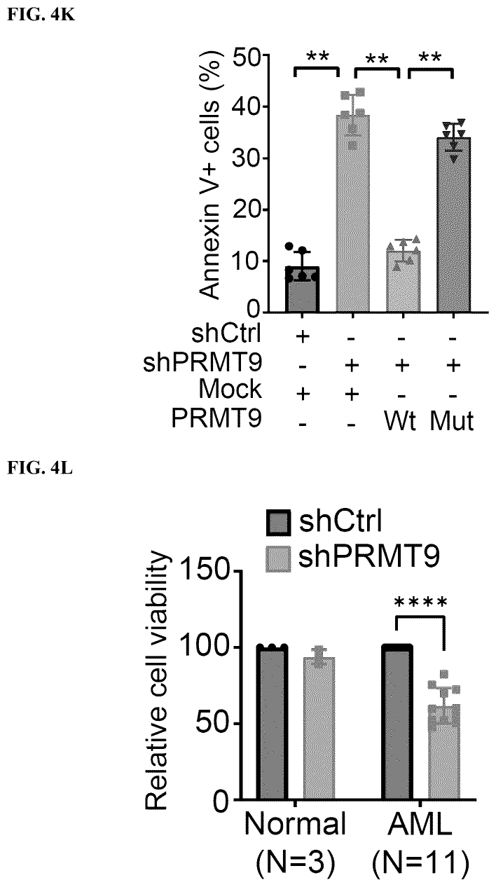

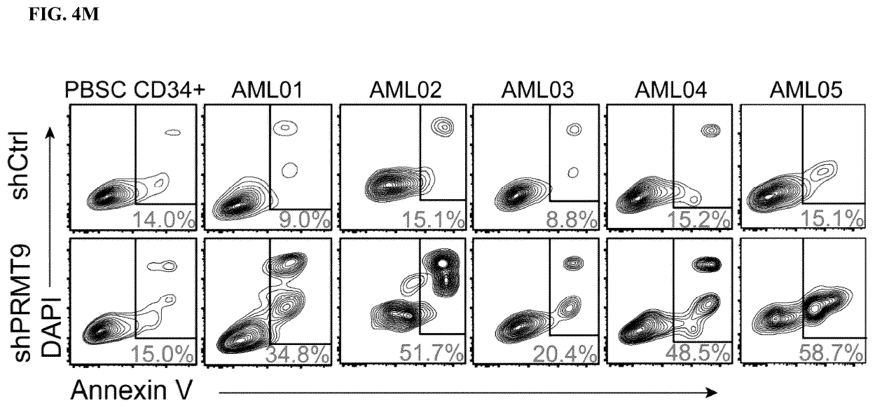

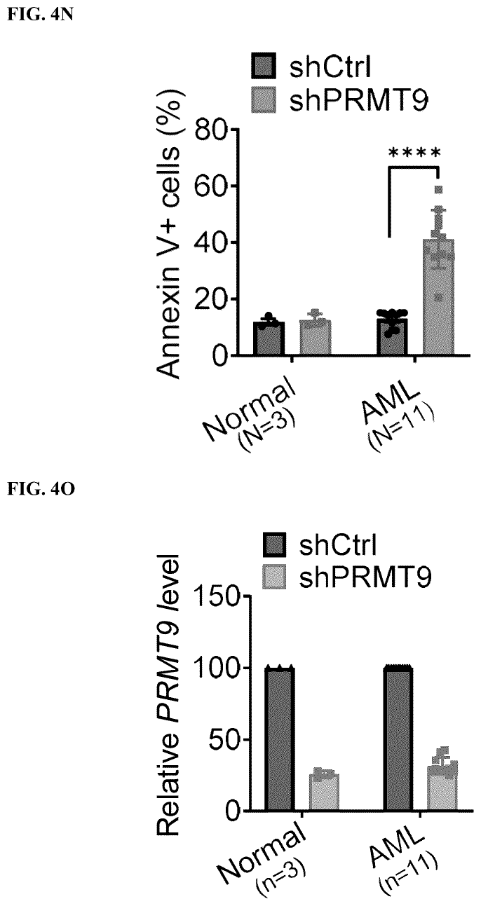

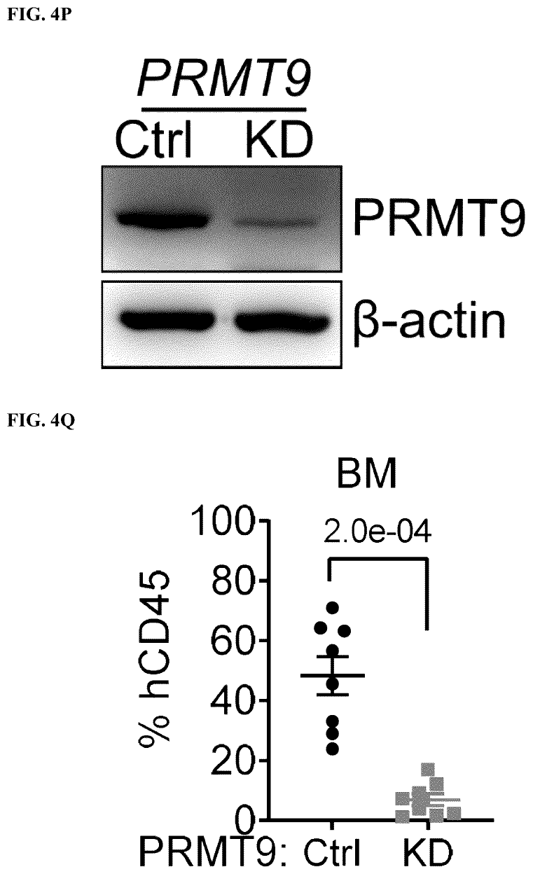

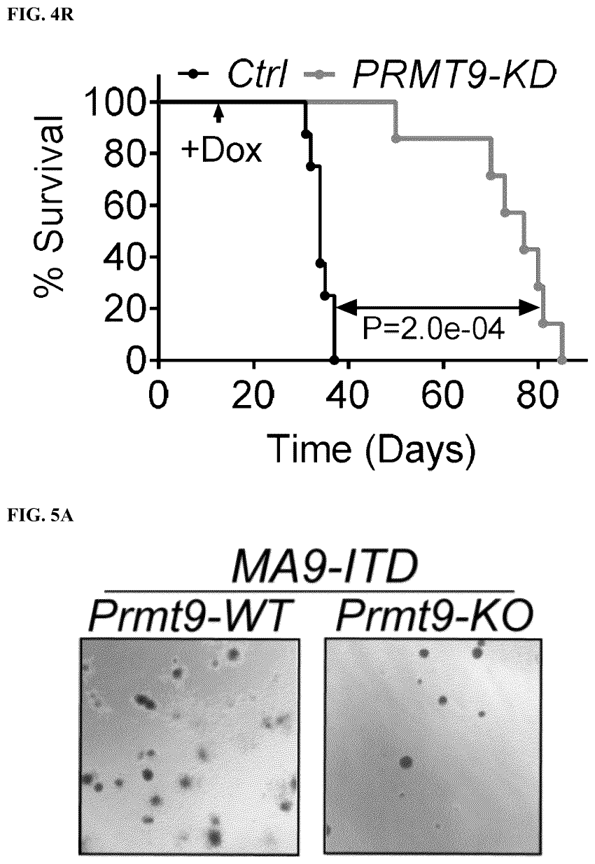

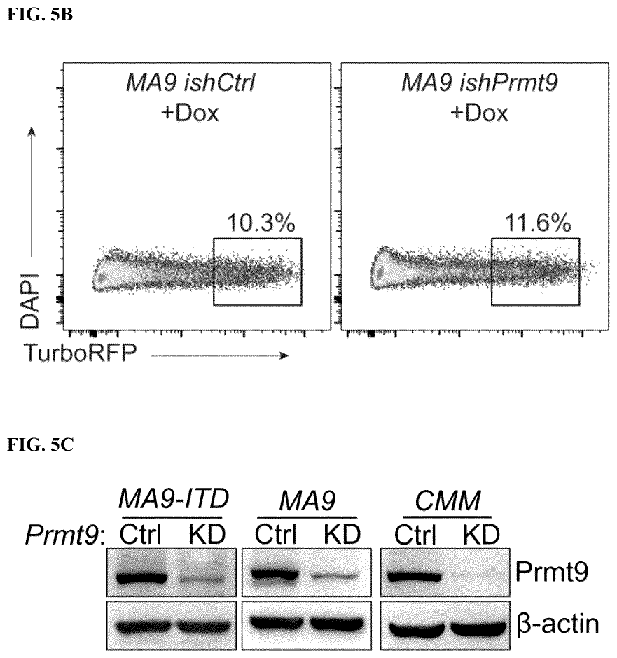

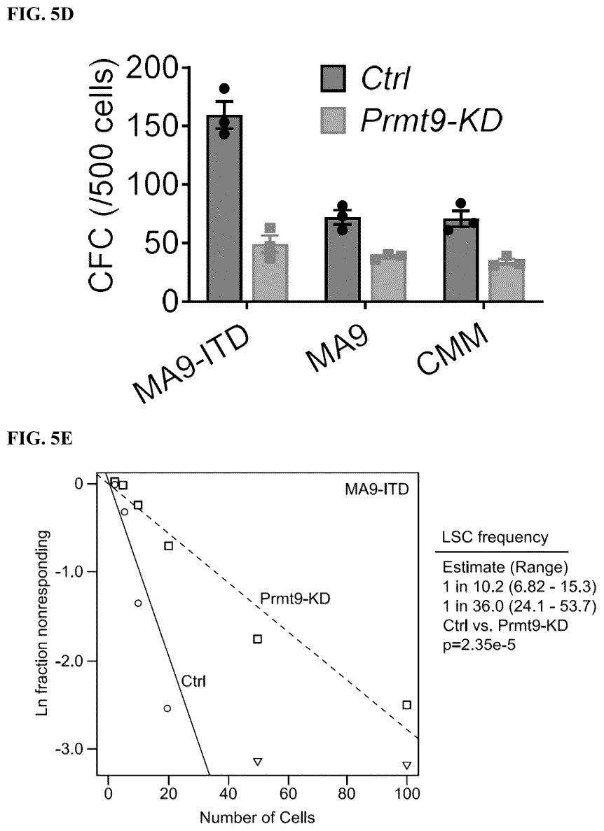

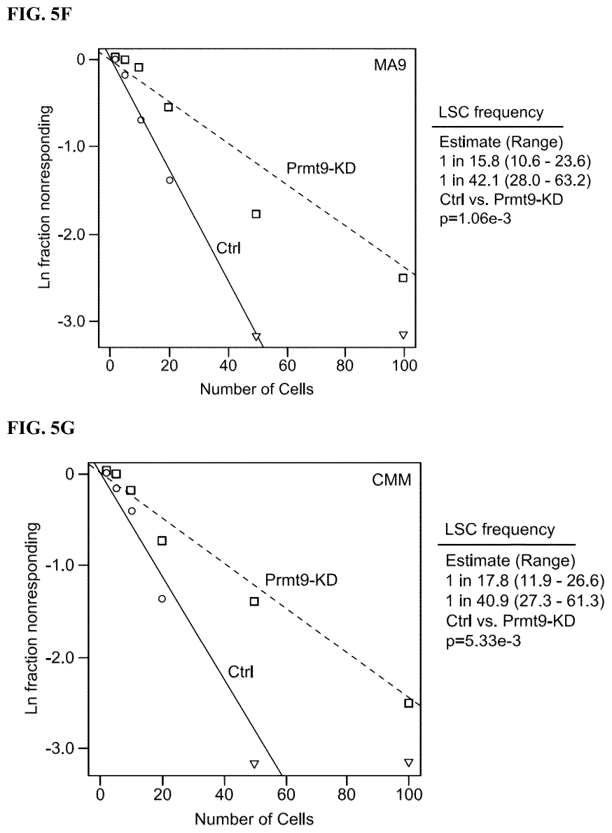

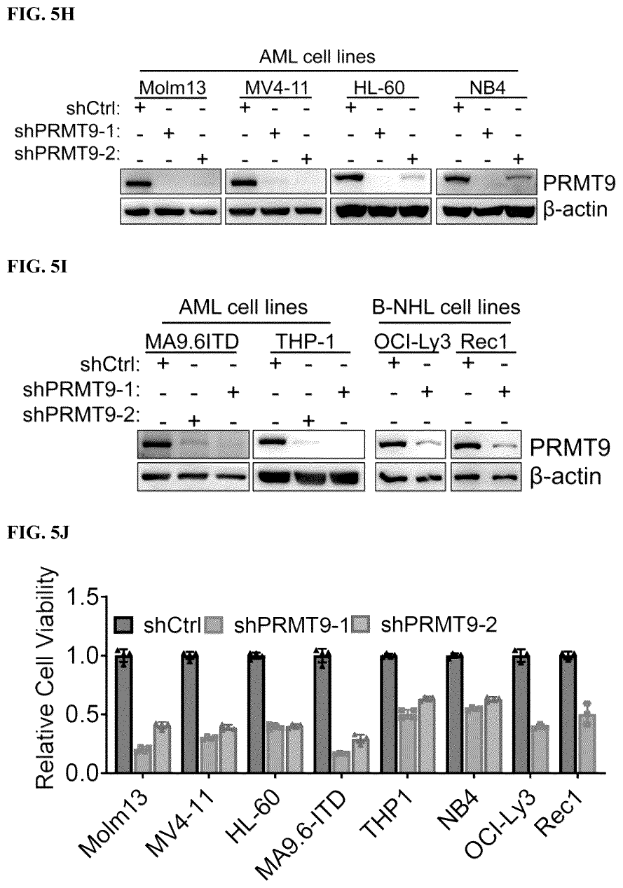

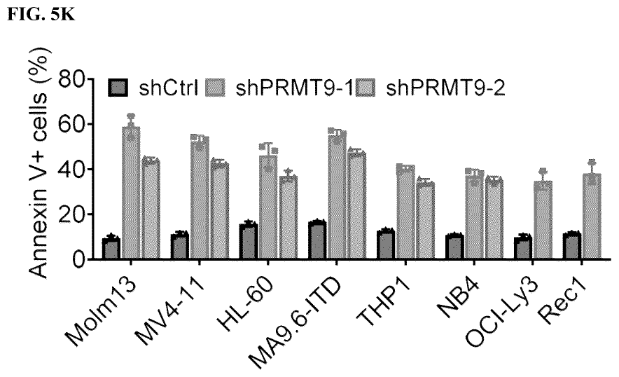

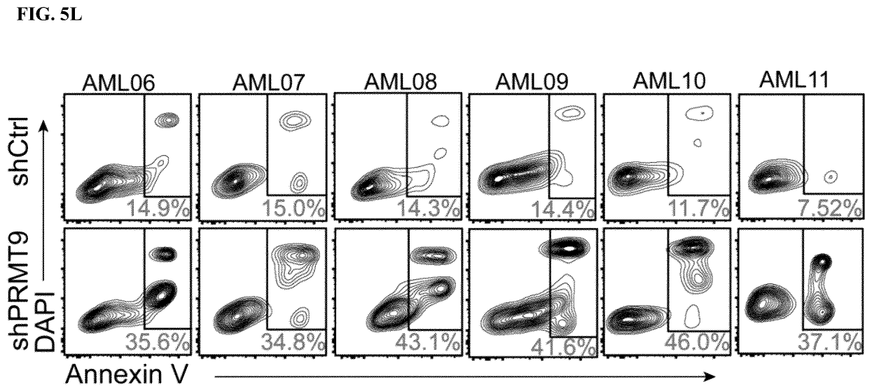

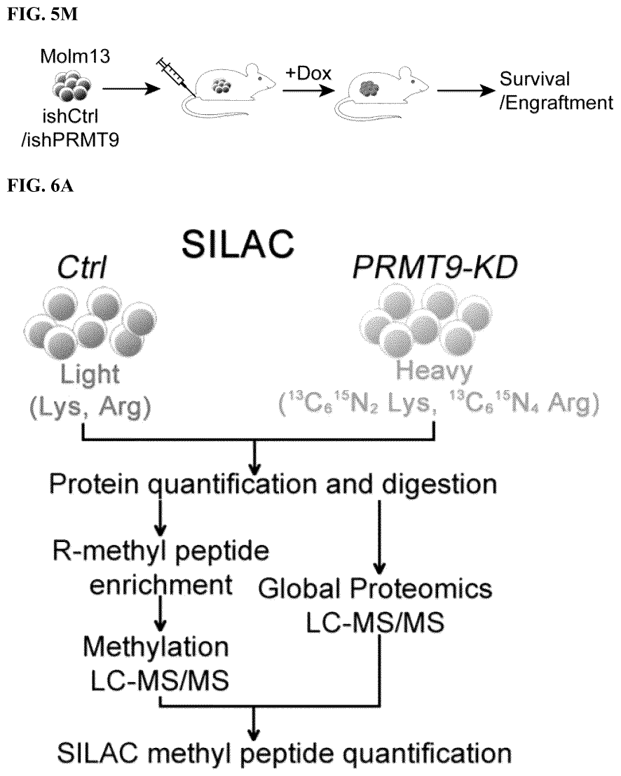

To determine PRMT9 function in AML, loss-of-function studies were performed using AML mouse model. Prmt9-cKO was first crossed with MLL-AF9 (MA9) knock-in mice to generate Prmt9-cKO/MA9 mice. In MA9 mice, significantly elevated Prmt9 levels were observed in MA9 mouse BM cKit⁺ cells relative to their normal counterparts, recapitulating PRMT9 upregulation in human AML (FIG. 4A). Given that other oncogenes frequently cooperate with MA9 to induce AML, effects of Prmt9-KO were assessed in both MA9 single-hit and double-hit models; for the latter, FLT3-ITD was used as the second hit, due to its high frequently (about 14%) seen in human MLL-rearranged AML. Specifically, LSK BM cells were transduced from 8-week-old Prmt9-cKO/MA9 or control (Prmt9^{f/f}/MA9, Prmt9-WT/MA9) mice with either a lentiviral vector expressing FLT3-ITD and co-expressing GFP or empty vector and plated transduced cells for colony forming cell (CFC) assay. Relative to the controls, CFC growth was significantly inhibited in Prmt9 KO MA9⁺ BM cells (FIGS. 4B-4C). Notably, more potent CFC inhibitory effects were seen in MA9 FLT3-ITD⁺ cells than in MA9/MOCK cells (FIGS. 4B-4C, 5A). Additionally, a doxycycline (DOX)-inducible Prmt9 KD construct was also transduced into leukemia cells (from either MA9 single-hit or double-hit model). Those inducible constructs co-expressed RFP, enabled sorting the same intensity of RFP⁺ cells with comparable transduction efficiency and virus copy numbers (FIG. 5B) for functional analysis. Consistent with the effect of Prmt9-KO in MA9 cells, Prmt9-KD significantly inhibited CFC growth of MA9 and MA9 FLT3-ITD BM cells (FIGS. 5C-5D). Besides the MA9 model, outcomes were evaluated by inducible Prmt9-KD in another AML model-a double-hit inv(16) AML model. Specifically, BM cells were transduced from Cbfb-MYH11 (inv[16] derived fusion) knock-in mice resembling inv(16) AML, with a retroviral vector co-expressing thrombopoietin receptor (MPL) plus GFP genes to generate a transplantable Cbfb-MYH11 Mpl (CMM) double-hit model. Interestingly, Prmt9 levels were significantly elevated in CMM cells (FIG. 4A). Similar to that shown in MA9 model, Prmt9-KD inhibited CFC growth of CMM AML cells (FIGS. 5C-5D). To test whether PRMT9 inhibition impairs LSC self-renewal, an in vitro limiting dilution assay using AML cells from MA9, MA9 FLT3-ITD⁺ and CMM models engineered to undergo inducible Prmt9 KD. Notably, PRMT9 KD decreased LSC frequency in all three models, as evidenced by in-vitro dilution assays (FIGS. 5E-5G).

To assess whether Prmt9 is required for leukemogenesis in vivo, MA9 FLT3-ITD double-hit Prmt9 cKO cells or corresponding controls were transduced with a luciferase reporter enabling in-vivo bioluminescence imaging prior to transplanting them into congenic, irradiated CD45.1-expressing recipients. Following donor cell engraftment, Prmt9 deletion was induced by pIpC administration. Bioluminescence analysis revealed that compared to control mice, Prmt9-deficient mice had a much slower AML progression and lower tumor burden (FIGS. 4D-4E). Prmt9-deficient leukemic mice also showed decreased splenomegaly (FIG. 4F) and decreased leukemic chimerism (FIG. 4G) relative to controls. Notably, recipients bearing Prmt9-KO MA9 FLT3-ITD transplants survived significantly longer than did mice receiving Prmt9 intact leukemic transplants (FIG. 4H).

PRMT9 function in human cancers was next assessed. To do so, human AML and B-NHL cell lines were first transduced with lentivirus vectors co-expressing PRMT9 shRNAs (shPRMT9-1, shPRMT9-2) or control shRNA (shCtrl) together with RFP (FIGS. 5H-5I). Knockdown (KD) of PRMT9 inhibits cancer cell growth and decreases viability (FIG. 5J-5K). Molm-13 cells were also engineered to express either a PRMT9 WT or corresponding catalytically dead mutant construct (LDIG [aa182-aa185] to AAAA), both were designed to resist PRMT9 shRNA-1 (namely, PRMT9/WT-R, PRMT9/MUT-R (FIG. 4I)). Notably, unlike transduction with PRMT9/MUT-R, expression of PRMT9/WT-R completely rescued growth/survival inhibition seen following PRMT9-KD (FIGS. 4J-4K), indicating that PRMT9 catalytic activity is required for cell viability. Moreover, PRMT9-KD inhibited survival and growth of primary AML CD34⁺ cells more potently than it did with counterparts from normal PBSC donors (FIGS. 4L-4N, 5L). Q-PCR analysis confirmed that PRMT9 mRNA levels were efficiently lowered in shRNA-transduced primary AML cells than in normal control cells (FIG. 4O). Finally, to evaluate in-vivo growth, Molm13 cells with the DOX-inducible shPRMT9 construct or a control vector were transplanted into NSG mice (FIG. 5M). When engraftment (>1% in PB), mice was treated with DOX to induce shRNA and RFP expression (FIG. 4P). Notably, mice transplanted with shPRMT9 cells showed significantly reduced leukemia burden in BM as well as prolonged survival, compared with mice transplanted with control cells (FIGS. 4Q-4R). Collectively, these results indicate that PRMT9 promotes survival of both LSCs and non-LSCs.

PRMT9-Mediated Methylation of Downstream Substrates Promotes Cancer Cell Maintenance

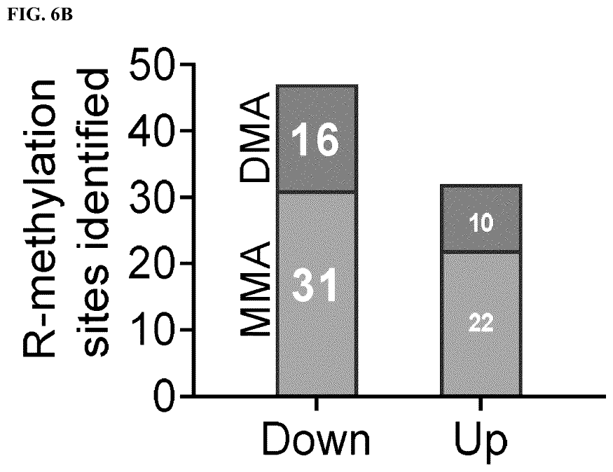

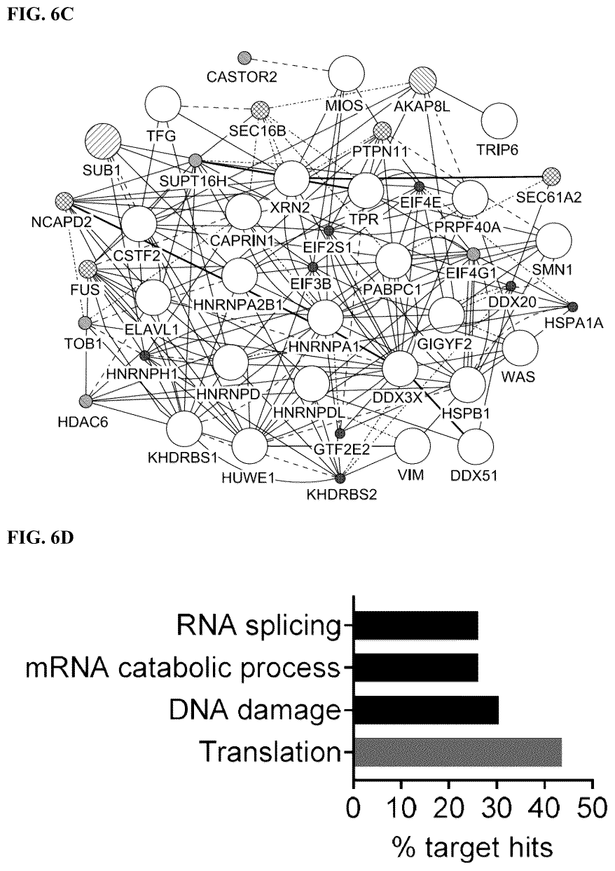

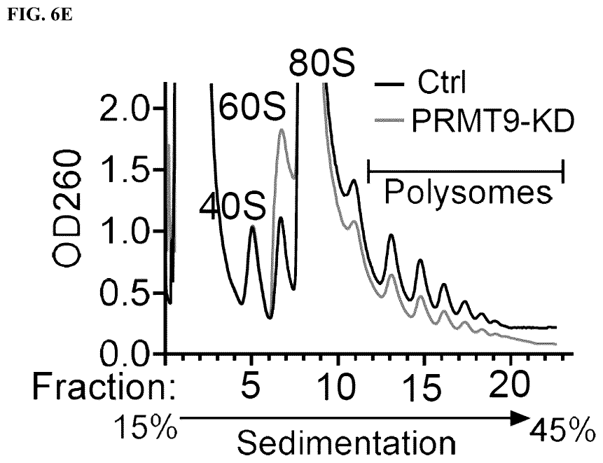

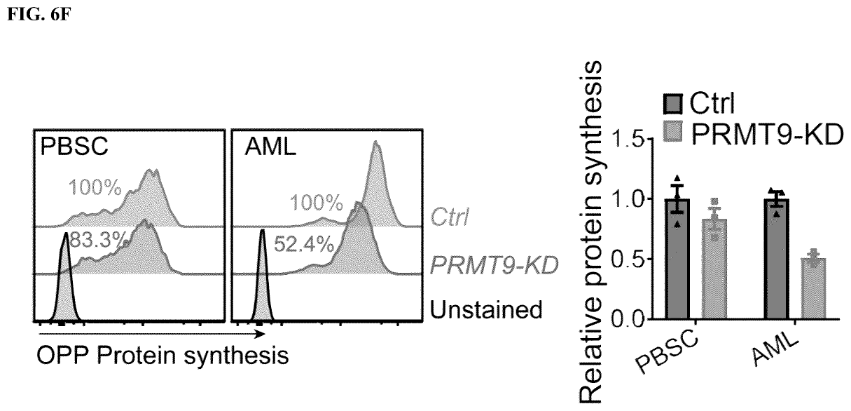

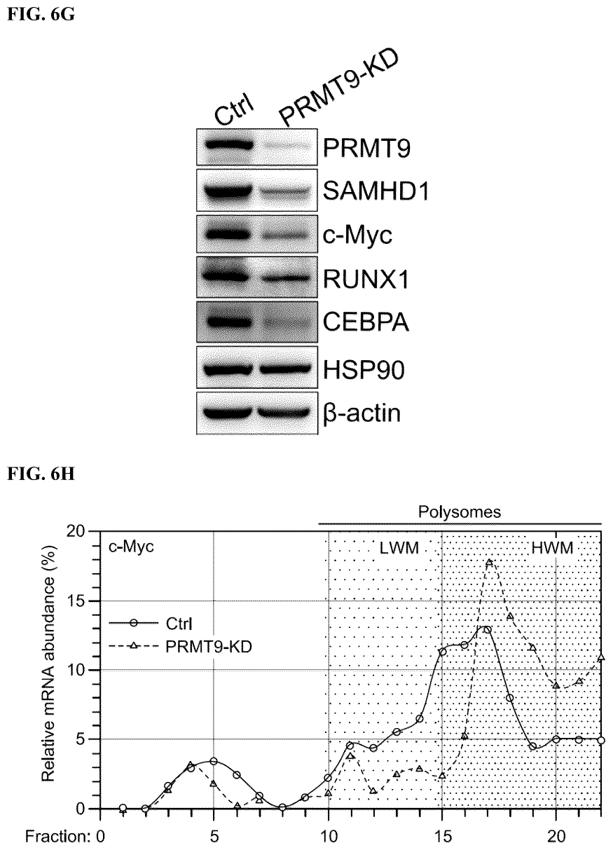

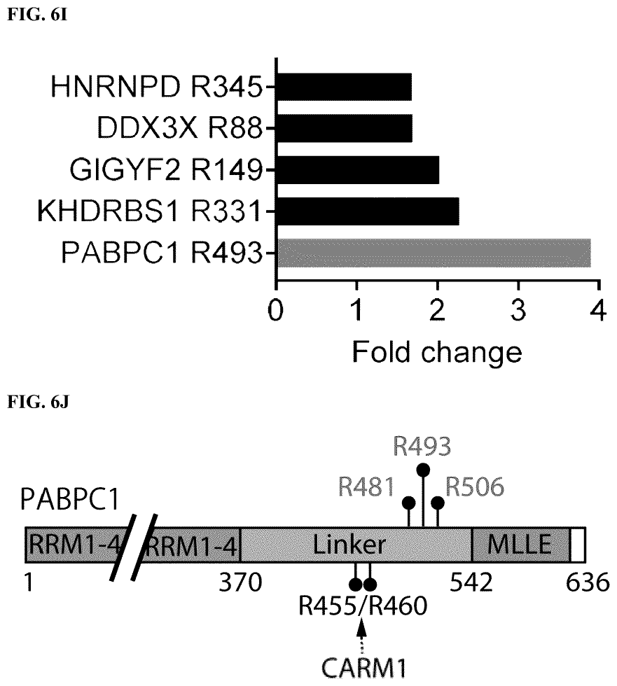

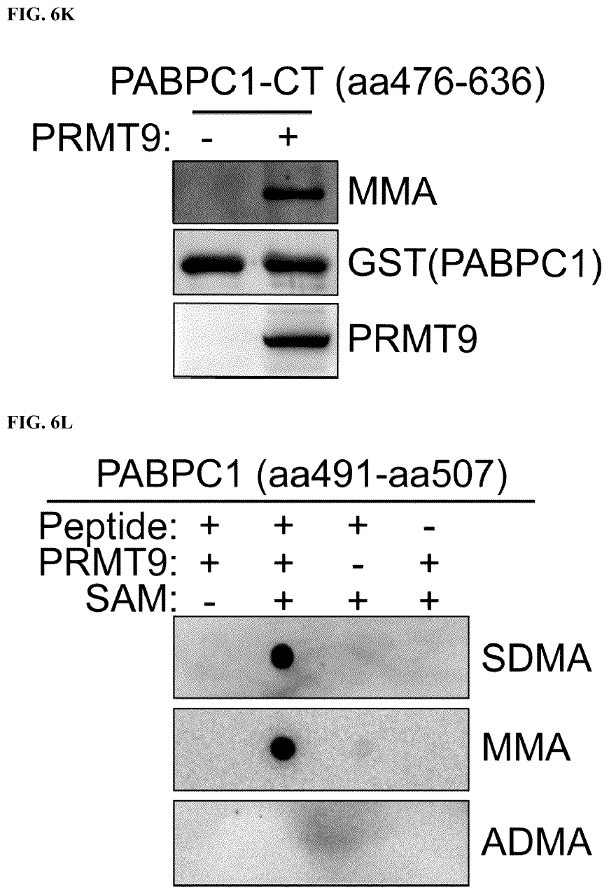

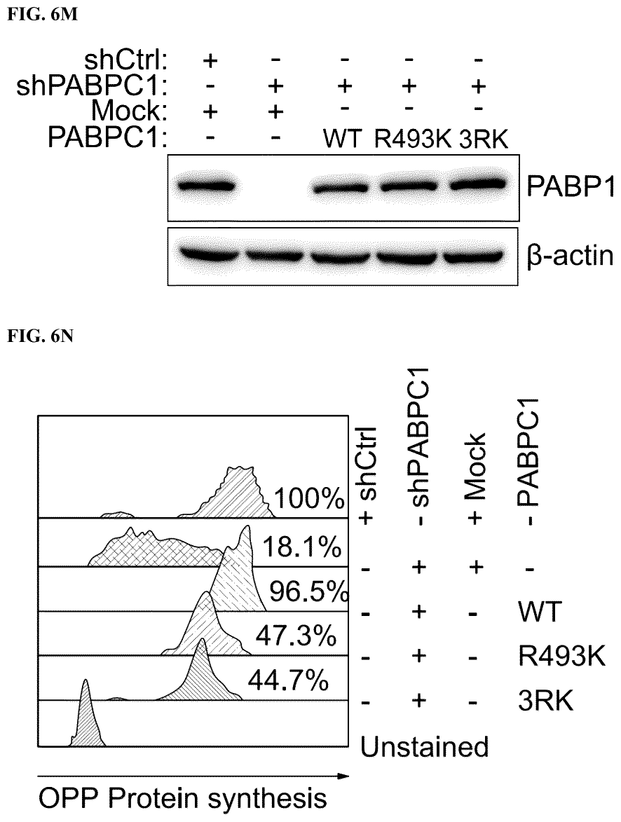

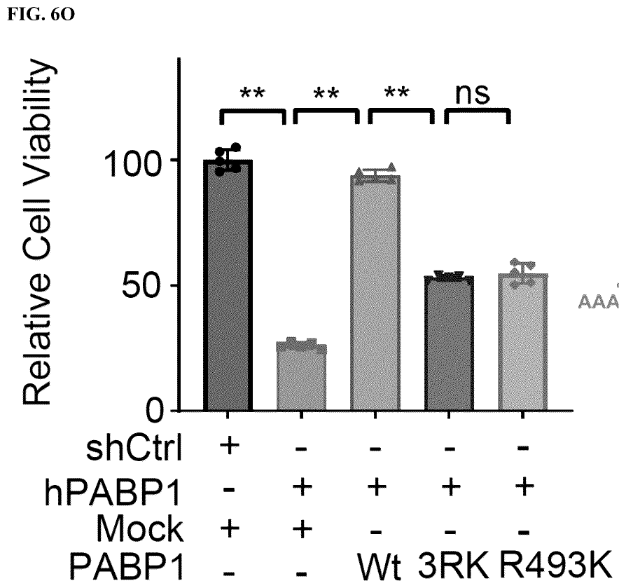

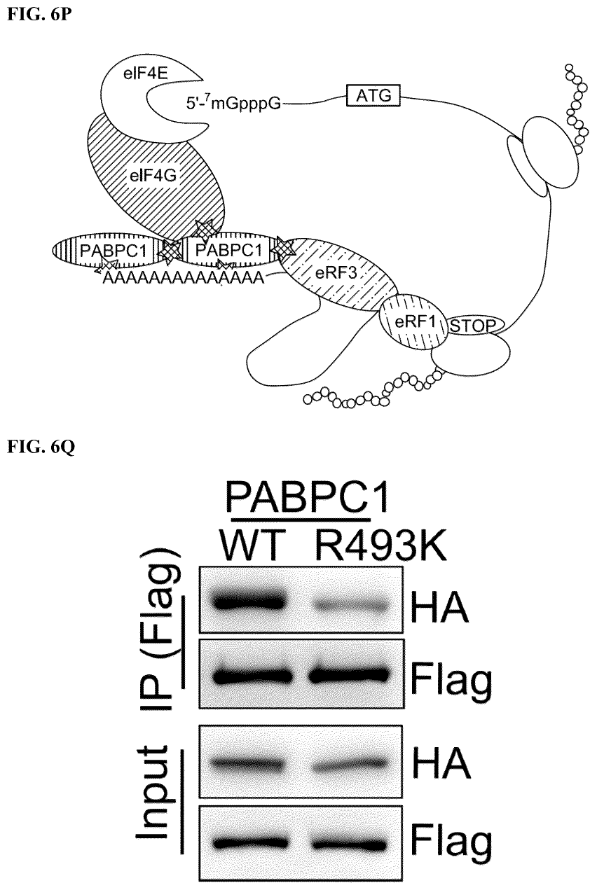

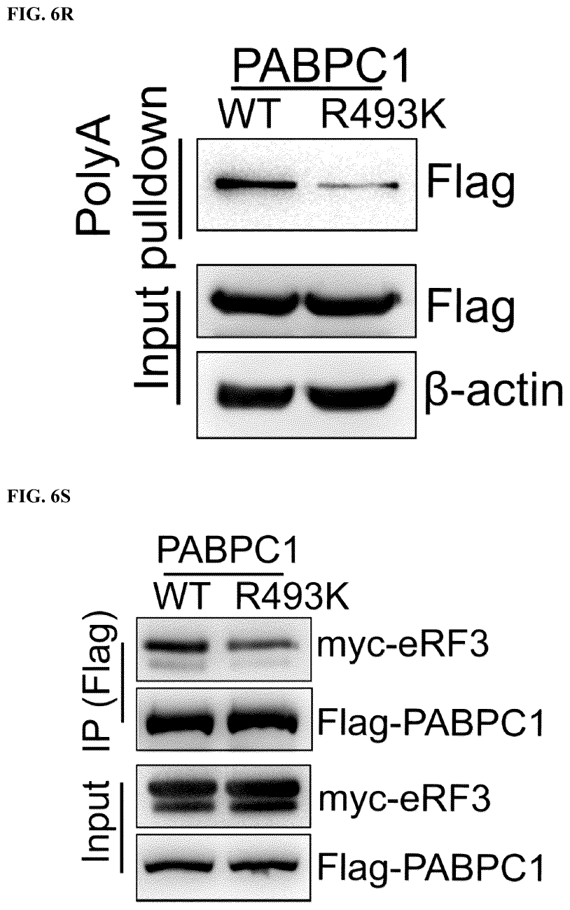

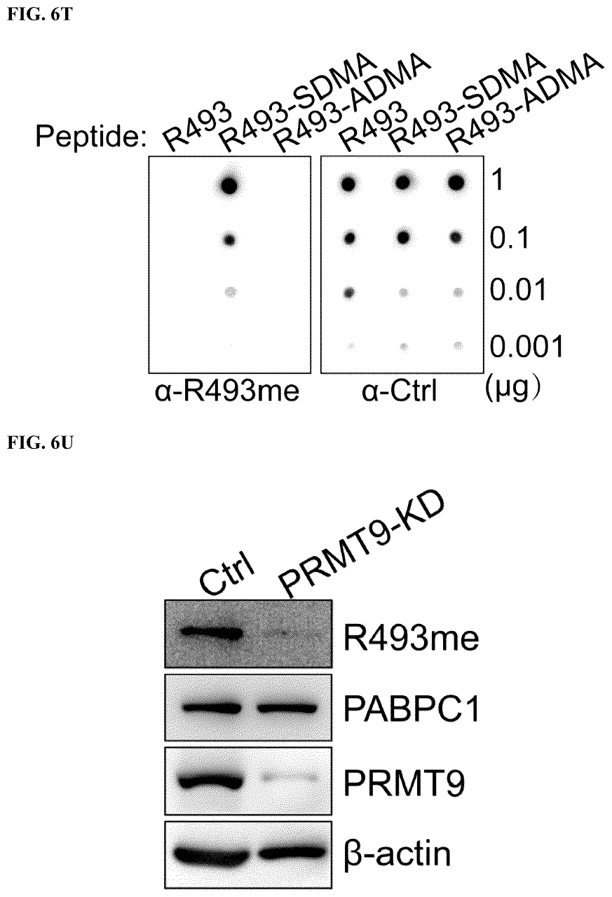

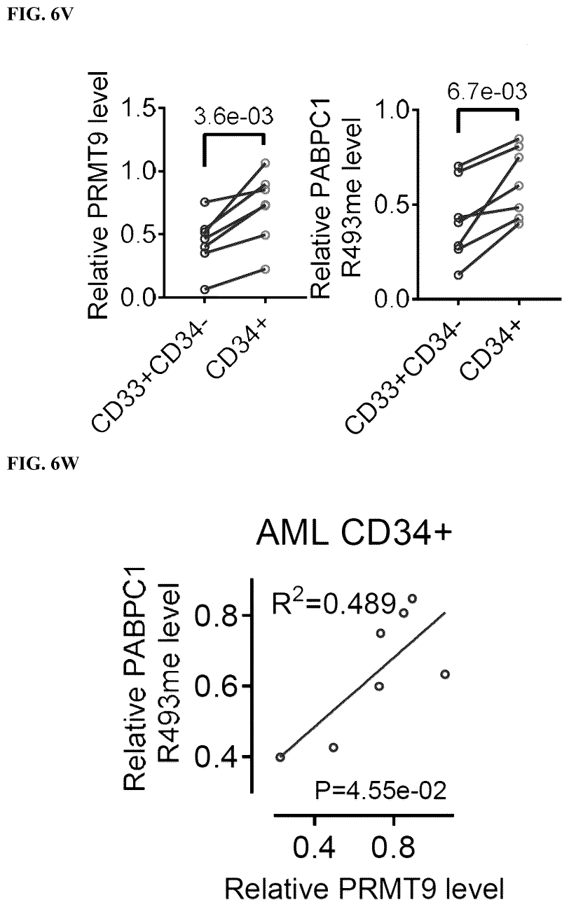

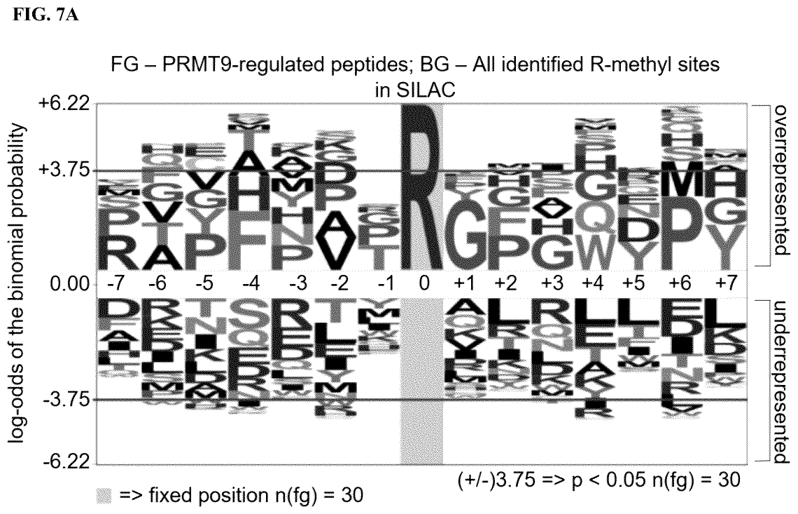

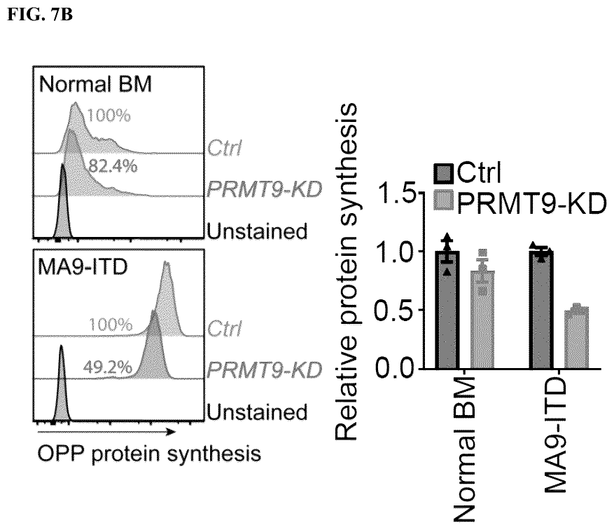

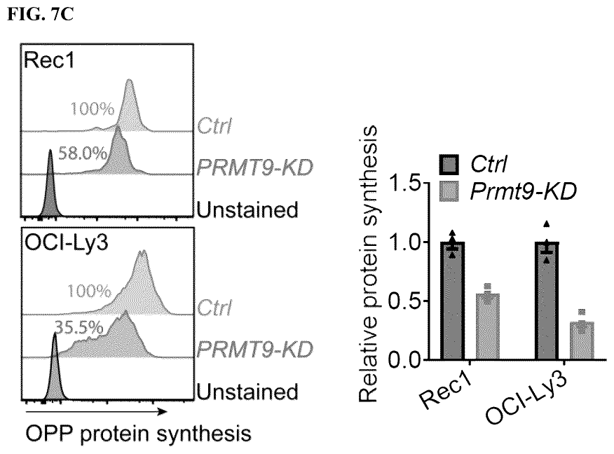

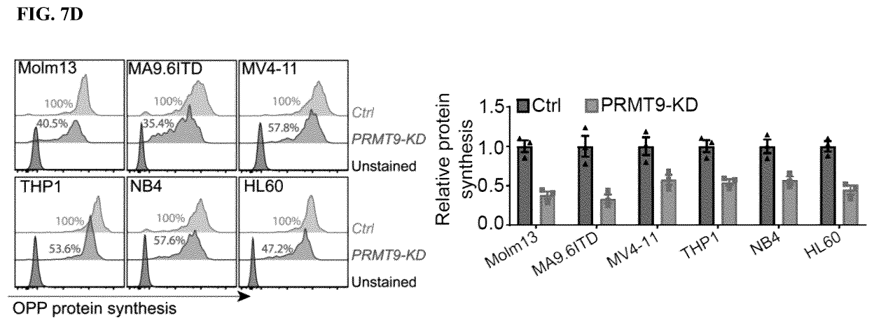

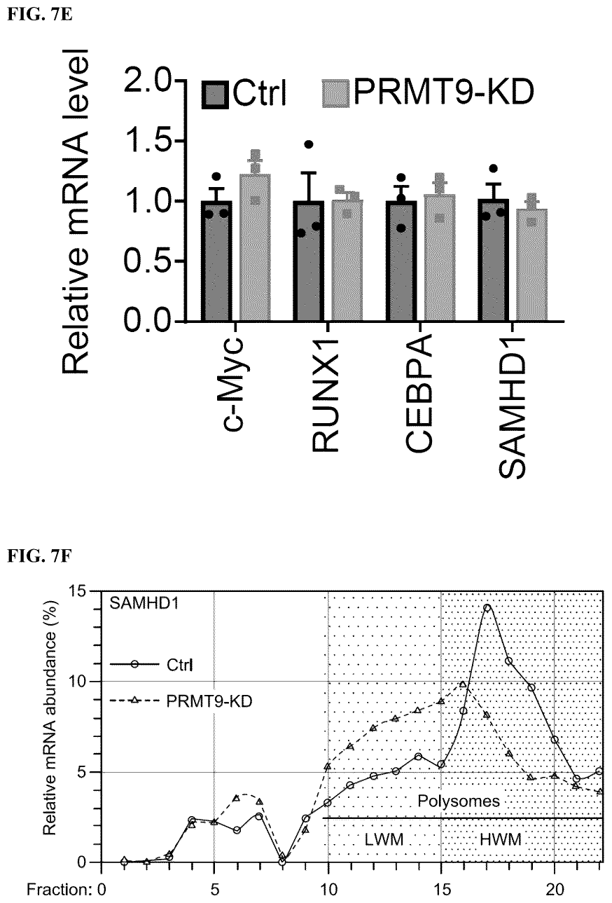

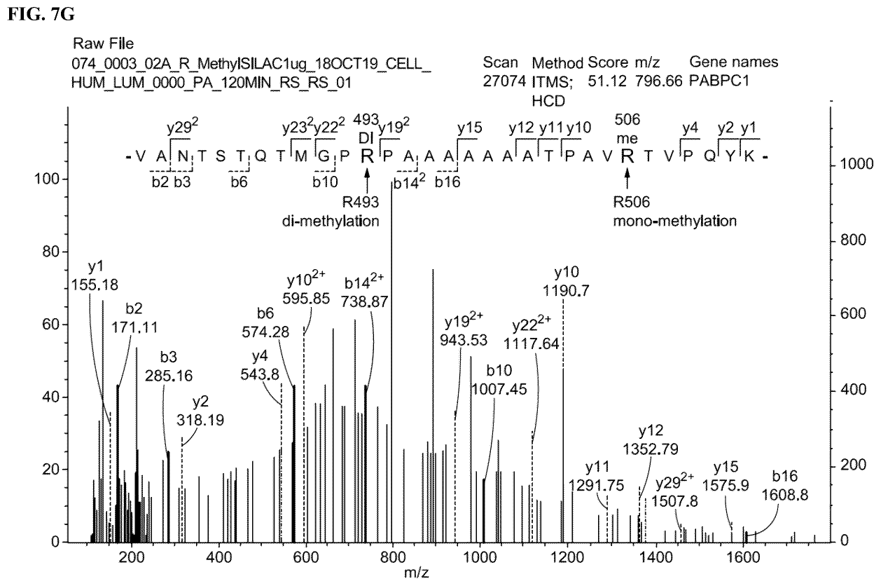

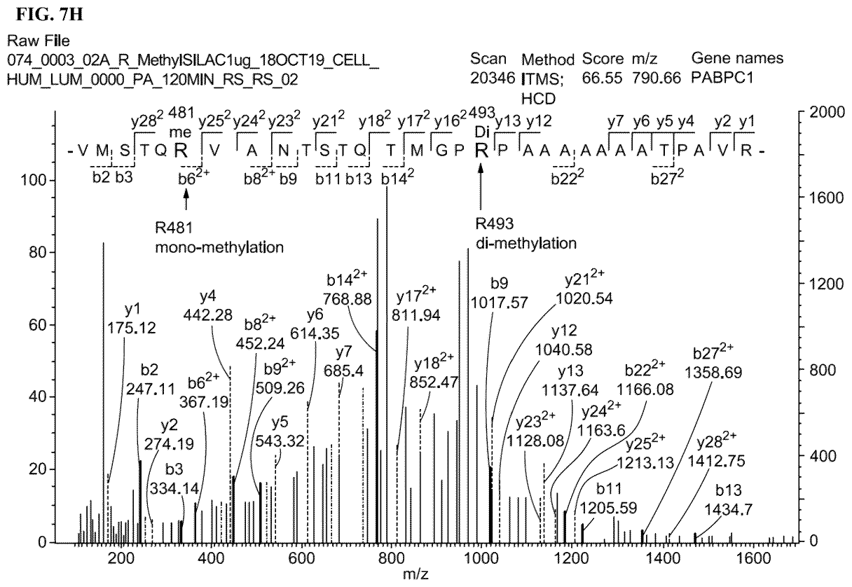

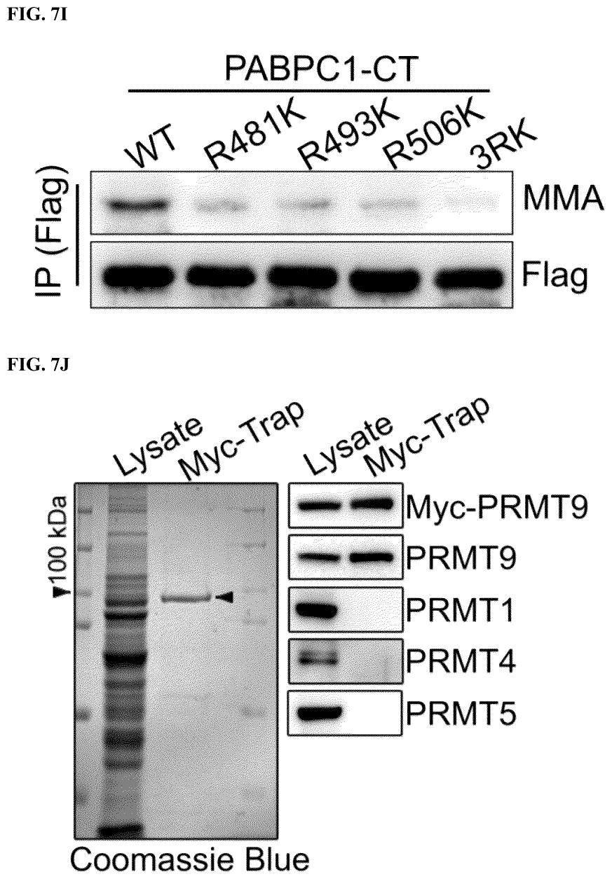

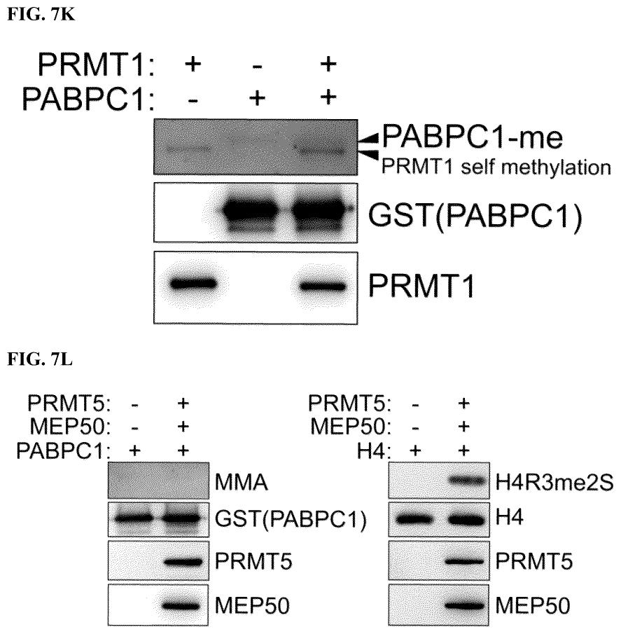

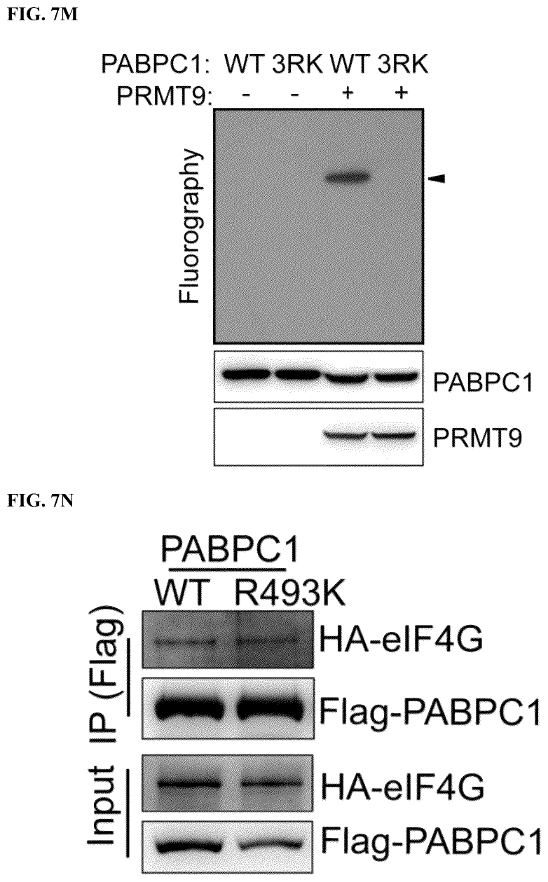

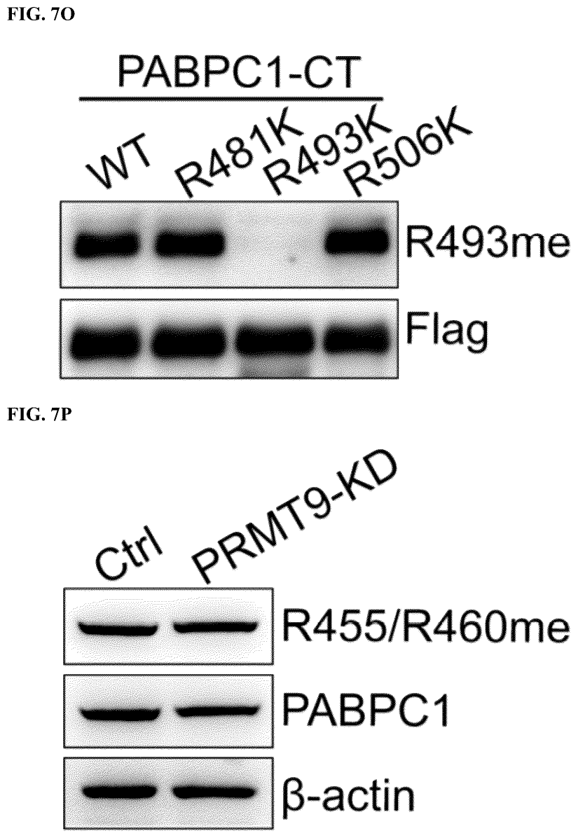

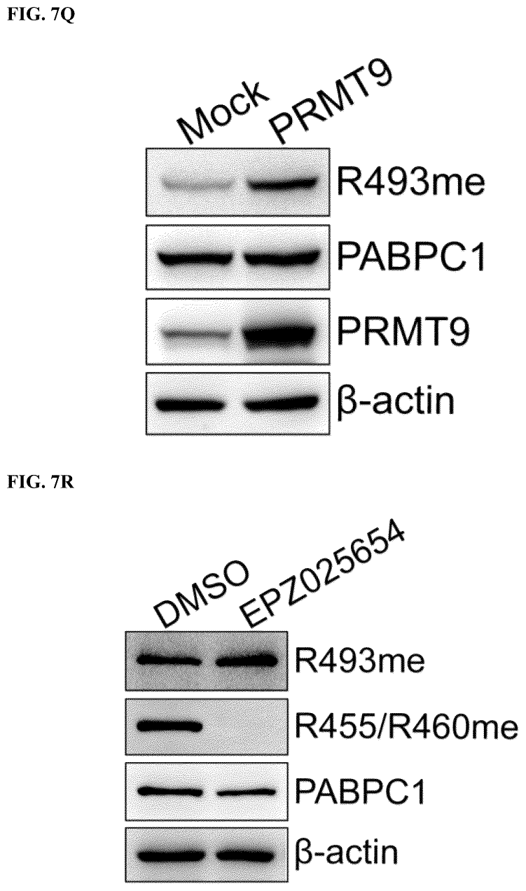

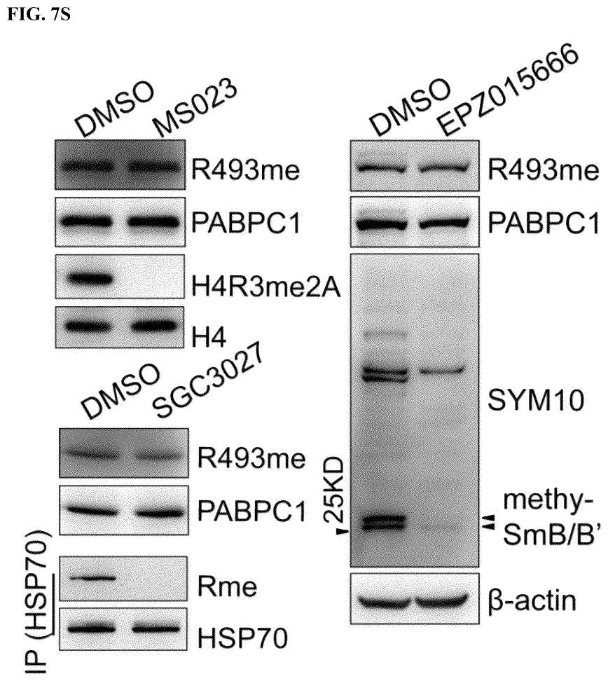

Given that PRMT9's ability to promote AML cell growth requires its catalytic activity (FIGS. 4J-4K4K), it was assumed that methylated substrates mediate those outcomes. Thus, to identify proteins showing altered arginine methylation after PRMT9 KD, SILAC based proteomics analysis was performed on inducible shPRMT9- or shCtrl-transduced Molm-13 cells (FIG. 6A). To do so, control and PRMT9-KD cells were first labeled with either heavy or light L-arginine and L-lysine and then mixed lysates from equal amounts of control or PRMT9-KD cells for subsequent digestion. Besides 5% of total peptides for global proteomics, 95% of the peptides were enriched by antibodies specific for pan-SDMA (Symmetric Di-Methyl Arginine) and pan-MMA (mono-methyl arginine) proteins, and then analyzed by multidimensional LC/MS-MS. Methylated peptide ratios were normalized based on total protein abundance. The analysis of PRMT9-KD cells revealed 315 unique arginine mono-methylation sites and 109 arginine di-methylation sites. Notably, PRMT9-KD resulted in marked downregulation of 16 (14.7%) DMA and 31 (9.8%) MMA sites (FIG. 6B, FC>1.5) in 23 unique proteins (FIG. 6C). However, motif analysis using iceLogo could not detect any consensus sequences enriched in methyl-peptides regulated by PRMT9 KD (FIG. 7A), likely due to the limited number of regulated methylation peptides. Among 23 proteins, 10 function in RNA translation (i.e., CAPRIN1, DDX3X, ELVL1, GIGYF2, HNRNPD, HSPB1, KHDRBS1, PABPC1, TPR, VIM), 7 were known to regulate the DNA damage response (i.e., DDX3X, ELAVL1, HNRNPD, HUWE1, KHDRBS1, TPR, XRN2), and 6 were related to RNA catabolism or splicing (FIG. 6D).

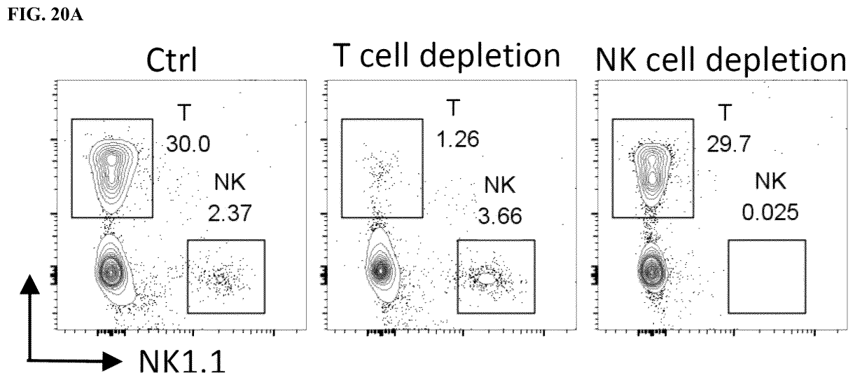

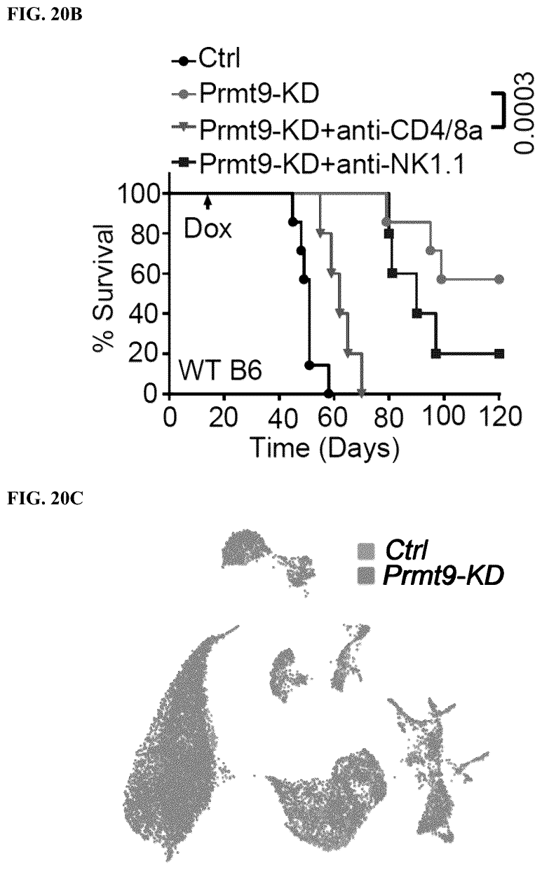

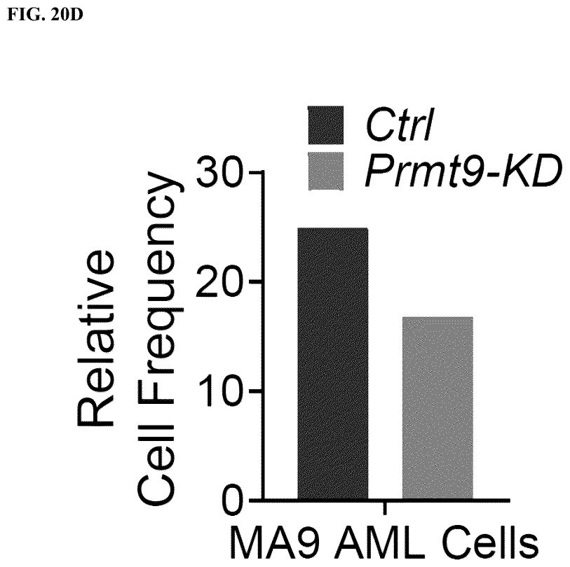

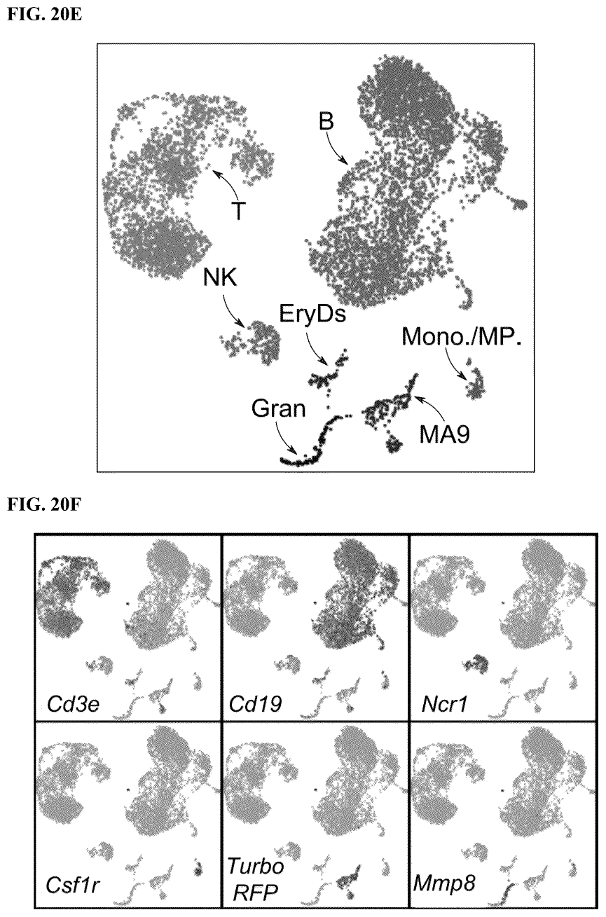

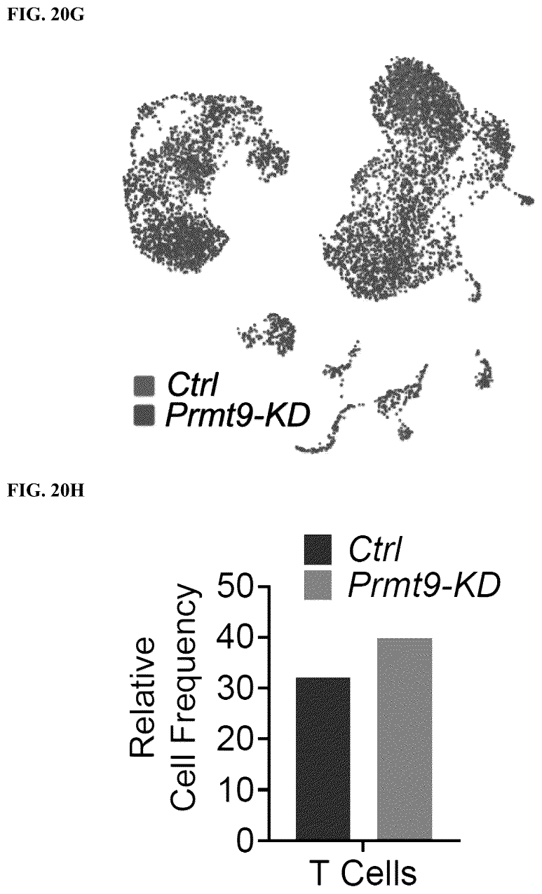

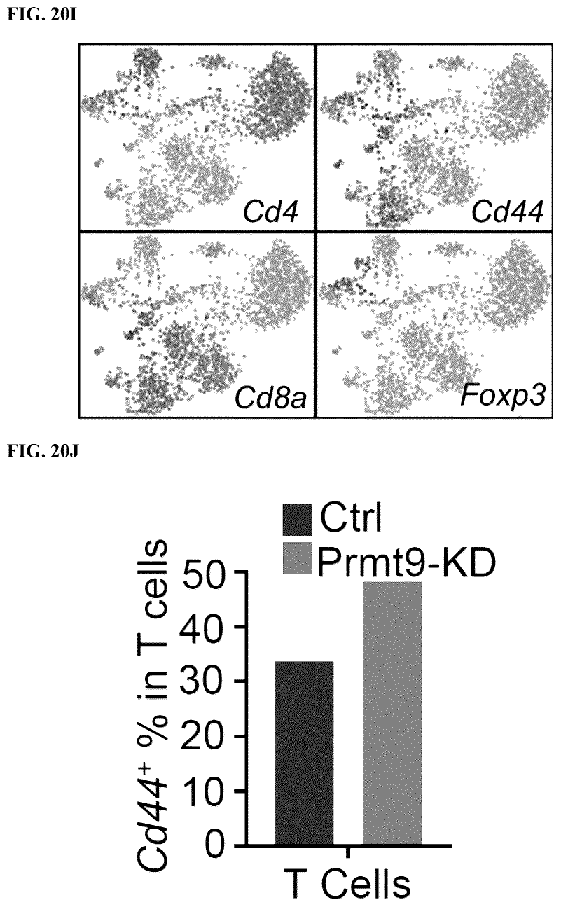

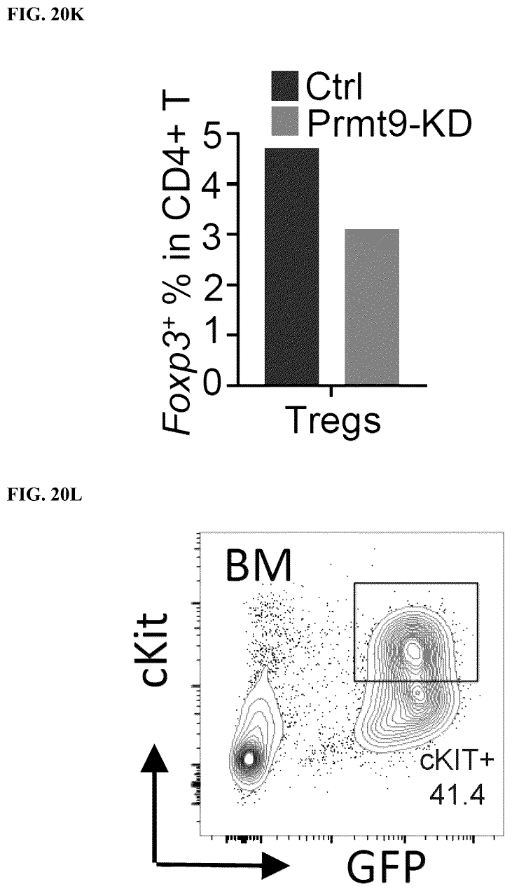

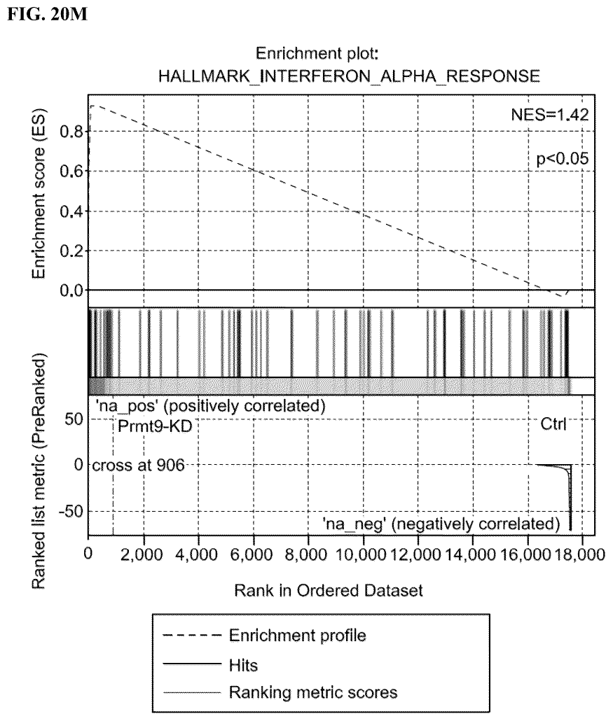

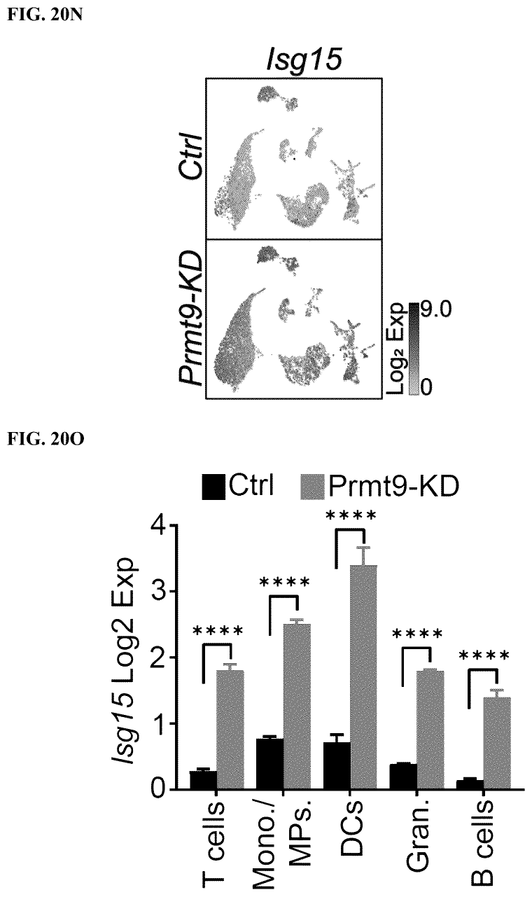

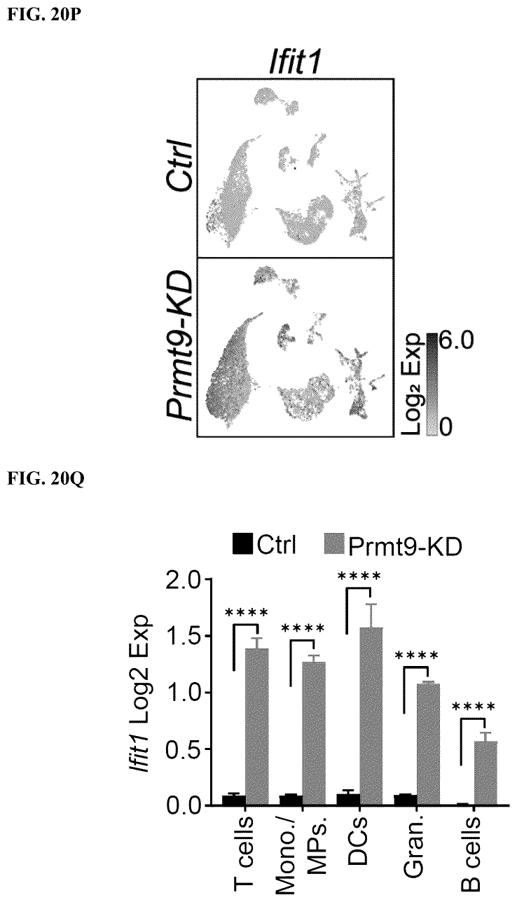

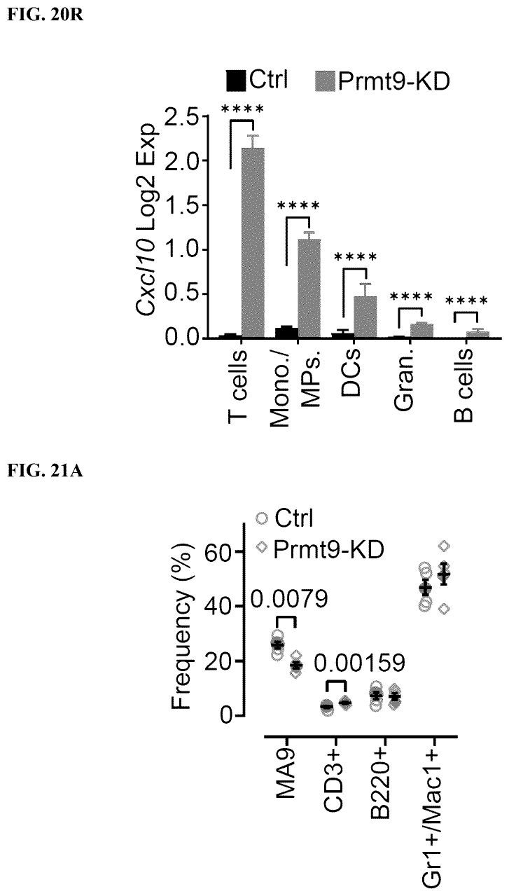

It was next asked whether PRMT9 regulates protein translation by performing polysome profiling. Specifically, cell extracts from Molm13 cells were applied with PRMT9-KD to a sucrose density gradient assay (SDG). PRMT9-KD reduced levels of polysome-associated mRNAs and increased accumulation of monosome-associated mRNAs, indicative of poorly translated mRNA and consistent with a translation defect (FIG. 6E). Next, to assess protein synthesis in PRMT9 KD cancer cells using an O-propargyl-puromycin (OP-Puro)-based flow cytometry assay, decreased global protein synthesis was found following PRMT9-KD in AML CD34⁺ cells, cancer cell lines (FIGS. 6F, 20B-20D). Conversely, PRMT9 KD only modestly altered global protein synthesis in hematopoietic CD34⁺ cells from normal PBSC or BM (FIGS. 6F, 7B). PRMT9 KD also downregulated levels of short-lived proteins, including c-Myc and others (CEBPA, SAMHD1, RUNX1) also seen in the global proteomics analysis, while their mRNA levels were unchanged (FIGS. 6G, 7E). Finally, when levels of mRNA encoding were determined these factors in gradient fractions collected from polysome profiling, it was observed that PRMT9 KD shifted c-Myc and SAMHD1 transcripts from high-molecular-weight (HMW) to low-molecular-weight (LMW) polysomes, indicating decreased translation (FIGS. 6H, 7F).

Among down-regulated methylated peptides identified from the SILAC analysis, the methylated PABPC1 peptide with dimethyl-R493 (R493me2) at its C-terminus, was most significantly depleted by PRMT9 KD (FIG. 6I). Interestingly, PABPC1 reportedly initiates translation by binding to the mRNA poly(A) tail, and posttranslational modifications of PABPC1 are critical for its function in translation. PABPC1 was next defined as a potential PRMT9 substrate. Analysis of all PABPC1 peptides in PRMT9-KD vs. PRMT9-WT cells revealed two more arginine methylation sites, mono-methylation at R481 (R481me) and R506 (R506me), enriched in PRMT9-WT cells (FIGS. 6J, 7G-7H), although with a lower confidence score relative to that of R493me2. To validate the modifications, a Flag-tagged PABPC1 C-terminal fragment (aa476-636, PABPC1-CT) was constructed containing R481, R493, and R506 as well as a corresponding methylation-deficient (R to K) construct for each individual residue (R481K, R493K, R506K) or for all three residues (3RK). HEK293T cells were transfected with each individual constructs, pulled down exogenous PABPC1 protein with anti-Flag beads, and assessed methylation using a pan-MMA antibody. Each single site mutation decreased PABPC1-CT methylation levels, while 3RK mutation completely depleted PABPC1-CT methylation relative to WT controls (FIG. 7I). To determine if PABPC1 was a direct PRMT9 substrate, an in vitro methylation assay was performed using a GST-PABPC1 construct containing the PABPC1-CT. After incubating the GST-PABPC1 fragment and full length myc-tagged PRMT9 (FIG. 7J) or other PRMTs with SAM, the reaction mixture was separated by SDS-PAGE and PABPC1-CT methylation was next assessed by western blot. Unlike PRMT1 and PRMT5 which specifically catalyzed methylation of their respective substrates, only PRMT9 promoted PABPC1-CT methylation (FIGS. 6K, 7K-7L). An ex-vivo tritium methylation assay was also performed using PABPC1-WT and corresponding PABPC1-3RK (R481K/R493K/R506K) immunoprecipitated from 293T cells. Incubation with PRMT9 methylated PABPC1-WT (FIG. 7M), an effect totally abolished in the presence of the 3RK mutant, indicating that these 3Rs are the only residues methylated by PRMT9. Moreover, when a synthesized peptide containing R493 was incubated with PRMT9 and SAM, the methylation signal was seen by both anti-SDMA and anti-MMA antibodies, but not by an anti-ADMA (Asymmetric Di-Methyl Arginine) antibody (FIG. 6L).

To assess function of PRMT9-mediated PABPC1 methylation, full length PABPC1-WT, PABPC1-3RK (R481K/R493K/R506K), or the PABPC1-R493K mutant were ectopically expressed which are designated to resist shPABPC1 into Molm13 cells, and further transduced those cells with shPABP1 to KD endogenous PABPC1 (FIG. 6M). Notably, unlike WT PABPC1, which fully rescued PABPC1 KD phenotypes, expression of 3RK or R493K marginally improved protein synthesis and cell viability seen following PABPC1-KD (FIGS. 6N-6O). Moreover, cells expressing R493K exhibited impaired protein synthesis and cell viability to the same extent as cells expressing 3RK, indicating that R493 methylation is critical for PABPC1-mediated RNA translation. Since R493 is located in the PABPC1 proline-rich linker region that mediates PABPC1 self-association on the 3′ RNA poly(A) tail, it was asked if R493 methylation favored PABPC1 self-interaction (FIG. 6P). To do so, Flag-tagged PABPC1-WT or R493K were co-expressed together with HA-tagged PABPC1 WT in HEK293T cells, followed by Flag-IP. Notably, R493K pulled down less HA-tagged PABPC1 than did PABPC1-WT (FIG. 6Q). Next, a Poly(A) agarose pull-down assay was performed to evaluate whether R493 methylation altered PABPC1 binding to poly(A) (FIG. 6P). Under medium stringency (200 millimolar (mM) NaCl) conditions, less Flag-tagged PABPC1 was pulled down from PABPC1-R493K-expressing cells than from the PABPC1-WT-expressing cells (FIG. 6R). Furthermore, since R493 is near the PABPC1 C-terminus (aa541-636), it was asked if R493 methylation facilitated binding of the PABPC1 C-terminus to other translational factors such as eRF3 to promote translation initiation (FIG. 6P). Interaction of Flag-tagged PABPC1 variants was thus analyzed with myc-tagged eRF3 by co-TP. Following Flag-IP, PABP1-R493K exhibited less affinity to eRF3 than did PABP1-WT (FIG. 6S). In contrast, the R493K mutation did not alter PABP1 interaction with eIF4G (FIG. 7N), because eIF4G specifically binds to the PABPC1 N-terminus (FIG. 6P).

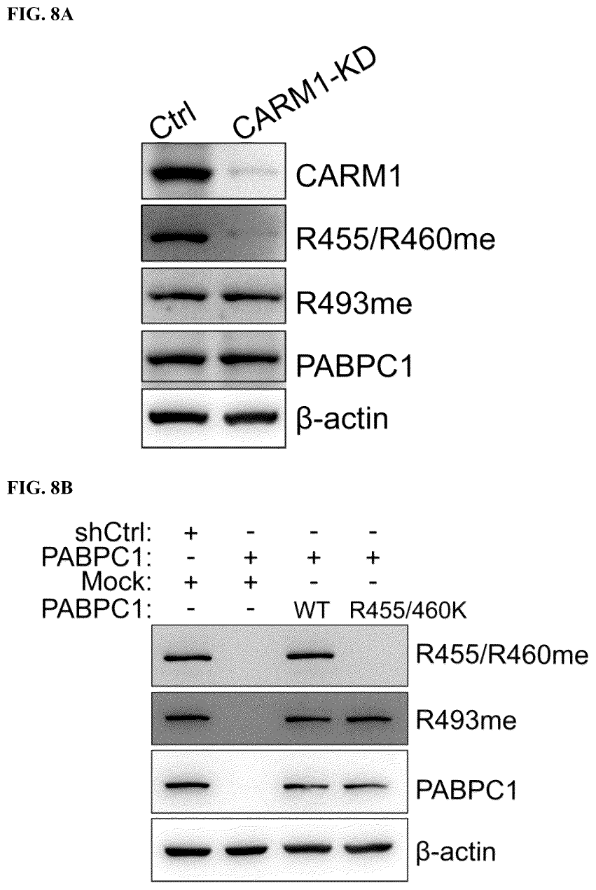

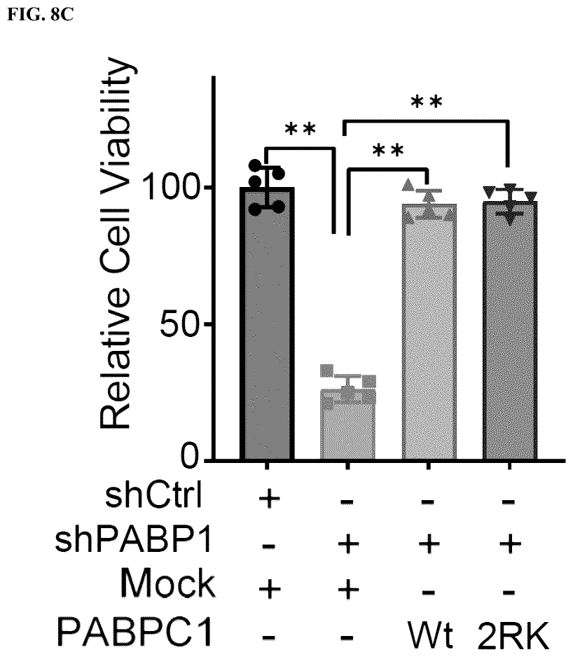

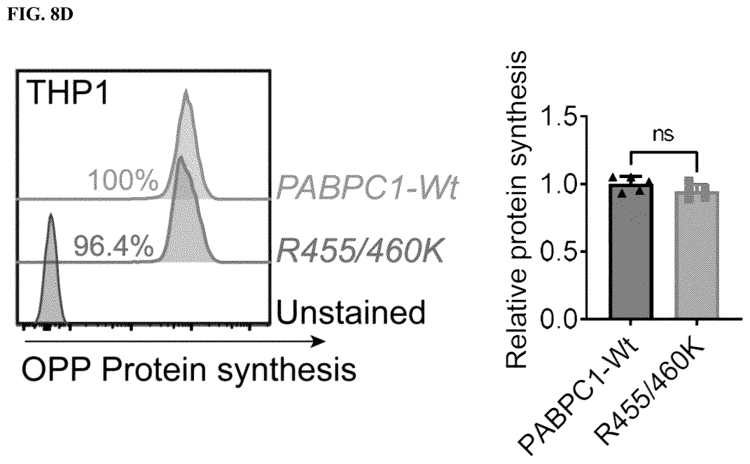

Next, an antibody was generated to detect R493 methylation (R493me) by immunizing rabbits with a synthesized symmetrically-di-methylated PABPC1 peptide (aa491-507). It was confirmed that the antibody specifically recognized symmetrically di-methylated R493me peptide but not unmodified or asymmetrically di-methylated forms of the peptide (FIG. 6T). Moreover, in 293T cells overexpressing PABPC1-CT, mutation of only R493, but not R481 or R506, completely abolished methylation signals, confirming antibody specificity (FIG. 7O). KD of endogenous PRMT9 also robustly blocked PABPC1 R493 methylation but not R455/R460 methylation (FIGS. 6U, 7P), while PRMT9 overexpression increased PABPC1 R493 methylation (FIG. 7Q). Moreover, cells with inhibitors of PRMT1, CARM1, PRMT5 or PRMT7 were treated; while all of these PRMT inhibitors inhibited their respective substrates, none altered R493 methylation levels (FIGS. 7R-7S), indicating specificity of PABPC1 R493-me by PRMT9. It was observed that CARM1 catalyzes PABPC1 R455/R460 methylation, but that CARM1-dependent PABPC1 methylation did not alter PRMT9-mediated PABPC1 R493 methylation (FIGS. 8A-8B). Moreover, consistent with a previous report by Brook et al. The Biochemical Journal 441, 803-812, doi:10.1042/BJ20111474 (2012), it was also found CARM1-dependent methylation did not alter PABPC1's function in RNA translation (FIGS. 8C-8D).

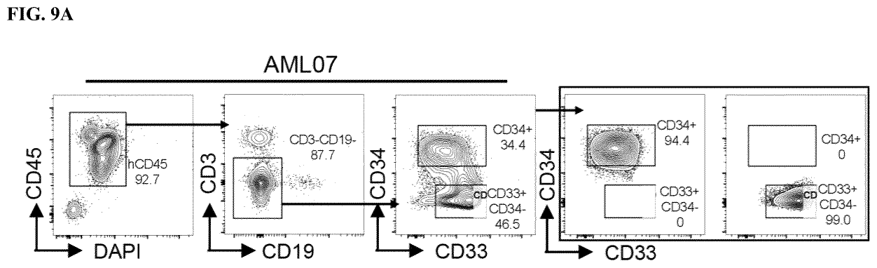

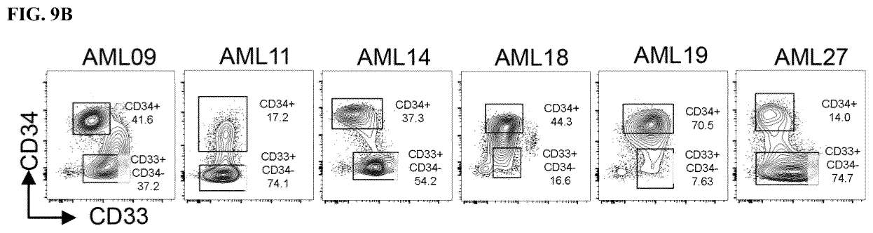

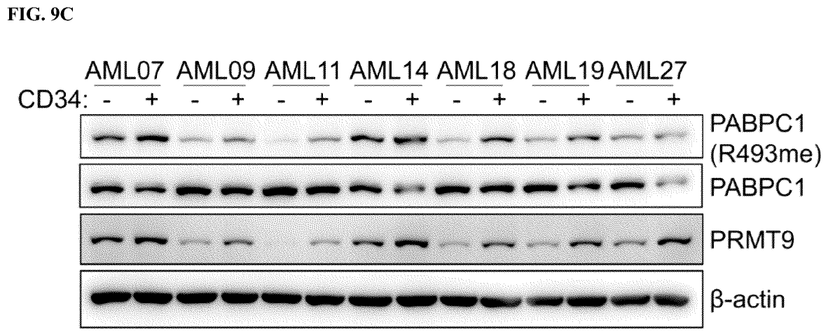

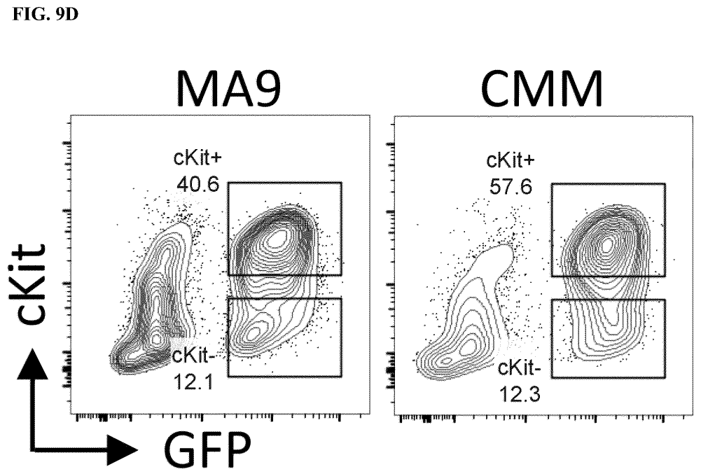

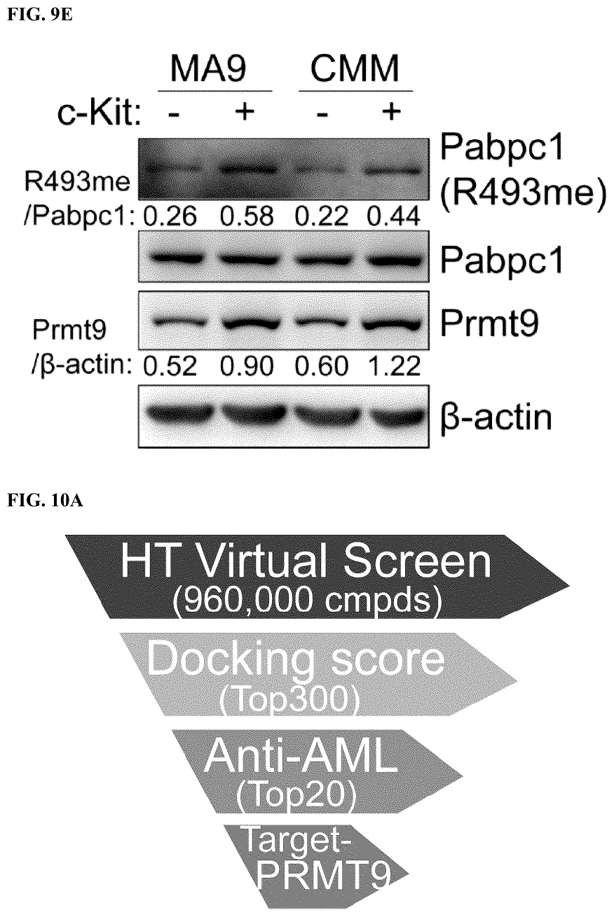

Next PRMT9 and PABPC1-R493 methylation (R493me) levels were correlated. To do so, the leukemia stem/progenitor-enriched CD34⁺ subset and CD34⁻CD33⁺ leukemia blasts were sorted from MNCs from human AML specimens (n=7, FIGS. 9A-9B) for western blot analysis (FIG. 9C). Notably, CD34⁺ cells expressed relatively higher PRMT9 and R493me levels than did blasts (FIG. 6V). R493me and PRMT9 levels were positively correlated in AML CD34⁺ cells (FIG. 6W). Similar analysis was conducted in MA9 (GFP⁺) and CMM (GFP⁺) cancer cells harvested from BM of AML-developed mice and observed higher Prmt9 and R493me levels in leukemia stem/progenitor-enriched cKit⁺ cells relative to cKit− blast cells (FIGS. 9D-9E).

Identification of a PRMT9 Inhibitor Via an R493 Methylation Assay.

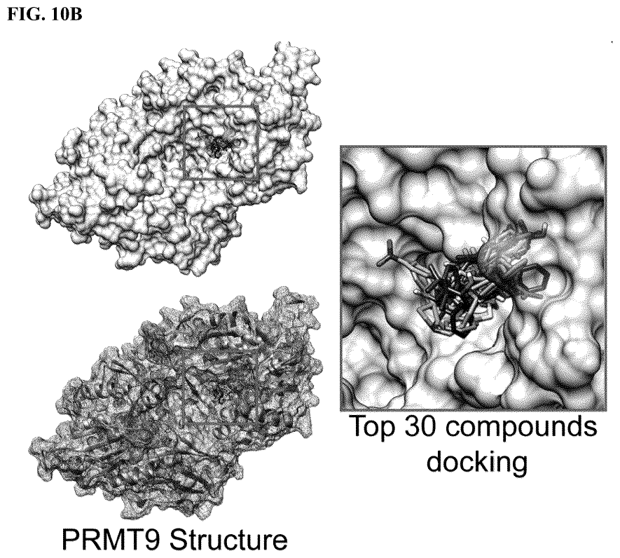

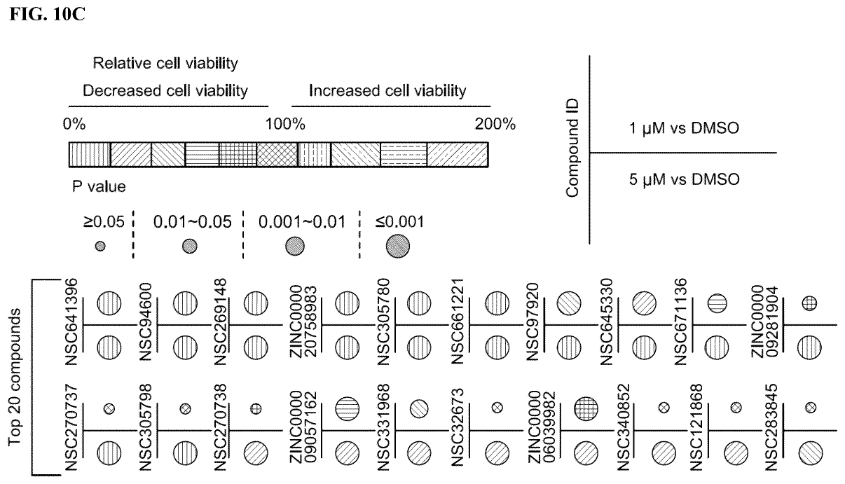

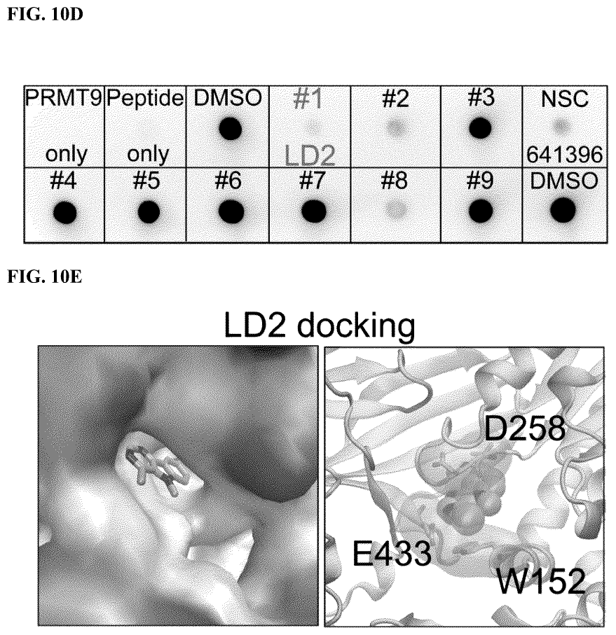

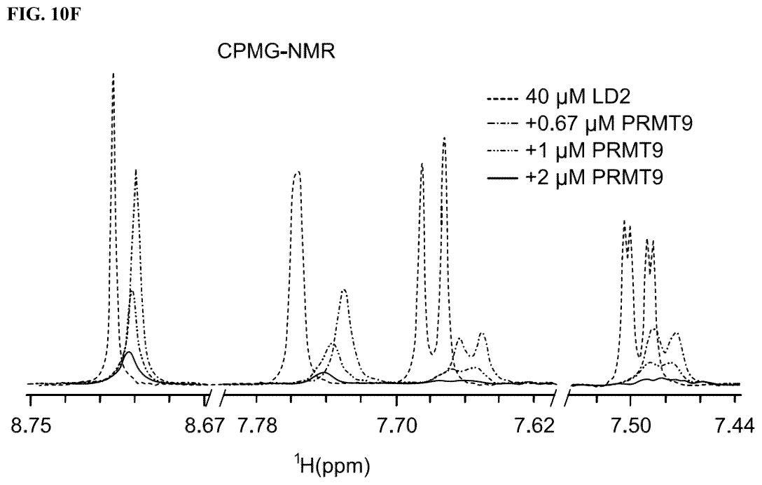

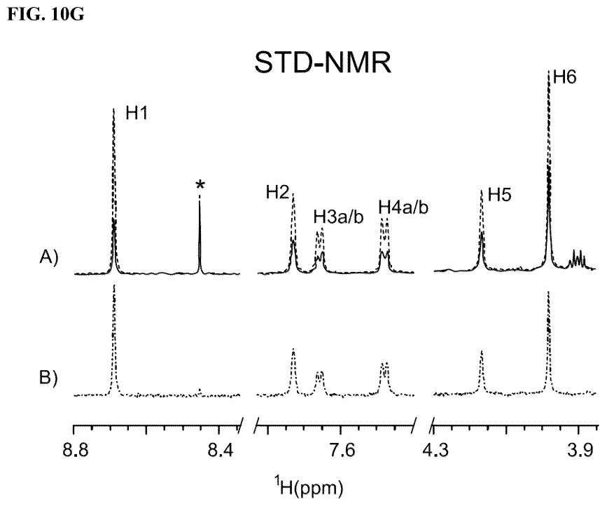

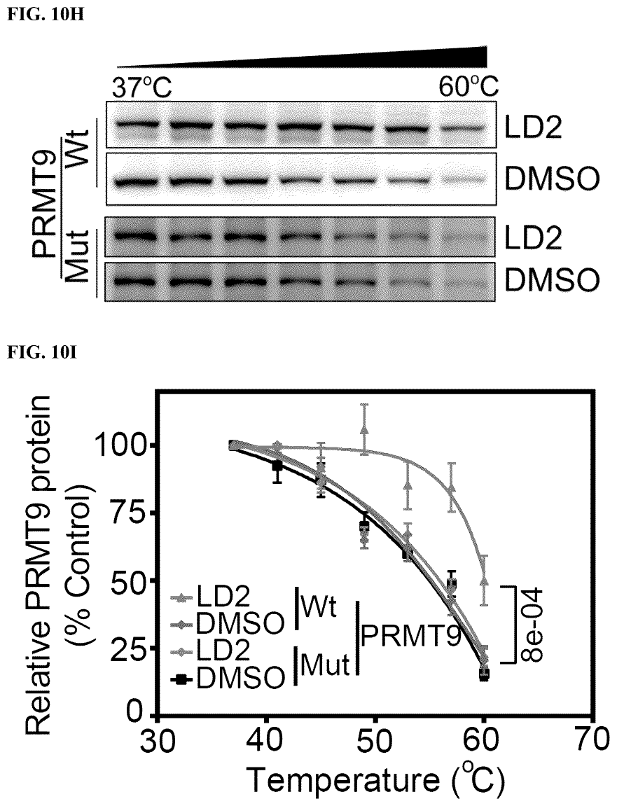

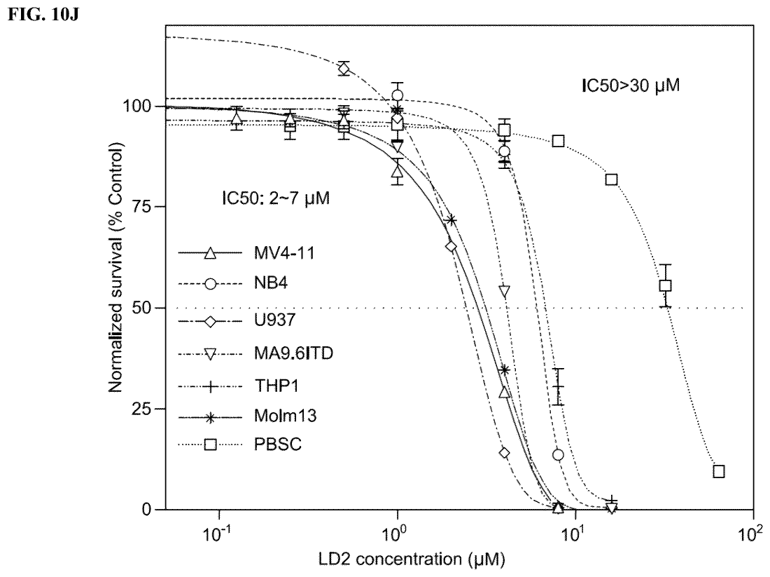

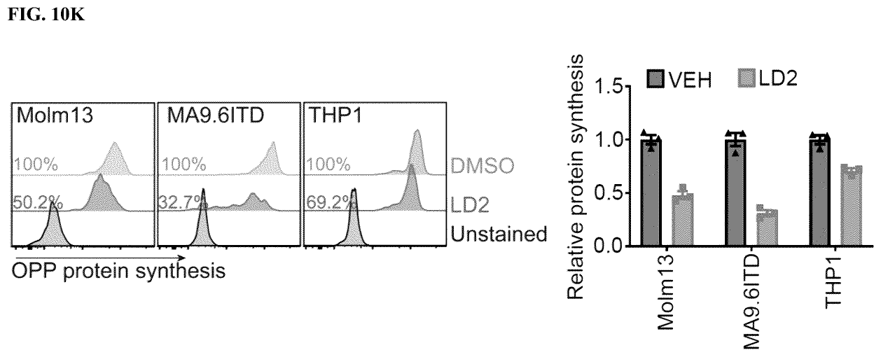

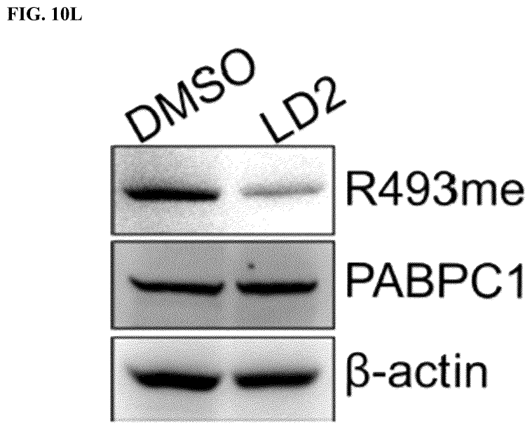

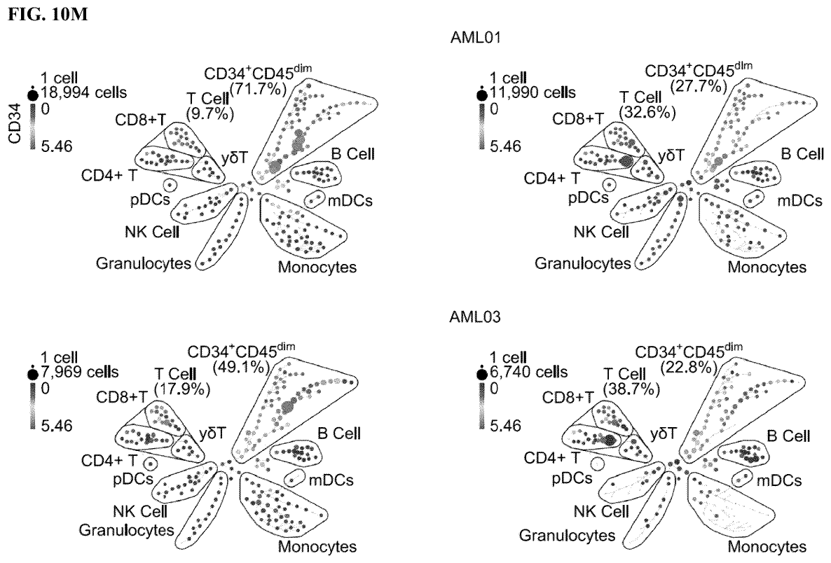

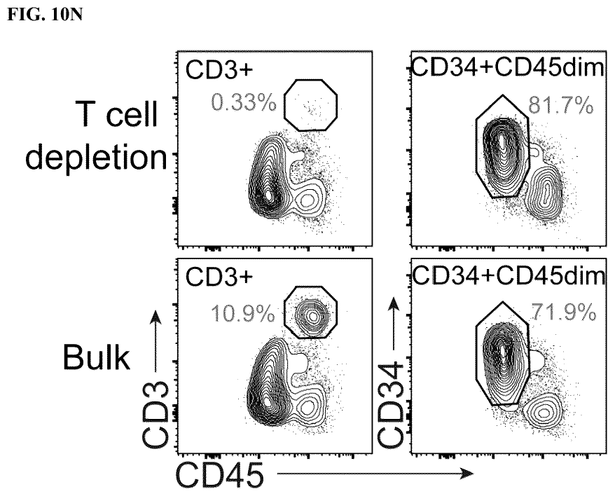

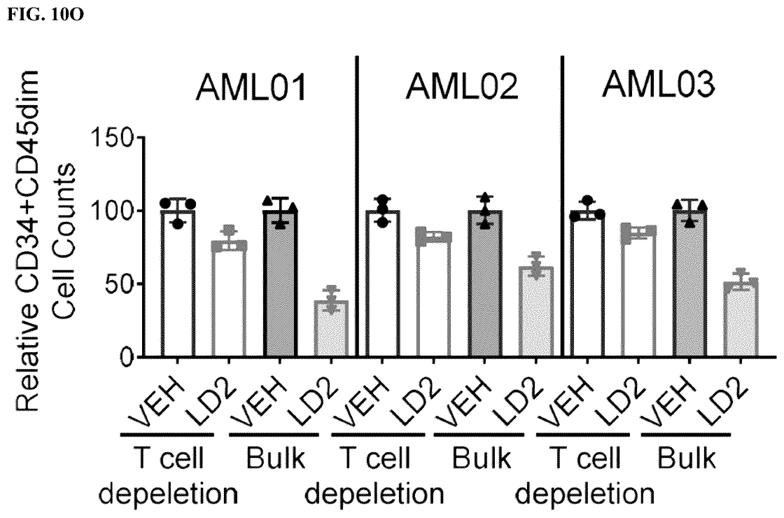

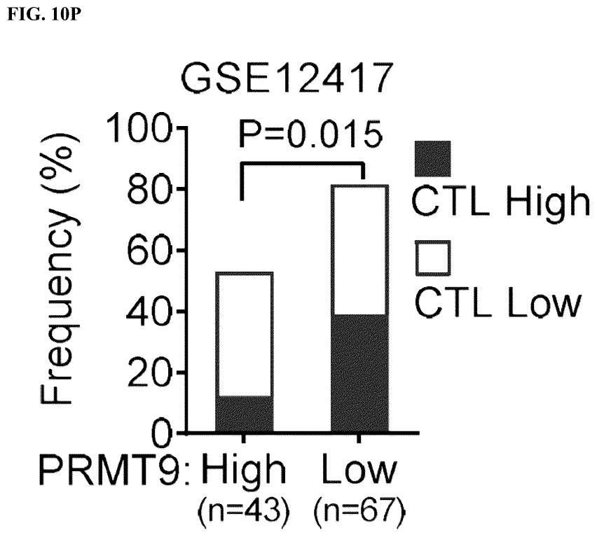

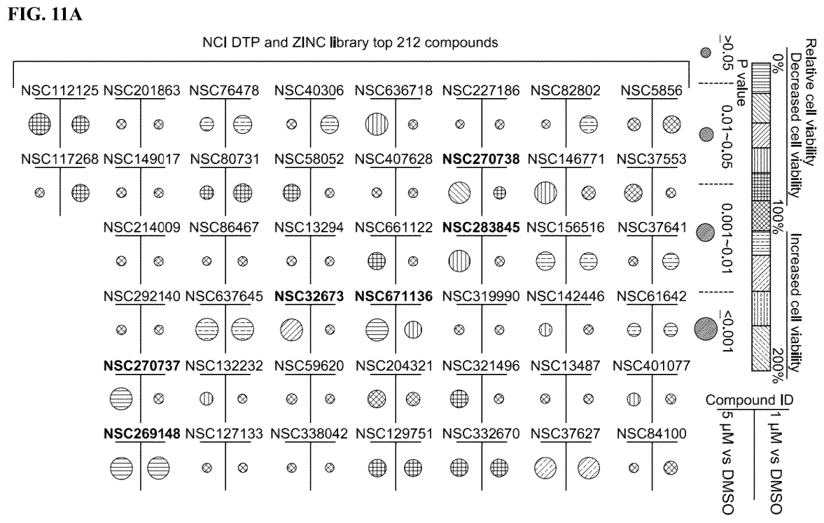

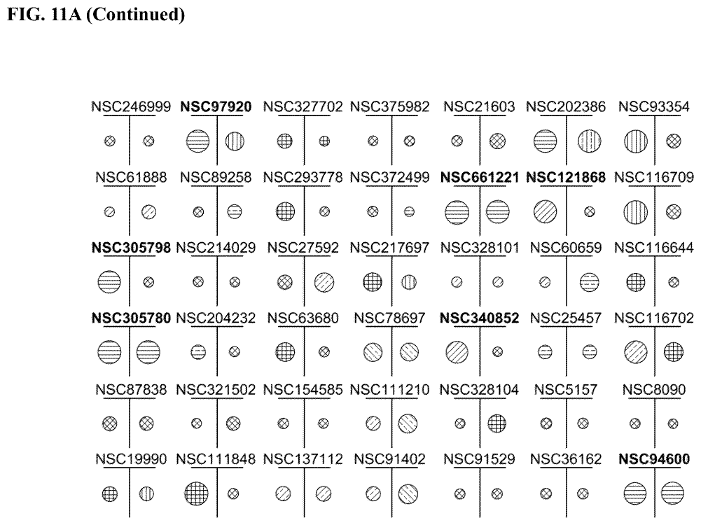

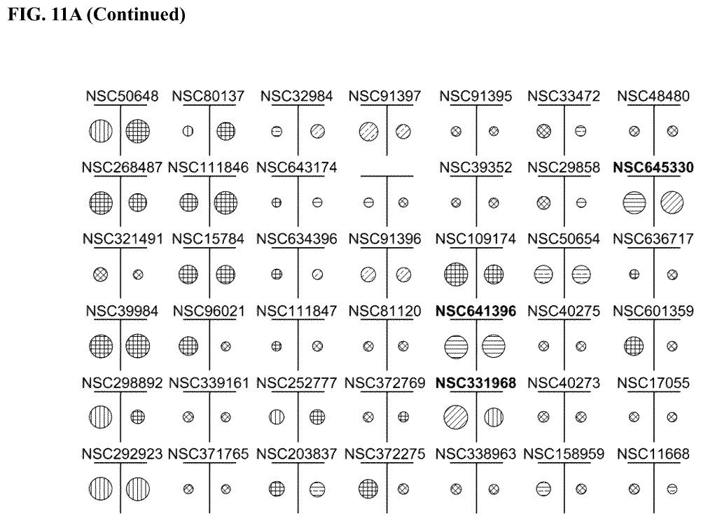

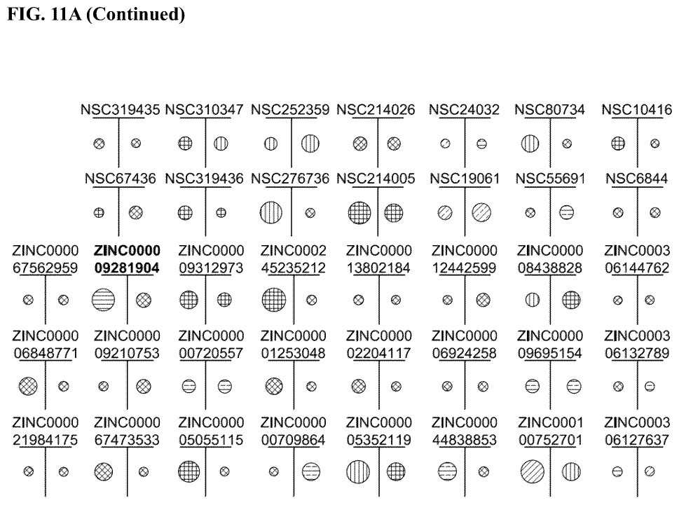

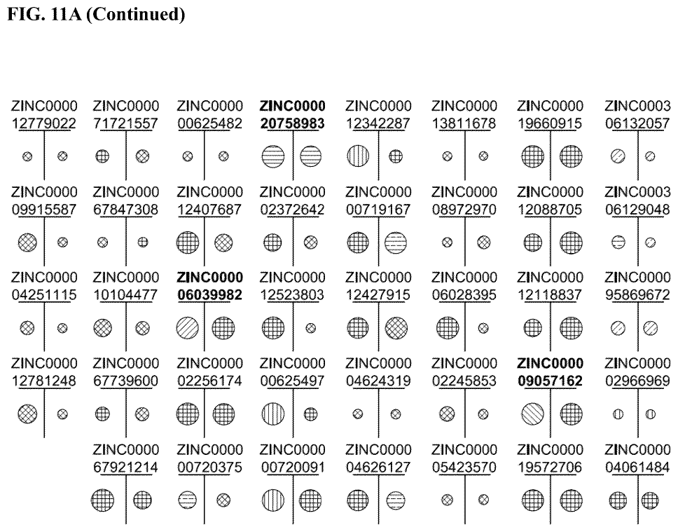

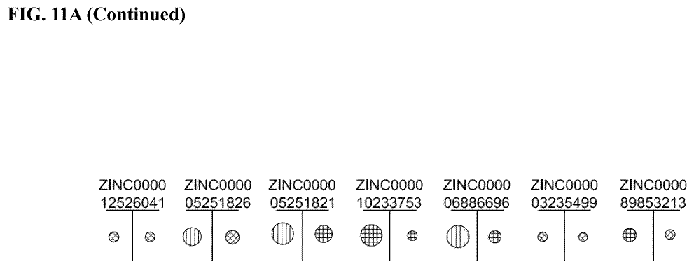

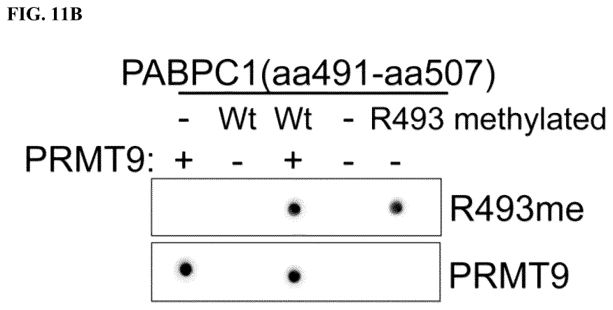

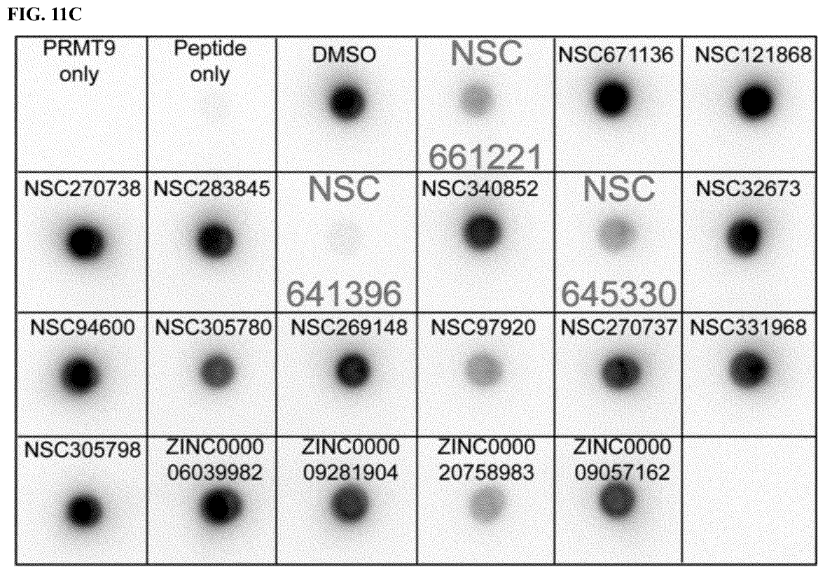

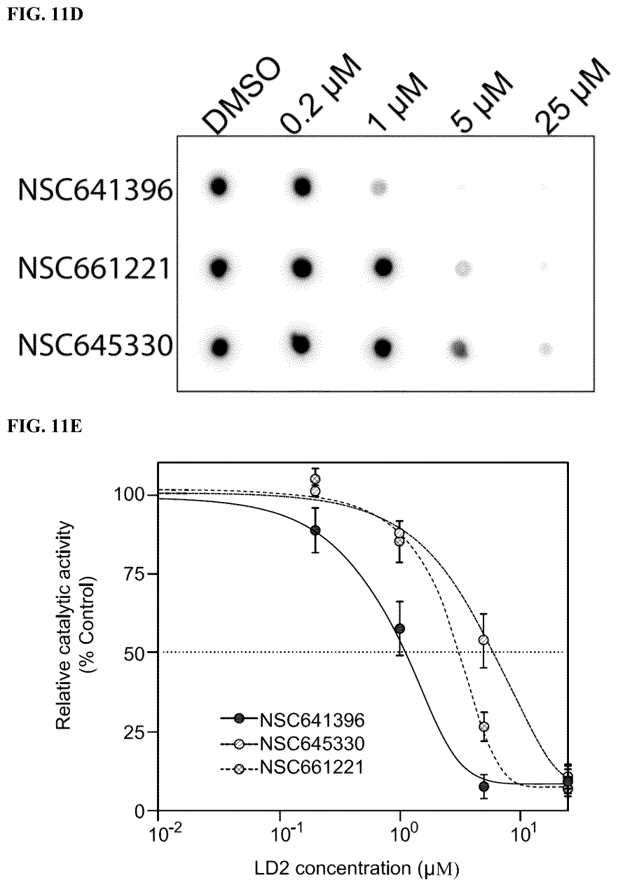

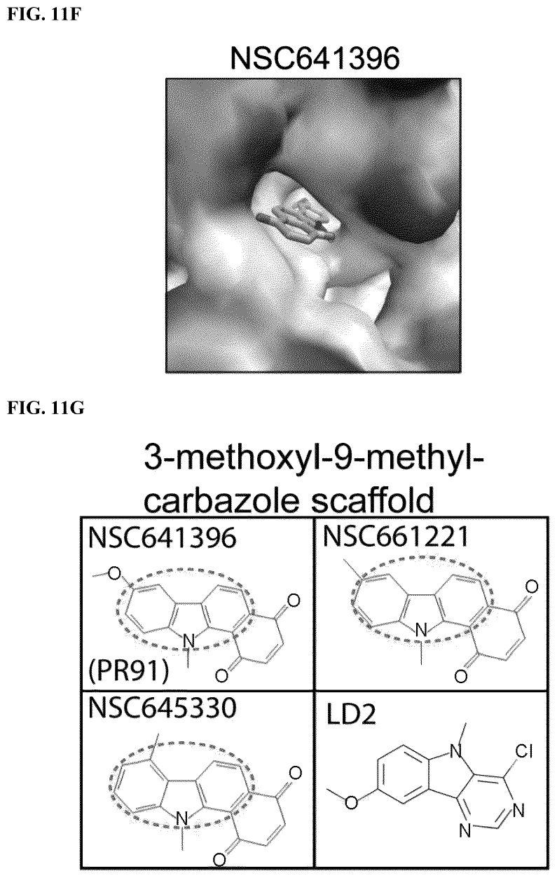

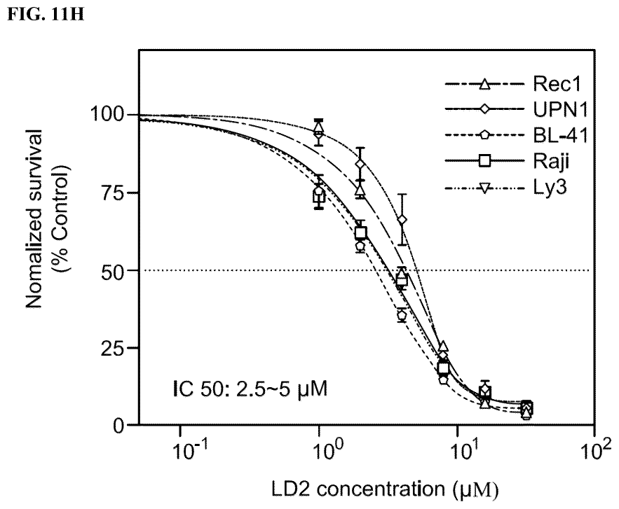

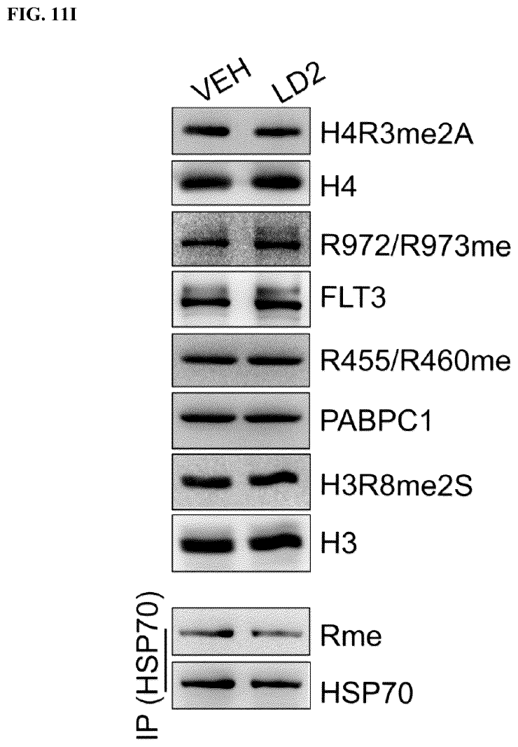

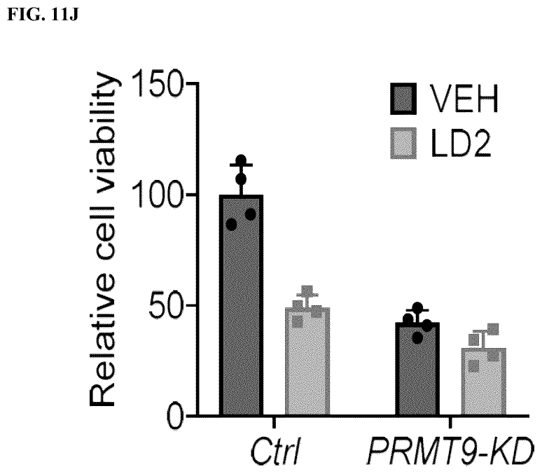

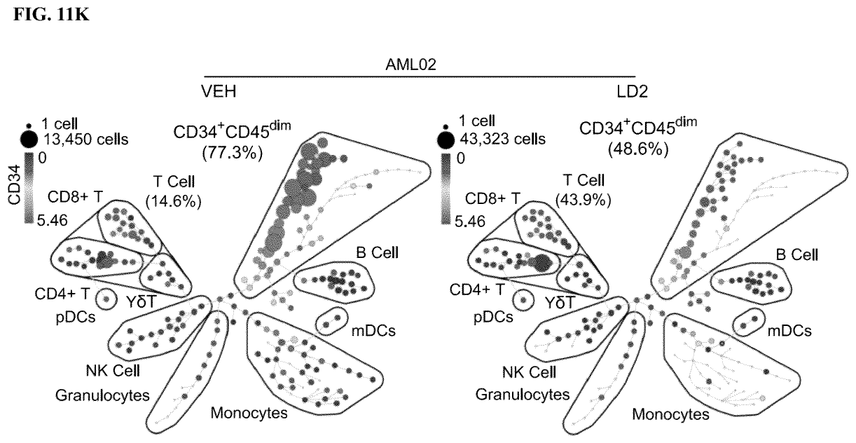

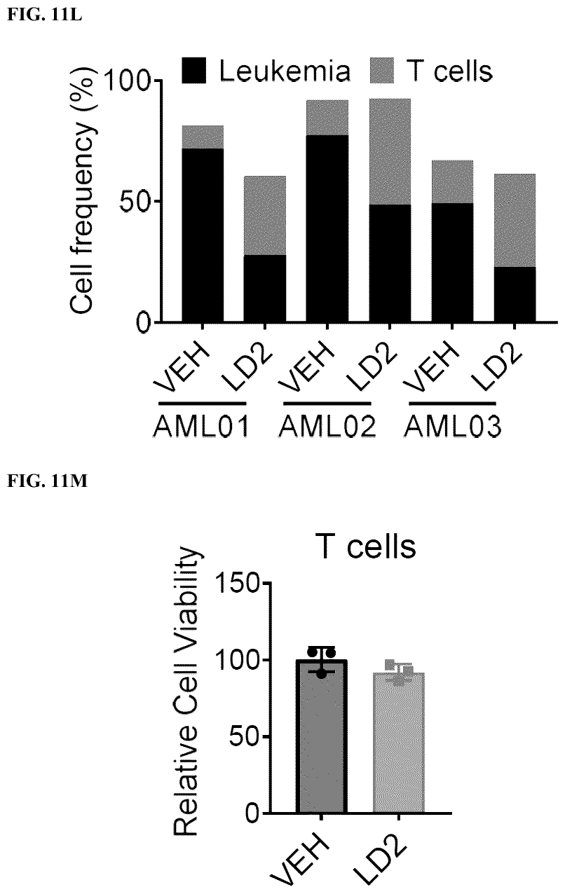

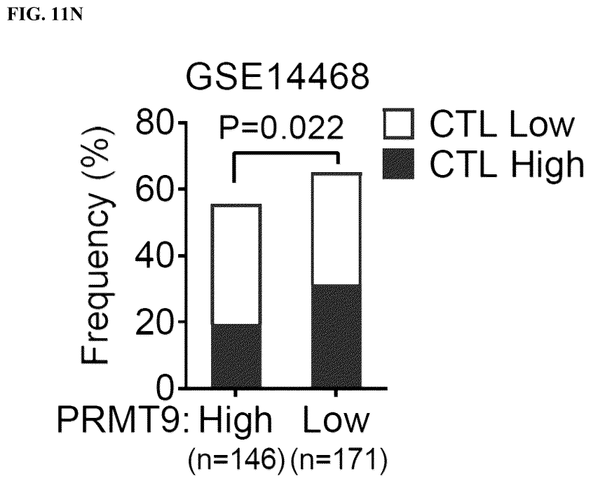

Structure-based virtual screening was next performed to identify PRMT9 inhibitors. Briefly, compounds were screened for binding affinity to PRMT9 using a parallel AutoDock Vina workflow; compounds were sourced from the NCI-DTP (260,000 compounds) and the ZINC library (700,000 compounds) (FIGS. 10A-10B). Candidates were ranked according to lowest binding energy scores (highest binding affinity) to the PRMT9 catalytic pocket (aa137-466) and then requested candidates from the NCI (142 of the top 300 were available) and ZINC (70 of the top 100 were available) to assess anti-AML activity. Doses of 1 and 5 μM were used in a Molm13 cell-based viability assay, given that Molm13 cells are most vulnerable to PRMT9 inhibition (FIG. 11A). Among compounds tested, the top 20 were selected exhibiting robust cell-inhibitory effects for further analysis (FIG. 10C). Specifically, their effects on PRMT9 catalysis were assessed using in-house R493me antibody-based assay (FIG. 11B). After incubating PRMT9, candidate compound with the PABP1 peptide containing unmodified R493 (aa491-507) plus SAM, antibody was added to assess the amount of newly catalyzed R493 methylation (FIG. 11B). Of 20 compounds tested, 3 structurally similar compounds (NSC641396, NSC661221, NSC645330) showed most potent PRMT9 inhibition (FIG. 11C). Among the three, NSC641396 exhibited the most potent PRMT9 inhibitory effects, as evidenced by greatest downregulation of PABP1 R493me levels at lower concentration (FIGS. 11D-11E).

The three compounds shared the same carbazole ring scaffold (FIG. 11F-11G). The NSC641396 docking pose indicated that the quinone ring next to the carbazole moiety extends outside the hydrophobic pocket and does not interact with the protein (FIG. 11F). To enhance NSC641396 binding affinity to the PRMT9 catalytic pocket, lead-optimization was next conducted. To do so, a Tanimoto-based 2-dimensional similarity search was conducted after removing the quinone ring and introducing heteroatoms at different locations of the carbazole moiety (FIG. 11G), which yielded 69 compounds in the PubChem structure database. The top 9 were purchased for wet lab assessment (FIG. 10D). Among them, only #1, #2, #8 showed PRMT9 inhibition efficacy superior to or similar to that of NSC641396, with #1 (thereafter called “LD2”) the most potent (FIGS. 10D-10E). Next, direct LD2 interaction was confirmed with PRMT9 protein by a biophysical approach, nuclear magnetic resonance (NMR). LD2-induced dose-dependent attenuation of signals were observed in Carr-Purcell-Meiboom-Gill (CPMG) NMR titration (FIG. 10F). Moreover, all protons in LD2 showed significant Saturation Transfer Difference (STD) values, and the large variations of STD values among the protons indicate specific interaction between compound/PRMT9 interaction (FIG. 10G). Intracellular interaction of the compound and PRMT9 protein was also assessed via a cellular thermal shift assay (CETSA). Specifically, Molm13 cells were engineered to overexpress Flag-tagged PRMT9-WT or PRMT9-Mut (W152A, D258A and E433A in one construct). Relevant to the mutant, all three residues are predicted drug/PRMT9 binding sites (FIG. 10E). Notably, LD2 treatment led to substantial shifts in thermal stability of Flag-PRMT9-WT but not Flag-PRMT9-mut (FIGS. 10H-10I), confirming LD2/PRMT9 interaction in vivo.

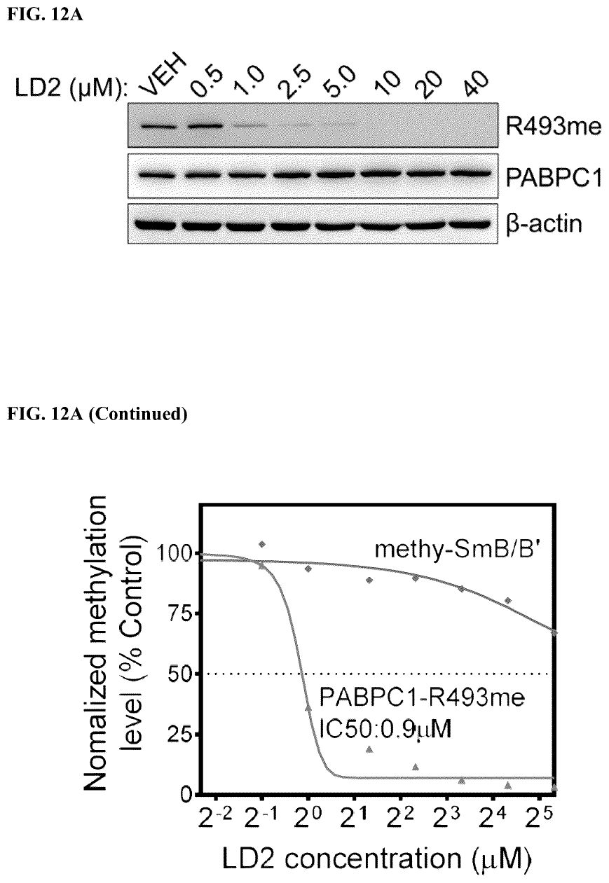

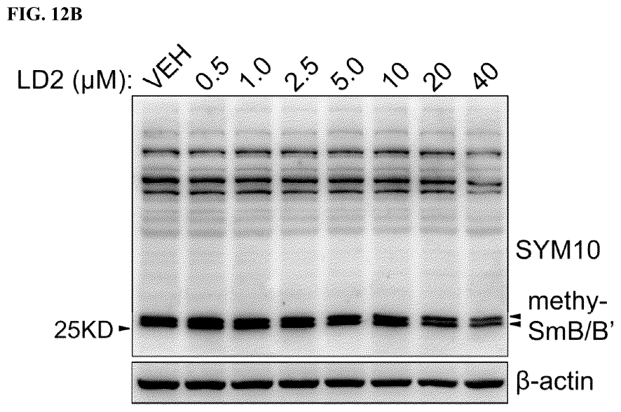

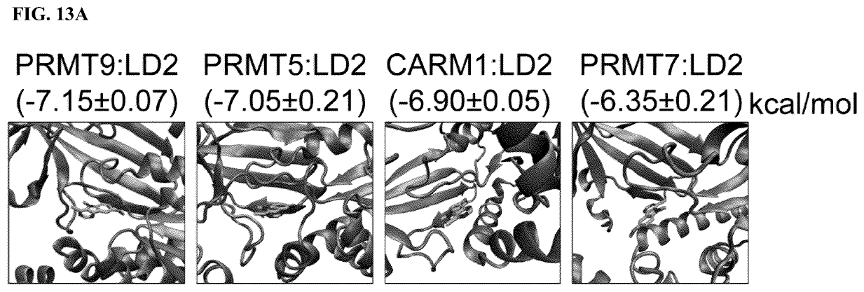

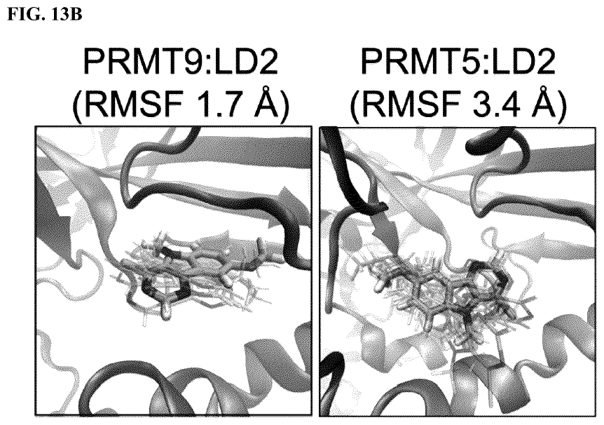

Next, human AML, B-Cell NHL lines and normal CD34⁺ cells from healthy PBSC donors were treated with LD2 ex-vivo. LD2 treatment preferentially inhibited viability of cancer cells rather than normal cells (FIGS. 10J, 11H). Consistent with PRMT9-KD effects, LD2 treatment inhibited protein synthesis in cancer lines (FIG. 10K). LD2 at a relatively low dose (2.5 μM) specifically decreased PRMT9 activity while sparing other PRMTs, such as PRMT1/4/5, based on downregulation of PABPC1 R493 methylation levels, but no changes were observed in methylation levels of specific substrates of other PRMTs (FIGS. 10L, 11I, 12A). At a relatively high dose (20 μM), LD2 treatment also slightly decreased PRMT5 activity (FIG. 12B). Vina docking of LD2 into human CARM1, PRMT5, PRMT7, and PRMT9 was also performed. While the top binding site and docking poses were similar among the four PRMTs, the relatively lower docking score indicated that LD2 binds PRMT9 (−7.15 kcal/mol) with greater affinity than other PRMTs tested (PRMT5, PRMT7, CARM1, FIG. 13A). Molecular dynamics simulation analyses confirmed that LD2 binds deeper in the PRMT9 binding pocket compared with PRMT5, as a smaller root-mean-square-fluctuation (RMSF) of the ligand (LD2) was observed in the PRMT9 (1.7 Å) relative to the PRMT5 (3.4 Å) pocket (FIG. 13B). Finally, PRMT9 KD in Molm13 cells reduced their sensitivity to LD2 (at 2.5 μM), indicating that LD2 effects at that dose are PRMT9 activity-dependent (FIG. 11J).

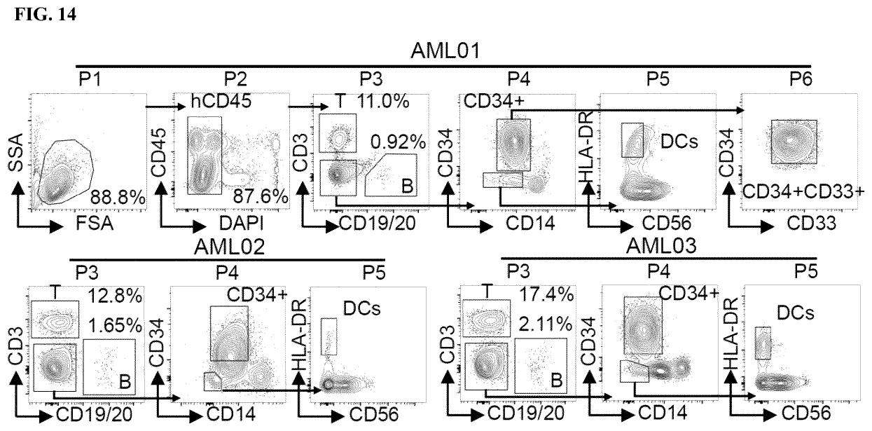

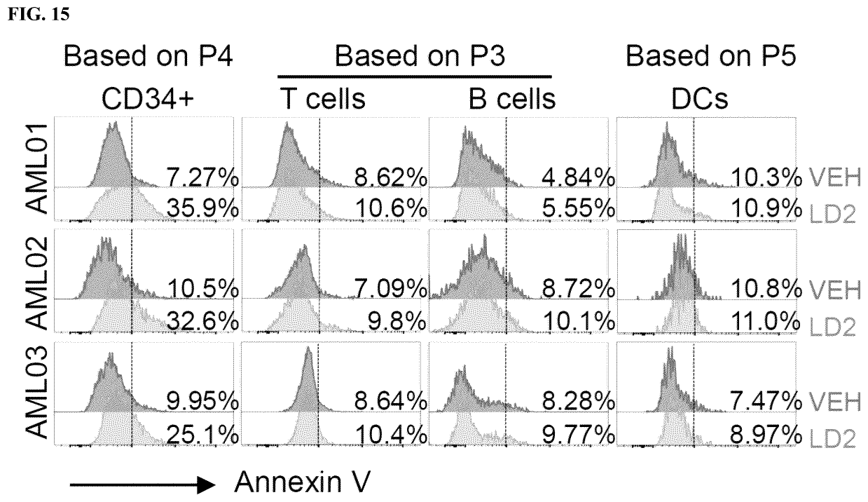

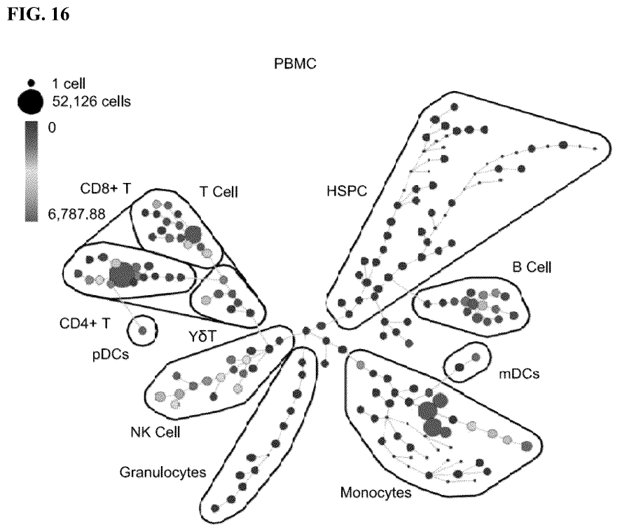

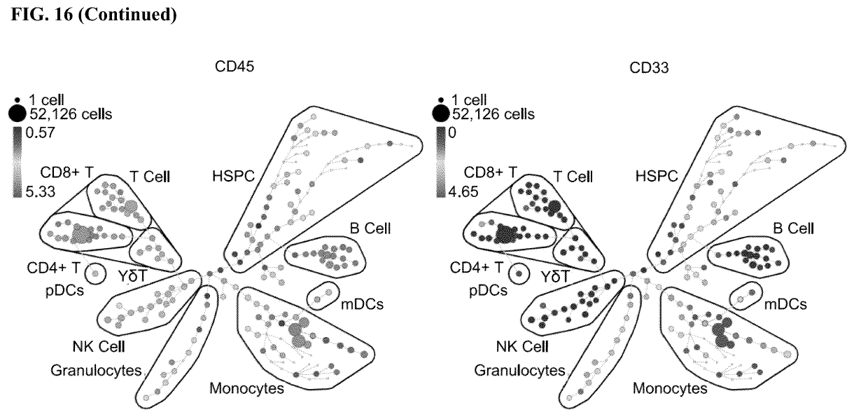

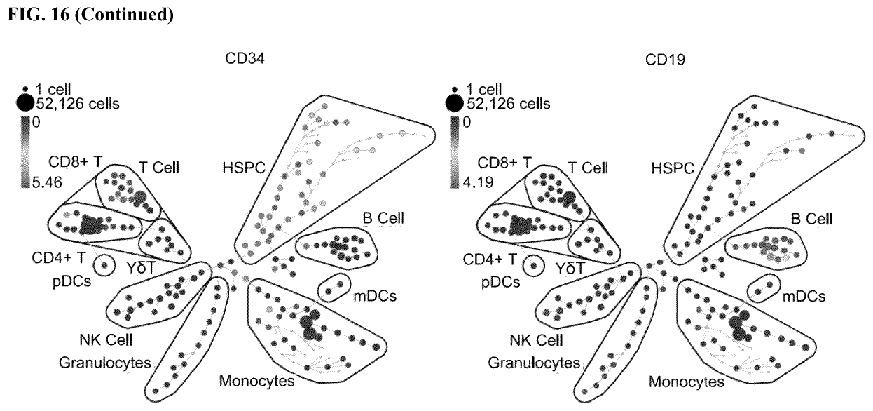

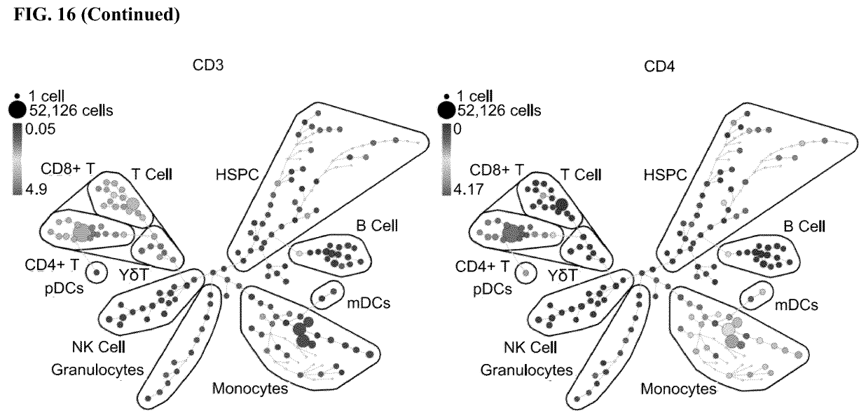

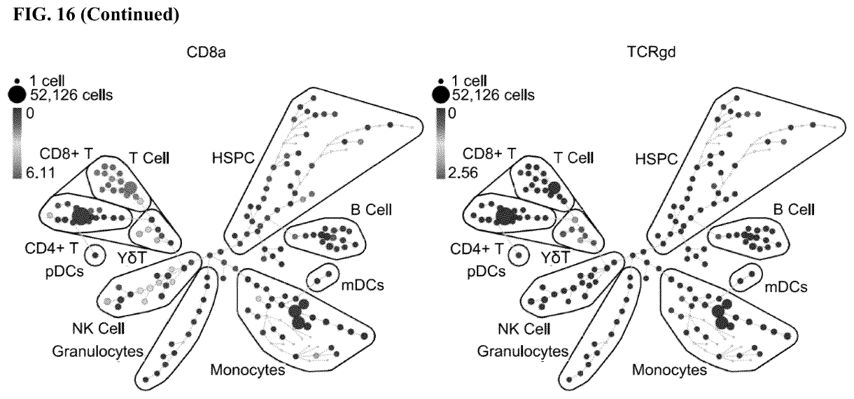

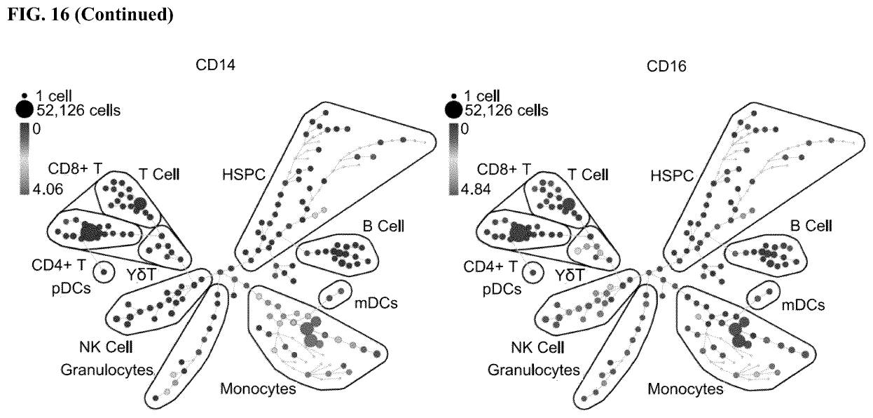

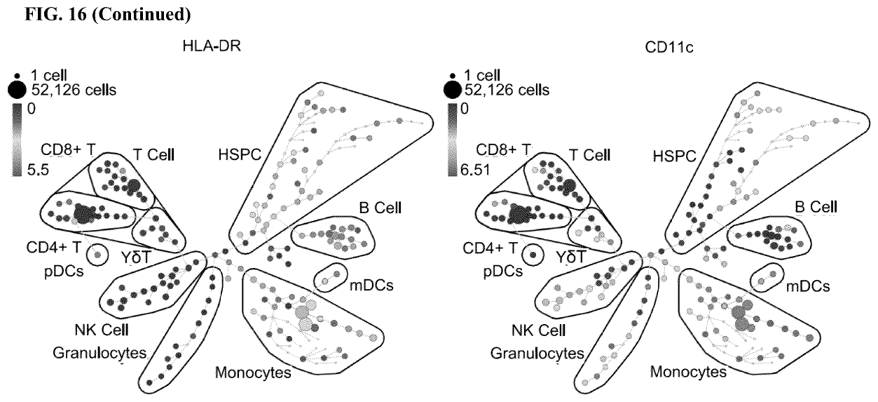

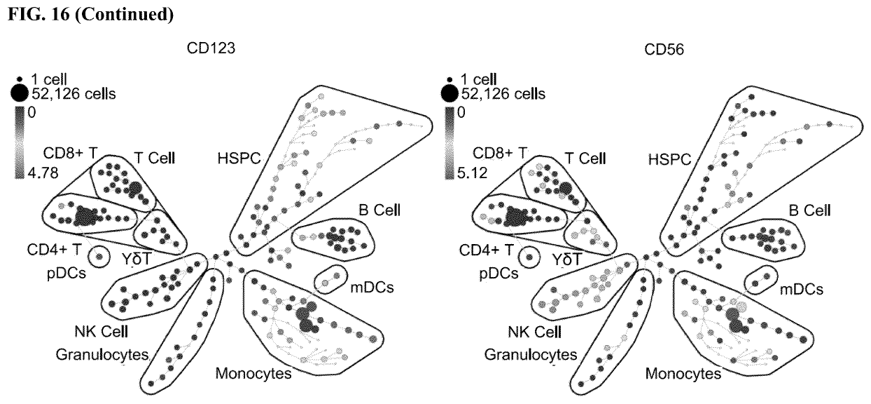

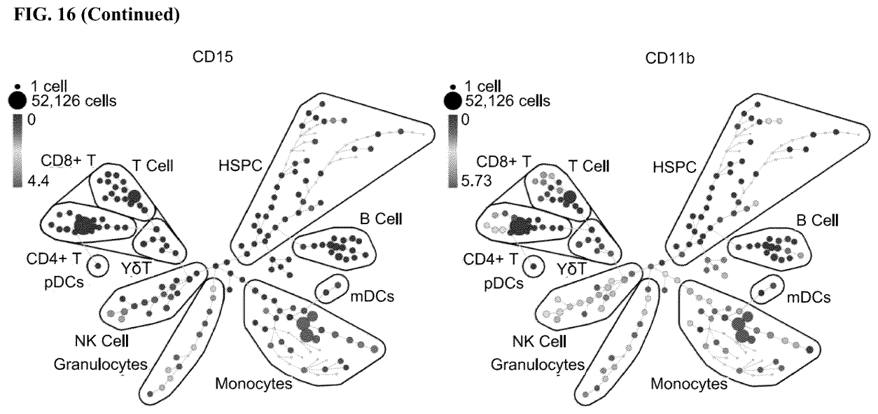

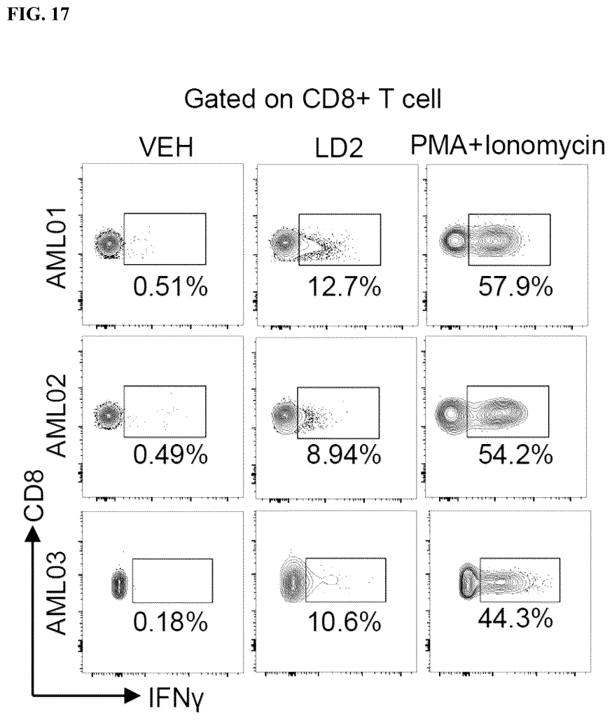

Next, fresh mononuclear cells (MNCs) from AML BM specimens (2 newly diagnosed, 1 relapsed/refractory) were treated for 4 days with LD2 (2.5 μM) under reported physiological cytokine conditions and then used mass-cytometry (CyTOF) to assess surface markers of immune subsets. In these culture conditions, BM subsets (FIGS. 14-15) in the vehicle group exhibited modest levels of apoptosis. An untreated PBMC sample from a healthy donor served as a control for phenotyping. Specifically, high-dimensional SPADE clustering was performed (FIG. 16), yielding cell groups corresponding to immunophenotypic subsets across normal hematopoiesis lineages. Interestingly, SPADE analysis (FIG. 10M) revealed that AML cells from patient samples (n=3) exhibited expansion of the immature CD34⁺CD45^{dim} subset, representing aberrant stem/progenitors. In these immune/leukemia cell co-cultures, LD2 treatment ablated leukemia cells and relatively increased T cell subset ratio (FIGS. 10M, 11K-11L), likely due to the treatment preferentially induced apoptosis in the leukemic subset while spared T cells (FIG. 15). In this ex-vivo culture, it was also found that LD2 treatment increased the number of IFN-γ-expressing T cells relative to vehicle controls (FIG. 17). To further determine the role of T cells in LD2 efficacy, autologous CD3⁺ T cells of AML samples (n=3) were depleted prior to treatment (FIG. 10N). Notably, LD2's AML (CD34⁺CD45^{dim}) inhibitory effects were partially impaired by T cell depletion (FIG. 10O), indicating that T cells function also contributes to LD2 mediated leukemia-ablation effects seen in the ex-vivo culture condition. Moreover, T cell viability was not affected by LD2 treatment (FIG. 11M). To further analyze the relationship between PRMT9 activity and T cell function in human AML, RNA-seq results from GSE12417 and GSE14468 were analyzed, both include large cohorts of AML cases. To do so, 5 genes (CD8A, CD8B, GZMA, GZMB, PRF1) were leveraged known to define a cytotoxic T lymphocyte (CTL) score, as reflective of CD8+ T cell function. Those scores were negatively correlated with PRMT9 levels (FIG. 10P, FIG. 11N), indicating PRMT9's role in immunosuppression.

PRMT9 Inhibition Eradicates AML In Vivo Via a Type-I IFN Response

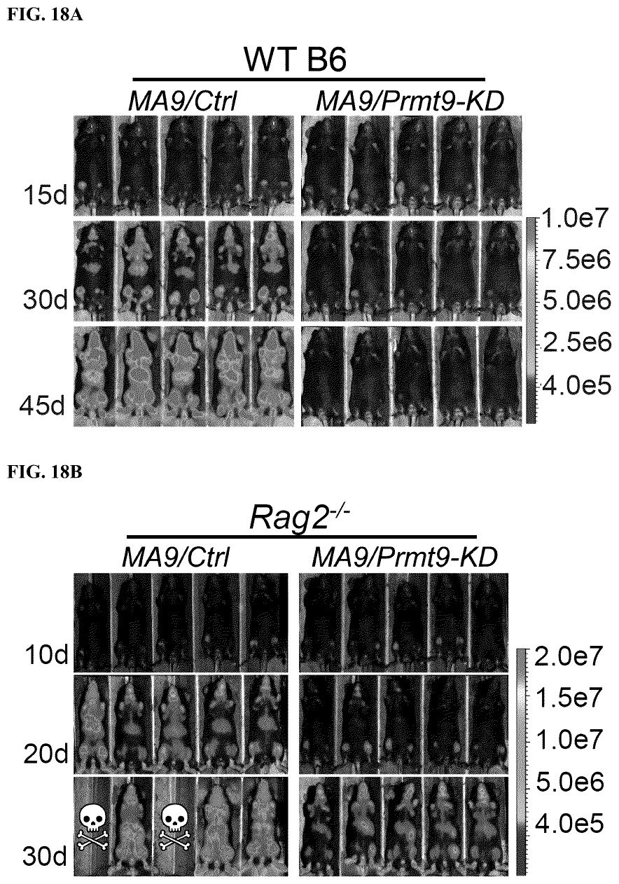

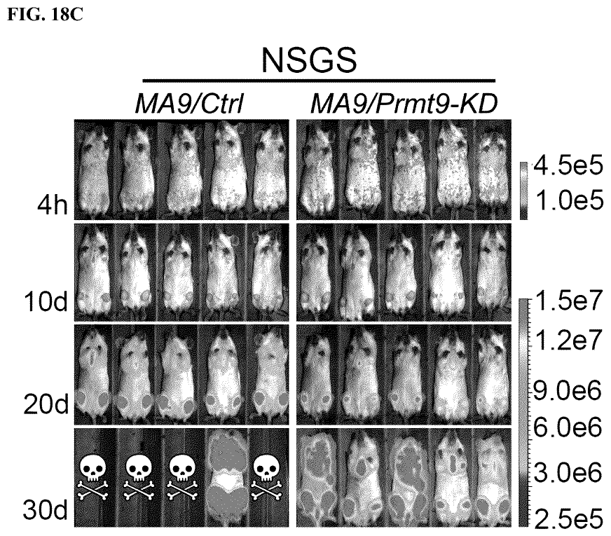

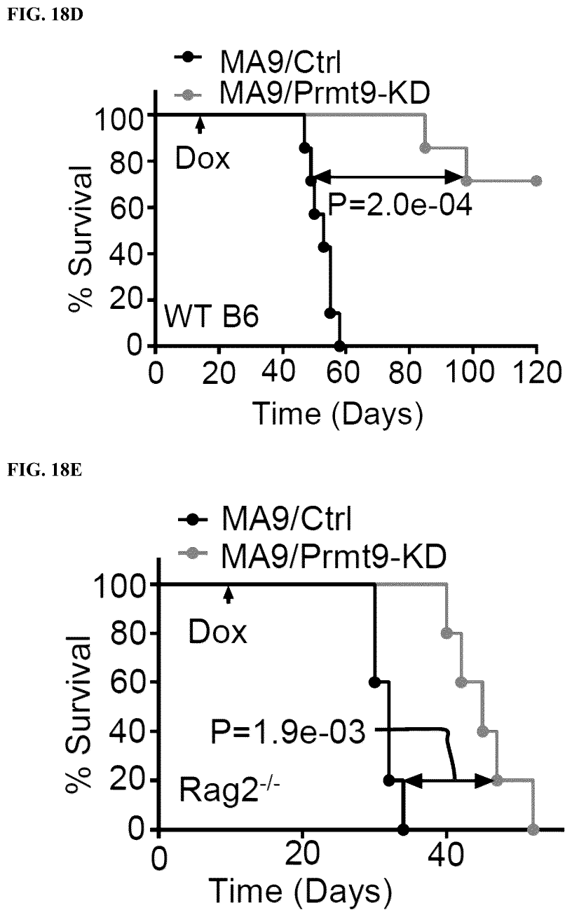

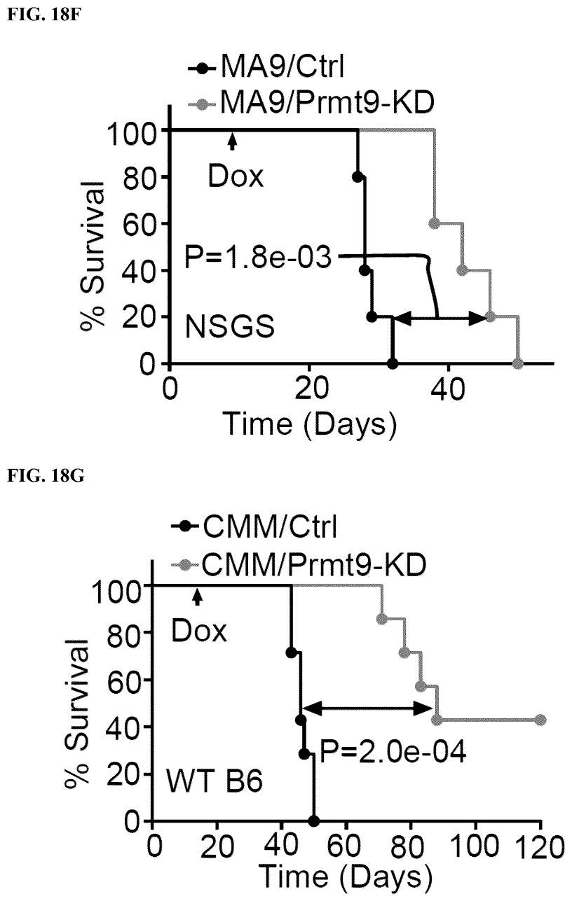

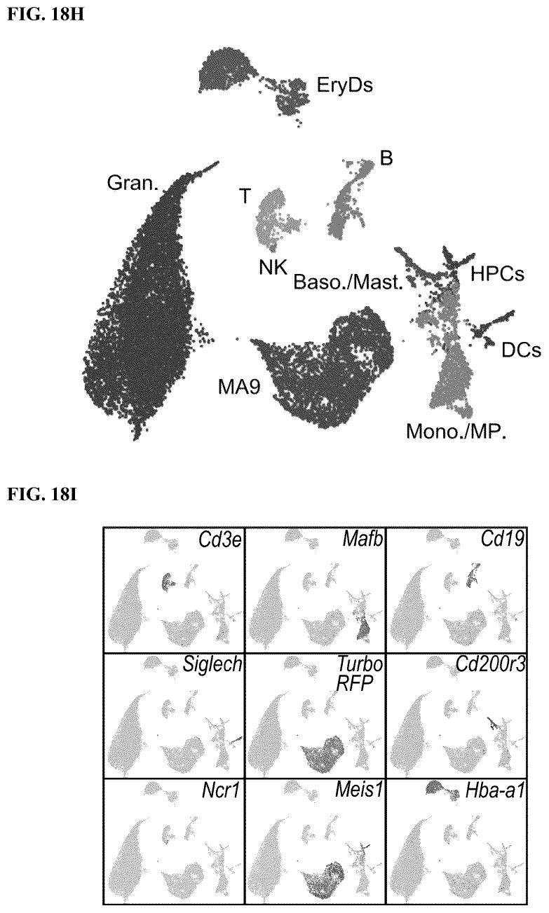

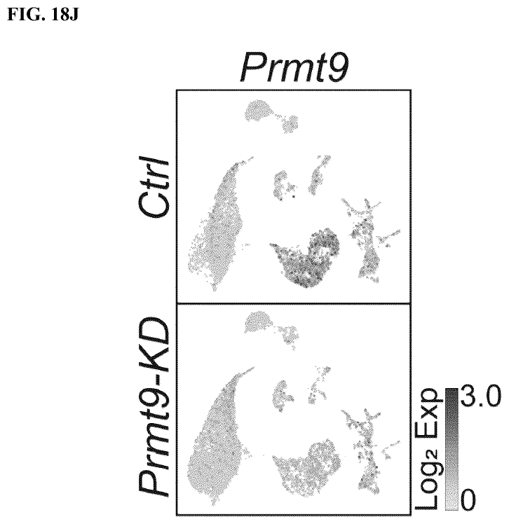

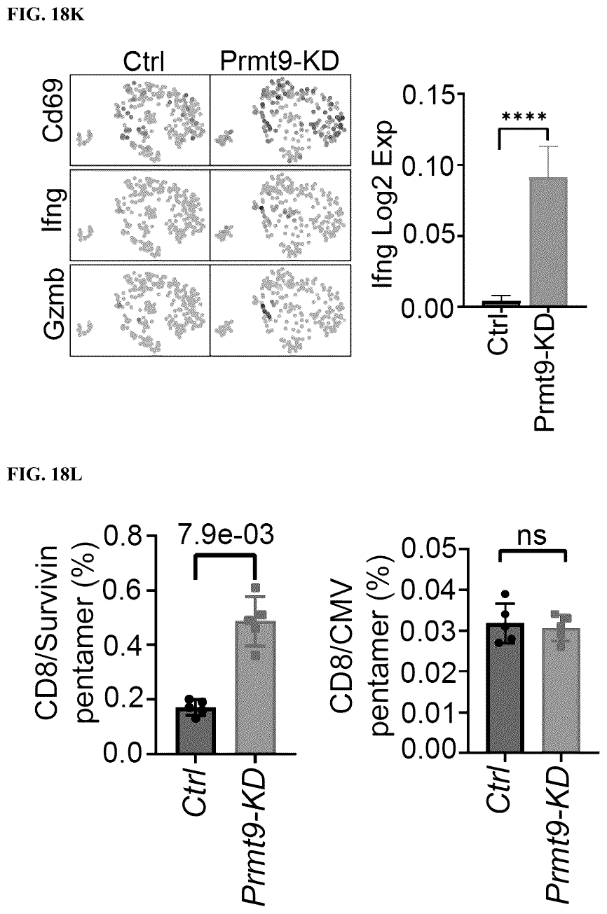

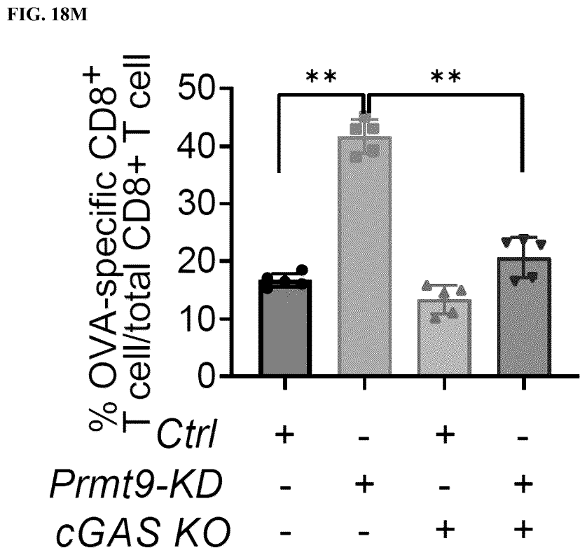

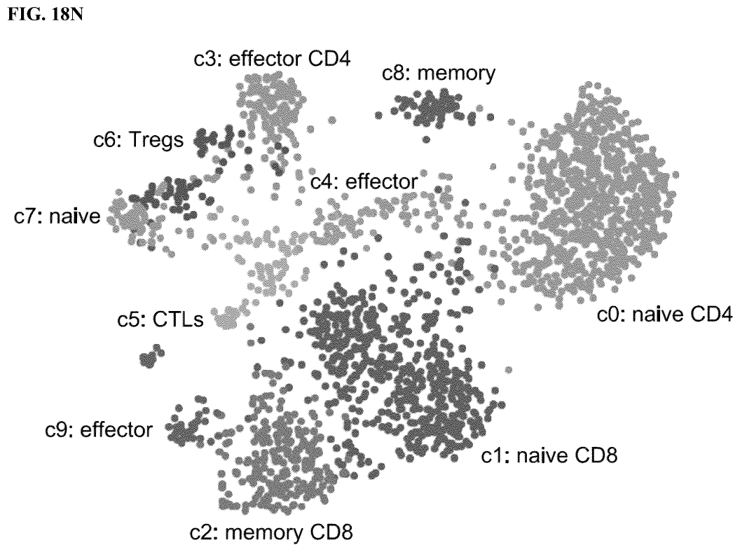

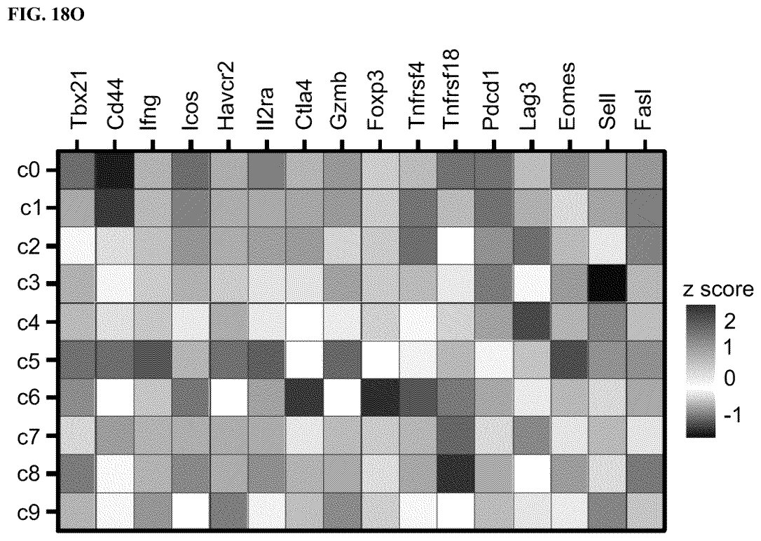

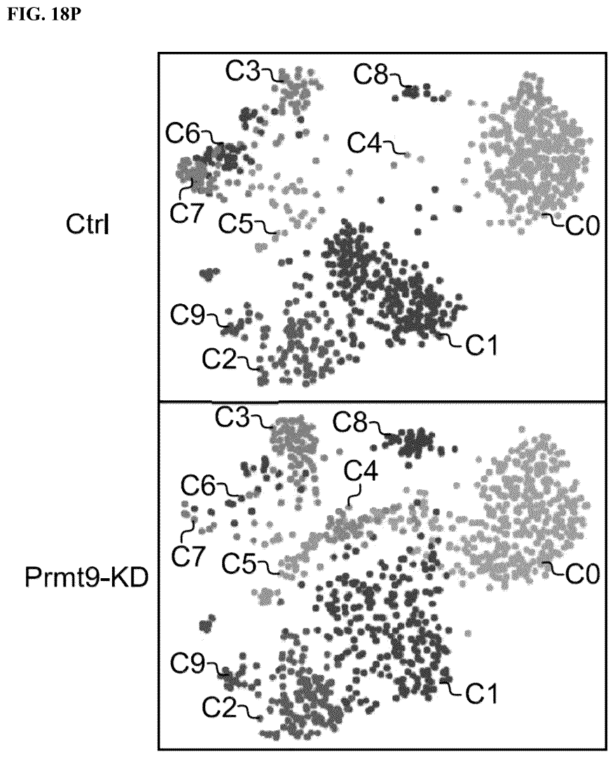

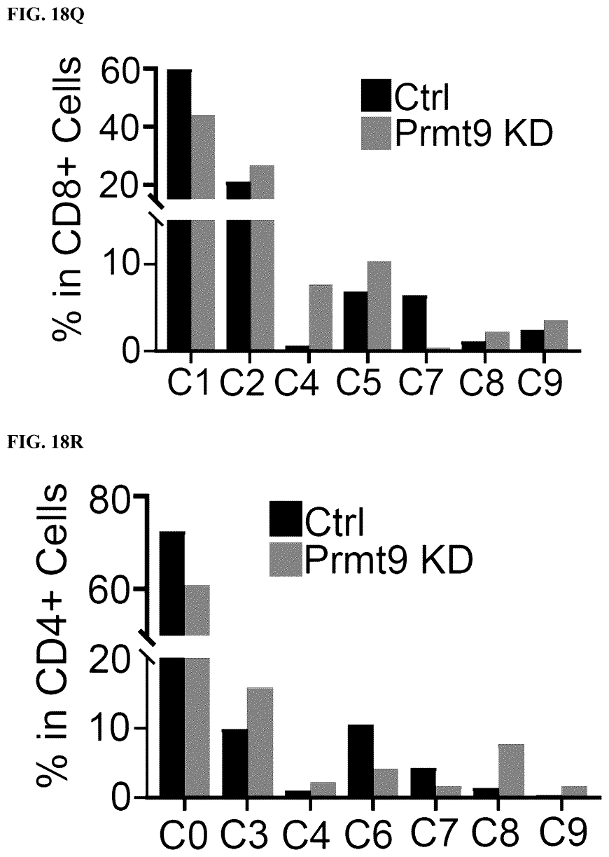

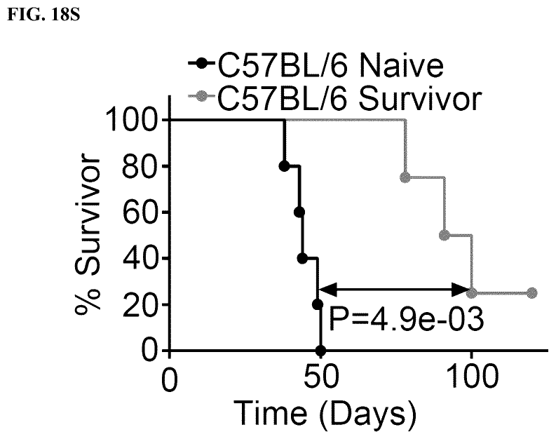

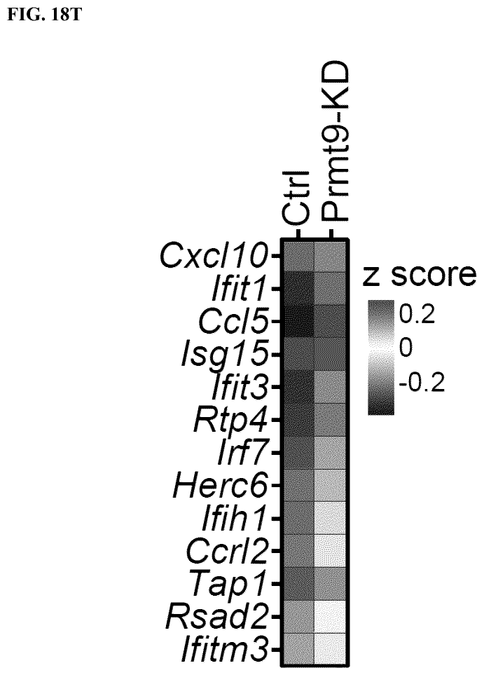

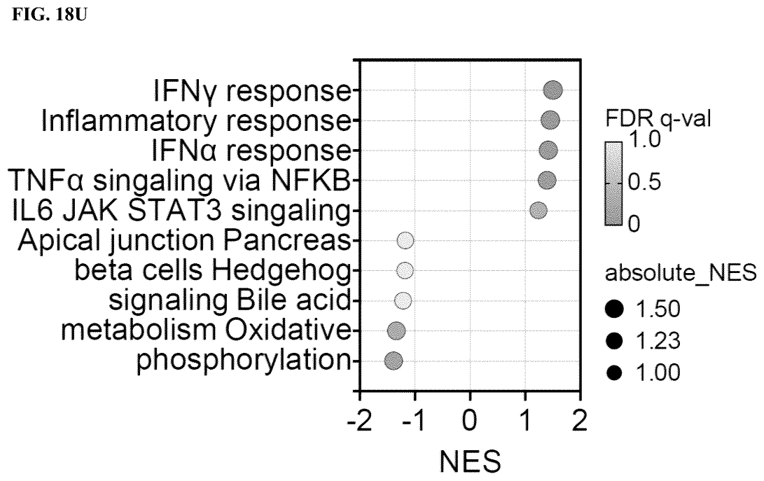

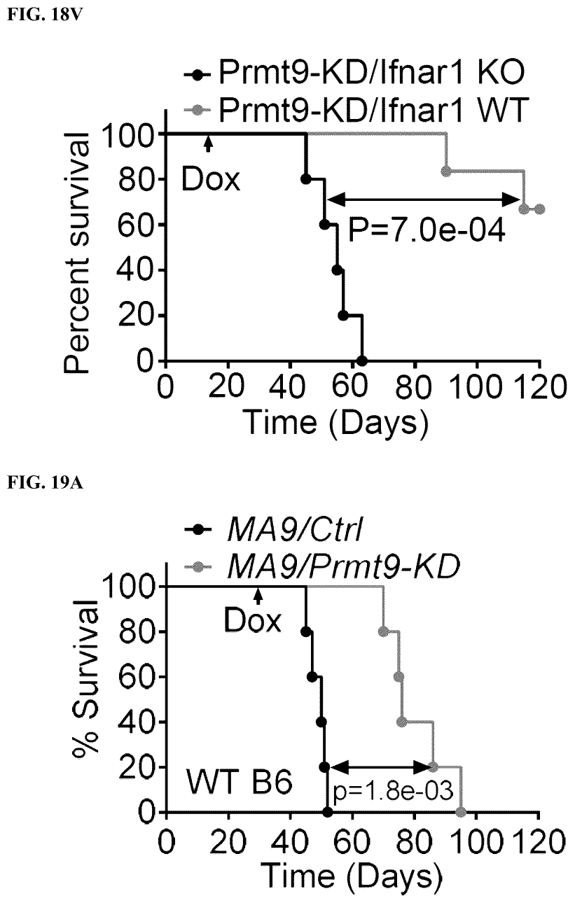

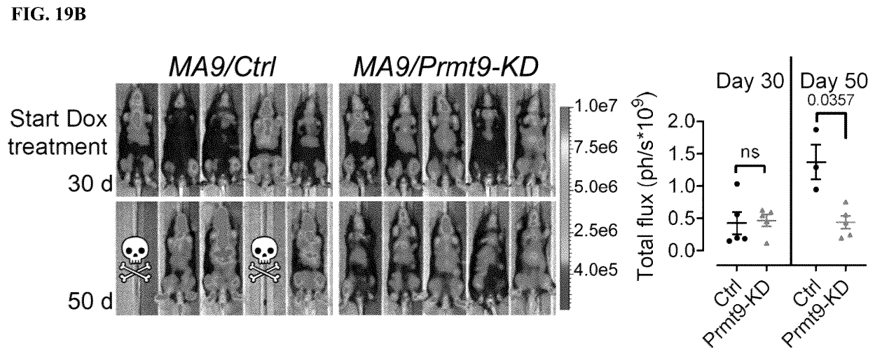

To assess whether cancer-intrinsic PRMT9-inhibition can induce adaptive immune responses, MA9 transduction AML transplant model (one million cells per transplant) was used which can be transplanted into WT immunocompetent recipient mice to induce leukemia (FIGS. 18A, 18D). Moreover, these MA9 AML cells have further been transduced with either inducible shPrmt9 or shCtrl construct co-expressing a luciferase reporter and the puro-resistance gene. Besides congenic WT B6 immunocompetent mice serving as transplant recipients, w transplants were also established with recipients of immunodeficient Rag2^{-/-} mice (FIG. 18B) or NSGS mice (FIG. 18C), to compare Prmt9 deletion effects in immunodeficient recipients vs those of immunocompetent. When leukemia cells engrafted in BM (FIGS. 18A-18C), mice were treated with DOX to induce Prmt9-KD (15d post-transplant for WT B6 recipients, 10d post-transplant for Rag2-i or NSGS recipients). Following induction, Rag2^{-/-} (FIGS. 18B, 18E) or NSGS (FIGS. 18C, 18F) mice bearing Prmt9-KD transplants survived significantly longer than did mice with Prmt9-WT transplants, but eventually succumbed to leukemia (<60d) (FIGS. 18E-18F). The results seen in these two immunodeficient models were similar (FIGS. 18E-18F). Unlike the immunocompromised mice, 5 of 7 WT recipients bearing Prmt9-KD AML survived the observation period (>120d) (FIG. 18D). Interestingly, luciferase-expressing leukemia cells nearly disappeared over time (FIG. 18A). Another cohort of MA9 leukemia transplants was also established using WT recipients, and induced PRMT9 deletion at day 30, a time mimicking the clinical situation in which AML burden is robust and BM tumors are likely at an advanced stage. As shown (FIGS. 19A-19B), PRMT9-KD decreased AML burden and modestly extended survival of leukemic mice. To determine which immune subset contributes to leukemia regression seen following PRMT9-KD (FIGS. 18A, 18D), antibody-based depletion of T or NK cells was performed prior to in-vivo DOX administration to KD PRMT9 mice (FIG. 20A). Compared to anti-IgG controls, CD4/CD8 T cell depletion significantly abolished Prmt9-KD induced AML regression, while NK-depletion had only modest effects on survival (FIG. 20B).

To verify the role of Prmt9 inhibition in a different AML model, CMM AML cells were also transplanted to form a clonal leukemia disease in WT immunocompetent recipient mice. As a result, Prmt9-KD mediated leukemia elimination effects are comparable in CMA[models and MA9 model (FIG. 18G), confirming Prmt9-KD induces immune elimination of AML. Given that LSC ablation is critical to achieve a cure, it was asked whether Prmt9 KD impairs LSC activity in immunocompetent mice by harvesting BM cells from WT recipients bearing Prmt9 KD AML (FIG. 18D, FIG. 18G) and conducting in-vivo limiting dilution transplant assays. Notably, Prmt9 KD remarkably decreased leukemia initiating cell (LIC) frequency in both AML models.

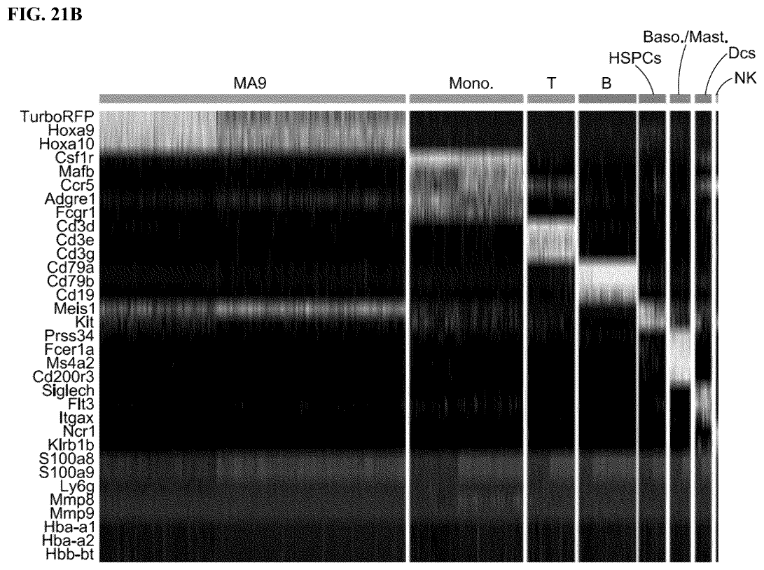

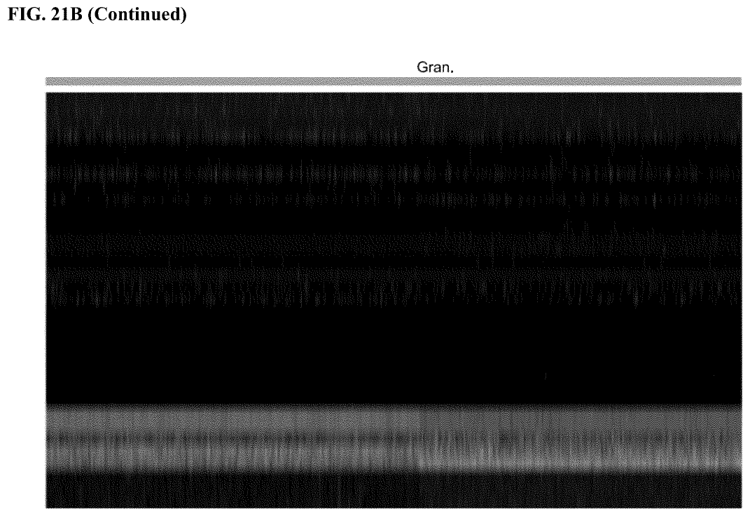

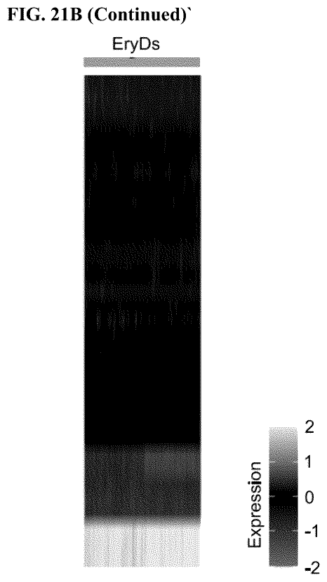

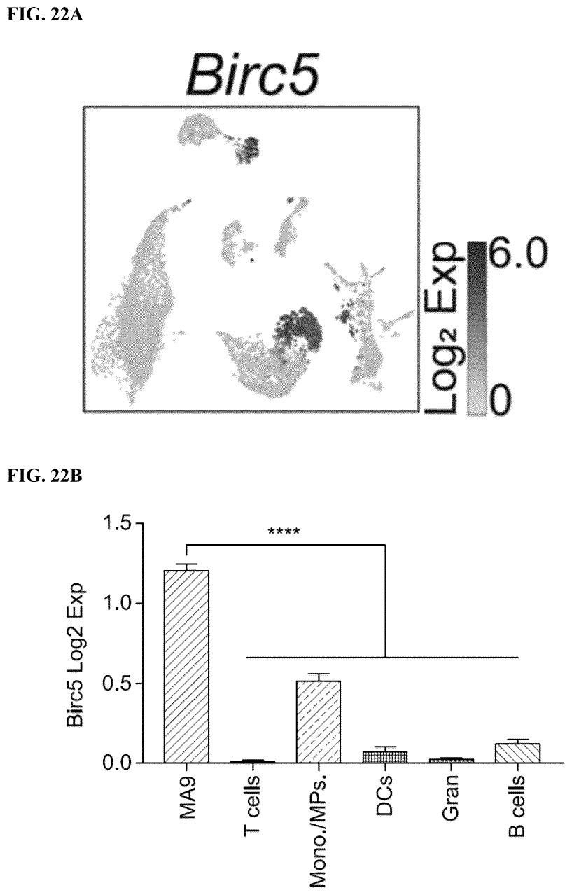

To provide unbiased assessment of immune responses upon Prmt9 inhibition, single-cell RNA sequencing (scRNAseq) analysis of the MA9 AML tumor micro-environment (TME) was performed. To do so, a different cohort of WT B6 mice transplanted with DOX-inducible Prmt9-KD or control was established and evaluated transcriptional status of all immune lineages in transplants 7 days after DOX treatment. At that time point, mice receiving Prmt9-KD cells begin to exhibit decreased leukemia cell engraftment and increased T cell frequency in BM (FIG. 21A). Then BM as well as spleen cells were collected from a representative mouse (leukemia engraftment at median level) in each group to scRNAseq analysis (FIG. 21A). Single-cell transcriptomes for 9,741 control and 11,291 Prmt9-KD BM cells were obtained. Following canonical correlation analysis, graph-based clustering and dimensionality reduction with UMAP was used to identify and visualize transcriptionally homogeneous cell clusters (FIGS. 18H, 21B). Cancer cells expressing TurboRFP, Hoxa9 an dMeis1 were identified; normal HSPCs also expressing Meis1 but without TurboRFP; T cells and innate lymphoid cells (ILCs) both expressing Cd3d, Cd3e and Cd3g, NK cells expressing natural cytotoxicity triggering receptor 1 (Ncr1), B cells expressing Cd19, Cd79a and Cd79b, myeloid populations such as monocytes/macrophages expressing Mafb and Csf1r, dendritic cells (DCs) expressing Siglech, Itgax (Cd11c) and Flt3, granulocytes expressing S100a9 and Ly6g, and basophils/master cells expressing Cd200r3 and Prss34 (FIGS. 18H-18I, 21B). Notably, Prmt9 levels seen were significantly higher in leukemic cells relative to normal lineages; Prmt9 KD decreased tumor cell frequency relative to the controls (FIGS. 18J, 20C-20D). Interestingly, relative to the controls, Prmt9 KD elicited T cell activation with increased Cd69, Granzyme B and Ifng expression (FIG. 18K). To verify T cell function, leukemia-specific T cell responses were assessed following Prmt9 KD using MHC-survivin peptide pentamer analysis (MHC-CMV peptide as a control) as described by Stroopinsky et al. Haematologica 106, 1330-1342, doi:10.3324/haematol.2020.259457 (2021). Elevated expression of Birc5 (encoding Survivin) was confirmed in MA9 leukemia cells relative to other immune subsets (FIG. 22). Using new cohorts of MA9 transplant mice, it was observed that after 7 days of DOX treatment cells from Prmt9-KD mice showed a significantly expanded BM population of CD8⁺ T cells recognizing tumor survivin relative to Prmt9-WT controls (FIG. 18L). In contrast, LD2 treatment did not increase CMV-specific CD8⁺ T cell frequency. Cancer-specific T cell responses following Prmt9 KD using MA9/OVA cells was also confirmed (FIG. 18M), which are engineered to harbor the OVA gene to track antigen-specific T cells with an MHC-OVA peptide (H-2K^{b}-OVA₂₅₇₋₂₆₄) tetramer during tumor development.

To precisely define T cell subpopulations, scRNA-seq results of spleen was analyzed, in which T cells are more abundant (20-30%). Specifically, the transcriptomes were generated based on 8,792 cells from control group and 8,598 cells from Prmt9-KD group. Cell clusters are annotated using the same strategy as BM samples (FIGS. 20E-20G). As a result, Prmt9 KD modestly increased T cell frequency (FIG. 20H). Then, the focus was shifted on T cells, and they were reanalyzed at higher granularity based on levels of classical markers (e.g., Cd44, Sell, and Foxp3) (FIGS. 18N-18, 20I). Accordingly, 10 distinct T cell subpopulations (c0-9) were characterized, including c0 (naïve CD4⁺), c1 (naïve CD8⁺), c2 (memory CD8⁺), c3 (effector CD4⁺), c8 (memory T), c4 and c9 (effector T), c5 (CTLs), and c6 (Tregs) (FIGS. 18N-18O). Notably, Prmt9 KD altered proportions of subpopulations and their transcriptional profile (FIGS. 18O-18R). Cells in c0 and c1 with either Cd4 or Cd8, are considered to be Naïve T cells, since they are expressing naive T cell marker Sell (CD62L) but lack of expression of effector/memory marker Cd44 and T cell activation genes such as Ifng and Il2ra (FIG. 18O). Prmt9KD decreased numbers of naïve T cells (c0, c1) and increased effector/memory T cell subsets (c2, c3, c4, c8 and c9) (FIGS. 18Q-18R). Prmt9 KD also increased CTLs (c5) and decreased Tregs (c6) (FIGS. 18Q-18R). In agreement with the observations from spleen, Prmt9-KD also expanded Cd44-expressing T cells and decreased Foxp3-expressing Tregs in BM (FIGS. 20J-20K).

To verify whether Prmt9 KD induced immune memory, primary WT B6 mice was selected that survived from previous MA9 leukemia transplantation and had shown complete regression of MA9 tumors upon Prmt9-KD. These as well as another control cohort composed of naïve mice never exposed to MA9 tumor cells, were re-challenged, by injection with comparable numbers of MA9 tumor cells. In the latter, tumors grew aggressively (FIG. 18S). Although tumor-free mice exhibited a remarkable survival benefit following MA9 cell re-challenge, most died of leukemia (FIG. 20L), indicating a modest induction of antigen-specific immune memory after Prmt9 inhibition.

Moreover, T cell transcriptomic analysis revealed that Prmt9 KD upregulated interferon-stimulated gene (ISG) levels (FIG. 18T). GSEA analysis also highlighted transcriptional activation of the IFNα/b response pathway in T cells (FIGS. 18U, 20M). Similarly, ISGs including Isg15 (FIGS. 20N-200), Ifit1 (FIGS. 20P-20Q), and Cxcl10 (FIG. 20R) upregulation were seen in other immune cell types, including monocytes, granulocytes, B cells and DCs. To confirm that type-I IFN responses underlined Prmt9-KD effects, Prmt9-KD MA9 cells was implanted into WT recipient or type-I IFN receptor KO (Ifnar1-KO) mice. Following leukemia cell engraftment, Prmt9 KD was induced and monitored leukemia development. Interestingly, anti-AML effects of Prmt9-KD were significantly abolished on an Ifnar1-KO background (FIG. 18V), indicating that these effects require IFN-I signaling.

Anti-Tumor Immunity Following PRMT9 Inhibition Requires cGAS Activity in Cancer Cells

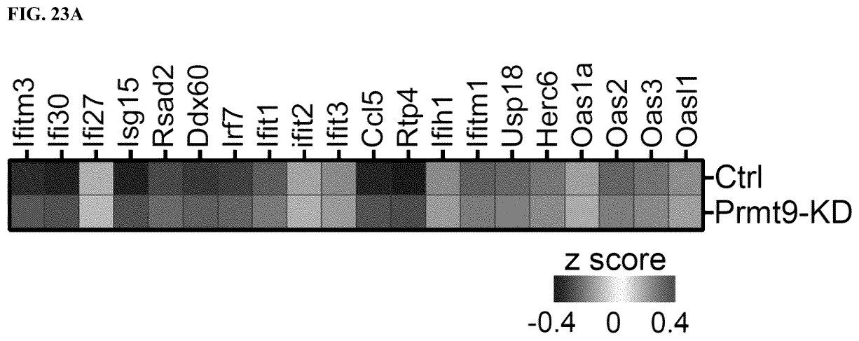

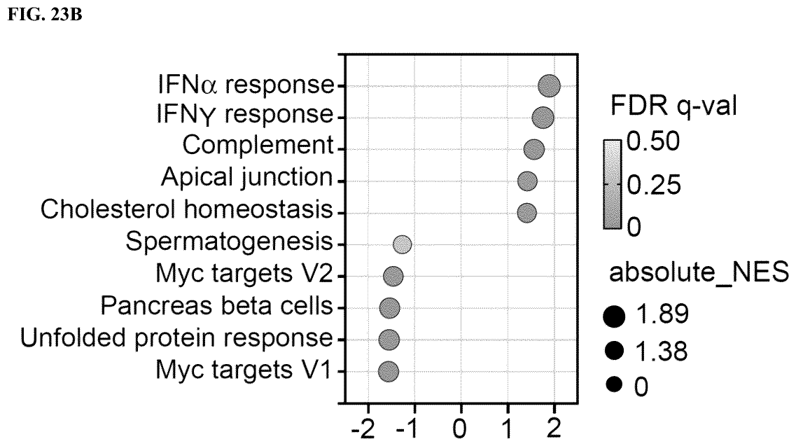

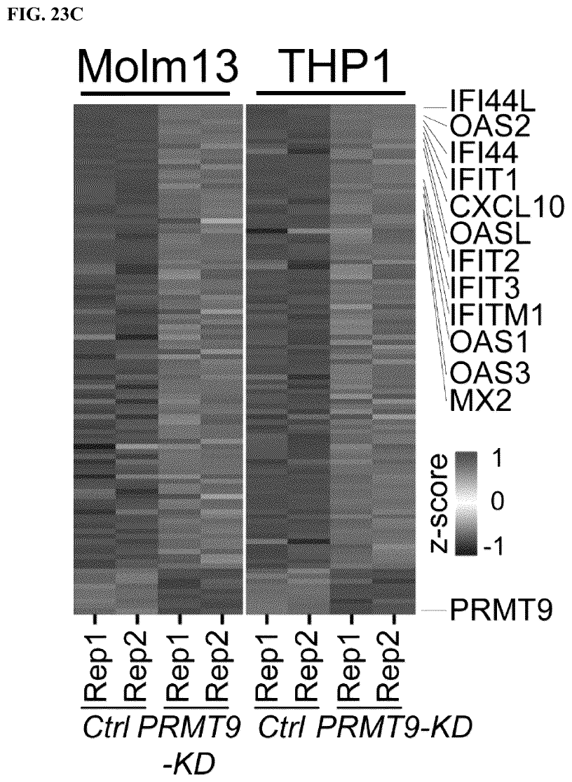

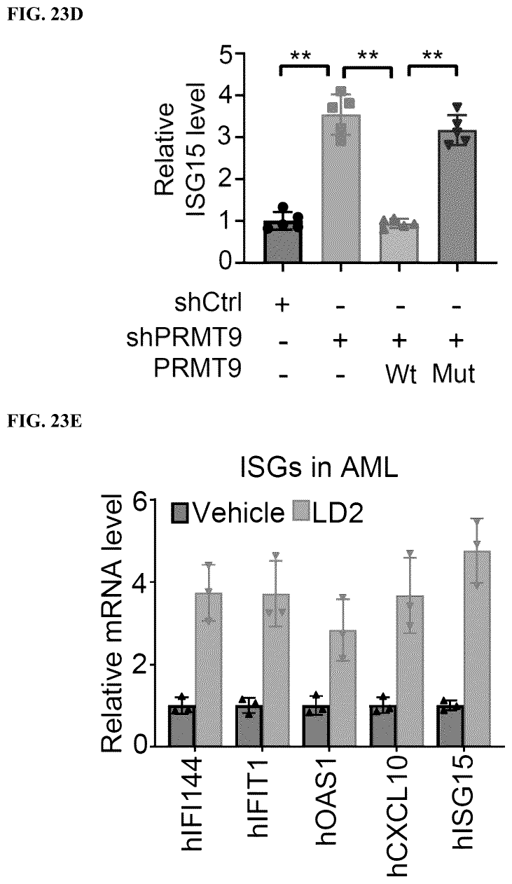

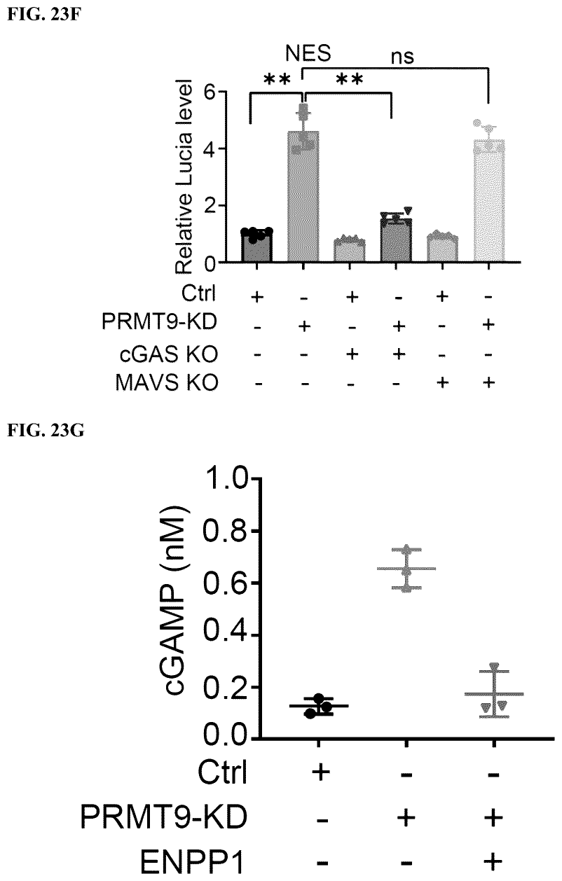

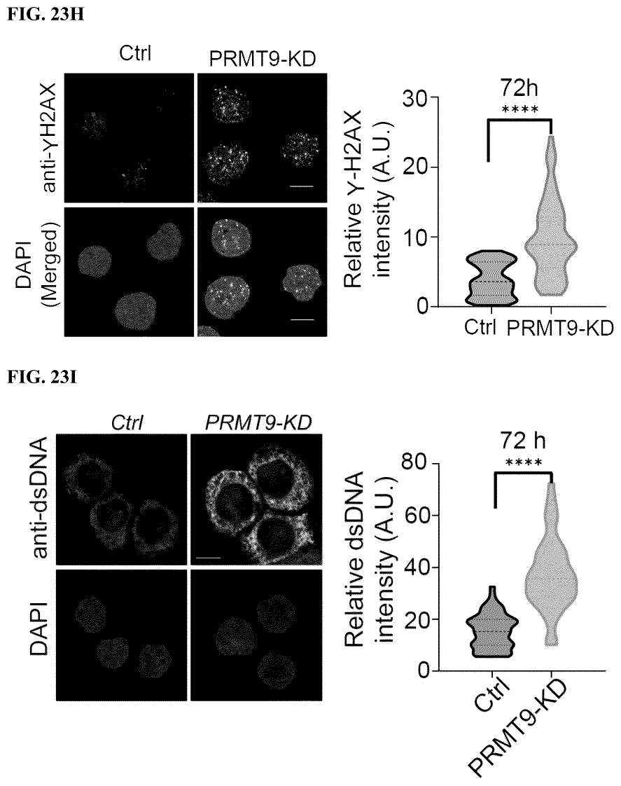

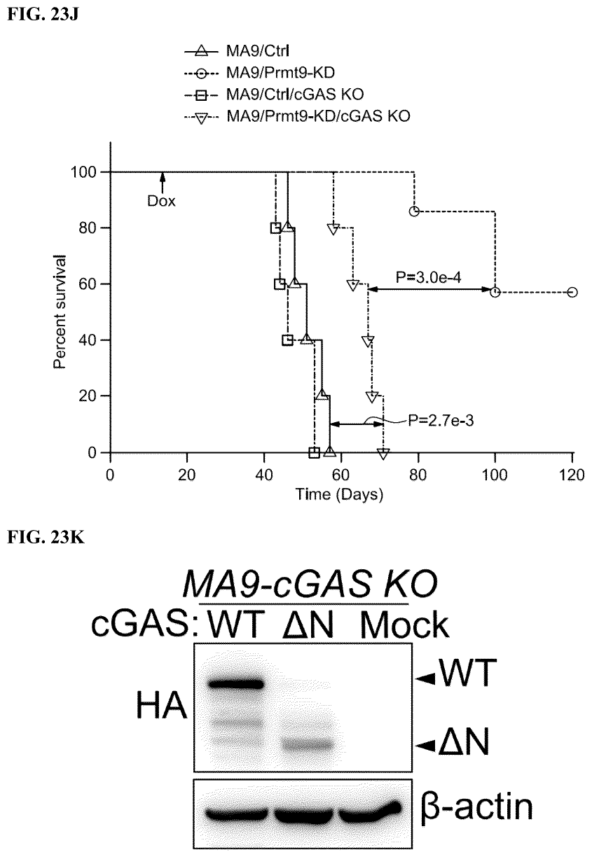

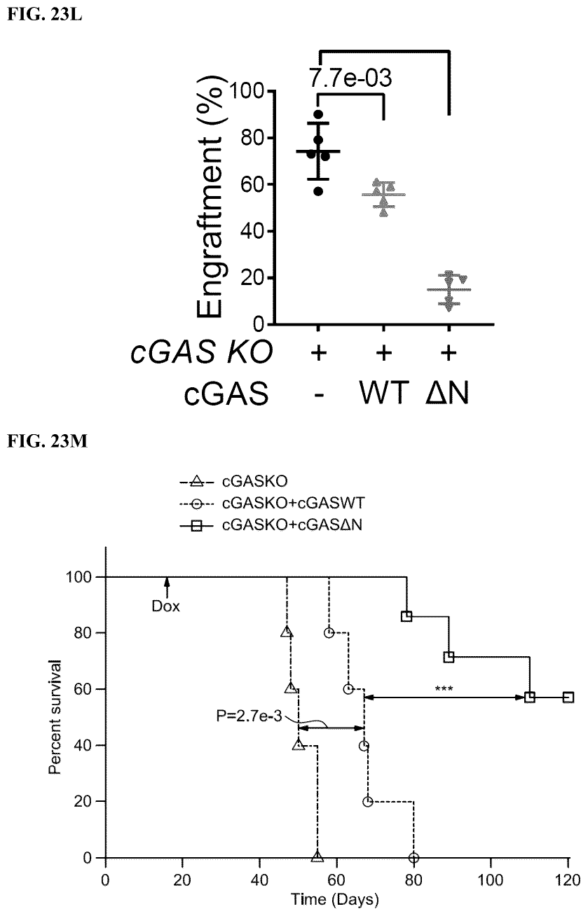

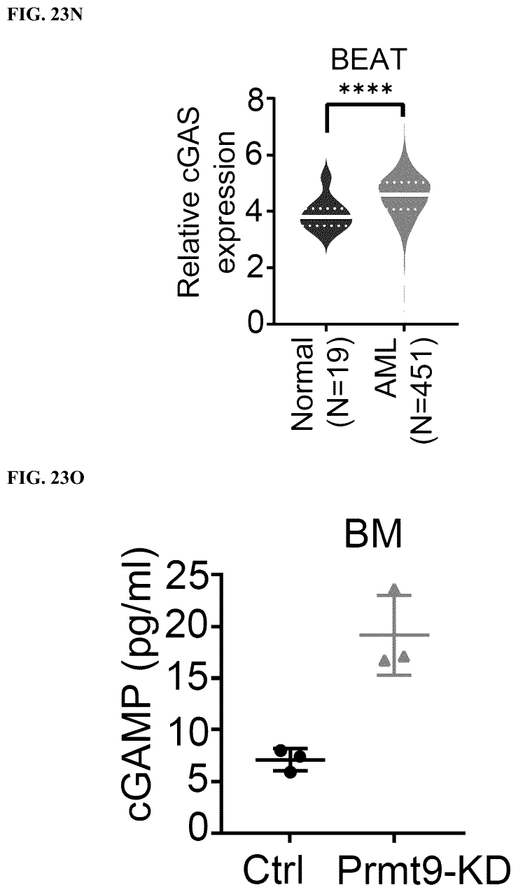

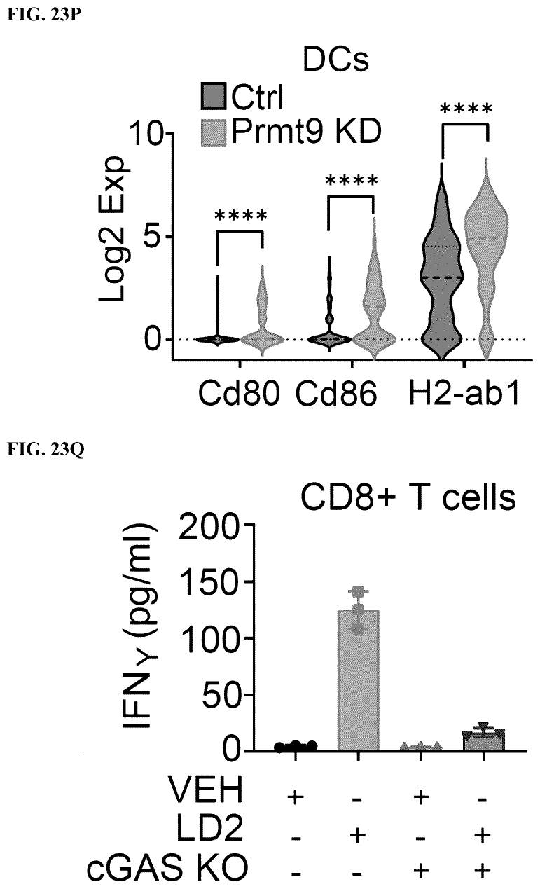

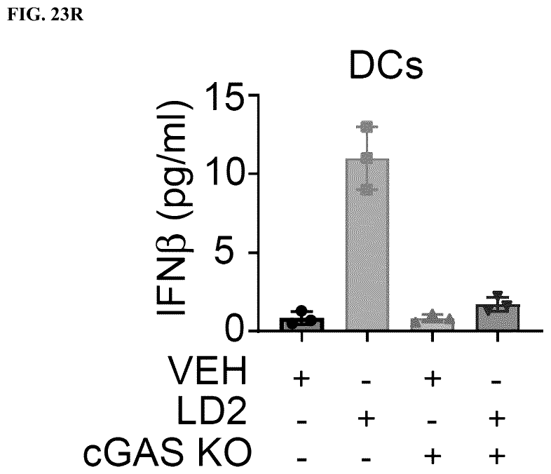

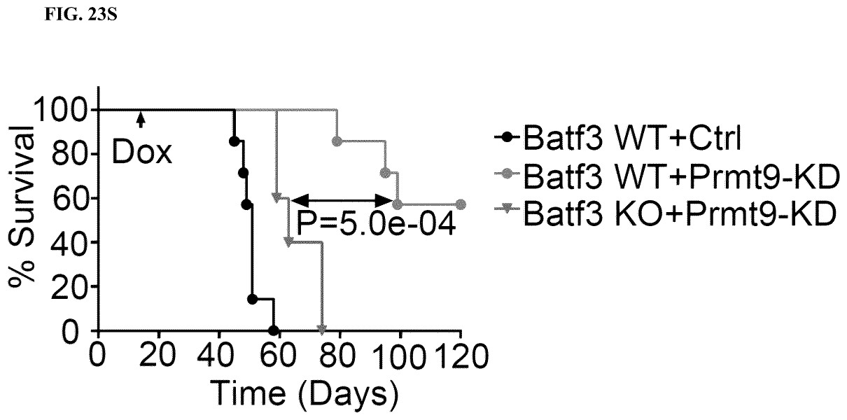

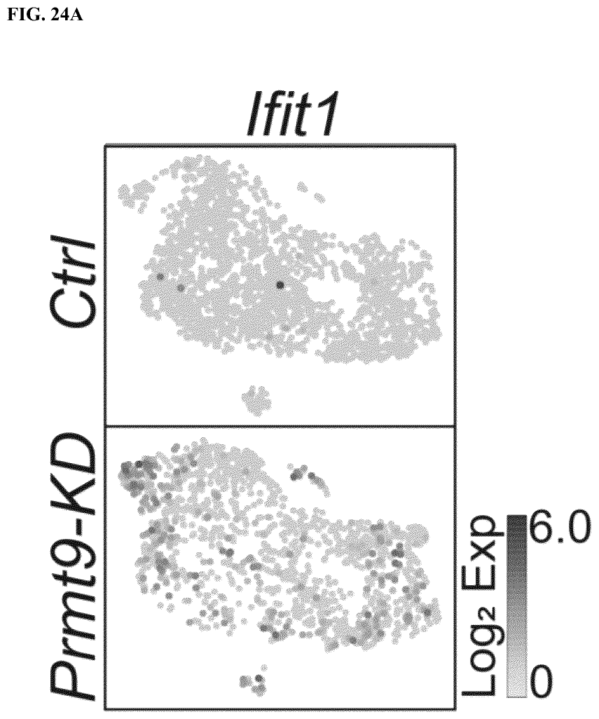

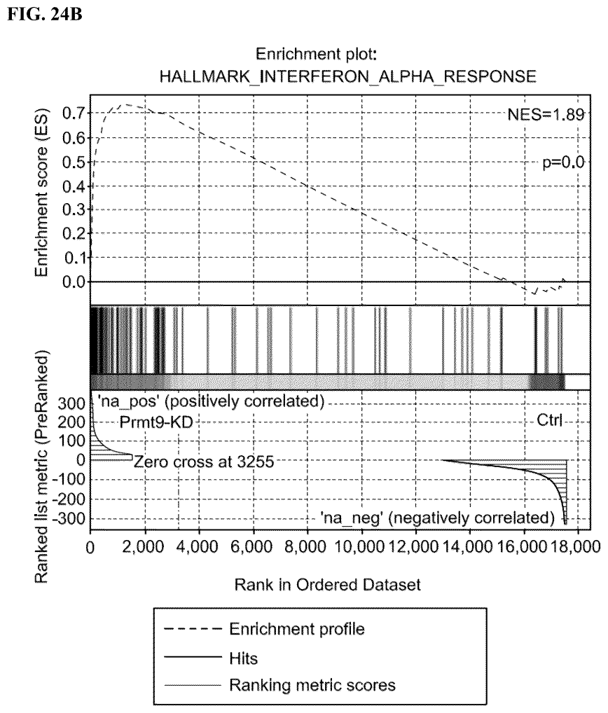

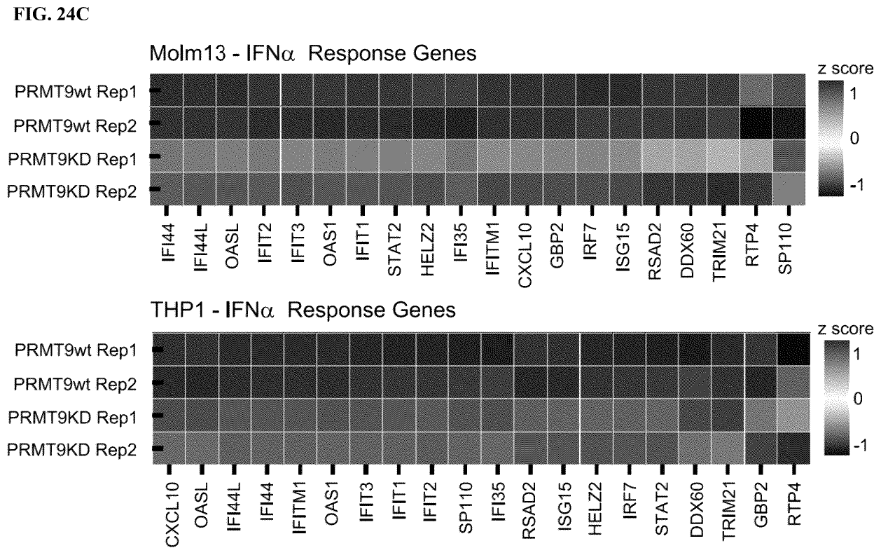

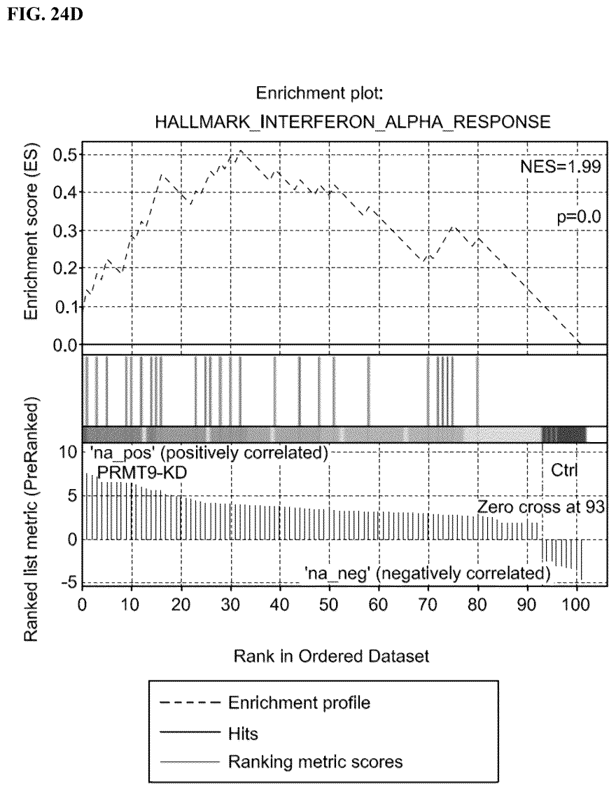

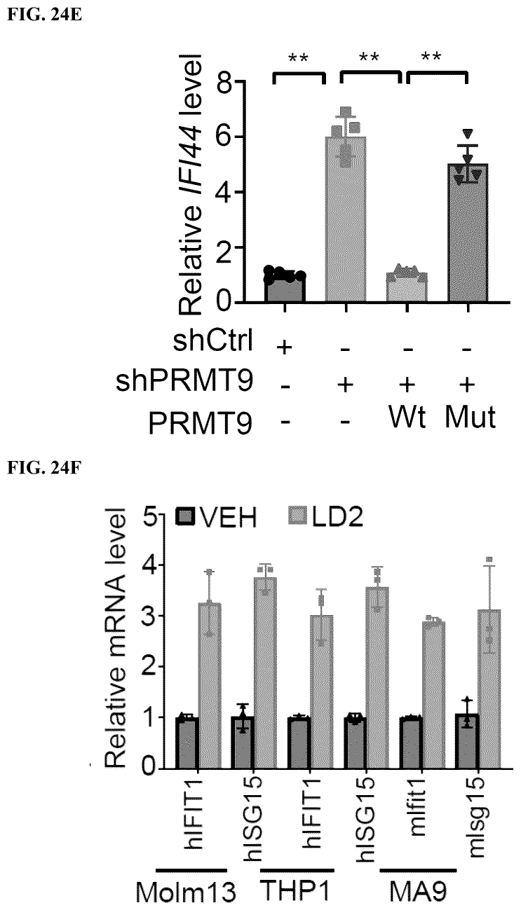

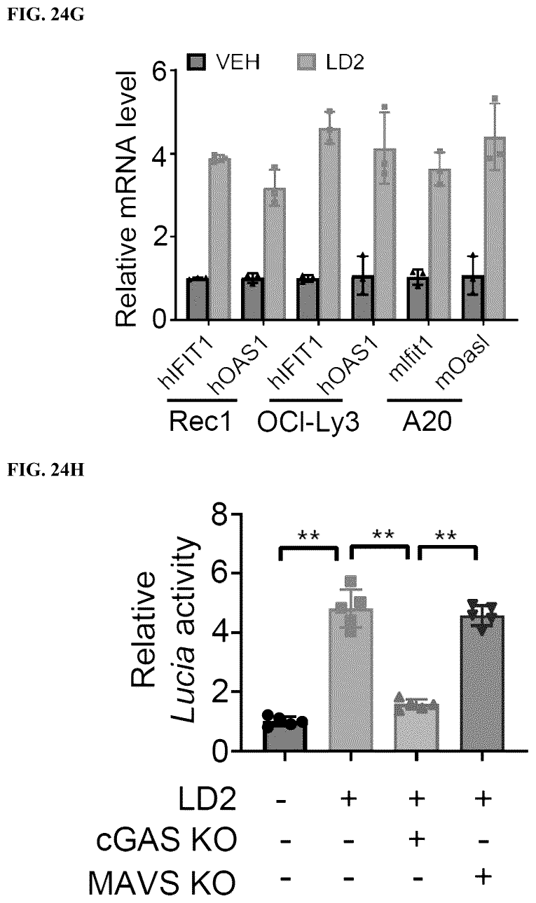

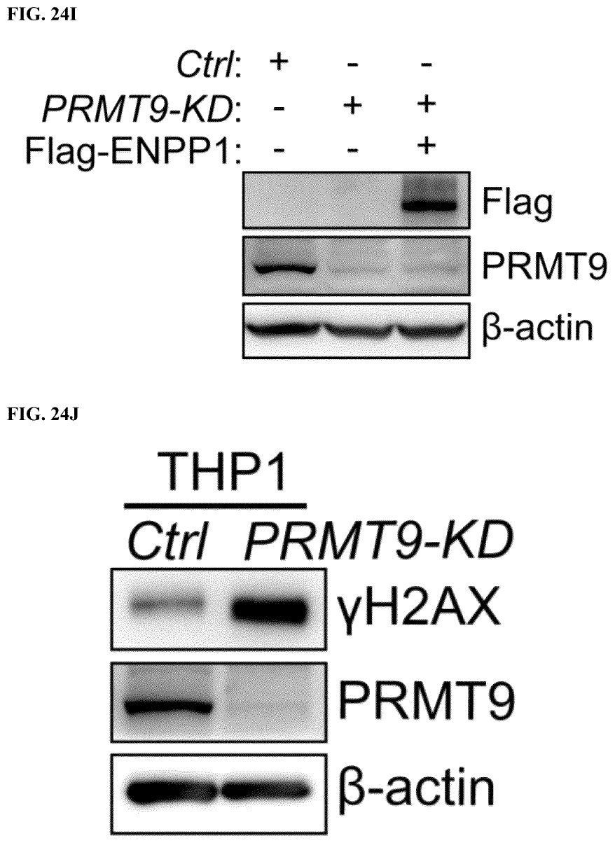

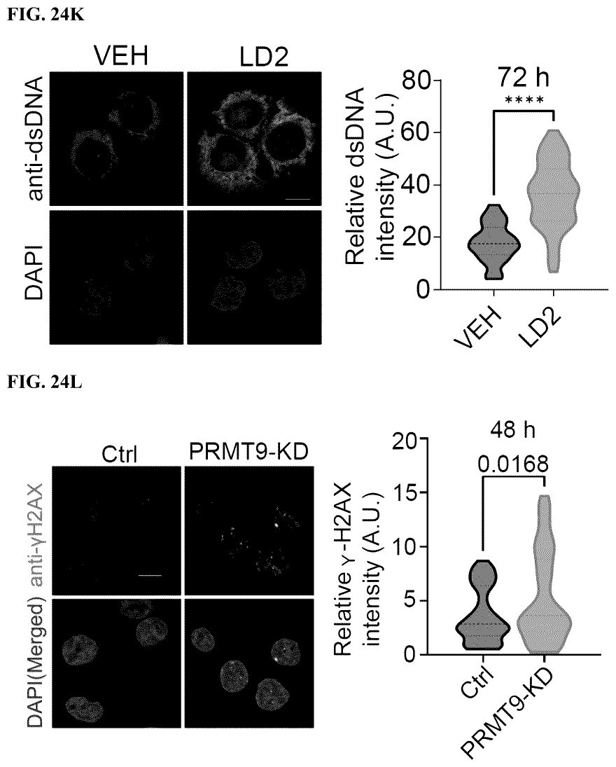

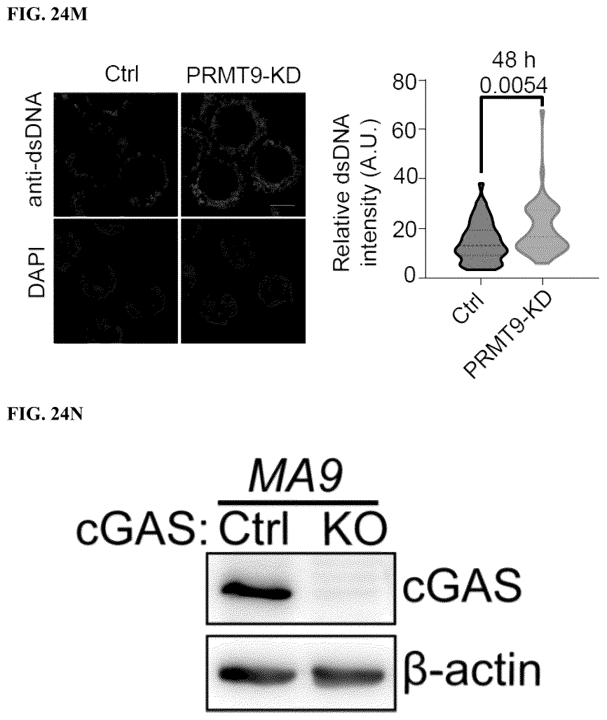

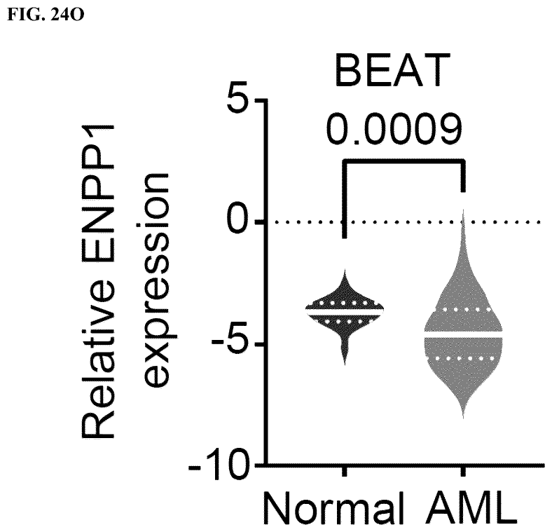

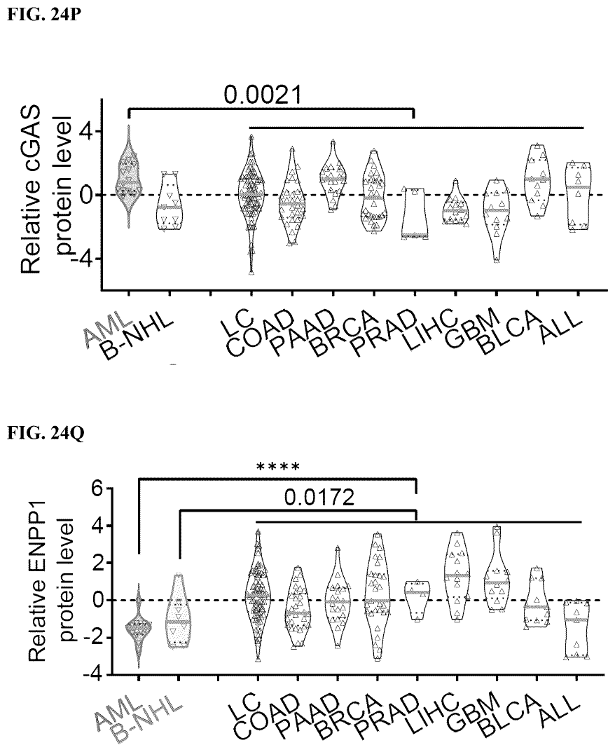

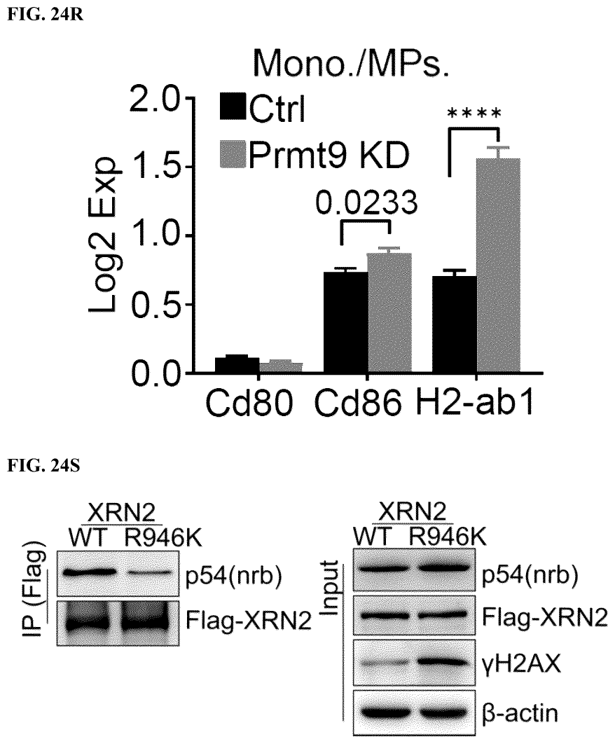

Next, scRNAseq transcriptomes of MA9 cells were analyzed and observed striking upregulation of multiple ISGs such as Ifit1 following Prmt9 KD (FIGS. 23A, 24A). GSEA analysis showed top enrichment of IFNα and IFNγ pathways following Prmt9 KD (FIGS. 23B, 24B). Consistently, transcriptome profiling of other AML lines (Molm-13, THP-1) with inducible Prmt9 KD confirmed increased expression of multiple ISGs, in the absence of exogenous type I IFN, indicating innate immune signaling activation by targeting Prmt9 (FIGS. 23C, 24C). GSEA analysis also indicated IFNα pathway activation (FIG. 24D). To determine if innate immune activation is associated with PRMT9 catalytic activity, ISG expression in Molm13 cells overexpressed WT or catalytic mutant PRMT9 (both constructs are resistant to shPRMT9) was compared following KD of endogenous PRMT9. Notably, only expression of PRMT9-WT, but not the catalytic mutant rescued ISG upregulation following endogenous PRMT9-KD (e.g., ISG15, IFI44) (FIGS. 23D, 24E). LD2 treatment of AML and lymphoma lines or primary AML CD34⁺ cells also stimulated ISGs expression, as indicated (FIGS. 23E, 24F-24G).

To further assess outcomes following PRMT9 KD, the THP1-Lucia line was utilized to monitor IRF signaling downstream of major innate immune sensors, including the double-stranded (ds) DNA sensor cGAS or dsRNA sensors RIG-I and MDA-5. As expected, PRMT9-KD or LD2 treatment of the reporter line increased luciferase signals (FIGS. 23F, 24H), an increase was blocked by deletion of cGAS but not Mavs. PRMT9 KD in THP1 cells also enhanced cGAS catalytic activity, as evidenced by increased cGAMP levels (FIG. 23G). Moreover, overexpression of ENPP1, an ectonucleotidase that degrades cGAMP, abrogated this effect (FIGS. 23G, 24I). Accordingly, in the same line, PRMT9-KD increased γH2AX levels, indicative of DNA damage (FIGS. 23H, 24J-24L) and promoted accumulation of cytoplasmic dsDNA (FIGS. 23I, 24K-24M), which triggers cGAS activity. To determine whether tumor-intrinsic cGAS activity was required for Prmt9 KD-mediated immunity, cGAS in MA9-OVA cells was knocked out (FIG. 24N), then transduced cells with either inducible shPrmt9 or shCtrl and implanted them in WT B6 recipients. Following engraftment and KD Prmt9 by DOX treatment, it was observed that, unlike controls, cGAS-KO mice did not exhibit Prmt9 KD-induced increases in tumor specific CD8⁺ T cells in BM (FIG. 18M). Mouse survival advantages were also abolished on a cGAS-KO background (FIG. 23J). To test outcomes of cGAS activation in cancer cells, cGAS-KO MA9 cells were transduced with an inducible cGAS activating mutant (ΔN) or corresponding cGAS-WT (FIG. 23K) and then implanted parental (cGAS-KO), cGAS-WT or ΔN expressing cells into WT B6 mice to analyze leukemogenesis. Following leukemia engraftment in about 2 weeks (>1% in PB), DOX was administrated to continuously induce expression of cGAS variants till the end point. A significantly decreased leukemia burden was observed in mice harboring ΔN transplants (FIG. 23L). Mice with AML cells exhibiting cGAS activation also showed significantly extended survival relative to other groups (FIG. 23M), indicating that AML-intrinsic cGAS activation is sufficient to induce a leukemia regression phenotype. The analyses of BEAT-AML dataset showed that cGAS levels were remarkably high in AML relative to normal donors (FIG. 23N), while ENPP1 levels were lower in AML samples than in samples from normal donors (FIG. 24O). Moreover, datamining of Depmap dataset revealed that among the deadly cancers, AML cells exhibit the highest cGAS levels and lowest ENPP1 levels (FIGS. 24P-24Q).

Events downstream of cGAS activation in Prmt9-KD AML cells were next analyzed. IFNβ was not detectable in MA9 cells supernatants (data not shown). Thus, it was hypothesized that T cell priming effects seen following Prmt9-KD could be mediated by increases in the immune-transmitter cGAMP. Indeed, it was observed that elevated cGAMP levels in BM fluid from Prmt9-KD MA9 mice relative to control MA9 mice (FIG. 23O). Moreover, single-cell transcriptomes of DCs and macrophages revealed increased levels of Cd80, Cd86 and MHC class II (H2-ab1), indicating activation of APC (FIG. 23P, FIG. 24R), which are known to sense extracellular cGAMP levels. To determine whether T cell priming effects seen following Prmt9-inhibition are due to antigen cross-presentation by APCs, LD2-pretreated cGAS-KO or cGAS-WT MA9/OVA cells were co-cultured with BM-derived DCs, and then purified DCs, which were then co-cultured with I OT-I transgenic CD8+ T cells. An increased IFN-γ production by CD8+ T cells was observed after co-culture with DCs purified from the LD2-pretreated AML cell co-culture group, indicating DC mediated T cells prime (FIG. 23Q). cGAS KO antagonized DC-mediated cross-priming capacity, based on decreased IFN-γ production by T cells (FIG. 23Q). IFN-β production by DCs was then assessed after co-culture with MA9-OVA cells pretreated with LD2 and observed increased IFN-β production, an effect abolished by cGAS KO, indicating that tumor cell cGAS activation underlies IFN-I signaling in DC. DC function in Prmt9-KD-induced AML regression was further assessed using Batf3-KO mice, as Batf3-dependent DCs are critical for priming of antigen-specific CD8+ T cells. Specifically, Batf3-WT or KO mice was implanted with Prmt9-KD or -WT MA9+ AML cells, and then evaluated AML progression in three groups: Prmt9-KD/Batf3-KO, Prmt9-KD/Batf3-WT, and Prmt9-WT/Batf3-WT. Relative to Batf3-WT mice, Batf3-KO partially decreased the PRMT9-KD-induced survival advantage, indicating a critical role for DCs in PRMT9-KD induced outcomes (FIG. 23S).

Loss of XRN2 methylation underlies cGAS activation in cancer cells

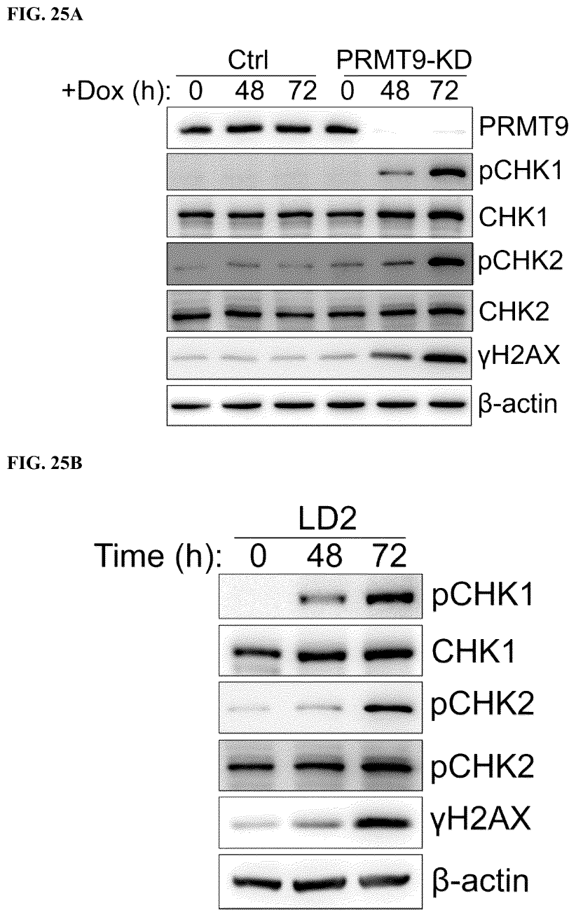

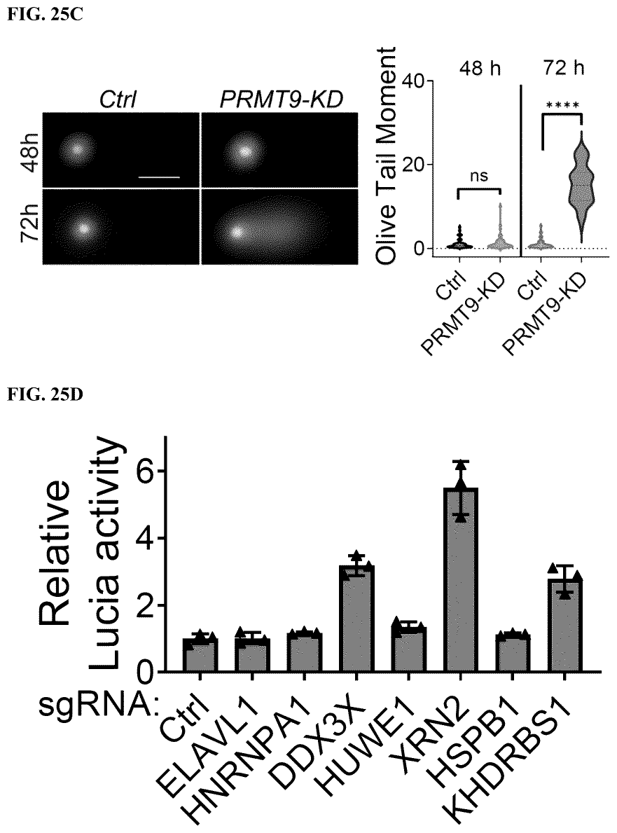

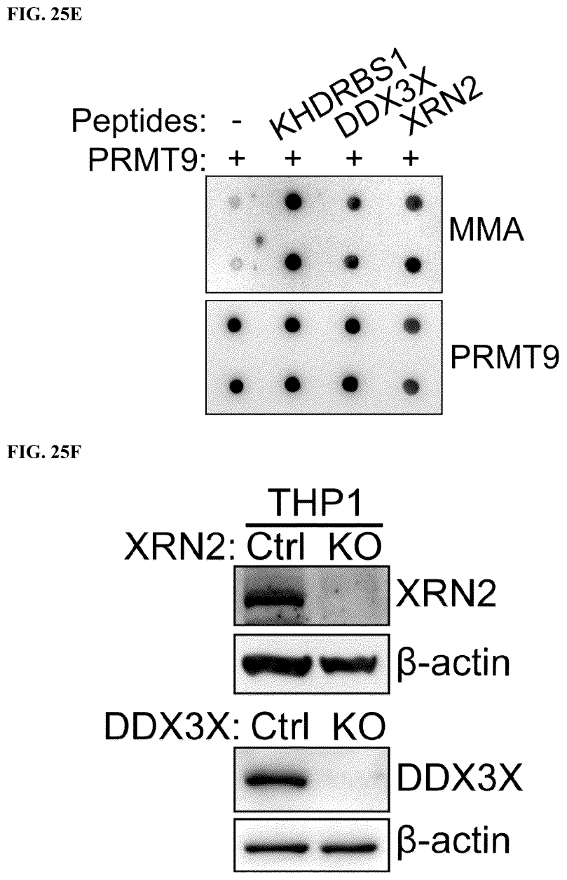

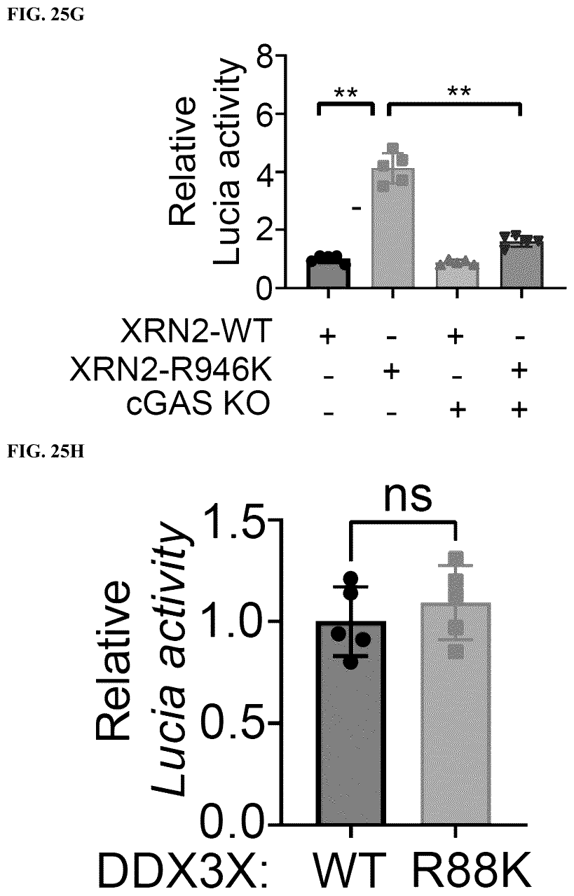

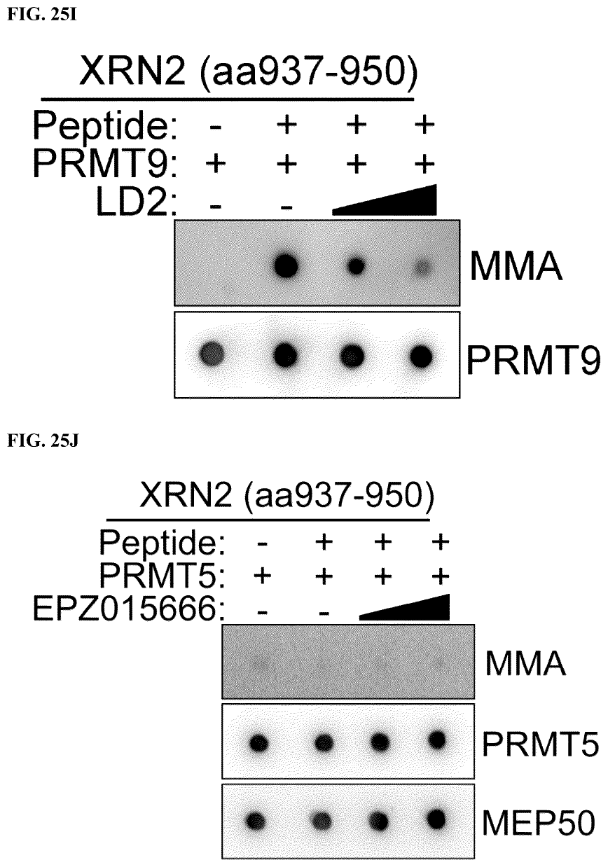

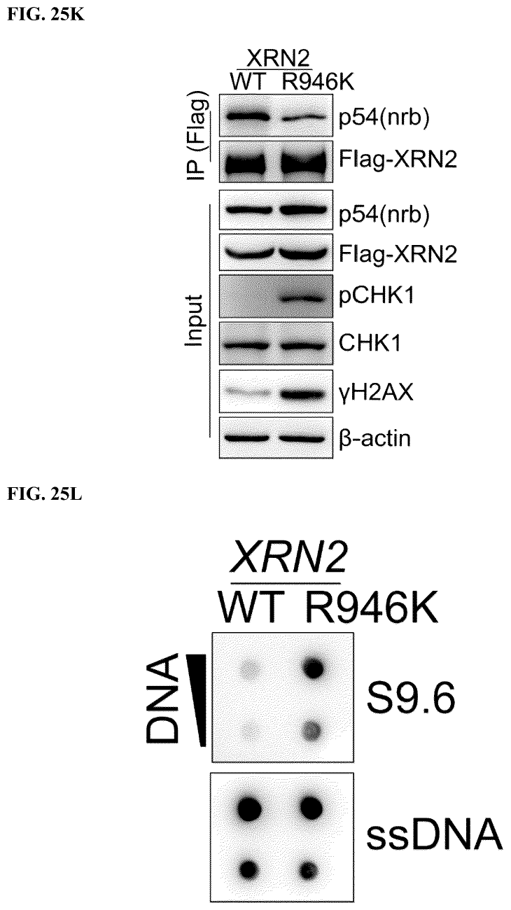

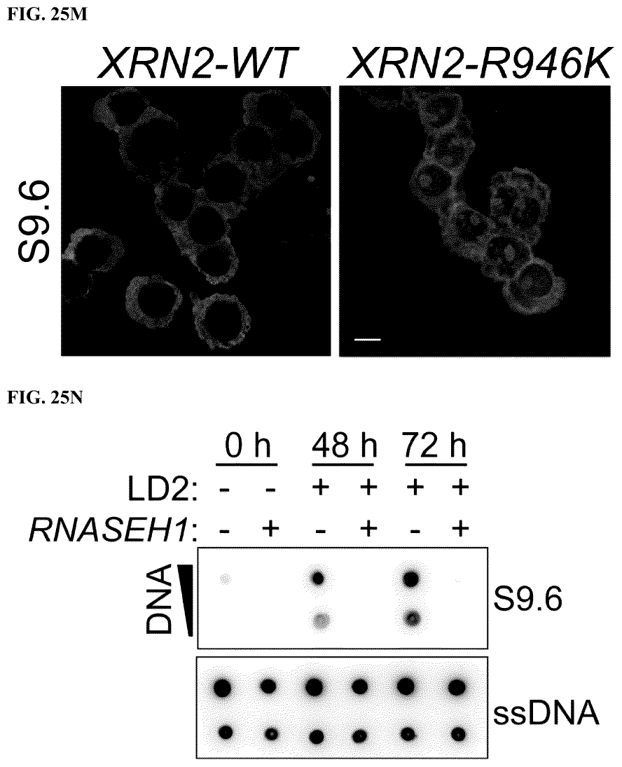

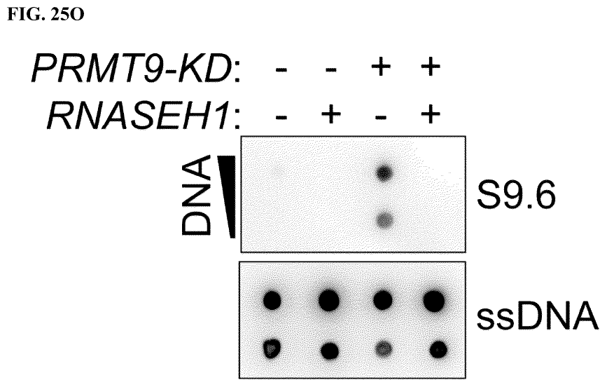

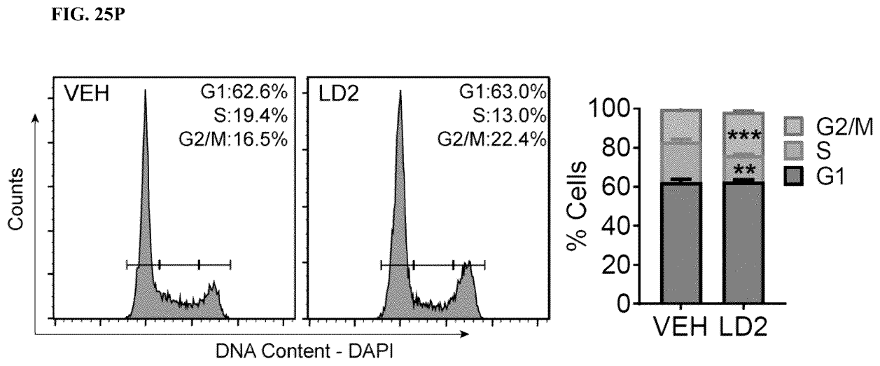

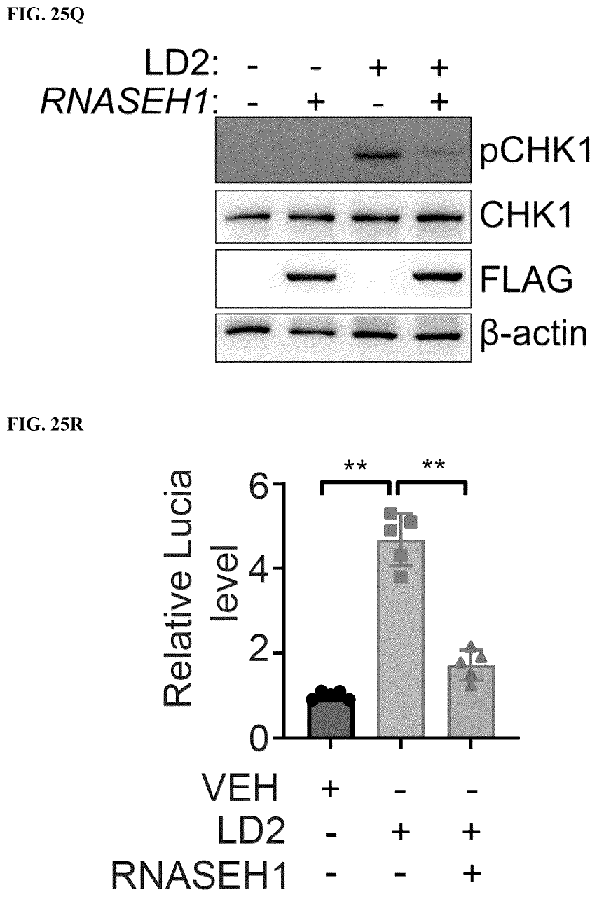

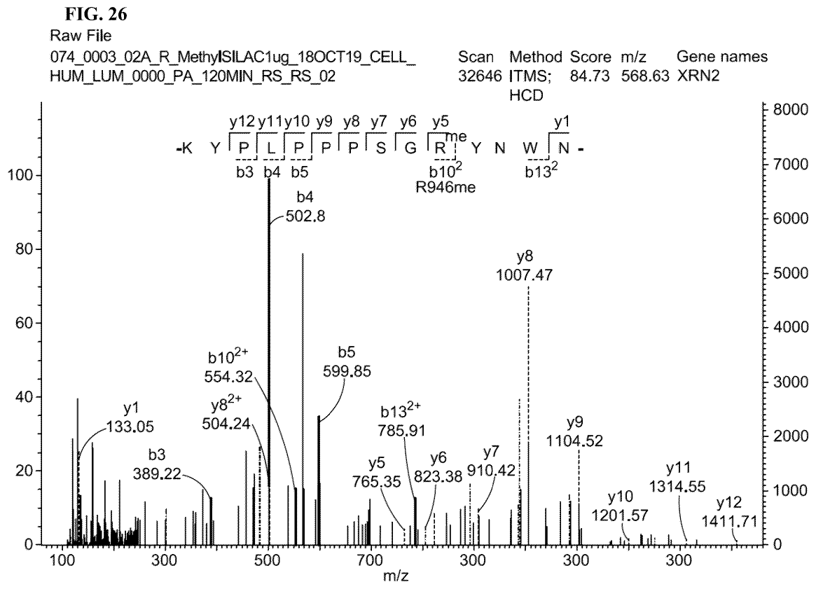

Next, DDR signaling in AML cells were characterized. Notably, PRMT9 inhibition in THP1 cells via LD2 (48 h) or shRNA significantly increased ATR signaling, based on pCHK1 upregulation, whereas γH2AX elevation and changes in ATM signaling, as evidenced by pCHK2 levels, were modest at that time point (FIGS. 25A-25B). However, remarkably increased levels of γH2AX and pCHK2 were observed after 72 h of PRMT9 inhibition (FIGS. 23H, 25A-25B), corresponding with formation of double strand-breaks (DSBs) detected via a comet assay (FIG. 25C). These findings indicate that PRMT9 inhibition triggers a genome insult that selectively activates ATR signaling.

It was next asked whether any PRMT9 substrate functions in the DDR, and whether its loss underlies ATR activation and cGAS stimulation. SILAC analysis showed that 7 of the 23 most downregulated methylated proteins after PRMT9-KD (FIG. 6C) regulate the DDR (FIGS. 6D, 8). To determine whether KO of any of the 7 phenocopied PRMT9 inhibition-induced immune activation, THP1-Lucia cells were electroporated with ribonucleoprotein complexes (RNP) composed of Cas9 protein and bound gRNA targeting respective candidate genes, a well-characterized protocol that does not stimulate innate immunity. Kim et al, Genome research 24, 1012-1019, doi:10.1101/gr.171322.113 (2014). Of the 7 genes, KO of XRN2, DDX3X or KHDRBS1 increased reporter activity (FIG. 25D), and an in-vitro methylation assay confirmed those proteins' methylation by PRMT9 (FIG. 25E). Of the top 2 candidates (XRN2 and DDX3X), KO of either gene robustly activated the reporter (FIGS. 25D, 25F). ectopically expressed gRNA-resistant full-length XRN2-WT or DDX3X-WT cDNAs or corresponding methylation-deficient constructs (XRN2-R946K or DDX3X-R88K) in THP1-Lucia cells as well as the corresponding cGAS-KO/THP1-Lucia line and electroporated cells with RNPs to KO corresponding endogenous genes. Notably, XRN2 R946K expression increased THP1 reporter activity, an effect blocked by cGAS deletion (FIG. 25G). By contrast, methylation-deficient DDX3X did not increase reporter activity (FIG. 25H). It was also confirmed that XRN2-R946 is specifically methylated by PRMT9 but not PRTM5, based on in-vitro methylation assays and responses to LD2 treatment (FIGS. 25I-25J).

Next, the focus was shifted on the exoribonuclease XRN2 whose C-terminus interacts with p54nrb to form complexes accumulating at the 3′ end of transcribed genes to prevent R-loop formation. SILAC analysis revealed that among all XRN2 R residues, only R946 methylation levels are altered by PRMT9-KD. To determine if R946 methylation promotes XRN2 recruitment by p54nrb, co-IP analysis of THP1 cells was performed ectopically expressing Flag-tagged XRN2-WT or the R946K mutant. Indeed, Flag-tagged XRN2 interaction with p54nrb decreased in the presence of R946K (FIGS. 25K, 24S), indicating a critical role for R946K methylation in XRN2 complex formation. More importantly, relative to XRN2-WT, expression of the gRNA-resistant XRN2 R946K mutant in THP1 cells engineered to lack endogenous XRN2 promoted R-loop formation, based on increased S9.6 staining (FIGS. 25L-25M), resulting in ATR activation and γH2AX elevation (FIG. 25K). Consistently, LD2 treatment or PRMT9-KD in THP1 cells promoted excess R-loop formation and activated ATR signaling as early as 48 h after treatment (FIGS. 25A-25B, 25N-250). Cell cycle analysis following LD2 treatment showed an increased percentage of G₂/M cells and a corresponding decrease in the percentage of S-phase cells (FIG. 25P), indicating that R-loop accumulation could be due to transcription-replication conflicts during S phase. Finally, RNASEH1 overexpression to resolve R-loops (FIG. 25N) decreased ATR/CHK1 activation, partially rescuing THP1-Lucia reporter activity induced by LD2 treatment (FIG. 25Q-25R). Collectively, these results indicate that loss of XRN2 methylation contributes to increases in DNA damage seen in PRMT9-ablated cells, highlighting the role of R-loop formation in cancer cell-intrinsic cGAS activation after PRMT9 inhibition.

Combining LD2 with an Immune Checkpoint Inhibitor Ablates PRMT9-Proficient Cancers

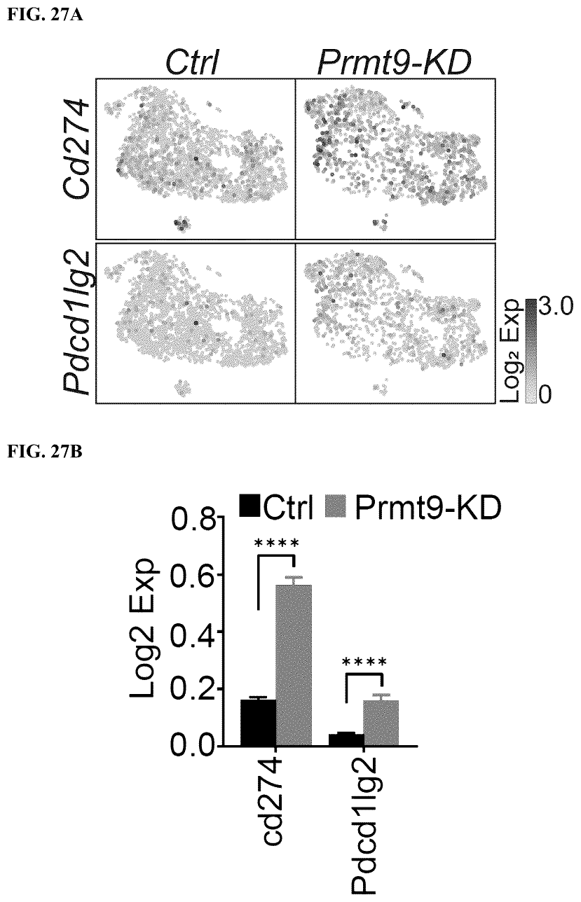

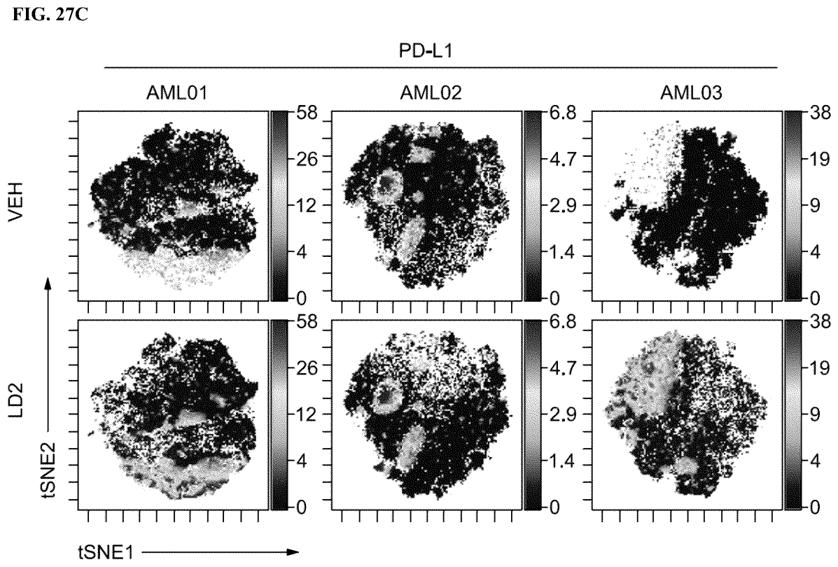

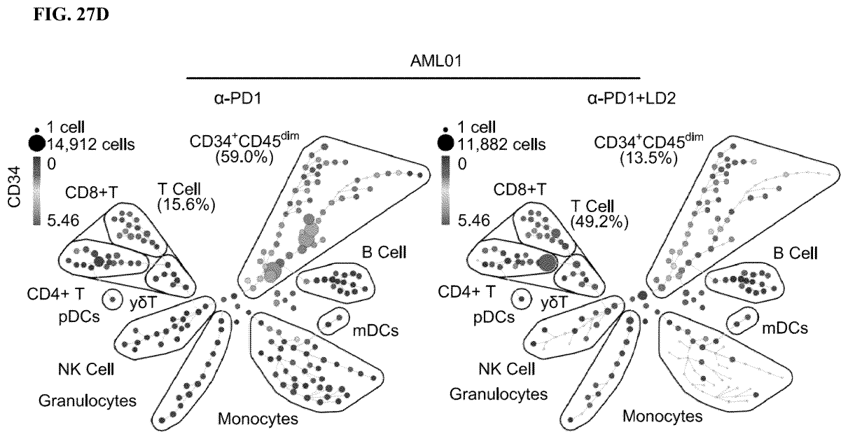

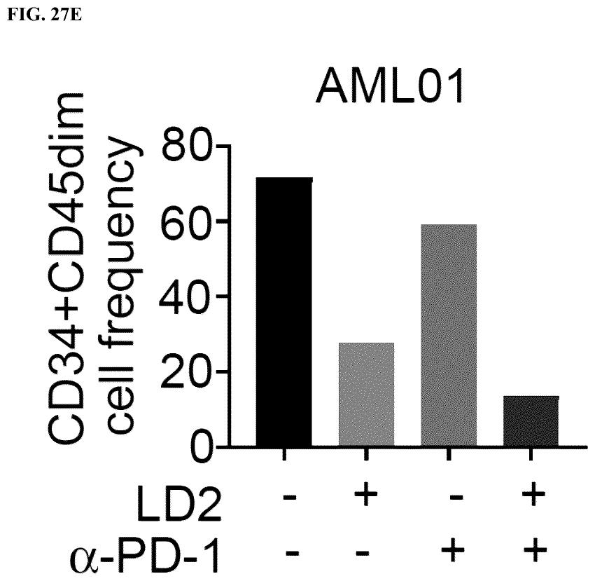

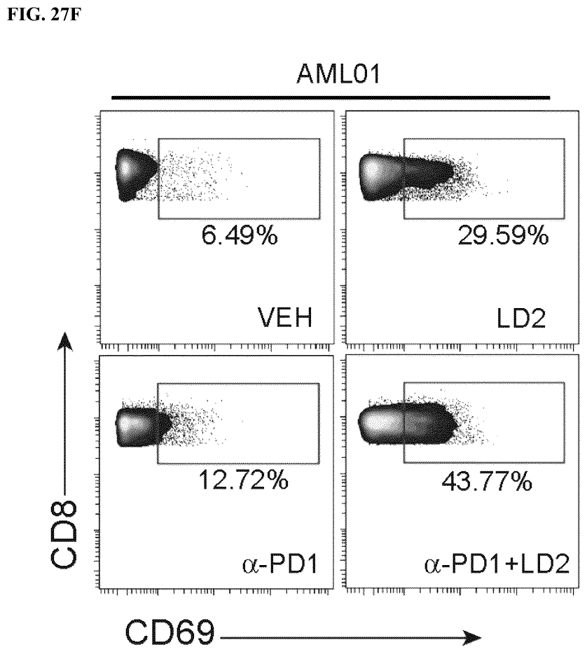

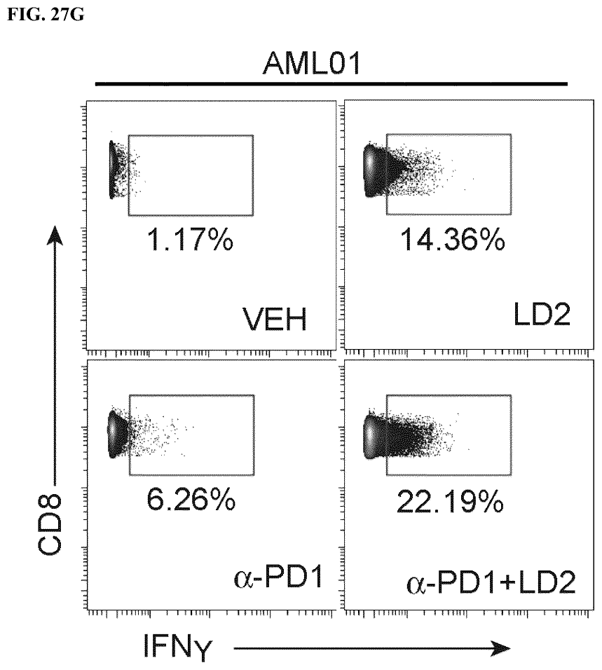

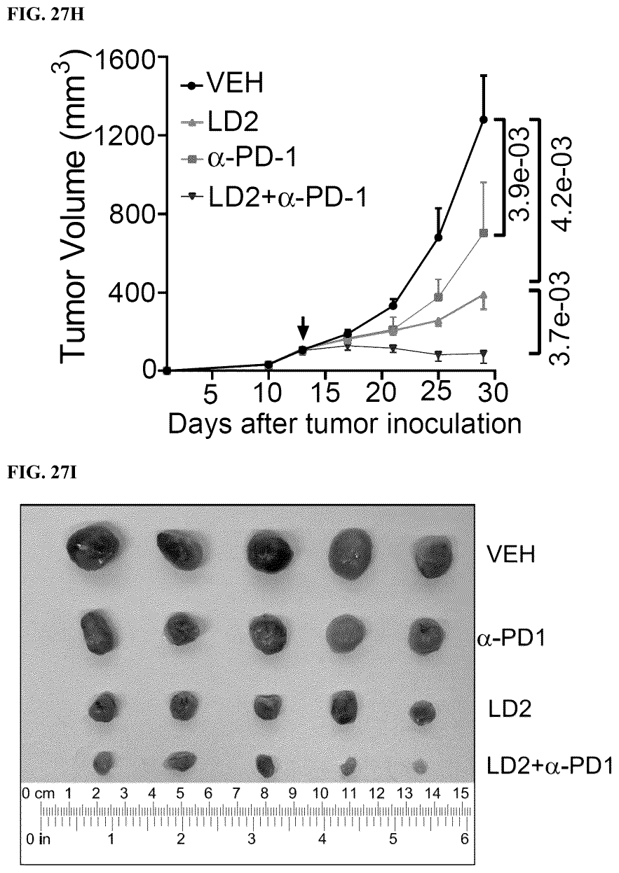

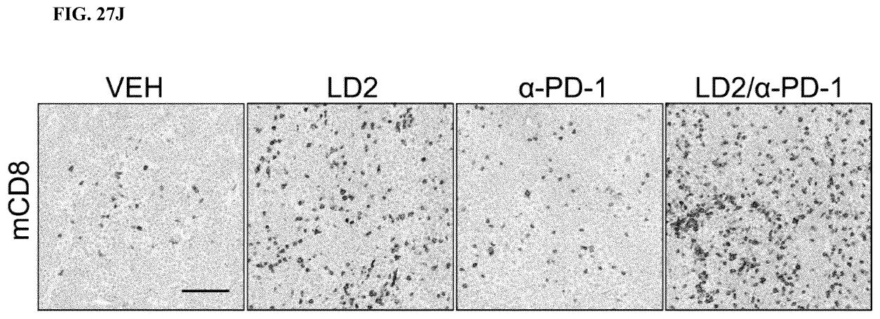

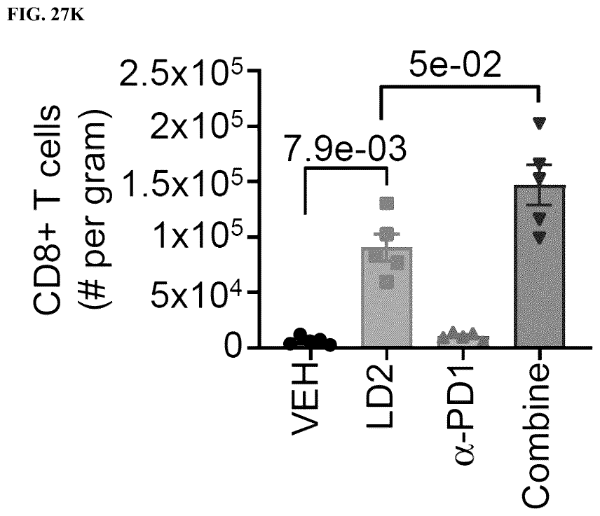

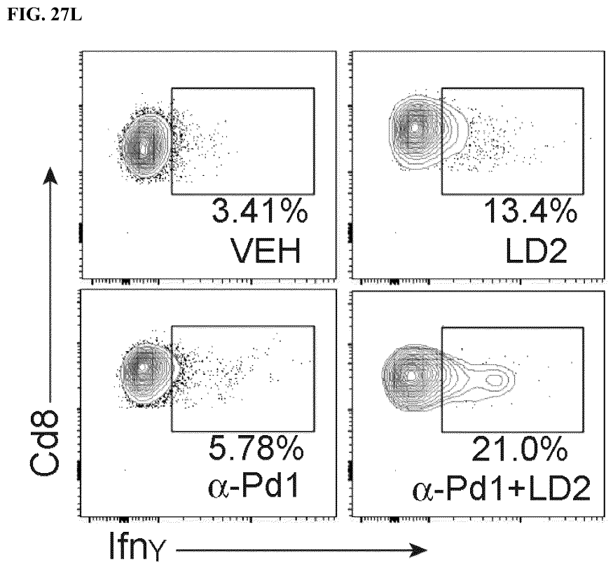

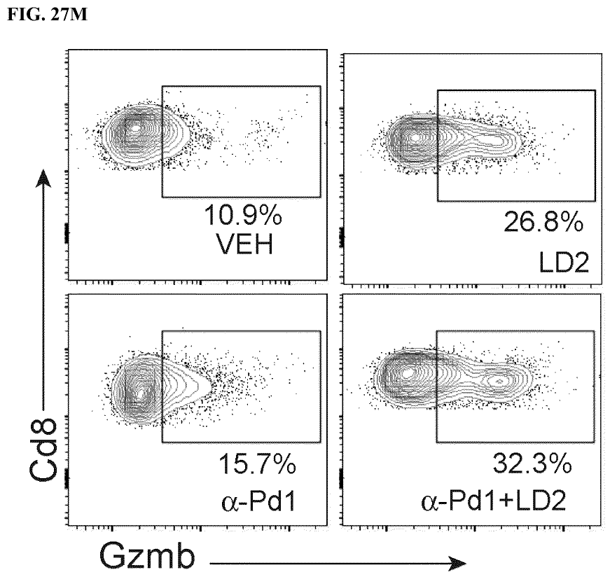

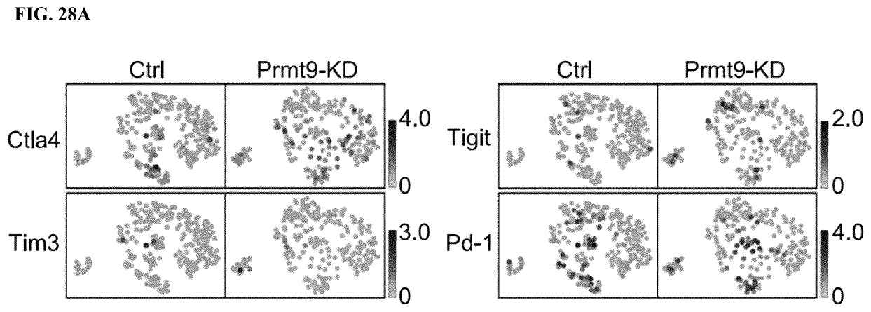

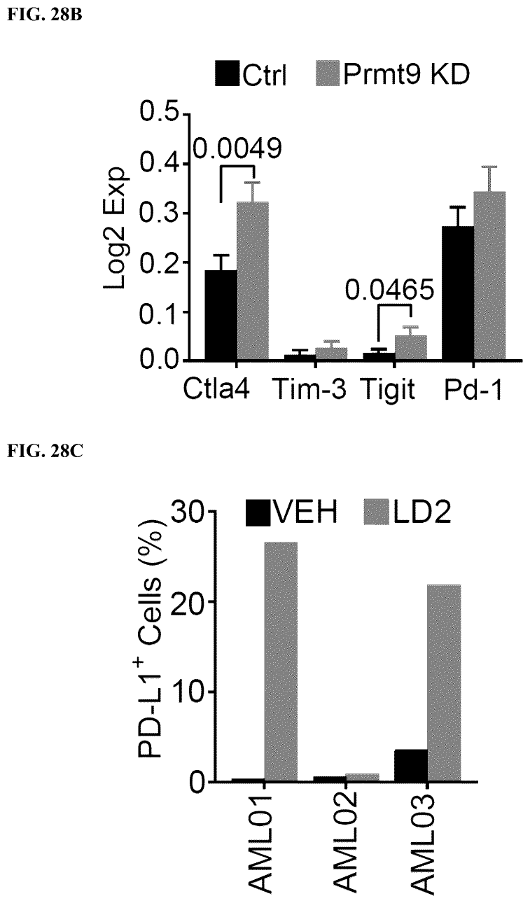

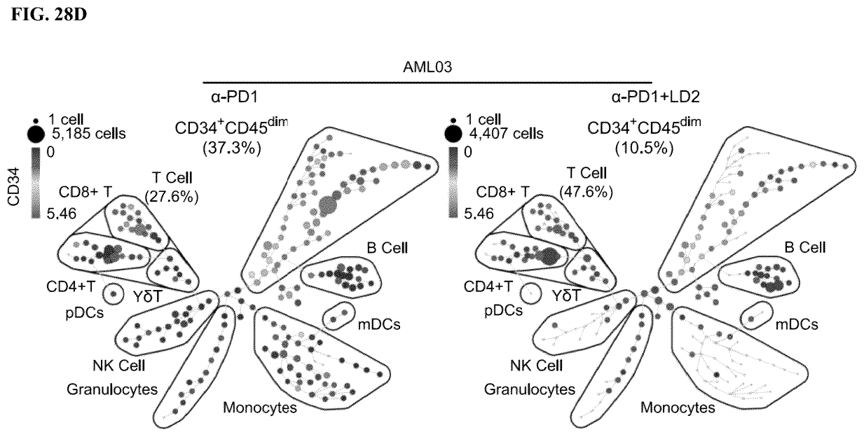

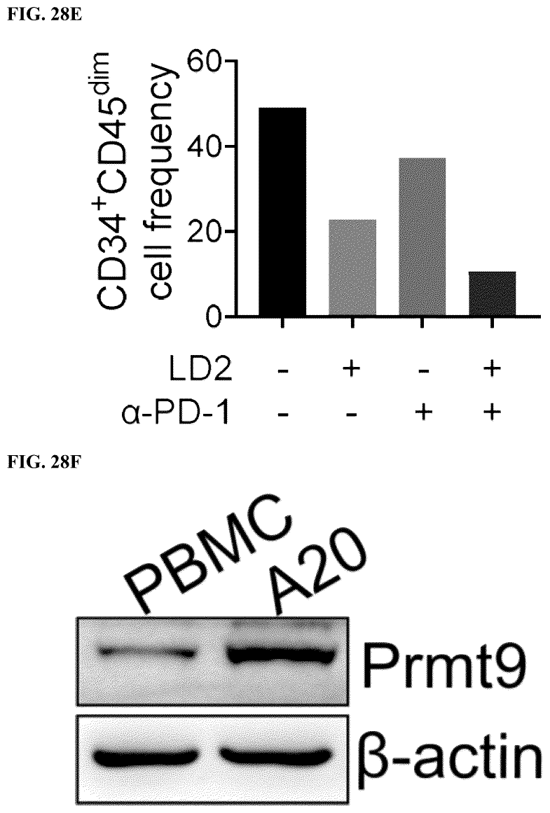

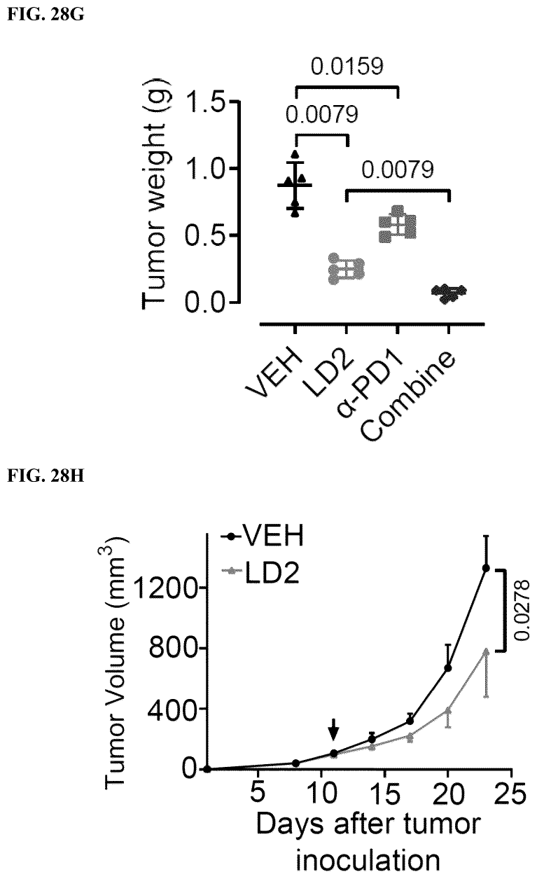

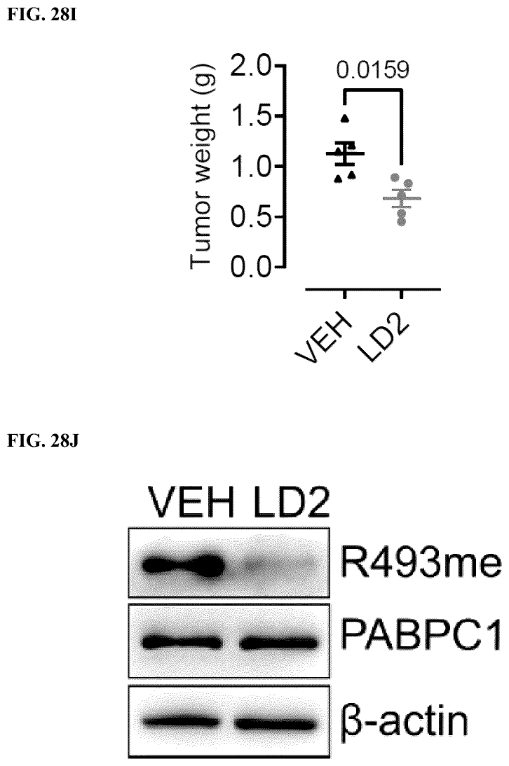

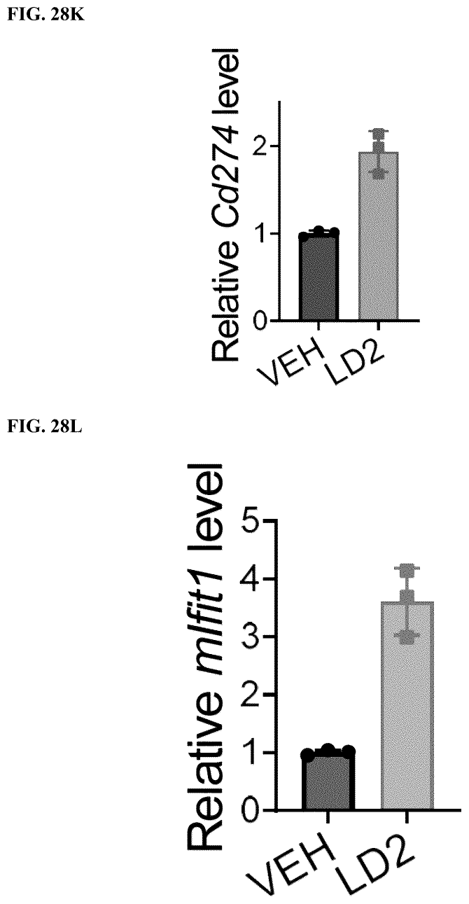

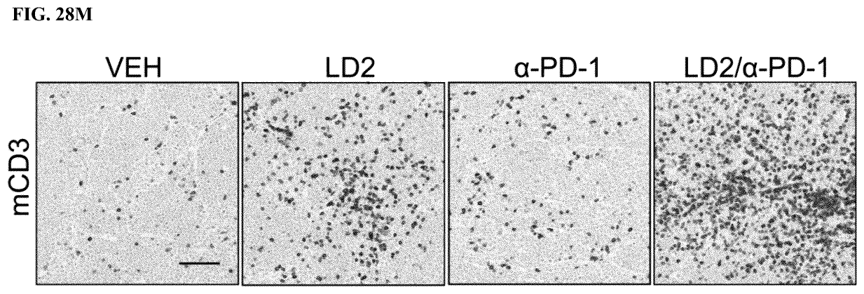

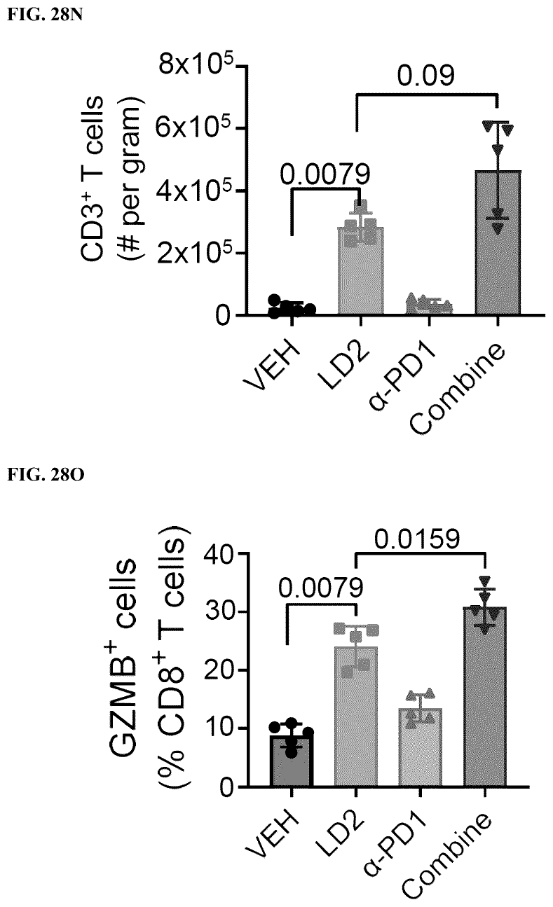

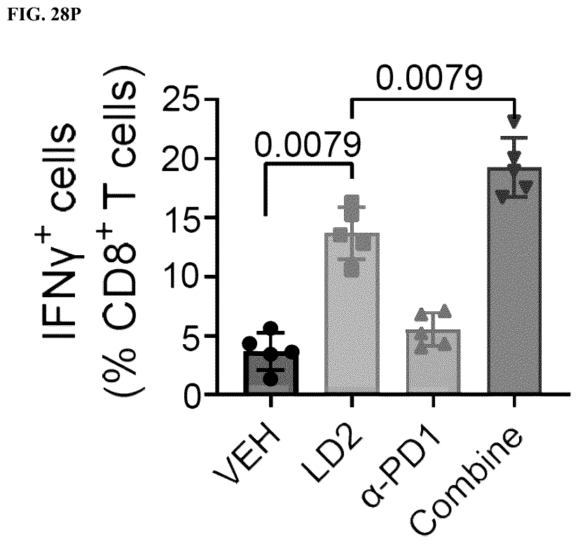

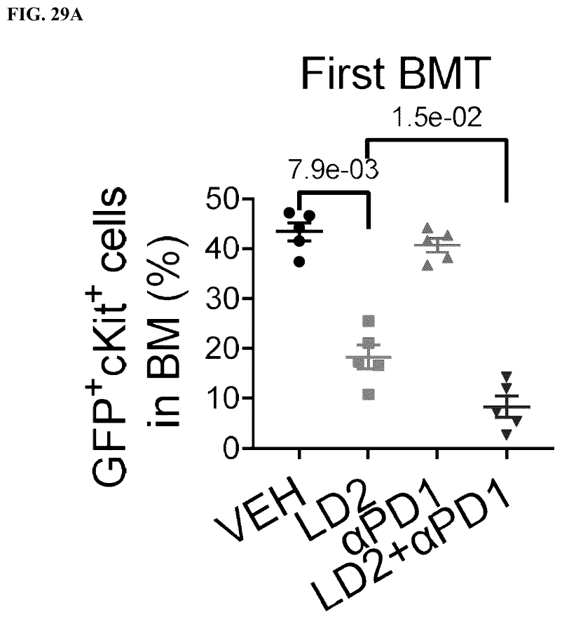

To test novel PRMT9 inhibition-based drug combinations, MA9 leukemic BM scRNA-seq results were first analyzed to identify whether adaptive immune tolerance develops upon Prmt9-inhibition. Among reported leukemia relevant immune checkpoint proteins, including those expressed in cancer cells (PD-L1, PD-L2) or in T cells (CTLA-4, TIM3, TIGIT, PD-1), Prmt9-KD significantly upregulated PD-L1 (FIG. 25A-25B), although PD-L2 and CTLA-4 were also modestly upregulated (FIGS. 27A-27B, FIGS. 28A-28B). Notably, in 2 of 3 primary AML co-cultures, mass-cytometry analysis revealed significant upregulation of PD-L1 upon Prmt9 inhibition (FIGS. 27C, 28C), indicating that adaptive PD-1/PD-L1 signaling may contribute to adaptive resistance to Prmt9 ablation, in agreement with the reportedly immunosuppressive role of cGAS-STING agonists.

To determine if a PRMT9 inhibitor synergizes with PD-1 mAb treatment, AML samples were treated 4 days ex-vivo with vehicle/control, LD2, αPD-1, or combined LD2/αPD-1. αPD-1 treatment alone marginally decreased the proportion of tumor cells, while combination treatment elicited T cell expansion and potently reduced tumor cell frequency (FIGS. 27D-27E, 28D-28E). Significantly higher numbers of CD69 or IFN-γ-expressing T cells were also found among combination-treated cells compared with control cells (FIGS. 27F-27G).

Next, the cooperation between PRMT9 inhibitor and αPD-1 treatment was investigated using a preclinical A20 lymphoma syngeneic mouse model, as A20 cells exhibit higher PRTM9 levels than murine PBMCs (FIG. 28F). Once A20 tumors reached 100 mm³ in size in this model, tumor-bearing mice were treated with isotype control (vehicle), anti-PD1 mAb (BE0146 [BioXCell], 10 mg/kg, i.p. every other day for 2 weeks), LD2 (100 mg/kg, i.t., daily for 2 weeks) or combined LD2/anti-PD1, until a humane endpoint was met. Tumor volumes were monitored through the study's end (FIG. 27H). A humane endpoint was reached in a vehicle group mouse on day 29 (16 days after treatment). The tumor size of the combination group was significantly smaller than that of the vehicle/isotype control treated group (Ctrl) starting on day 17 (day 4 of treatment). LD2 administration alone also significantly decreased tumor size relative to controls after day 21 (FIGS. 27H-27I, 28G). LD2 single treatment effects were also evaluated in an immunodeficient NSGS mouse xenografted with A20 cells; as a result, the treatment modestly decreased A20 tumor growth (FIGS. 28H-28I), likely due to its effects on tumors cells proliferation. Indeed, its effect on reduction of PABP1 R493me levels was observed (FIG. 28J). Notably, A20 tumor weight significantly decreased in the combination group compared to vehicle controls (FIGS. 27I, 28G). It was confirmed that LD2 treatment upregulates ISGs and PD-L1(Cd274) (FIGS. 28K-28L), consistent with effects seen in AML upon PRMT9 inhibition ex vivo and in vivo.

The number of tumor-infiltrating T cells was also determined in mice implanted with A20 cells following combination treatment, given that pre-existing functional T cells in tumors are required for robust responses to anti-PD-1/PD-L1 treatment. An increased number of tumor-infiltrating CD3⁺ T cells were observed after LD2 treatment alone or combination with PD-1 mAb (FIGS. 28M-28N). Notably, CD8⁺ T-cell infiltration in tumors was also substantially increased following LD2 treatment alone and was higher in the combination group (FIGS. 27J-27K). In contrast, CD8⁺ T-cell infiltration was marginally increased following PD-1 mAb single treatment compared with controls (FIGS. 27J-27K). Moreover, a remarkable increase in the number of CD8⁺ T cells was observed expressing granzyme B and IFNγ in the LD2 single treatment group, effects enhanced by combination treatment (FIG. 27L-27M, 280-28P), consistent with T cell priming effects seen following cGAS-STING activation.

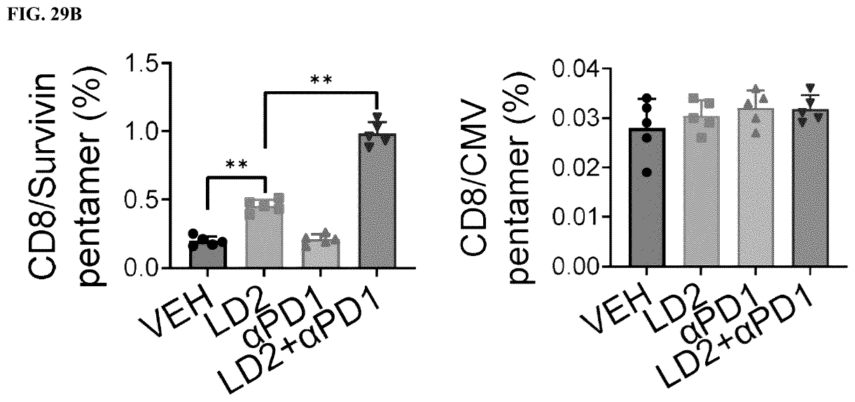

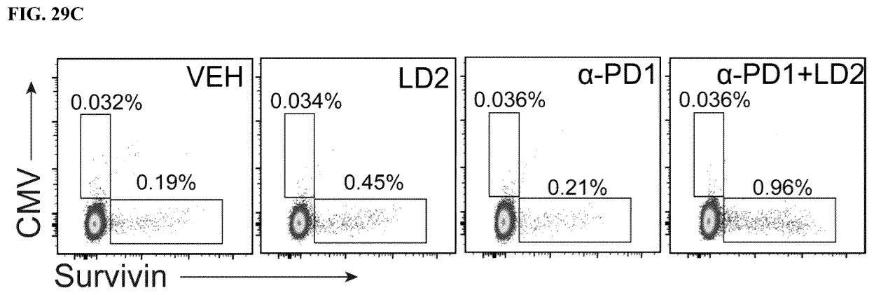

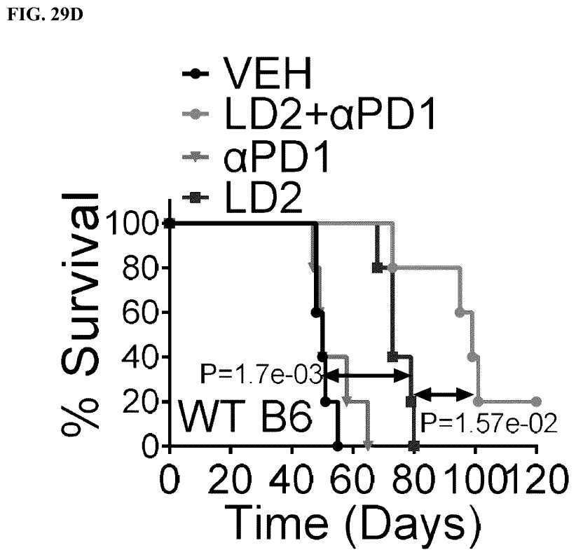

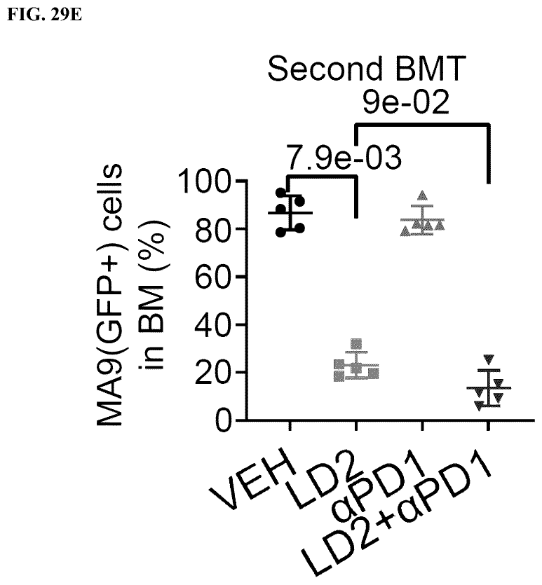

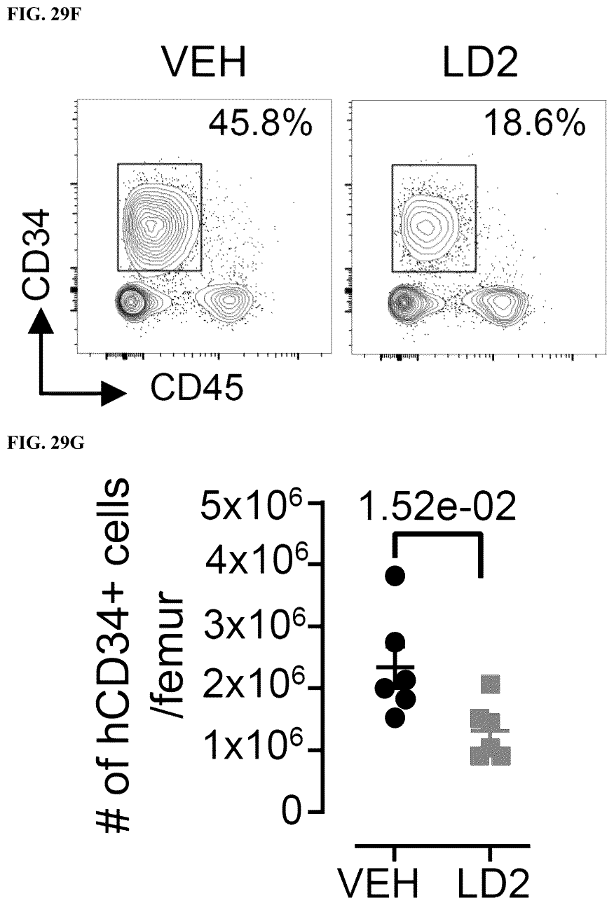

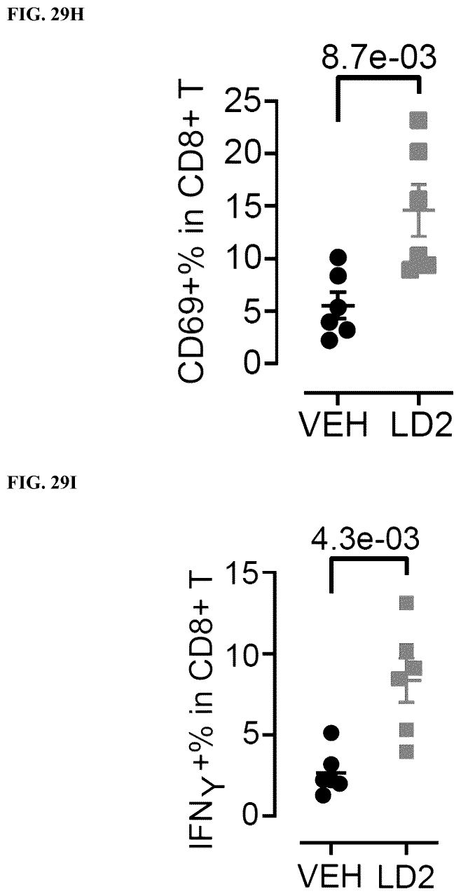

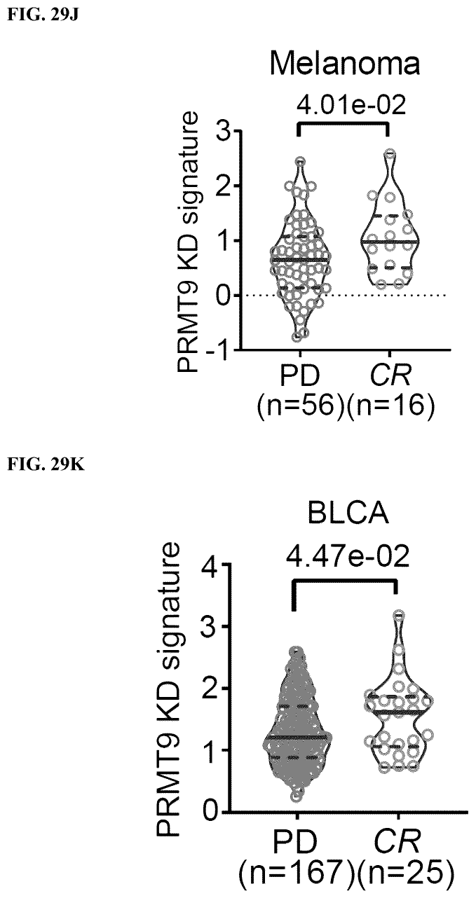

Next, the combination strategy in an MA9 AML syngeneic transplant model was evaluated. While MA9 leukemia cells engrafted (about 1% in PB), AML-bearing mice were treated for a period of 3 weeks with vehicle, anti-PD1 mAb (10 mg/kg/QOD/i.p.), LD2 or LD2 plus anti-PD1. LD2 was administered at 10 mg/kg/i.v./BID. After treatment, compared to LD2 only, the combination significantly decreased leukemic progenitor (GFP⁺ cKit⁺) engraftment and expanded tumor-specific T cells in BM (FIGS. 29A-29C). Notably, combination treatment extended AML mouse survival and decreased LSC activity, as evidenced by minimal residual leukemia cells detected in secondary transplants (FIGS. 29D-29E).

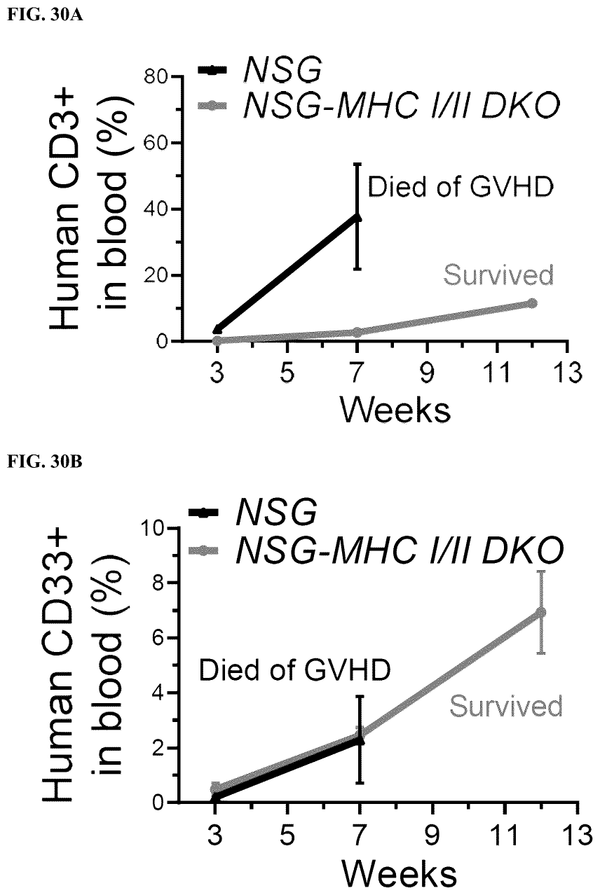

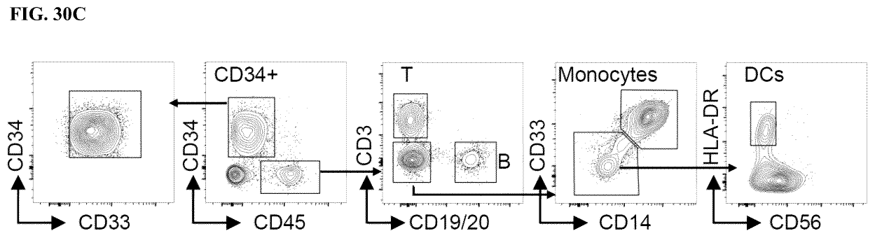

To better analyze human AML immune responses, a humanized AML model was established. Specifically, in a cohort of MHC1/2 double-KO (DKO) NSG mice, two million MNCs from an AML specimen were implanted by intra-femoral injection of each irradiated DKO mouse. 12 weeks post-transplant, MHC-deficient mice showed long-term (in PB) engraftment of T and CD33+ cells (FIGS. 30A-30B), without developing acute GVHD, consistent with a previous report. Importantly, BM engraftment of human hematopoietic subsets was confirmed including T cells, monocytes and DCs (FIG. 30C) and observed selective expansion of the immature CD33⁺CD34⁺CD45^{dim} subset (FIG. 30C), which is frequently seen in human AML BM specimens. Mice were then divided into vehicle and LD2 treatment groups. After 3 weeks of treatment, decreased numbers/frequency of leukemic CD34⁺ cells (FIGS. 29F-29G) and increased numbers of CD8+ T cells expressing CD69 and IFNγ (FIGS. 29H-29I) in LD2 relative to control groups were observed.

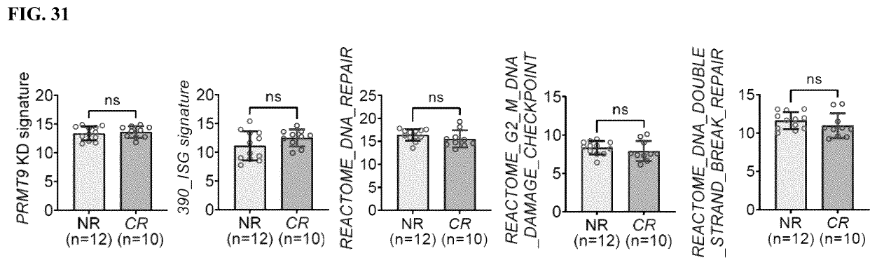

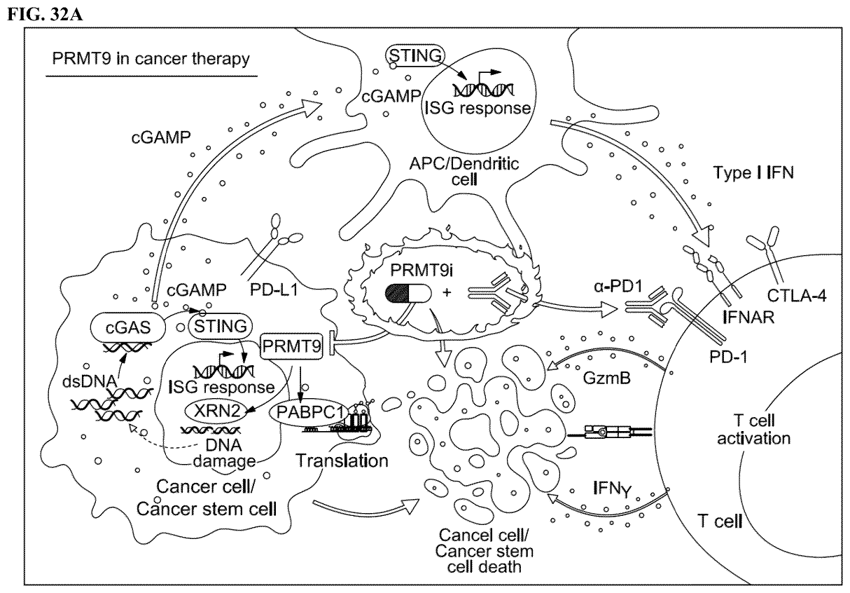

Also, the correlation between PRMT9 activity and the clinical response of PD-1/PDL1 inhibitors was assessed using clinical trial datasets. Kumar et al. Cancer discovery 11, 2050-2071, doi:10.1158/2159-8290.CD-20-1144 (2021); Liu et al, Nat Med 25, 1916-1927, doi:10.1038/s41591-019-0654-5 (2019); Mariathasan et al. Nature 554, 544-548, doi:10.1038/nature25501 (2018). To do that, PRMT9-KD gene signature was defined which was obtained from RNA-seq analysis of PRMT9-KD vs Ctrl AML lines (FIG. 23C). The signature is composed of 102 differentially expressed genes common to 2 AML cell lines (fold change >2, P<0.05). Notably, higher levels of PRMT9-KD gene expression signature were positively associated with complete response (CR) to ICB vs progressive disease (PD) in two clinical cohorts (melanoma, bladder cancer) (FIGS. 29J-29K). Relevant to AML, the only available RNA-seq dataset (GSE183415) related to PD1 inhibitors was based on a phase 2 trial of high-dose cytarabine followed by PD1 inhibitor treatment in refractory/relapsed AML cases (n=37). However, no significant correlation of the PRMT9-KD gene expression signature could be detected with clinical responses to PD-1 inhibitors using this dataset, possibly due to confounding factors associated with the mixed cytarabine/ICB regimen (FIG. 31).

Discussion

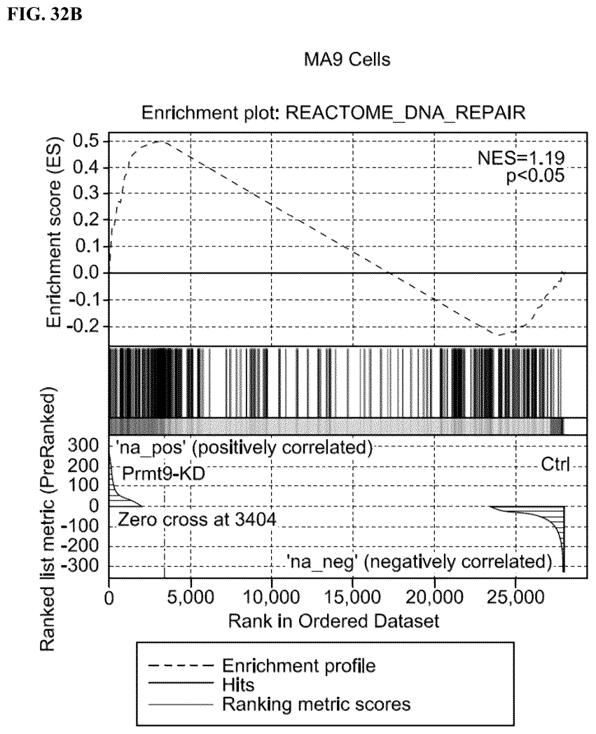

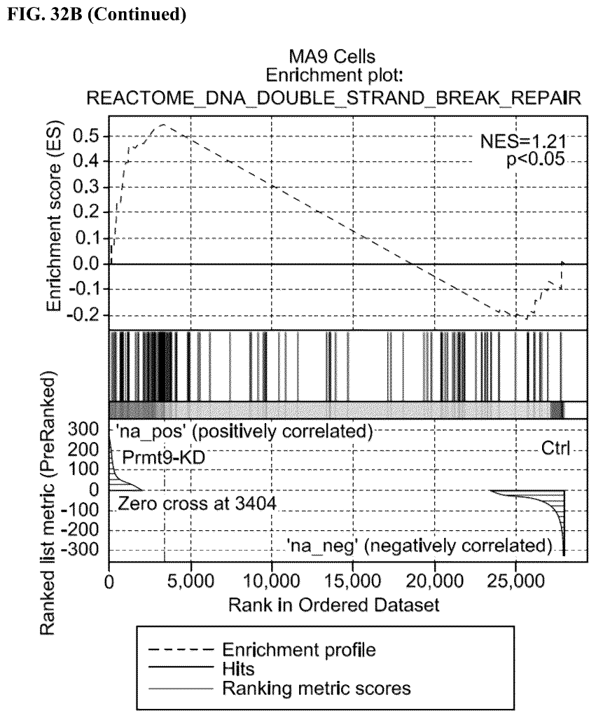

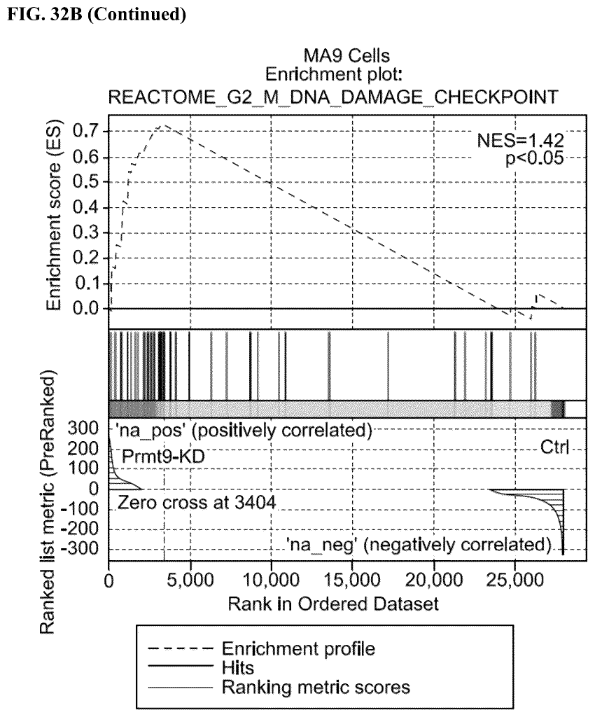

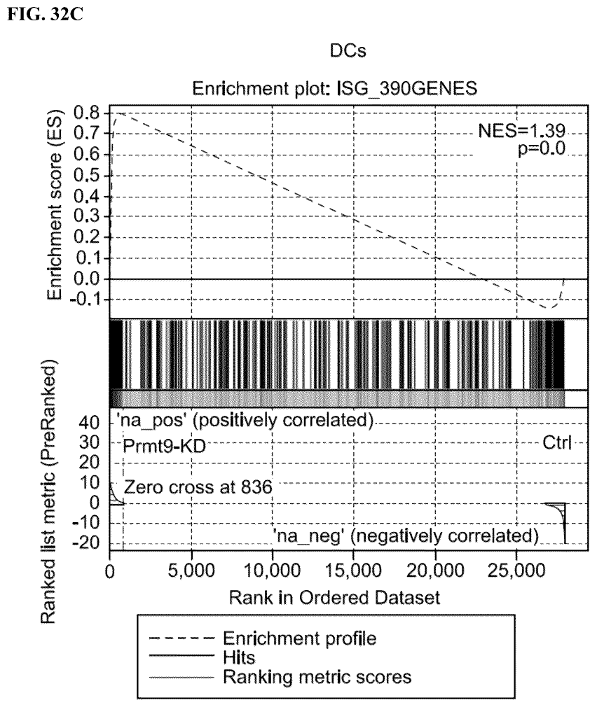

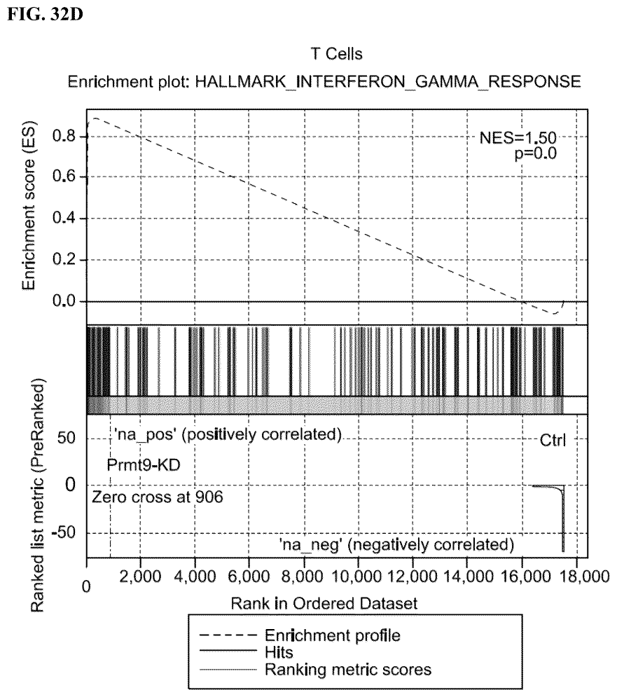

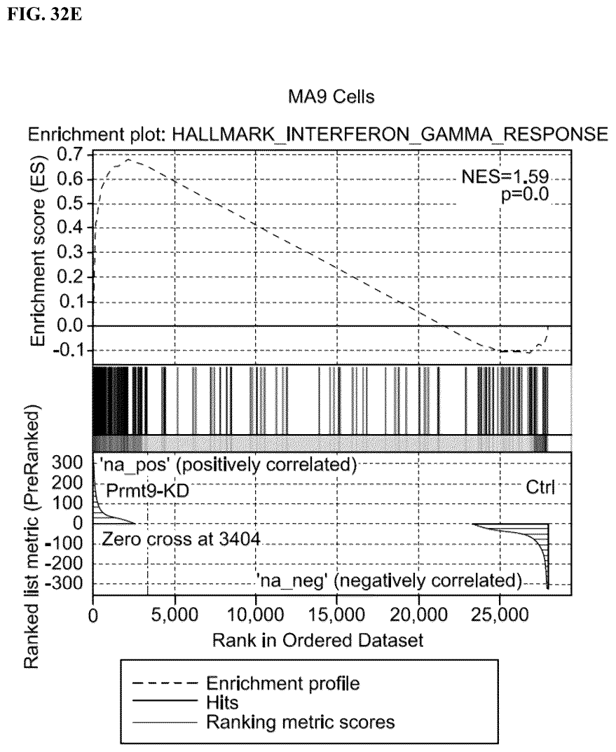

Currently, immune-activating strategies that leverage T cells against cancers, including T cell-engaging antibodies, ICIs and CAR-T cells, have shown success in clinical trials for leukemia such as B-cell precursor acute lymphoblastic leukemia (B-ALL). However, translation of these strategies to AML treatment is challenging. AML is propagated by LSCs; thus PRMT9-high LSCs give rise to highly proliferative and immune-evasive leukemia blasts. The observation of increased PRMT9 levels in AML relative to normal hematopoietic cells is likely due to its high-expression levels in LSCs. Herein, the results highlight the importance of a novel PRMT9 targeting strategy that not only ablates LSCs but stimulates an anti-cancer immune response to achieve maximal therapeutic effects. This strategy, when combined with an immune checkpoint inhibitor, could approach a disease cure, while current overall outcomes for AML patients remain poor. Specifically, the approach targets the arginine methyltransferase PRMT9 to ablate AML LSCs and lymphoma cells via downregulating synthesis of short-lived oncoproteins; targeting PRMT9 also induces DNA damage-mediated activation of cGAS and release of cGAMP, thereby cross-priming T cells via a type I interferon (IFN-I) response. Moreover, the lead compound LD2 was identified as a potent inhibitor of PRMT9 catalytic activity that promotes robust anti-AML activity. Finally, a strong anti-tumor synergy was observed in preclinical tumor models when a PD-1 inhibitor was combined with LD2, despite single-agent immune checkpoint inhibitors has only limited clinical activity against AML or advanced B-Cell NHL. The work represents an approach that could be rapidly translated into clinics (FIG. 32A).

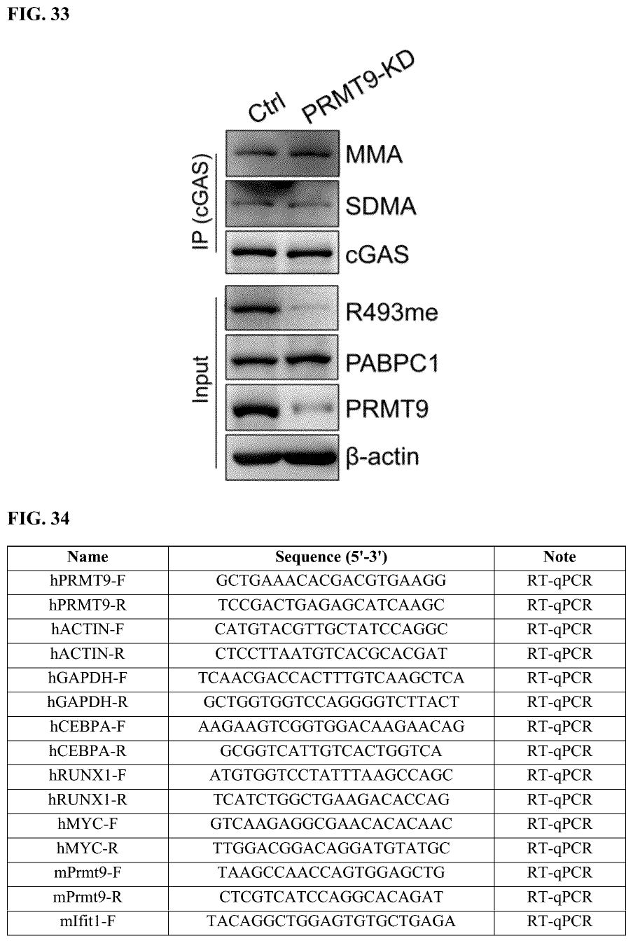

PRMT9, one of two SDMA-forming PRMTs, is characterized by a unique duplicated methyltransferase domain, possibly accounting for its minimal activity on substrates of other PRMTs. To date, PRMT9's only known catalytic substrate is splicing factor SAP145. Here, a high-resolution MS-based quantitative proteomic study was used to profile changes in global MMA and DMA upon PRMT9 knockdown and identified previously unknown PRMT9 targets. Specifically, methylation at residue R493 enables PABPC1 protein to efficiently bind to the mRNA poly (A) tail, promoting translation initiation in LSCs. Moreover, XRN2 methylation at R946 may allow complex formation with p54nrb to prevent DNA double-strand breaks (DSBs), associated with XRN2's role in resolving R-loop (RNA/DNA hybrid) structure. Indeed, PRMT9 inhibition (either by LD2 or shRNA) or expression of XRN2-R946K in AML cells promoted R-loop formation and ATR signaling, which underlies cGAS activation in cancer cells. Unlike PRMT5, which directly methylates cGAS, PRMT9 did not catalyze cGAS methylation (FIG. 33).

The study demonstrates that tumor elimination induced by PRMT9 deletion relies on type I IFN responses and T cell immunity. Among all immune cells within the TME, single cell analysis as well as further functional assay revealed the changes in T cell subpopulations seen following PRMT9-KD associated with immune memory, indicating that T cells are essential for long-term tumor suppression. Previous leukemia studies have relied on xenograft models lacking an intact immune system, excluding analysis of innate immune responses. Other studies have used high-dose cytotoxic chemotherapies like cytarabine that directly and efficiently kill tumor cells but also dampen immune responses. Interestingly, it was found that neither PRMT9-KO nor LD2 treatment perturbed T cell function. Also, in this study, advantage of syngeneic mouse models was taken with intact immune systems as well as co-cultures of primary AML cells with immune cells. Both models are clinically relevant and enabled the complete evaluation effects of PRMT9 inhibition on host immune responses within the TME.

How does PRMT9-inhibition in cancer cells elicit a distinct response in T cells? Here, it was shown that cGAS-dependent dsDNA sensing by cancer cells is critical for T cell priming effects. Notably, leukemia or lymphoma cells express more abundant levels of cGAS relative to normal counterparts from healthy donors. Upon PRMT9-KD, cancer cells accumulate cytosolic dsDNA, providing abundant substrate for cGAS catalysis (FIG. 23I). Such changes in dsDNA are partially due to DNA damage induced by loss of XRN2 methylation seen following PRMT9-inhibition (FIG. 25). Interestingly, GSEA analysis of single-cell-transcriptomes from PRMT9-KD vs control MA9 cells showed significant enrichment of DNA damage response gene signatures (FIG. 32B), confirming an association between PRMT9 inhibition and DNA damage in cancer cells.

Moreover, PRMT9 inhibition also downregulates SAMHD1 (FIG. 6G) which antagonizes cGAS-STING activity as previously reported. Consequently, cGAS-activating cancer cells can produce high levels of the immunotransmitter cGAMP. Moreover, among all cancer types, leukemia or lymphoma cells express the lowest levels of ENPP1 which hydrolyzed cGAMP (FIG. 24Q. and FIG. 24R), allowing sustained cGAMP production in blood cancer cells. As many reports, extracellular cGAMP may be transferred via gap junctions from cancer cells to DCs, activating STING in DCs and inducing production of type I IFN. Indeed, single-cell transcriptome analysis of the MA9 model revealed ISG upregulation in DCs (FIG. 32C). Moreover, subsequent GSEA analysis showed upregulation of the IFNα response pathway in T cells from PRMT9-KD AML BM (FIG. 20M), in line with observations that intra-tumoral or systemic application of STING agonists triggers an anti-tumor IFN-I response. As an outcome of the IFN-I response, T cells (FIG. 18K) exhibited IFN-γ upregulation after Prmt9-KD. Significant enrichment of IFN-γ response genes was also observed in MA9 cancer cells and in T cells (see FIGS. 32D-32E). Although it was confirmed that DCs contribute to PRMT9-KD outcomes (FIG. 23S), other APCs such as macrophages may also underlie these phenotypes.

To date, most of AML patients are not responsive to anti-PD-1/PD-L1 monotherapy. Currently, it is well accepted that lack of proper innate sensing may limit T cell activation within the TME, highlighting the pressing need for novel combination therapies targeting both innate and adaptive immunity. Accordingly, PD-1/PD-L1 axis ablation stimulates adaptive immune responses, while the newly developed PRMT9 inhibitor can induce innate sensing like cGAS signaling. Indeed, the study showed that combined administration of PRMT9 and PD-1 inhibitors not only augments overall T cell responses but acts synergistically against tumors.

Overall, the results reveal a previously undefined biological role for PRMT9 in cancer, and a novel small molecule inhibitor has been developed blocking PRMT9 activity which is elevated in AML LSCs. The study also prompts an appraisal of anti-cancer drugs with consideration of their impact on immune cells within the TME and provides a rationale for further evaluating PRMT9 inhibition combined with a PD1/PD-L1 inhibitor against AML, which is considered a hard-to-cure disease.

Methods

Patient Samples. Peripheral blood (PB) or bone marrow (BM) samples were obtained from AML patients at City of Hope (COH) Comprehensive Cancer Center. Patient characteristics are shown in Table 1. Normal peripheral blood mononuclear cells (PBMCs) were obtained from the blood donor center at the cancer center. Cord blood (CB) was obtained from StemCyte. All subjects signed informed consent forms. Sample acquisition was approved by the COH Institutional Review Board in accordance with the Helsinki Declaration. Mononuclear cells were isolated by Ficoll-Hypaque (GE) centrifugation. CD34⁺ cell enrichment or CD3⁺ T cell depletion was performed using immunomagnetic columns (Miltenyi Biotec).

Cell Culture. Human AML lines, including Molm13, MV4-11, THP1, NB4, U937, HL-60 and MA9.6ITD, were cultured in RPMI 1640 medium with 10% FBS and 1% penicillin-streptomycin (Thermo Fisher). MA9.6ITD cells (MLL-AF9 plus FLT3-ITD) were established by Dr. James Mulloy. Human primary normal and AML CD34⁺ cells used for transduction were maintained in StemSpan Serum-Free Expansion Media (SFEM, Stemcell Technologies) supplemented with 50 ng/mL recombinant human stem cell factor (SCF), 100 ng/mL Flt3 ligand (Flt3L), 100 ng/mL thrombopoietin (TPO), 25 ng/mL IL-3, and 10 ng/mL IL-6 (Peprotech Inc.). Murine MA9-transformed cells, MA9/ITD and CMM cells were cultured in RPMI 1640 medium with 10% FBS supplemented with murine growth factors (mIL-3, 10 ng/mL; mIL-6, 10 ng/mL; mSCF, 30 ng/mL). NHL lines, including RAJI, UPN1, BL41, Rec1, OCI-Ly3, and A20, were cultured in RPMI 1640 medium with 10% FBS and 1% penicillin-streptomycin. All other cancer lines, including DMS273, DMS114, SW1573, A549, SW620, HCT116, HepG2, PC3, DU145, MDA-MB-231, HT1197, A172, MIAPACA2, and HT1080, were cultured in DMEM with 10% FBS and 1% penicillin-streptomycin, as were HEK293T cells.

Mice. WT C57BL/6J (JAX #000664), Rag2^{-/-} (B6(Cg)-Rag2^{tm1.1Cgn}/J, JAX #008449), Ifnar1^{-/-} (B6(Cg)-Ifnar1^{tm1.2Ees}/J JAX #028288), MLL-AF9 knockin (Kmt2a^{tm2(MLLT3)Thr}/KsyJ, JAX #009079), Batf3^{-/-} (B6.129S(C)-Batf3^{tm1Kmm}/J, JAX #013755), NSG (NOD.Cg-Prkdc^{scid} Il2rg^{tm1Wjl}/SzJ, JAX #005557), NSGS (NOD.Cg-Prkdc^{scid} Il2rg^{1Wjl} Tg(CMV-IL3,CSF2,KITLG)1Eav/MloySzJ, JAX #013062), and NSG-MHC I/II DKO (NOD.Cg-Prkdc^{scid} H2-K1^{tm1Bpe} H2-Ab1^{em1Mvw} H2-D1^{tm1Bpe} Il2rg^{tm1Wjl}/SzJ, JAX #025216) mice were purchased from the Jackson Laboratory. B6-Ly5.1 (CD45.1, NCI 564) and BALB/c (NCI 028) mice were purchased from Charles River. Mice purchased were acclimated to housing conditions for at least one week at the COH Animal Resthece Center prior to experiments. Colonies for each mouse strain were maintained in the same animal facility. Mouse care and experimental procedures complied with established institutional guidelines and protocols approved by the Institutional Animal Care and Use Committee at COH National Medical Center.

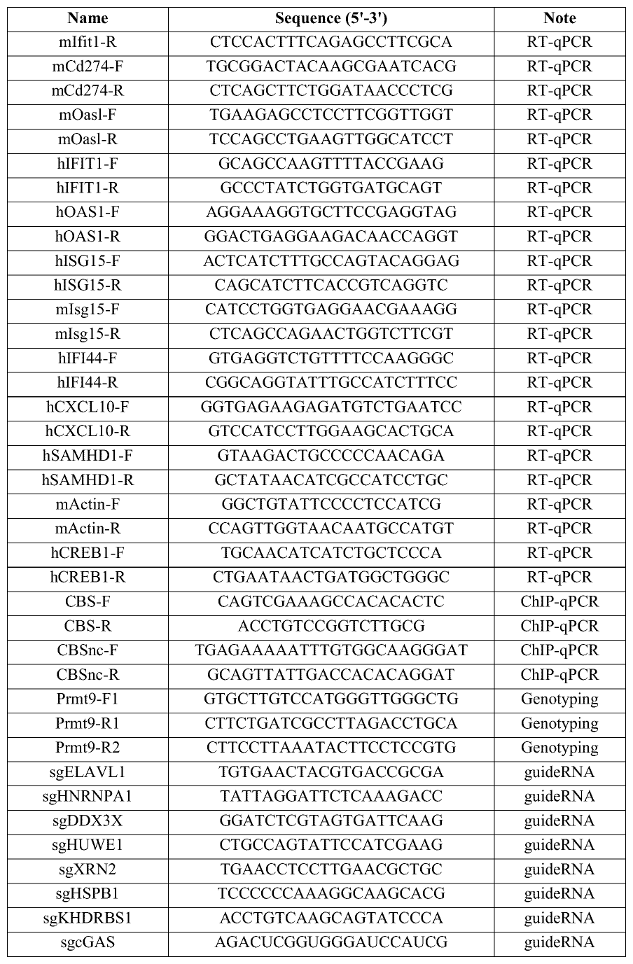

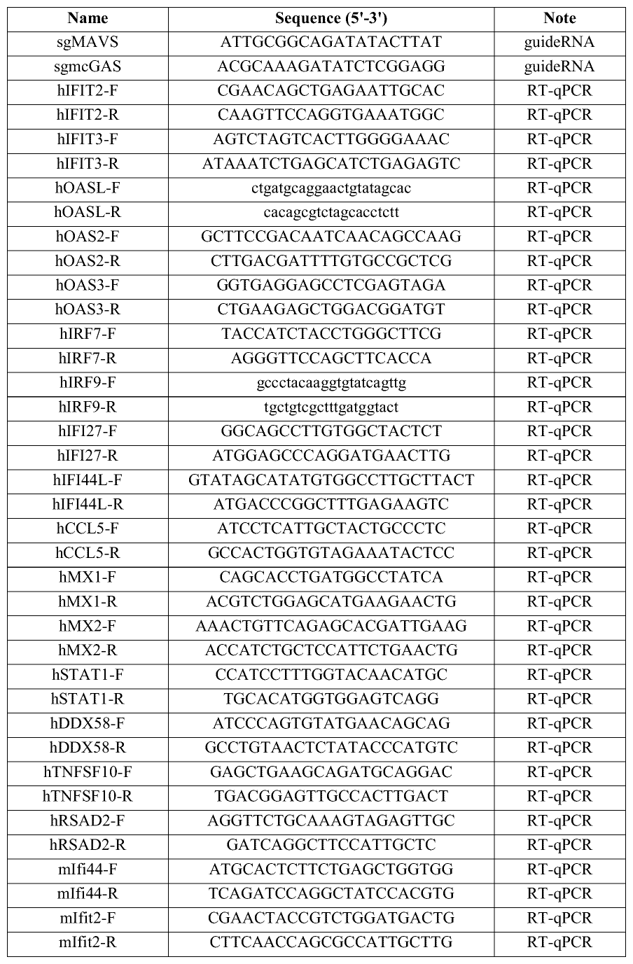

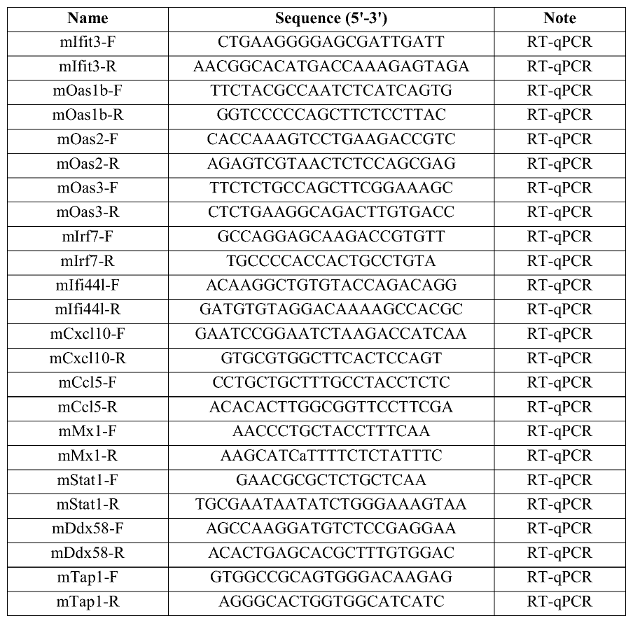

DNA Constructs and Oligos. CD530-EF1A-IRES-GFP vectors were purchased from System Biosciences Inc. CD530-EF1A-T2A-GFP vectors were modified from CD530-EF1A-IRES-GFP to replace IRES with T2A sequences. Full length WT or LDIG-to-AAAA mutant PRMT9 were cloned into CD530-EF1A-IRES-GFP vectors. Flag-tagged WT or R946K mutant XRN2, Flag-tagged WT or mutant R88K DDX3X, and Flag-tagged either full-length WT or C-terminal (aa436-636) PABPC1 or R493K, R481K, R506K, or 3RK mutants were cloned into CD530-EF1A-T2A-GFP vector. All plasmids were synthesized by Genscript. Inc. shRNAs targeting hPRMT9, mPrmt9, PABPC1, and CREB1 were purchased from Sigma-Aldrich (Mission shRNA) and cloned into pLKO-SFFV-RFP, as described by Sun et al. Cell Stem Cell 23, 355-369 e359, doi:10.1016/j.stem.2018.07.018 (2018). cGAS-WT and the activation mutant ΔN were purchased from addgene and constructed into a Dox-inducible expression vector. SMARTvectors with shPRMT9 were purchased from Dharmacon (Horizon, PerkinElmer). Oligos used are in FIG. 34.

Compounds. Compounds from the NCI Developmental Therapeutics Program (DTP), ZINC libraries or MolPort were dissolved in DMSO and stored at −20° C. PEGylated liposomes packaging of LD2 used for animal treatment were prepared using the thin film hydration method described by Bangham et al Journal of Molecular Biology 13, 238-252, doi:10.1016/s0022-2836(65)80093-6 (1965). Lipids (DSPC, cholesterol, and DSPE-PEG2000 at a ratio of 3:1:0.2) plus LD2 were dissolved in chloroform and then organic solvent was removed in vacuo to form a thin film, which was stored overnight in vacuo to remove residual chloroform. Subsequently, lipids were hydrated in PBS, pH 7.4, at 60° C. to form liposomes.

Lentivirus Production and Transduction. Virus production was described previously by Li et al. Cancer Cell 21, 266-281, doi:10.1016/j.ccr.2011.12.020 (2012). Briefly, HEK293T cells were transfected with pMD2.G and psPAX2 packaging vectors plus lentivectors designed to overexpress or knock down genes using the calcium-phosphate co-precipitation method. Supernatants containing virus particles were filtered and concentrated. After lentivirus titration, cells were exposed to virus (MOI=1-5 for cell lines, MOI=20-40 for primary AML cells) in the presence of 8 μg/mL polybrene via spinoculation and then sorted by flow cytometry based on RFP or GFP expression or selected in puromycin (2 μg/mL). For primary cell transduction, cells were stimulated in SFEM supplemented with high concentrations of growth factors as described above, followed by two exposures to virus-containing supernatants in the presence of Transdux (System Biosciences) via spinoculation.

Real-Time Q-PCR analysis. RNA was extracted from cells using Trizol reagent (Invitrogen) according to the manufacturer's protocol. First-strand cDNA was generated using SuperScript III reverse transcriptase (Invitrogen). Quantitative real-time PCR was performed using SYBR Green master mix (Life Technologies) with gene-specific primers. Signals were detected with a QuantStudio 7 Flex Real-Time PCR system (Life Biotechnology). Relative expression levels were determined by normalizing to GAPDH or β-actin levels. Primers were synthesized by Integrated DNA Technologies, Inc.

Immunoprecipitation (IP) and Western Blotting. Cells were lysed in buffer containing 50 mM Tris, pH 7.4, 150 mM NaCl, 1 mM EDTA, 0.5% NP40, and 0.5% sodium deoxycholate supplemented with protease and phosphatase inhibitors. Cell lysates were incubated with anti-Flag beads (Sigma) overnight. After washing, precipitates were denatured in 2× Laemmli sample buffer (Bio-rad), resolved on SDS-PAGE and transferred to nitrocellulose (NC) membranes (Bio-Rad). Proteins of interest were sequentially probed with primary and HRP-conjugated secondary antibodies (Jackson Immuno Research Laboratories, Westgrove, PA). Primary antibodies are listed in Table 10. Signals were detected using SuperSignal West Pico or Femato kits. Results were imaged by G:BOX Chemi XX6 gel doc systems (Syngene) and visualized using GeneSys image acquisition software (Syngene). Protein levels were determined by densitometry with ImageJ software (NIH, Bethesda, MD).

Chromatin Immunoprecipitation (ChIP)-qPCR Analysis. Cells were fixed in formaldehyde and lysed based on the SimpleChIP® Plus Enzymatic Chromatin IP Kit (Cell Signaling Technology, #9005). After chromatin digestion, immunoprecipitations were performed using the ChIP grade antibodies Anti-CREB1 (Santa Cruz, SC-240) and Anti-H3K27Ac (ab4729), plus ChIP-Grade Protein G Magnetic Beads. After reversing protein-DNA cross-links, DNA was purified using spin columns, followed by quantitative real-time PCR. Fold-enrichment was calculated based on the formula: 2^{-(Ct IP Sample-Ct IP IgG)}.

Flow Cytometry. BM was obtained from mouse tibias and femurs of both legs by aspiration. Spleens were removed from mice and pressed with the end of a syringe. Cells were washed with PBS containing 1% FBS and then passed through a 70-μm cell strainer and subjected to lysis of red blood cells. Before flow cytometry analysis, cells were washed twice in PBS containing 1% FBS and stained with indicated antibodies in the same buffer for 25 minutes (min) at 4° C. Flow cytometry analysis was performed on a BD LSRFortessa™ X-20 Analyzer using BD FACSDiva Software (version 8.0). Data analysis was performed using the FlowJo 10 application. Dead cells were excluded by DAPI (ThermoFisher) staining. Live single cells were plotted against the intensity of surface/intracellular markers. Isotype controls were used to determine the boundary between positive and negative cells. Anti-human CD45 antibody was used to determine human Molm13 cell engraftment in NSGS mice. Anti-mouse CD45.1 and CD45.2 antibodies were used to determine engraftment of CD45.2+ donor cells in CD45.1-expressing transplants. Mouse HSPC percentages were evaluated by staining with anti-mouse c-Kit, Sca-1, CD16/32, and CD34 antibodies after incubation with a biotin-linked lineage antibody cocktail (including anti-mouse CD3, CD4, CD8, CD11b, CD11c, CD19, CD41, Ter119, B220, IgM, NK1.1, Gr-land IL7Ra, eBioscience) and staining with FITC- or PE-labeled streptavidin. Mouse BM cell differentiation was assessed using anti-mouse Mac1, Gr-1, B220, and Ter 19. To detect antigen-specific T cells in tumors, samples were stained with iTAg Tetramer/H-2Kb-OVA (SIINFEKL) (MBL) for 1 h on ice. For intracellular staining, cells were incubated 15 min with fixation buffer (Biolegend) on ice and then washed twice with 1× Intracellular Staining Permeabilization Wash Buffer (10×) (Biolegend). Antibodies against IFN-γ (Clone XMG1.2) and Granzyme B (Clone: QA16A02) were added, and samples were then incubated for 1 h on ice. For human primary AML samples, the following markers of human hematopoietic subsets was used: T cells (CD3+), B cells (CD19+/CD20+), monocytes (CD14+) and DCs (HLA-DR+CD34−CD33−CD3−CD19−CD20−CD14−CD56−) as well as the immature CD33+CD34+CD45dim subset. T cell activation was assessed by CD69 and IFN-γ staining, and apoptosis by Annexin V staining. For cell cycle studies, THP1 cells were treated 48 h with LD2, harvested, fixed, stained with DAPI for DNA content and analyzed by flow cytometry. Flow data was analyzed using FlowJo software.

Competitive Transplantation Assay. BM cells (0.5×10⁶, CD45.2⁺) from 6-8 week-old Prmt9^{fl/fl}MxCre⁺ or Prmt9^{fl/fl}MxCre⁻ mice were mixed with an equal number of CD45.1⁺ BM cells from 6-8 week-old B6-Ly5.1 (CD45.1) mice and then transplanted into lethally-irradiated (900cGy) B6-Ly5.1 mice by intravenous (i.v.) injection. Four weeks later, PB samples were collected and assessed by flow cytometry with CD45.1 and CD45.2 antibodies. Mouse recipients were induced with pIpC (InvivoGen) intraperitoneally (i.p.) at a dose of 15 mg/kg every other day for 7 doses, and CD45.2⁺ chimerism in PB was monitored by flow cytometry every 4 weeks.

Limiting Dilution Assays. For limiting dilution assays (LDAs) to evaluate the LSC frequencies of MA9, MA9 FLT3-ITD⁺ and CMM AML cells in vitro, cells were suspended in CFC growth medium with Dox to induce Prmt9 KD and plated in 48-well plates at a limiting dilution manner, e.g. 100 cells/well, 50 cells/well, 20 cells/well, 10 cells/well, 5 cells/well, and 2 cell/well. Twelve wells for each group were used. The number of wells containing spherical colonies was counted at day 7 to calculate the LSC frequency. To evaluate the LIC frequency in vivo, the BM cells isolated from Ctrl or Prmt9-KD MA9 AML mice were injected into sub-lethally irradiated 8- to 10-week-old B6 recipient mice via i.v. at a limiting dilution manner as mentioned in Table 7. The number of recipient mice developed full-blown leukemia within two months was counted in each group. LSCs and LICs frequency was calculated using the ELDA software.

AML Mouse Model and In vivo Bioluminescence Imaging. To track the effect of Prmt9 KO/KD in vivo, MA9 (Prmt9^{fl/fl}MxCre⁺), MA9-ITD (Prmt9^{fl/fl}MxCre⁺) or CMM (inducible shPrmt9) cells or their co-responding control cells were transduced with lentiviral vectors harboring a luciferase reporter plus GFP marker. Cells were then sorted based on GFP levels and used for intravenous inoculation into sub-lethally irradiated CD45.1 B6 mice. To track the MA9-luciferase cells (inducible shPrmt9 or control) in vivo, cells were injected intravenously into WT B6, Rag2^{-/-}, or NSGS mice. For in vivo bioluminescence imaging, mice were injected intraperitoneally with 150 mg/kg D-luciferin (Goldbio) in PBS and then anesthetized with isoflurane, followed by imaging analysis using Lago X (Spectral Instruments Imaging). Bioluminescent signals were quantified using Aura imaging software (Spectral Instruments Imaging). Total flux values were determined by drawing regions of interest and are presented as photons/second/cm²/steradian. To identify immune subsets contributing to leukemia regression after Prmt9-KD, antibody-based depletion was performed with an initial dose of combined anti-CD4/CD8 treatment or anti-NK1.1 treatment administered 1 day prior to in-vivo DOX administration to KD Prmt9 mice. Antibodies (400 μg) were injected i.p. twice the first week, and then at 200 μg twice weekly to maintain NK or T cell depletion. To assess DC function in Prmt9-KD outcomes, Batf3-WT or KO mice was implanted with Prmt9-KD or -WT MA9+ AML cells and evaluated AML progression.

Analysis of Cell Viability, Apoptosis and Colony Formation. Cell viability was assessed using the Cell Titer-Glo Luminescent Cell Viability Assay Kit (Promega). Apoptosis was assessed by Annexin V/DAPI staining followed by flow cytometry. Colony formation capacity based on colony number was determined in methylcellulose progenitor assays as described by Sun et al. Cell Stem Cell 23, 355-369 e359, doi:10.1016/j.stem.2018.07.018 (2018) and He et al. Blood 134, 548-560, doi:10.1182/blood.2019001282 (2019).

SILAC-Based Quantitative Proteomics Analysis

Proteomics sample preparation. For SILAC, Molm13 cells were cultured in SILAC RPMI 1640 medium (Thermo Fisher Scientific, 88365) supplemented with 10% dialyzed FBS (Thermo Fisher Scientific, A3382001) and either “light” L-lysine [89987] and L-arginine [89989] for control cells, or “heavy” ₁₃C_{6 15}N₂ L-lysine [88209] and ₁₃C_{6 15}N₄ L-arginine [89990] for inducible shPRMT9 cells, for at least 10 passages to ensure full incorporation of light or heavy L-lysine and L-arginine. Note that after labeling, cell growth, viability, and overall morphology were comparable to normal Molm13 cells.

After 3 days of Dox induction in both control and PRMT9-KD cells, equal numbers of light- and heavy-labeled cells were mixed at a ratio of 1:1. Cells were washed with ice cold PBS twice and centrifuge at 300×g for 5 mins. Cell pellets were lysed in in 9 M Urea with protease and phosphatase inhibitors (1 mM Sodium Orthovanadate, 2.5 mM Sodium pyrophosphate, 1 mM 0-glycerophosphate) in 20 mM HEPES (pH 8.0) buffer. Samples underwent four cycles of sonication for 30 second each using a microtip sonicator (VCX130 VibraCell™, Sonics) operating at 50% amplitude. Lysates were clarified by centrifugation at 20,000×g for 15 min and protein quantification were performed using BCA (bicinchoninic acid) assay (Pierce) as per manufacturer's protocol. Equal amount of extracted protein from heavy and light SILAC culture was combined for in-solution digestion. Sample was first reduced by incubation with dithiothreitol (5 mM final concentration) at 55° C. for 30 min and then alkylated by incubation with iodoacetamide (10 mM final concentration) for 30 min in the dark. Sample was diluted 4-fold prior to sequential digestion first with LysC (Wako; enzyme: substrate ratio of 1:50) for 2 h. and then overnight with trypsin gold (Promega; enzyme: substrate ratio of 1:100). Digestion was quenched by acidification with TFA (final concentration of 1%) and the sample was desalted using 0.7 milliliters (m1) Sep-Pak Classic C18 column (Waters). Eluted peptides were speedvac'd to dryness and reconstituted in 1.4 mL IAP buffer followed by peptide quantification using BCA assay. 5% of peptides were subjected to global quantitative proteomics analysis and the rest to two steps of methyl-R peptide enrichment using sequential incubation of peptides (˜4.3 mg) with anti-MMA antibody beads (PTMScan® Mono-Methyl Arginine Motif Kit #12235) and anti-SDMA antibody beads (PTMScan® Symmetric Di-Methyl Arginine Motif Kit #13563). Enriched peptides were reconstituted in 10 μl loading solvent (98% water, 2% acetonitrile, 0.1% formic acid) and transferred to autosampler vials. One microgram of non-enriched peptides was taken up for global protein identification.

LC-MS/MS Data Acquisition. Mass spectrometry data were either acquired on Orbitrap Fusion Lumos (methylated peptides) or Orbitrap Eclipse with FAIMS Pro interface (unmodified peptides) coupled to a U3000 RSLCnano LC system running binary solvent A (water, 0.1% formic acid) and B (acetonitrile, 0.1% formic acid) at 300 nl/min. Methylated peptides (5 μl injection volume) were directly loaded on a 25 centimeters (cm) EasySpray C18 column (75 μM ID, 2 μm particle size, 100 Å pore size, ThermoFisher Scientific), and eluted over 120 minute gradient as follow: 2-19% B in 80 min, 19%-30% B in 20 min, 30-98% B in 5 min, followed by 2 min of high organic wash and return to initial conditions in 1 minute and column equilibration for 12 min. Unmodified peptides (1 microgram (μg) peptides, 5 microliters (μl) injection volume) were directly loaded on a 50 cm EasySpray C18 column (75 μM ID, 2 μm particle size, 100 Å pore size, ThermoFisher Scientific) and eluted over 240 min using the following gradient: 2-5% B in 12 min, 5-19% B in 158 min, 19-30% B in 40 min, 30-90% B in 9 min followed by 4 min of high organic wash and return to initial conditions in 2 min and column equilibration for 15 min. Using Data-Dependent Acquisition (DDA), full scans were performed in the Orbitrap at a resolution of 120K over a mass range of 375-1500 m/z. Using a duty cycle of 3 second (Lumos) or 1 second (Eclipse) per FAIMS CV (−40/−60/−80), most abundant precursors with charge state between 2-7 were fragmented by HCD (32% NCE on Eclipse and 35% NCE on Lumos) and measured in the iontrap. Dynamic exclusion was set to 60 seconds to prevent resampling of previously analyzed precursors.

Proteomics Data Analysis. MS RAW files were searched against human Uniprot protein database (downloaded 2020, 42373 entries) and a common contaminant database using MaxQuant v1.6.17.0. Search parameters include: fully tryptic peptides with up to 2 missed cleavages, fixed modification of Cys carbamidomethylation, dynamic modification on Arg mono/di-methylation, Met oxidation and N-terminal acetylation. Protein identification required at least 1 unique peptide and results were filtered to 1% Protein and Site False Discovery Rate (FDR). Resulting methyl peptides SILAC ratios obtained from MaxQuant evidence.txt output file were normalized to their protein SILAC ratios prior to further analyses. Musiani et al. Science signaling 12, doi:10.1126/scisignal.aat8388 (2019)

R-methyl Peptide Motif Analysis. Motif analysis of R-methyl sites was performed using the iceLogo web application, which allows visualization of statistically significant enrichment variations between a set of PRMT9-regulated sequences and unchanging methyl peptides in a background set. A p value threshold was set to 0.05.

Polysome Profiling. The protocol of Weng et al, Cell Stem Cell 22, 191-205 e199, doi:10.1016/j.stem.2017.11.0162018, was used with the following modifications. Molm13 cells were transduced with inducible shCtrl or shPRMT9 SMART Vector (Horizon) lentivirus and selected with puromycin (2 microgram per milliliter (g/mL)). Cells were then induced 3 days with 2 μg/mL Dox, and before collection, treated 5 min with 100 μg/mL cycloheximide (CHX). Fifty million cells from each group were harvested, washed with ice-cold PBS containing 100 μg/mL CHX and frozen on dry ice before lysis. Lysis buffer was formulated as 10 mM HEPES, pH7.4, 100 mM KCl, 5 mM MgCl₂, 100 μg/ml CHX, and 2% Triton X-100, with freshly added protease inhibitor (Roche) and 40 units per milliliter (U/m1) SUPERasin (Ambion). Sucrose density gradients (15-45% (w/v)) were freshly prepared in SW41 ultracentrifuge tubes (Backman) using a Gradient Master (BioComp Instruments). Then, 500 microliters (l) supernatant from cell lysates was loaded onto gradients and centrifuged for 2.5 h at 35,000 revolutions per minute (rpm) (minimal brake) at 4° C. in a SW41 rotor. Samples were collected as 30 fractions (0.5 mL per fraction) and analyzed using Gradient Station (BioCamp) equipped with an ECONOUV monitor (BioRad, Hercules, CA) and a Gilson FC203B fraction collector (Mandel Scientific, Guelph, Canada). RNA purified from fractions was subjected to qPCR analysis.

O-propargyl-puromycin (OP-Puro) Protein Synthesis Assay. A protein synthesis assay was performed using protocols outlined in the Click-iT® Plus OPP Alexa Fluor™ 647 Protein Synthesis Assay Kit (ThermoFisher), with modifications. Briefly, after treatment, cells were incubated 30 min with 20 μM Click-iT OPP, washed with PBS and fixed 15 min in 3.7% PFA/PBS. After permeabilizing 15 min in 0.5% Triton X-100/PBS, cells were incubated 30 min in Click-iT Plus OPP reaction cocktail, washed twice with PBS/1% FBS, and analyzed by flow cytometry.

In vitro Methylation Assay. An in vitro methylation assay was carried out in a 30 μl reaction in 50 mM Tris HCl, pH 7.4, 50 mM NaCl, 50 mM KCl, 1 mM MgCl2, and 1 mM DTT buffer at 30° C. for 3 h. For each reaction, 1 μg purified PABPC1-CT protein or synthesized peptides, 1 μg purified PRMT9 protein, and 5 μM of S-adenosyl methionine (SAM, Cayman) were mixed simultaneously. Methylated proteins/peptides were detected by western or dot blot assays using anti-pan-SDMA, anti-pan-MMA, anti-pan-ADMA or the in-house PABPC1 R493me antibodies. The R493me antibody was generated by Genemed Synthesis. For the ex vivo tritium-labelling methylation assay, 1 μg purified PRMT9 protein, 1 μg HA-tagged PABPC1 WT or corresponding PABPC1-R481K/R493K/R506K (3RK) protein which were immunoprecipitated from 293T cells, and 1 μl S-adenosyl-1-[methyl-3H] methionine (78 Ci/mmol, PerkinElmer Life Sciences) was added to a 30 μl reaction mixture at 30° C. for 1 h. Samples were separated by SDS-PAGE, transferred to polyvinylidene membranes, and exposed to an X-ray film from overnight to 3 days at −80° C.

PRMT9 Structure-based Virtual Screening. To identify potential PRMT9 inhibitors, a structure-based virtual screening (VS) was performed. The crystal structure of human PRMT9 (PDB ID 6PDM, resolution 2.45 A) was used for virtual screening. Missing loops were added using MOE loop modeler. A box size of 25×21×27 Å\hat{ }3 centered around the co-crystalized chemical probe was used for screening, which includes both the S-adenosyl methionine (SAM) binding pocket and the substrate binding pocket in the N-terminal methyltransferase domain (aa150-520). To ranking the binding affinity, parallel AutoDock Vina runs were conducted on a local computer cluster. 700,000 compounds from ZINC library were selected using the following criteria: molecular weight 350 to 450, LogP <3, and total charge −2e to +2e, and availability. In addition, National Cancer Institute library (NCI DTP 260,000 compounds) was also screened. Each ligand was docked for ten times and ranked by the lowest binding energy score. Based on the virtual screening results, the top 300 candidates were requested (142 of them were available) from the NCI DTP and top 100 candidates (70 of them were available) from ZINC library to assess anti-AML activity. To estimate lead compound selectivity, Vina docking of LD2 was also performed into human CARM1 (5U4X), PRMT5 (PDB ID 4X61), PRMT7 (PDB ID 4M38), and PRMT9. To compare LD2 binding to PRMT5 vs PRMT9, two replicas of 100 nanosecond molecular dynamics simulation of LD2 docked into each were carried out. Protein-ligand complexes were prepared in solvated 150 mM KCl solution using CHARMM-GUI. CHARMM36 parameter sets were used for the protein and ions, and TIP3P for water. The CHARMM general force field (CGenFF) was used for the LD2 molecule. MD runs were conducted using AMBER20 pmemd.cuda on RTX2080Ti GPU cards for each ligand-protein complex.

Saturation Transfer Difference (STD) and Carr-Purcell-Meiboom-Gill (CPMG) Nuclear Magnetic Resonance (NMR) assays. MBP-tagged PRMT9 core methyl-transferase domain (CMTD, 150-474) protein was expressed and purified by Genscript. Briefly, the PRMT9-CMTD sequence was inserted into the pMAL-c5X vector between Nde I and EcoR I sites. Tagged protein was expressed in BL21 (DE3) cells and purified on a MBP column, followed by Superdex 200 and Q Sepharose columns. Proteins were sterile-filtered and lyophilized after extensive dialysis against NMR buffer (50 mM NaH₂PO₄, pH7.5). D₂O-based 50 mM sodium phosphate buffer, pH 7.1, was used with 5% DMSO-d₅. For the STD NMR assay, the molar ratio of LD2 vs PRMT9 was 60:1 in which the concentration of PRMT9 is 0.67 μM. Fifty M 3-(Trimethylsilyl)-propionic-2,2,3,3,-d₄ acid sodium (TSP-d₄ from Sigma-Aldrich) is used as internal reference. The same buffer conditions were used for the CPMG NMR study. The molar ratio between PMRT9 and LD2 are 1:20, 1:40 and 1:60 in addition to a control sample with free LD2. LD2 concentration in CPMG experiments was 40 uM. NMR STD experiments were carried out at 25° C. on a 700 MHz Bruker Ascend system equipped with a 5 mm triple resonance cryogenic probe. The spectral width was 14 ppm with 32 k data points. Saturation frequency was set at 0.2 ppm, and the reference experiment frequency was set at −30 ppm. The 50 ms gauss pulse saturation train was 3.8 second long with a field strength of 86 Hz. The T₂ filter spin-lock was 70 ms long with a field strength of 4960 Hz. The total number of scans was 1920, and the saturation and reference experiments were acquired in an interleaved manner using the Bruker stddiffgp19.3 pulse sequence. For the CPMG experiment, the Periodic Refocusing Of J Evolution by Coherence Transfer (PROJECT) method was used in addition to a pre-saturation to acquire the 1D ¹H data. The spectrum width was 14 ppm, and the recycle delay and acquisition time were 1.5 and 3.3 seconds, respectively. The CPMG duration was 86 ms with a 1.2 ms delay between pulses in the CPMG echo. Data was analyzed using Bruker Topspin 3.6.

Cellular Thermal Shift Assay (CETSA). To determine whether LD2 binds to PRMT9 directly in vivo, CETSA was performed as described previously by Jafari et al. Nature :rotocols 9, 2100-2122, doi:10.1038/nprot.2014.138 (2014) and Su et al. Cancer Cell 38, 79-96 e11, doi:10.1016/j.ccell.2020.04.017 (2020). Molm13 cells were first engineered to overexpress Flag-tagged PRMT9-WT or PRMT9-Mut (W152A, D258A and E433A; all three residues are predicted drug/PRMT9 binding sites). Five million cells were pretreated with 2.5 uM LD2 overnight. DMSO was used as control. The cells were washed with ice cold PBS and re-suspended in 1 mL PBS supplemented with protease inhibitor cocktail. The cell suspension was aliquoted into 7 PCR tubes with 100 μl in each tube and heat shocked in the Bio-Rad T100 Thermal Cycler at indicated temperatures for 5 min to denature proteins. The cells were lysed by using freeze-thaw cycles with dry ice and centrifuged at 20,000 g for 20 min at 4° C. The supernatant was denatured with 2× Laemmli Sample Buffer (Bio-Rad) for Western blot assay. The bands were quantified using Image-J software and plotted from three biological replicates.

Primary AML MNC culture, Mass Cytometry (CyTOF) Staining, Acquisition and Analysis. Two million MNCs from AML BM specimens were cultured per well in 24-well plates in IMDM plus 20% FBS under physiological cytokine conditions (200 pg/mL GM-CSF, 1 ng/mL G-CSF, 200 pg/mL SCF, 1 ng/mL IL-6, 200 pg/ml MIP-1α, and 50 pg/ml LIF). Then the EasySep™ Dead cell removal kit (STEMCELL) was used to ensure >95% living cells before culture. Cells were treated with VEH (DMSO), 2.5 μM LD2, anti-PD-1 (pembrolizumab, 10 pg/ml, SIM0010, BioXCell), or LD2 plus anti-PD-1 for 4 days at 37° C. On day 4, cells were pre-treated 6 h with BFA and subjected to CyTOF immunostaining with customized surface or intracellular marker antibodies, according to Fluidigm CyTOF protocols (PN400279A4). An untreated PBMC sample from a healthy donor served as a control for phenotyping. Samples were acquired on Fluidigm Helios. Data were normalized and saved as FCS files before analysis using Cytobank software (https://premium.cytobank.org/). After data was cleaned up, SPADE analyses were used for clustering of AML cells and immune cell subpopulations based on median marker expression level of each node.

In silico Analysis of Cytotoxic T lymphocyte (CTL) Levels in Primary AML Samples. Average expression levels of CD8A, CD8B, GZMA, GZMB and PRF1 were used to estimate CTL levels in AML samples. In silico tests were carried out to determine the proportion of PRMT9-high and -low patients exhibiting high versus low CTL scores using both GSE144688, including 526 AML patient samples, and GSE12417, including 163 patient samples. For each patient, high versus low CTL scores were evaluated based on an absolute cut-off of 0.5 for the z-score. The significance of differences between proportions of PRMT9-high versus-low patients was evaluated using Fisher's Exact Test.

Single-Cell RNA Sequencing

Experimental Protocol and Library Preparation. BM cells in MA9 transplanted mice, and BM and spleen cells in Ctrl and Prmt9-KD mice administered Dox in drinking water over 7 days were collected for analysis. Cells were resuspended as single cells in the presence of 0.4% BSA and loaded onto a 10× Genomics Chromium instrument to generate an emulsion of single-cell gel beads (GEMs). Approximately 5,000-10,000 cells were loaded per channel. scRNA-seq libraries were prepared using the Chromium™ Single Cell 3′ Library & Gel Bead Kit v2 (PN-120237), Single Cell 3′ Chip Kit v2 (PN-120236) and i7 Multiplex Kit (PN-120262) (10× Genomics, Pleasanton, CA, USA), following the Single Cell 3′ Reagent Kits v2 User Guide (Manual Part #CG00052 Rev A). Libraries were sequenced on an Illumina HiSeq 4000 system (SY-401-4001, Illumina) as 2×150 paired-end reads, one full lane per sample, for >90% sequencing saturation.

Data Processing The Cell Ranger Single Cell Software Suite, version 6.1.1, was implemented to perform single-cell 3′ gene counting and aggregation of multiple samples for generating raw counts, cell barcodes and gene features. The R package “Seurat” (version 4.0) was run as the platform to implement all data processing procedures.

Quality control, Normalization and Batch removal To ensure that all cellular barcode data was associated with viable cells, cell QC was executed based on three QC covariates: the minimum detected genes (3) in each cell, the minimum cells (200) related to each gene and the maximum fraction (0.2%) of counts from mitochondrial genes per cell barcode. The high-count depth threshold (2,000) was used to filter out potential doublets. Then, the count matrix was log-normalized with a scale-factor of 10,000 to obtain the correct relative gene expression abundance between cells. After that, the R package “Harmony” was applied to remove batch effects due to biological differences between cell types or states. In the end, a high-quality preprocessed gene expression matrix was created.

Feature selection, dimension reduction and visualization To retain informative genes with high variability, genes with small variations (below 2) among all cells were filtered out. Then, the dimensions of count matrices were reduced using dedicated dimension reduction algorithms, such as UMAP and t-SNE. Reduced dimensions were used as coordinates on scatter plots to reflect visual representation and global structure of count data. Two-dimensional visualization outputs were then generated using the leading reduced components in UMAP and t-SNE plots.

Clustering and Annotation UMAP-related processed data was regarded as input of cell-clustering. Neighborhood distances among all cells were calculated to infer identity of each cell. Then, clusters were obtained by grouping cells based on similarity of gene expression profiles via specified distance metrics (Euclidian distance). Furthermore, for each cluster, R package “MAST” was implemented to deduce significant differentially-expressed genes (DEs). These DEs were considered markers of a cluster and were used for annotation purposes. Annotations were manually conducted by comparing marker genes with the literature and arranging cell categories. In addition, automatic annotation of cell clusters was done by applying R package “SingleR”, which relied on comparing external gene expression profiles of annotated clusters to those of tested individual cells. By combining both annotation styles, final cell-type labels of each cluster were acquired.

Gene Set Enrichment Analysis (For T-cell and MA9 clusters) For cell-type clusters interested, GSEA was performed on a list of pre-ordered genes ranked by “MAST”-derived (−log 10 (adjusted p-value)×sign (logFC)) with 1000 permutations. The gene sets of hallmark, kegg, cgp and GO-BP categories of MSigDB were considered as the signatures. Finally, specific enriched genes within a cluster were visualized by averaging their expression among all cells in that cluster. Key enriched gene expression was rescaled by Z-scores and visualized in the heatmap.

T-cell subset Identification ScRNAseq uncovered 10 distinct T cell clusters (c0-c9). c0 cells expressed Cd4 and the naïve T-cell marker Sell (CD62L) but not the effector/memory T cell marker Cd44 or T-cell activation genes, including Ifng, Icos, Havcr2 (Tim-3), Ctla4, Gzmb, Tnfrsf4, Tnfrs18, Pdcd1, and Lag3. Thus c0 was defined as naive CD4+ T-cells. Similarly, c1 cells expressed Cd8a and Sell but not Cd44 or other T-cell activation markers and were defined as naïve CD8+ T-cells. c2 cells expressed Cd8a, Cd44 and Sell and intermediate levels of the T-cell differentiation genes Tbx21 (T-bet) and Eomes and represented a “memory” CD8+ T-cell population. c3 cells expressed high levels of Cd4, Cd44 and the T-cell activation genes Icos, Ctla4, Tnfrsf4 and Pdcd1, but did not express Sell and were defined as activated/effector CD4+ T cells. c5 cells expressed Cd44 and showed highest levels of the T-cell activation genes Ifng, Gzmb, Icos, Tim-3, Il2ra, Tnfrsf18, and Lag3, indicating that these cells are highly differentiated CTLs. c6 cells were defined as regulatory T cells (Tregs), as they expressed high levels of the classic Treg markers Cd4, Il2ra (Cd25), and Foxp3. c4, c7, c8 and c9 contained both CD4+ and CD8+ T cells. c4 and c9 showed much lower levels of activation markers, and lower Sell and higher Cd44, indicating they represent “effector” T-cell populations. c7 expressed only the naive T-cell marker Sell, indicating a naïve population, while c8 expressed lower Sell and higher Cd44, but did not express other T-cell activation markers, indicating it represents a memory cell population.

Bulk RNA-Seq analysis. Total RNA was isolated from cell lines or primary AML cells using Trizol Reagent (Life technologies) following the manufacturer's instructions. RNA quality (the RNA integrity number [RIN]) was assessed using an Agilent Bioanalyzer, and all samples were evaluated as RIN >8. RNA sequencing libraries were prepared using a Kapa mRNA HyperPrep kit (Roche) and sequenced on a HiSeq 2500 system (Illumina). RNA-Seq reads were aligned to the human hg38 reference genome assembly using Tophat2 (v2.0.8) with default settings. Gene expression levels were counted to obtain raw counts with HTSeq (v0.11.2). The counts data were normalized using the trimmed mean of M values (TMM) method, implemented in the Bioconductor package edgeR (v.3.30.3) to obtain the normalized RPKM (Reads Per Kilobase of transcript, per Million mapped reads) value. Genes were considered differentially-expressed if the fold-change was <1.5 or <0.67, FDR <0.05, and at least one sample showed RPKM>1. Hierarchical clustering of differentially-expressed genes was performed using Cluster v3.0 with Pearson correlation distance and average linkage, and visualized using Java Treeview. Enrichment analysis on pathways of Hallmark, kegg and cgp (chemical genetic perturbation) was performed using Gene Set Enrichment Analysis (GSEA), implemented in GSEA v.4.0.3. Accession numbers for RNA-Seq data will be granted.

Detection of cGAMP Levels. cGAMP levels were detected as reported by Yu et al. Cell 183, 636-649 e618, doi:10.1016/j.cell.2020.09.020 (2020) and Gyori et al Redox biology 2, 457-465, doi:10.1016/j.redox.2013.12.020 (2014) with some modifications. THP1 cells were Dox-treated to induce PRMT9 KD for 2 days, media were changed to serum-free phenol red RPMI (ThermoFisher) for 24 h. Conditioned media were collected and cGAMP levels were detected by DetectX® 2′,3′-Cyclic GAMP Enzyme Immunoassay Kit (Arbo Assay) according to the manufacturer's protocol. To determine cGAMP levels in the BM microenvironment of control and PRMT9-KD mice, BM fluid was collected by centrifuging tibias and femurs at 8,000 rpm for 15 seconds. cGAMP levels were detected using the DetectX® 2′,3′-Cyclic GAMP Enzyme Immunoassay Kit (Arbo Assay).

Lucia Reporter Assay. WT (thpd-nfis, InvivoGen), cGAS KO (thpd-kocgas, InvivoGen) and MAVS KO (thpd-komavs, InvivoGen) THP1-Dual™ cells were used for the Lucia reporter assay. THP1-Dual™ cells (InvivoGen) were derived from the human THP-1 monocyte line harboring the Lucia gene, a secreted luciferase reporter, under control of the ISG54 minimal promoter flanked by five IFN-response elements. Cells were transduced with inducible shPRMT9 or control lentivirus and selected in puromycin to generate inducible PRMT9 or control KD lines. After Dox treatment to KD PRMT9 or LD2 to inhibit PRMT9 in these cells, Lucia activity was determined as described by the manufacturer (InvivoGen) by adding QUANTI-Luc™ reagents and read using a FilterMax F5 microplate reader (Molecular Device).

Immunofluorescence Assays. Cells were spun onto glass coverslips, fixed with 4% paraformaldehyde, permeabilized with 0.1% Triton X-100, blocked and incubated with primary anti-dsDNA (AE-2), γH2AX or S9.6 antibodies. Slides were then incubated with secondary anti-rabbit-Alexa 488 (Life Technologies), mounted in 90% glycerol solution containing DAPI (Life Technologies) and examined under a Zeiss LSM 880 confocal microscope.

Neutral Comet Assays. Neutral comet assays were performed using the OxiSelect Comet Assay Kit (Cell Biolabs, Inc.). Briefly, THP1 cells after PRMT9-KD for 48 or 72 h were resuspended in ice-cold PBS, mixed with pre-warmed (37° C.) Comet Agarose at a 1:10 ratio (v/v), rapidly loaded onto the top of the Comet Agarose Base Layer and cooled at 4° C. Slides were immersed 60 min in pre-chilled Lysis Buffer at 4° C., which was then replaced with pre-chilled Alkaline Solution for another 30 min. After 3 washes with pre-chilled TBE, slides were subjected to electrophoresis at 1 Volt/cm for 15 min, and then rinsed twice with DI H2O, which was replaced with cold 70% Ethanol for 5 min. Slides were then air-dried and stained with diluted Vista Green DNA Dye for 15 min. Comets were examined under an Widefield Zeiss Observer 7 fluorescence microscope. About 50 cells were analyzed for each independent experiment, and DNA damage parameters were calculated using OpenComet software in Image J and shown as olive tail moments.

Gene Knockout in THP1 Cells. THP1 reporter cells were electroporated with ribonucleoprotein complexes (RNPs) composed of Cas9 protein and guide RNAs using the Neon Transfection System Kit (Thermo Fisher), following the manufacturer's instructions. Twenty mol/L gRNA (as listed in Table 10) was mixed at an equimolar ratio with Cas9 protein and used to transduce 10⁵ THP1 cells. Gene editing efficiency was determined by western blot analysis.

In vitro Co-cultures of Bone Marrow Dendritic Cells (BMDCs) and T-cells Single-cell suspensions of bone marrow (BM) cells were collected from tibias and femurs of C57BL/6 mice. Cells were placed in 10 cm dishes and cultured with complete RPMI 1640 medium containing 20 ng/mL recombinant mouse GM-CSF (Peprotech Inc.). Fresh medium was added to cultures on days 3 and 6. BMDCs were harvested at Day 7. CD8+ T-cells were isolated from lymph nodes and spleens of OT-1 transgenic mice using a negative CD8+ T-cell isolation kit (StemCell). MA9-OVA cells were pretreated 2 days with 2.5 μM LD2, and then drug was washed out and tumor cells were cultured for 3 days and then co-cultured overnight with harvested BMDCs. Supernatants were collected for IFN-β ELISA analysis (PBL Assay Science). BMDCs were sorted using a CD11c positive selection kit (Stemcell) and co-cultured 48 h with OT-1 CD8+ T-cells. Supernatants were collected and IFN-γ was assayed using a Mouse IFN-γ Flex Set cytometric bead array (CBA) assay (BD Biosciences).

MA9 AML Model in-vivo Treatment and Assessment of Leukemia-specific Immunity. As leukemia cells engrafted, MA9 syngeneic transplant mice were treated 3 weeks with: vehicle control, LD2, single anti-PD1 mAb (BE0146 [BioXCell], 10 mg/kg, i.p. every other day) or LD2 plus anti-PD-1 antibody. LD2 was administered at 10 mg/kg/i.v./BID, based on preliminary PK/PD results. Mice were assessed for overall survival, or culled directly to assess MA9 cell engraftment in BM and perform staining with survivin-specific pentamers to assess MA9-specific immunity as described⁵⁹. Briefly, MA9 mice BM was stained with anti-CD8 together with murine survivin-specific APC-conjugated pentamers ATFKNWPFL (ProImmune, Inc; Sarasota, FL, USA). Cytomegalovirus (CMV)-specific PE-conjugated pentamers HGIRNASFI served as negative controls. The percentage of survivin or CMV pentamer-positive CD8 T cells was assessed by flow cytometry. Secondary transplantations were performed to evaluate LSC activity in each group by assessing MA9 cell engraftment in BM.

Humanized AML Mouse Model. The humanized AML model was established using MHC1/2 double-KO (DKO) NSG mice. To do so, two million MNCs from AML specimens were implanted intra-femorally into an irradiated DKO NSG mouse. After transplant, MHC-deficient mice showed long-term (˜12 weeks in PB) engraftment of T and CD33+ cells without developing acute GVHD. A panel of human lineage and progenitor cell markers (CD45, CD33, CD34, CD14, CD19, CD20, CD3, CD56, HLA-DR) was used to identify T cells, B cells, monocytes and DCs and the immature CD33+CD34+CD45dim subset in the BM. Mice were divided into 2 groups and treated with VEH and LD2. Three weeks later, the number/frequency of leukemic CD34+ cells and the number of CD8+ T cells expressing CD69 and IFNγ were checked by flow cytometry.

NHL Tumor Models. A20 cells (3×10⁶) in 100 μL PBS were injected subcutaneously into syngeneic Balb/c mice. When tumor volume reached to 100 mm³, mice with similar tumor burden were randomized into treatment groups. Tumor-bearing mice were treated with isotype control (vehicle), anti-PD1 mAb (BE0146 [BioXCell], 10 mg/kg, i.p. every other day for 2 weeks), LD2 (100 mg/kg, i.t., daily for 2 weeks) or a combination of LD2 with anti-PD1. Tumor volume (calculated as V=π/6×L×W²) was monitored through the end of the study when a humane endpoint was reached. Microenvironmental components of tumors were analyzed by immunohistochemistry (IHC) staining and intracellular staining followed by flow cytometry.

Immunohistochemistry. A20 tumors were fixed overnight in 10% formalin and embedded in paraffin. Four-micron thick sections on slides were incubated 1 h at 60° C., deparaffinized and then rehydrated prior to Immunohistochemistry (IHC) staining. Slides were blocked for peroxidase activity with 3% H₂O₂ for 10 min and washed 5 min under running water. Slides were subjected to antigen retrieval for 15 min at 120° C. in Citrate Buffer, treated with Tris buffered saline (TBS), and incubated 1 h with anti-mCD3 or anti-mCD8 antibody. After washing, slides were incubated 30 min with hP-conjugated secondary antibody. All incubations were carried out in a humid chamber at room temperature. Slides were developed using 3,3′-diaminobenzidine (DAB) as substrate and counter-stained with Mayer's Hematoxylin. Slides were scanned by Whole Wliding Imaging and analyzed by NDP.view2 software (HAMAMATSU).

Flow Cytometry Analysis of Tumor-Infiltrating Immune Cells. Portions of fresh A20 tumors were cut into small pieces with sterile scalpels in serum-free RPMI 1640. Tissue was then dissociated in 1 mg/mL Collagenase Type IV (Sigma, C5138), 20 U/mL DNAse Type IV (Sigma, D5205), and 0.1 mg/mL Hyaluronidase Type V (Sigma, H6254) using GentleMACS C tubes on a GentleMACS Dissociator (Miltenyi Biotec), followed by incubation at 37° C. for 30 min. Cell suspensions were passed through a 70-μm strainer and centrifuged at 300×g for 5 min. Cells were washed in PBS and stained 30 min at RT using a Live-or-Dye™ 330/410 Fixable Viability Stain Kit (Biotium, #32018, BUV395). After two washes with PBS, cells were stained with immune cell surface markers (mCD45-APC, mCD3-APC/Cy7, mCD4-AF700, and mCD8-BV605) at RT for 30 min. After two washes with FACS buffer (PBS with 1% BSA and 1 mM EDTA), cells were fixed 10 min in 2% PFA at RT. After two washes with permeabilization buffer (eBioscience), cells were intracellularly stained with mouse IFNg-PE and GZMB-FITC antibodies in permeabilization buffer at RT for 30 min. After two washes with FACS buffer, samples were analyzed using a BD LSRFortessa™ X-20 Analyzer and BD FACSDiva Software (version 8.0). Data analysis was performed using FlowJo 10 software.

Statistics and Reproducibility. Unless specified, data from independent experiments are reported as means±SD or SEM, and statistical analyses were performed using unpaired two-tailed Student's t-test, two-sided Mann-Whitney test Kruskal-Wallis test, two-way analysis of variance (ANOVA) and two-sided Fisher's exact test. Results from survival experiments were analyzed with log-rank (Mantel-Cox) test and expressed as Kaplan-Meier survival curves. All the statistical analyses were performed with GraphPad Prism software and the detailed methods were described in each individual figure legend.

Precise p values are either included in figures or legends. Number of replicates (n) have been identified in figure legends or defined in the figures plotting the individual data points. Moreover, all numerical sthece data (replicates, BM subset cell/sample/mouse number) have been provided as supplemental excel tables. “n” indicates replicates, repeats and BM subset cell number; in some figures, “n” also represents the number of samples or animals used in indicated experiments. The following experiments use primary human samples: FIG. 1G (1 normal, 3 AML); FIG. 1H (10 normal, 30 AML); FIGS. 4M, 4N and 5L (3 normal, 11 AML); FIGS. 10M, 11K-11L, 27C-27G, and 28C-28E (1 normal, 3 AML); FIGS. 9A-9C (7 AML). Mouse experiments were performed once in FIGS. 4D-4E, 4H (5 mice per group); FIGS. 4F, 4G (5 mice per group); FIG. 4Q (8 mice per group); FIG. 4R (8 mice for Ctrl, 7 mice for PRMT9-KD); FIG. 3P (6 mice for Prmt9-WT, 9 mice for Prmt9-KD); FIG. 3Q (8 mice for Prmt9-WT, 15 mice for Prmt9-KD); FIG. 3R (7 mice per group); FIG. 18D (7 mice per group); FIG. 18E (5 mice per group); FIG. 18F (5 mice per group); FIG. 18G (7 mice per group); FIG. 18S (5 mice for Naïve mice, 4 mice for Survivors); FIG. 18V (5 mice for Ifnar1 KO, 6 mice for Ifnar1 WT); FIG. 23J (7 mice for Prmt9-KD group, 5 mice for each of the other three groups); FIG. 23M (7 mice for Cgas KO+Cgasδn group, 5 mice for each of the other two groups); FIG. 23S (7 mice for each Batf3 WT groups, 5 mice for Batf3 KO group); FIG. 20B (7 mice for Ctrl and Prmt9-KD groups, 5 mice for T cell and NK cell depletion groups); FIGS. 27I, 28G (5 mice per group); and FIGS. 28H-28I (5 mice per group). Single cell and bulk RNAseq were performed one time per sample and are shown in FIGS. 1E, 18H, and 23C. If not specified, in vitro experiments were repeated at least three times.

For violin plots, the center line denotes the median, and dashed lines denote upper and lower quartiles (FIGS. 1B-1C, 1I, 23H-23I, 23N, 23P, 25C, 29J-29K, 2A-2O, 2Q-2R, 3A-3D, 3I, 24K-24M, 24O-24Q, 29J-29K). Significance in survival studies was determined using Kaplan-Meier analysis with the log-rank (Mantel-Cox) test (FIGS. 4H, 4R, 18D-18G, 18S, 18V, 23J, 23M, 23S, 29D, 20B, 19A). The impact of PRMT9 expression levels on AML prognosis was determined by univariate Kaplan-Meier analysis with a log-rank test (FIGS. 1J-1K, 3F). Pearson correlation coefficient analysis was performed to evaluate correlations between levels of PRMT9 and CREB1, STAT3, STAT5A or GATA2 in TCGA AML and DLBCL cohorts sourced from GEPIA (http://gepia.cancer-pku.cn/) (FIGS. 3G-3H), as well as PRMT9 and PABPC1 R493 methylation levels in AML CD34+ subsets (FIG. 6W). Differential gene expression was analyzed by Empirical Bayes Statistics, after fitting with a linear model (FIG. 23C, Bulk RNAseq DEG analysis). Importantly, p values <0.05 were considered statistically significant. *p<0.05; **p<0.01; ***p<0.001; ****p<0.0001; ns: no significance. Data shown was normally distributed, and variation within and between groups was not estimated. Sample size was not pre-selected, and no inclusion/exclusion criteria were used.

It is understood that the examples described herein are for illustrative purposes only and that various modifications or changes in light thereof will be indicated to persons skilled in the art and are to be included within the spirit and purview of this application and scope of the appended claims. All publications, patents, and patent applications cited herein are hereby incorporated by reference herein in their entirety for all purposes.

What is claimed is:

1. A method of treating a hematological cancer in a subject in need thereof, the method comprising administering to the subject a therapeutically effective amount of a protein arginine methyltransferase 9 inhibitor.

2. The method of claim 1, wherein the protein arginine methyltransferase 9 inhibitor is a short-hairpin RNA, a small interference RNA, a piwi-interacting RNA, a microRNA, a CRISPR Cas guide RNA, an antisense oligonucleotide, a small molecule compound, or an anti-PRMT9 antibody.

3. The method of claim 2, wherein the CRISPR Cas guide RNA is CRISPR Cas 12 guide RNA or a CRISPR Cas 9 guide RNA.

4. The method of claim 1, wherein the protein arginine methyltransferase 9 inhibitor is an anti-protein arginine methyltransferase antibody.

5. The method of claim 1, wherein the protein arginine methyltransferase 9 inhibitor is an anti-di-methylated poly (A) binding protein cytoplasmic 1 antibody.

6. The method of claim 1, wherein protein arginine methyltransferase 9 inhibitor is a compound of Formula I, Formula (II), or Formula (III): 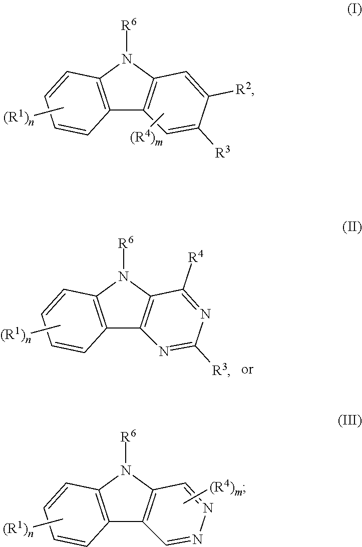
wherein:
R¹ is halogen, —CX¹₃, —CHX¹₂, —CH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}R^{2C}CNOR^{2D}, —NR^{2B}C(O)OR^{2D}, —NR^{2B}OR^{2D}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NH₂, —C(O)R^{3D}, —C(O)OR^{3D}, —C(O)NH₂, —OR^{3A}, —NR^{3B}SO₂R^{3A}, —NR^{3B}C(O)OR^{3D}, —NR^{3B}OR^{3D}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R² and R³ are optionally joined to form a substituted or unsubstituted cycloalkyl, substituted or unsubstituted heterocycloalkyl, substituted or unsubstituted aryl or substituted or unsubstituted heteroaryl;
R⁴ is halogen, —CX⁴₃, —CHX⁴₂, —CH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, —OCX⁴₃, —OCHX⁴₂, —OCH₂X⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, and R^{4D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n1, n2, n3, and n4 is an integer from 0 to 4;
m1, m2, m3, m4, v1, v2, v3, and v4 are independently 1 or 2;
X¹, X², X³, and X⁴ are independently halogen;
n is an integer from 1 to 4; and
m is an integer from 1 to 2.

7. The method of claim 6, wherein the protein arginine methyltransferase 9 inhibitor is a compound of Formula (Ia): 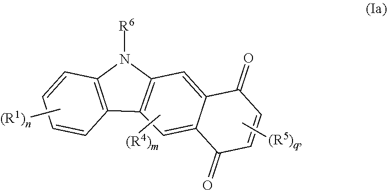
wherein
q is an integer from 1 to 2;
R⁵ is halogen, —CX⁵₃, —CHX⁵₂, —CH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCX⁵₃, —OCHX⁵₂, —OCH₂X⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{5A}, R^{5B}, R^{5C}, and R^{5D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n5 is an integer from 0 to 4;
m5 and v5 are independently 1 or 2; and
X⁵ is halogen.

8. The method of claim 1, wherein protein arginine methyltransferase 9 inhibitor is a compound having a Formula:

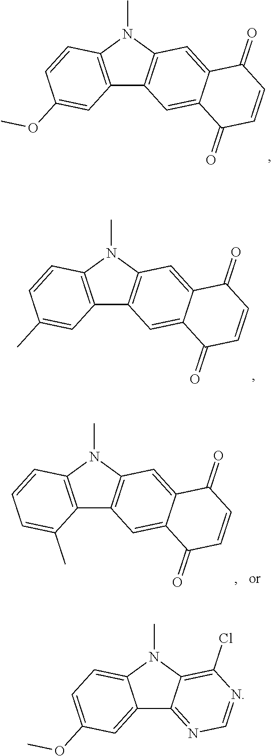 [SMILES: COc1ccc2c(c1)c1cc3c(cc1n2C)C(=O)C=CC3=O ; Cc1ccc2c(c1)c1cc3c(cc1n2C)C(=O)C=CC3=O ; Cc1cccc2c1c1cc3c(cc1n2C)C(=O)C=CC3=O ; COc1ccc2c(c1)c1ncnc(Cl)c1n2C]

9. The method of claim 1, wherein protein arginine methyltransferase 9 inhibitor is a compound having a Formula:

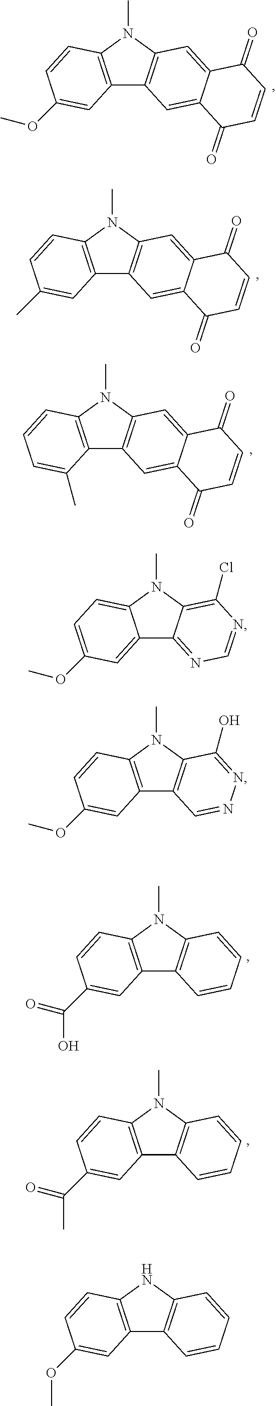

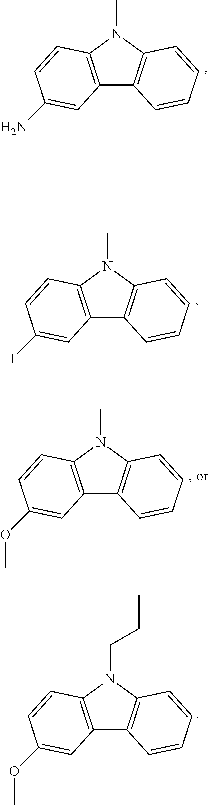 [SMILES: Cn1c2ccccc2c2cc(N)ccc21 ; Cn1c2ccccc2c2cc(I)ccc21 ; COc1ccc2c(c1)c1ccccc1n2C ; CCCn1c2ccccc2c2cc(OC)ccc21]

10. The method of claim 1, wherein the hematological cancer is leukemia, lymphoma, multiple myeloma, myelodysplastic syndrome, or myeloproliferative disorder.

11. The method of claim 1, wherein the hematological cancer is chronic lymphocytic leukemia, chronic myelocytic leukemia, acute lymphoblastic leukemia, acute myeloid leukemia, chronic myelomonocytic leukemia, chronic myelogenous leukemia, chronic neutrophilic leukemia, immunoblastic large cell leukemia, megakaryoblastic leukemia, acute megakaryocytic leukemia, promyelocytic leukemia, or erythroleukemia.

12. The method of claim 1, wherein the hematological cancer is Hodgkin lymphoma, diffuse large B-cell lymphoma, follicular lymphoma, small lymphocytic lymphoma, mantle cell lymphoma, marginal zone lymphomas, Burkitt lymphoma, lymphoplasmacytic lymphoma, hairy cell leukemia, primary central nervous system lymphoma, primary intraocular lymphoma, T-lymphoblastic lymphoma/leukemia, cutaneous T-cell lymphomas, adult T-cell leukemia/lymphoma, angioimmunoblastic T-cell lymphoma, extranodal natural killer/T-cell lymphoma, enteropathy-associated intestinal T-cell lymphoma, or anaplastic large cell lymphoma.

13. The method of claim 1, further comprising administering to the subject an effective amount of an anti-cancer agent.

14. A compound having Formula I, Formula II, or Formula III: 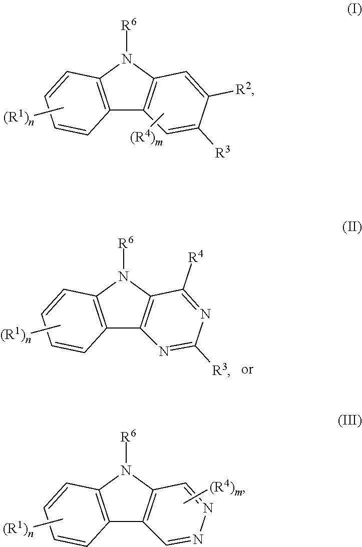
wherein
R¹ is halogen, —CX¹₃, —CHX¹₂, —CH₂X¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}R^{2C}CNOR^{2D}, —NR^{2B}C(O)OR^{2D}, —NR^{2B}OR^{2D}, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NH₂, —C(O)R^{3D}, —C(O)OR^{3D}, —C(O)NH₂, —OR^{3A}, —NR^{3B}SO₂R^{3A}, —NR^{3B}C(O)OR^{3D}, —NR^{3B}OR^{3D}, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R² and R³ are optionally joined to form a substituted or unsubstituted cycloalkyl, substituted or unsubstituted heterocycloalkyl, substituted or unsubstituted aryl or substituted or unsubstituted heteroaryl;
R⁴ is halogen, —CX⁴₃, —CHX⁴₂, —CH₂X⁴, —CN, —N₃, —SOₙ₄R^{4A}, —SOᵥ₄NR^{4B}R^{4C}, —NHNR^{4B}R^{4C}, —ONR^{4B}R^{4C}, —NHC(O)NHNR^{4B}R^{4C}, —NHC(O)NR^{4B}R^{4C}, —N(O)ₘ₄, —NR^{4B}R^{4C}, —C(O)R^{4D}, —C(O)OR^{4D}, —C(O)NR^{4B}R^{4C}, —OR^{4A}, —NR^{4B}SO₂R^{4A}, —NR^{4B}C(O)R^{4D}, —NR^{4B}C(O)OR^{4D}, —NR^{4B}OR^{4D}, —OCX⁴₃, —OCHX⁴₂, —OCH₂X⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, and R^{4D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n1, n2, n3, and n4 is an integer from 0 to 4;
m1, m2, m3, m4, v1, v2, v3, and v4 are independently 1 or 2;
X¹, X², X³, and X⁴ are independently halogen;
n is an integer from 1 to 4; and
m is an integer from 1 to 2.

15. The compound of claim 14, wherein the compound is of formula (Ia): 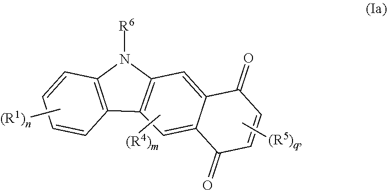
wherein:
q is an integer from 1 to 2;
R⁵ is halogen, —CX⁵₃, —CHX⁵₂, —CH₂X⁵, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCX⁵₃, —OCHX⁵₂, —OCH₂X⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{5A}, R^{5B}, R^{5C}, and R^{5D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n5 is an integer from 0 to 4;
m5 and v5 are independently 1 or 2; and
X⁵ is halogen.

16. The compound of claim 15, wherein:
R¹ is halogen, —NR^{1B}R^{1C}, —C(O)R^{1D}, —C(O)OR^{1D}, substituted or unsubstituted C₁-C₄-alkyl, or substituted or unsubstituted 1 to 4 membered heteroalkyl;
R² is hydrogen, substituted or unsubstituted C₁-C₄-alkyl, or substituted or unsubstituted 1 to 4 membered heteroalkyl, and R³ is hydrogen; or R² and R³ are joined to form a substituted or unsubstituted cycloalkyl, substituted or unsubstituted heterocycloalkyl, substituted or unsubstituted aryl or substituted or unsubstituted heteroaryl; and
R⁶ is hydrogen, substituted or unsubstituted C₁-C₄-alkyl, or substituted or unsubstituted 1 to 4 membered heteroalkyl.

17. The compound of claim 16, wherein R¹ is halogen, —NH₂, —C(O)CH₃, —COOH, unsubstituted methyl, or —OCH₃; R² is hydrogen; R³ is hydrogen; R⁴ is halogen; R⁵ is hydrogen; R⁶ is hydrogen or unsubstituted C₁-C₂-alkyl; n is 1; m is 1; and q is 1.

18. The compound of claim 16, wherein R¹ is halogen, —NH₂, —C(O)CH₃, —COOH, unsubstituted methyl, or —OCH₃; R² and R³ are joined to form an unsubstituted aryl or unsubstituted heteroaryl; R⁴ is halogen; R⁵ is hydrogen; R⁶ is hydrogen or unsubstituted C₁-C₂-alkyl; n is 1; m is 1; and q is 1.

19. The compound of claim 14, wherein the compound is:

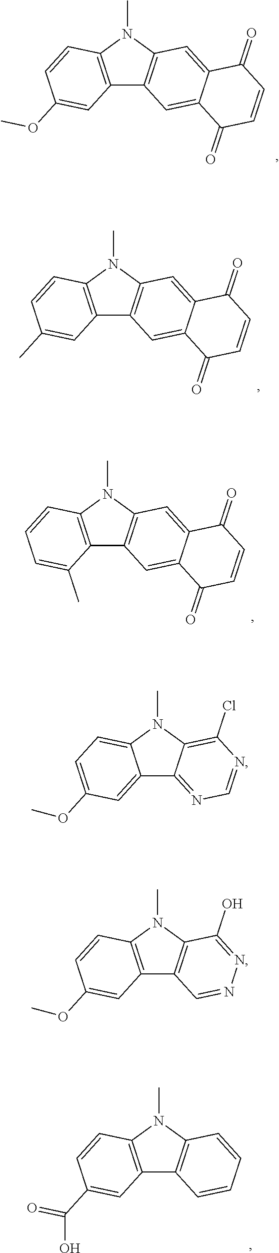 [SMILES: COc1ccc2c(c1)c1cc3c(cc1n2C)C(=O)C=CC3=O ; Cc1ccc2c(c1)c1cc3c(cc1n2C)C(=O)C=CC3=O ; Cc1cccc2c1c1cc3c(cc1n2C)C(=O)C=CC3=O ; COc1ccc2c(c1)c1ncnc(Cl)c1n2C ; COc1ccc2c(c1)c1cnnc(O)c1n2C ; Cn1c2ccccc2c2cc(C(=O)O)ccc21]

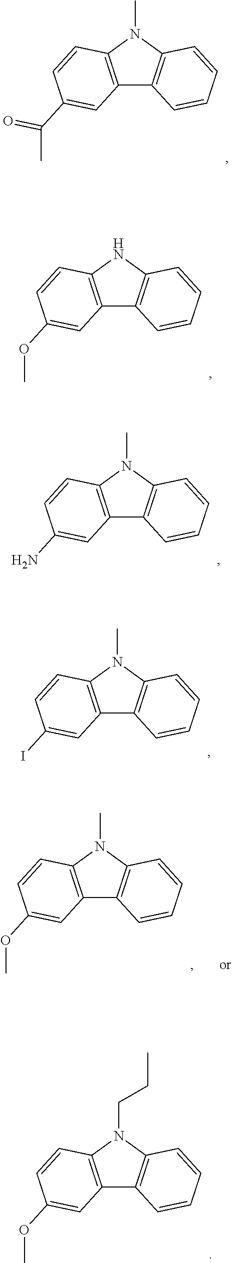 [SMILES: CC(=O)c1ccc2c(c1)c1ccccc1n2C ; COc1ccc2[nH]c3ccccc3c2c1 ; Cn1c2ccccc2c2cc(N)ccc21 ; Cn1c2ccccc2c2cc(I)ccc21 ; COc1ccc2c(c1)c1ccccc1n2C ; CCCn1c2ccccc2c2cc(OC)ccc21]

20. A pharmaceutical composition comprising the compound of claim 14 and a pharmaceutically acceptable excipient.
